(19) United States
(12) Patent Application Publication
(10) Pub. No.: US 2026/0240958 A1
(43) Pub. Date: Aug. 20, 2026

(54) REPRESSOR FUSION PROTEIN SYSTEMS

(71) Applicant: Scribe Therapeutics Inc., Alameda, CA (US)

(72) Inventors: Jason FERNANDES, Redwood City, CA (US); Sean HIGGINS, Alameda, CA (US); Sarah DENNY, San Francisco, CA (US); Ross WHITE, Concord, CA (US); Benjamin OAKES, El Cerrito, CA (US); Emeric Jean Marius CHARLES, Berkeley, CA (US); Addison WRIGHT, El Cerrito, CA (US); Brett T. STAAHL, Tiburon, CA (US); Fred DEITER, Alameda, CA (US)

(21) Appl. No.: 19/343,818

(22) Filed: Sep. 29, 2025

Related U.S. Application Data
(63) Continuation of application No. PCT/US2024/021866, filed on Mar. 28, 2024.
(60) Provisional application No. 63/492,845, filed on Mar. 29, 2023.
(60) Provisional application No. 63/505,906, filed on Jun. 2, 2023.

(51) Int. Cl. A61K 38/45; A61K 9/51; A61K 31/7105; A61K 48/00; C12N 9/10; C12N 9/22; C12N 15/11; C12N 15/88

(52) U.S. Cl. CPC A61K 38/45; A61K 9/5123; A61K 31/7105; A61K 48/0033; A61K 48/005; C12N 9/1007; C12N 9/222; C12N 15/11; C12N 15/88; C12Y 201/01037; C12N 2310/20; C12N 2310/315; C12N 2310/321

(57) ABSTRACT

Provided herein are gene repressor systems comprising repressor fusion proteins, such as repressor fusion proteins comprising DNA binding proteins, in some cases catalytically dead CRISPR proteins and guide nucleic acids (gRNA), useful in the repression of genes. Also provided are methods of using such systems to repress transcription of genes.

### CROSS REFERENCE TO RELATED APPLICATIONS

This application claims priority to, and benefit of, U.S. Provisional Application Nos. 63/492,845 filed on Mar. 29, 2023, and 63/505,906, filed on Jun. 2, 2023, the contents of each of which are incorporated by reference herein in their entireties.

### REFERENCE TO AN ELECTRONIC SEQUENCE LISTING

The contents of the electronic sequence listing (SCRB_054_02US_SubSeqList_ST26.xml; Size: 141,383,934 bytes; and Date of Creation: Mar. 26, 2026) are herein incorporated by reference in its entirety.

### BACKGROUND

Methods of modulating expression of a target gene in a cell are varied. In mammalian systems, cells use a system of chromatin regulators (CRs) and associated histone and DNA modifications to modulate gene expression and establish long-term epigenetic memory. This system is critical in development, aging, and disease, and may provide essential capabilities for incorporating regulation in synthetic biology. In experimental systems, methods such as RNA interference (RNAi) are useful for targeted-gene knockdown and have been widely used for large-scale library screens. RNAi, however, has several limitations. In particular, RNAi-based knockdown suffers from off-target effects, along with incomplete knockdown of the target (Jackson A L, et al. Expression profiling reveals off-target gene regulation by RNAi. Nat Biotechnol. 21:635 (2003)); Sigoillot F D, et al., A bioinformatics method identifies prominent off-targeted transcripts in RNAi screens. Nat Methods. 19:9(4):363 (2012)). Tailored DNA binding proteins such as zinc finger proteins or transcription activator-like effector (TALE) proteins linked to transcriptional repressor domains, while able to mediate selective gene suppression, are limited by the fact that each desired target gene necessitates the generation of a new protein.

The advent of DNA-editing systems and the programmable nature of these systems has facilitated their use as a versatile technology for genomic manipulation and engineering. In particular, CRISPR proteins are well suited for such manipulation. For example, certain Class 2 CRISPR/Cas systems have a compact size, offering ease of delivery, and the nucleotide sequence encoding the protein is relatively short, an advantage for its incorporation into viral vectors for cellular delivery. However, in certain disease indications, gene silencing, or transcriptional repression, is preferable to gene editing. The ability to render CRISPR nucleases such as Cas9 and CasX catalytically-inactive has been demonstrated (WO2020247882A1 and US20200087641A1, incorporated by reference herein), which makes these systems an attractive platform for the generation of fusion proteins with repressor domains capable of gene silencing. While certain repressor systems have been described, there remains a need for additional gene repressor systems that have been optimized and/or offer improvements over earlier generations of gene repressor systems, such as those based on Cas9, for utilization in a variety of therapeutic, diagnostic, and research applications.

Provided herein are systems and methods for targeting and repressing a gene in a cell by epigenetic modification, as well as delivery vectors and formulations, to the address this need.

### SUMMARY

Aspects of the present disclosure are directed to systems and methods of modulating expression of a target nucleic acid in a cell.

The present disclosure provides systems comprising or encoding fusion proteins comprising modified DNA-binding proteins and linked repressor domain proteins in defined configurations used, in some cases, with guide ribonucleic acids (gRNA), in the transcriptional repression and/or epigenetic modification of target nucleic acid sequences. The components of the systems can be modified for formulation for passive entry into target cells and are useful in a variety of methods for gene silencing or gene transcriptional repression in diseases where repression of a gene product is useful to reverse the underlying cause of the disease or to ameliorate the signs or symptoms of the disease, which methods are also provided. The systems of the disclosure can result in heritable epigenetic modification or silencing of a gene targeted by the system. The present disclosure provides methods of repressing transcription of a gene in a population of cells, the method comprising introducing into cells of the population systems comprising or encoding repressor fusion proteins comprising modified DNA-binding proteins and linked repressor domain proteins and, in some cases, gRNA, wherein transcription of the gene is repressed by the repressor fusion protein. The present disclosure also provides vectors and particle formulations (e.g., lipid nanoparticles, or LNP, and synthetic nanoparticles) encoding or encapsulating the system components for the delivery to cells for the silencing or transcriptional repression of the target nucleic acid in the cells.

In another aspect, provided herein are compositions comprising the systems or vectors encoding the systems for use in the manufacture of a medicament for the treatment of a disease in a subject in need thereof.

Further features and advantages of certain embodiments of the present disclosure will become more fully apparent in the following description of embodiments and drawings thereof, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of WO 2020/247882, WO 2020/247883, WO 2021/113772, WO 2022/120095, WO 2022/125843, WO 2022/261150, WO 2022/261149, WO 2023/049872, WO 2023/235818, and WO2023/049742, WO 2023/240162, which disclose CasX variants and gRNA variants, and methods of delivering same, are hereby incorporated by reference in their entirety.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a time course plot showing the percentage of mouse Hepa1-6 cells that stained negative for intracellular PCSK9 at 4, 12, 18, 24, 41, and 53 days post-delivery, as described in Example 1. Hepa1-6 cells were treated with LTRP5-ZIM3 or LTRP5-ADD-ZIM3 mRNA paired with the PCSK9-targeting gRNA with spacer 27.88. A non-targeting (NT) spacer was used as an experimental control.

FIG. 1B is a time course plot showing the percentage of mouse Hepa1-6 cells that stained negative for intracellular PCSK9 at 4, 12, 18, 24, 41, and 53 days post-delivery, as described in Example 1. Hepa1-6 cells were treated with LTRP5-ZIM3 or LTRP5-ADD-ZIM3 mRNA paired with the PCSK9-targeting gRNA with spacer 27.94. A non-targeting (NT) spacer was used as an experimental control.

FIG. 2A is a bar plot showing the quantification of normalized secreted PCSK9 levels at 4 days post-transfection in HepG2 cells lipofected with mRNA encoding CasX 676, dCasX fused to ZIM3-KRAB (dXR1), or LTRP5-ADD-ZIM3, when paired with the indicated targeting gRNAs, as described in Example 2. Secreted PCSK9 levels were normalized to total cell count. Naïve, untreated cells served as experimental controls.

FIG. 2B is a bar plot showing the quantification of normalized secreted PCSK9 levels at 4 days post-transfection in Huh7 cells lipofected with mRNA encoding CasX 676, dXR1, or LTRP5-ADD-ZIM3, when paired with the indicated targeting gRNAs, as described in Example 2. Secreted PCSK9 levels were normalized to total cell count. Naïve, untreated cells served as experimental controls.

FIG. 2C is a bar plot showing the quantification of normalized secreted PCSK9 levels at 4 days post-transfection in Hep3B cells lipofected with mRNA encoding CasX 676, dXR1, or LTRP5-ADD-ZIM3, when paired with the indicated targeting gRNAs, as described in Example 2. Secreted PCSK9 levels were normalized to total cell count. Naïve, untreated cells served as experimental controls.

FIG. 3 is a bar plot showing the quantification of secreted PCSK9 levels at 4, 14, and 27 days post-transfection in Huh7 cells lipofected with mRNA encoding CasX 676, dXR1, or LTRP5-ADD-ZIM3, when paired with the indicated targeting gRNAs, as described in Example 2. Quantification of secreted PCSK9 levels is shown as relative to the secreted levels detected in the naïve, untreated cells at the day 4 timepoint.

FIG. 4 illustrates the schematic of an LTRP5 molecule without the ADD domain of the DNMT3A, as described in Example 5. “D3A CD” and “D3L ID” denote the catalytic domain of DNMT3A and the interaction domain of DNMT3L respectively. “L1”, “L2”, “L3A”, and “L3B” are linkers. “NLS” is the nuclear localization signal. “RD1” denotes a repressor domain.

FIG. 5 is a bar graph illustrating the results of a time-course experiment comparing levels of B2M repression (represented as average percentage of HLA-negative cells) in HEK293T cells transfected with LTRP5 variant plasmids containing linker sets 1-11, as described in Example 5. Data for each timepoint (day 8, day 15, and day 45) are superimposed and presented as mean with standard deviation, N=3. A non-targeting spacer (NT) was included as experimental control.

FIG. 6 is a bar graph illustrating the results of a time-course experiment comparing the level of repression of target 1 (represented as percentage of total cells with knockdown of target 1) of LTRP5 variants containing linker sets 1-11 measured in HEK293T cells, as described in Example 5. Data for each timepoint (day 8, day 15, and day 45) are superimposed and presented as mean with standard deviation, N=3.

FIG. 7 is a bar graph illustrating the results of a time-course experiment comparing the level of repression of target 2 (represented as percentage of total cells with knockdown of target 2) by LTRP5 variants containing linker sets 1-11. The level of repression was measured in HEK293T cells, as described in Example 5. Data for each timepoint (day 8, day 15, and day 45) are superimposed and presented as mean with standard deviation, N=3. A non-targeting spacer (NT) was included as experimental control.

FIG. 8 is a bar graph illustrating the results of a time-course experiment comparing levels of B2M repression (represented as average percentage of HLA-negative cells) in HEK293T cells transfected with LTRP5 variant plasmids containing linker sets 12-28, as described in Example 5. Data for each timepoint (day 7 and day 17) are superimposed and presented as mean with standard deviation, N=3. A non-targeting spacer (NT) was included as experimental control. A non-targeting spacer (NT) was included as experimental control.

FIG. 9 is a bar graph illustrating the results of a time-course experiment comparing the level of repression of target 1 (represented as percentage of total cells with knockdown of target 1) by LTRP5 variants containing linker sets 12-28 measured in HEK293T cells, as described in Example 5. Data for each timepoint (day 7 and day 17) are superimposed and presented as mean with standard deviation, N=3. A non-targeting spacer (NT) was included as experimental control.

FIG. 10 is a bar graph illustrating the results of a time-course experiment comparing the level of repression of target 2 (represented as percentage of total cells with knockdown of target 2) by LTRP5 variants containing linker sets 12-28 measured in HEK293T cells, as described in Example 5. Data for each timepoint (day 7 and day 17) are superimposed and presented as mean with standard deviation, N=3. A non-targeting spacer (NT) was included as experimental control.

FIG. 11 is a bar plot showing the percentage of mouse Hepa1-6 cells, treated with either dXR1 or LTRP1-ZIM3 mRNA paired with the indicated PCSK9-targeting gRNAs, that stained negative for intracellular PCSK9 at day 6, as described in Example 7. Spacer 6.7 targeting the human PCSK9 locus served as a non-targeting control.

FIG. 12 is a time course plot showing the percentage of mouse Hepa1-6 cells, treated with dXR1 mRNA paired with the indicated PCSK9-targeting gRNAs, that stained negative for intracellular PCSK9 at 6, 13, and 25 days post-delivery, as described in Example 7. Spacer 6.7 targeting the human PCSK9 locus served as a non-targeting control, and treatment with water served as a negative control.

FIG. 13 is a time course plot showing the percentage of mouse Hepa1-6 cells, treated with LTRP1-ZIM3 mRNA paired with the indicated PCSK9-targeting gRNAs, that stained negative for intracellular PCSK9 at 6, 13, and 25 days post-delivery, as described in Example 7. Spacer 6.7 targeting the human PCSK9 locus served as a non-targeting control, and treatment with water served as a negative control.

FIG. 14 is a time course plot showing the percentage of mouse Hepa1-6 cells, treated with in-house in vitro transcription (IVT)-produced LTRP1-ZIM3 vs. LTRP5-ZIM3 mRNA paired with the indicated PCSK9-targeting gRNAs, that stained negative for intracellular PCSK9 at the indicated timepoints days post-delivery, as described in Example 7.

FIG. 15 is a time course plot showing the percentage of mouse Hepa1-6 cells, treated with LTRP1-ZIM3 vs. dCas9-ZNF10-DNMT3A/3L mRNA paired with the indicated PCSK9-targeting gRNAs, that stained negative for intracellular PCSK9 at the indicated timepoints post-delivery, as described in Example 7.

FIG. 16 is a plot illustrating percentage of HEK293T cells, transfected with a plasmid encoding the indicated CasX or LTRP:gRNA construct, that expressed B2M six days post-treatment with the DNMT1 inhibitor 5-azadC at varying concentrations, as described in Example 7.

FIG. 17 is a plot that juxtaposes the quantification of B2M repression in HEK293T cells transfected with a plasmid encoding the indicated CasX or LTRP:gRNA construct and cultured for 58 days, with the quantification of B2M reactivation upon treatment of transfected cells with 5-azadC, as described in Example 7.

FIG. 18 is a bar graph showing the quantification of secreted PCSK9 levels at 6, 18, and 36 days post-transfection in Huh7 cells lipofected with mRNA encoding CasX 676, dXR1, or LTRP5-ADD-ZIM3 when paired with the indicated targeting gRNAs, as described in Example 8. Secreted PCSK9 levels were normalized to total cell count. Naïve, untreated cells served as an experimental control.

FIG. 19 illustrates the schematics of various configurations of LTRP molecules with the DNMT3A ADD domain. “D3A ADD”, “D3A CD”, and “D3L ID” denote the ADD domain of DNMT3A, the catalytic domain of DNMT3A, and the interaction domain of DNMT3L respectively. “DBP” denotes the DNA binding protein. “L1”, “L2”, “L3A”, “L3B”, and “L4” are linkers. “NLS” is the nuclear localization signal. “RD1” denotes a repressor domain, and “RDla” and “RD1b” represent repressor domain variants.

FIG. 20A is a schematic illustrating versions 1-3 of chemical modifications made to gRNA scaffold variant 235, as described in Example 11. Structural motifs are highlighted. Standard ribonucleotides are depicted as open circles, and 2′OMe-modified ribonucleotides are depicted as black circles. Phosphorothioate bonds are indicated with * below or beside the bond. For the v2 profile, the addition of three 3′ uracils (3′UUU) is annotated with “U” s in the relevant circles.

FIG. 20B is a schematic illustrating versions 4-6 of chemical modifications made to gRNA scaffold variant 235, as described in Example 11. Structural motifs are highlighted. Standard ribonucleotides are depicted as open circles, and 2′OMe-modified ribonucleotides are depicted as black circles. Phosphorothioate bonds are indicated with * below or beside the bond.

FIG. 21 is a plot illustrating the quantification of percent knockout of B2Min HepG2 cells co-transfected with 100 ng of CasX 491 mRNA and with the indicated doses of end-modified (v1) or unmodified (v0) B2M-targeting gRNAs with spacer 7.37, as described in Example 11. Editing level was determined by flow cytometry as the population of cells with loss of surface presentation of the HLA complex due to successful editing at the B2M locus.

FIG. 22 is a schematic illustrating versions 7-9 of chemical modifications made to gRNA scaffold variant 316, as described in Example 11. Structural motifs are highlighted. Standard ribonucleotides are depicted as open circles, and 2′OMe-modified ribonucleotides are depicted as black circles. Phosphorothioate bonds are indicated with * below or beside the bond.

FIG. 23A is a schematic of gRNA scaffold variant 174 (SEQ ID NO: 1744), as described in Example 11. Structural motifs are highlighted.

FIG. 23B is a schematic of gRNA scaffold variant 235 (SEQ ID NO: 1745), as described in Example 11. Highlighted structural motifs are the same as in FIG. 20A. The differences between gRNA variant 174 and variant 235 lie in the extended stem motif and several single-nucleotide changes (indicated with asterisks). Variant 316 maintains the shorter extended stem from variant 174 but harbors the four substitutions found in scaffold 235.

FIG. 23C is a schematic of gRNA scaffold variant 316 (SEQ ID NO: 1746), as described in Example 11. Highlighted structural motifs are the same as in FIG. 20A. Variant 316 maintains the shorter extended stem from gRNA variant 174 (FIG. 23A) but harbors the four substitutions found in scaffold 235 (FIG. 23B).

FIG. 24 is a plot displaying a correlation between indel rate (depicted as edit fraction) at the PCSK9 locus as measured by NGS (x-axis) and secreted PCSK9 levels (ng/mL) detected by ELISA (y-axis) in HepG2 cells lipofected with CasX 491 mRNA and PCSK9-targeting gRNAs containing the indicated scaffold variant and spacer combination, as described in Example 11.

FIG. 25A is a plot depicting the results of an editing assay measured as indel rate detected by NGS at the human B2M locus in HepG2 cells treated with the indicated doses of LNPs formulated with CasX 491 mRNA and the indicated B2M-targeting gRNA, as described in Example 11.

FIG. 25B is a plot illustrating the quantification of percent knockout of B2M in HepG2 cells treated with the indicated doses of LNPs formulated with CasX 491 mRNA and the indicated B2M-targeting gRNA, as described in Example 11. Editing level was determined by flow cytometry as population of cells that did not have surface presentation of the HLA complex due to successful editing at the B2M locus.

FIG. 26A is a plot depicting the results of an editing assay measured as indel rate detected by NGS at the mouse ROSA26 locus in Hepa1-6 cells treated with the indicated doses of LNPs formulated with CasX 676 mRNA #2 and the indicated ROSA26-targeting gRNA with either the v1 or v5 modification profile, as described in Example 11.

FIG. 26B is a plot illustrating the quantification of percent editing measured as indel rate detected by NGS at the ROSA26 locus in mice treated with LNPs formulated with CasX 676 mRNA #2 and the indicated chemically-modified ROSA26-targeting gRNA, as described in Example 11.

FIG. 27 is a bar graph showing the results of the editing assay measured as indel rate detected by NGS as the mouse PCSK9 locus in mice treated with LNPs formulated with CasX 676 mRNA #1 and the indicated chemically-modified PCSK9-targeting gRNA, as described in Example 11. Untreated mice served as experimental control.

FIG. 28A is a schematic illustrating versions 1-3 of chemical modifications made to gRNA scaffold variant 316, as described in Example 11. Structural motifs are highlighted. Standard ribonucleotides are depicted as open circles, and 2′OMe-modified ribonucleotides are depicted as black circles. Phosphorothioate bonds are indicated with * below or beside the bond. For the v2 profile, the addition of three 3′ uracils (3′UUU) is annotated with “U” s in the relevant circles.

FIG. 28B is a schematic illustrating versions 4-6 of chemical modifications made to gRNA scaffold variant 316, as described in Example 11. Structural motifs are highlighted. Standard ribonucleotides are depicted as open circles, and 2′OMe-modified ribonucleotides are depicted as black circles. Phosphorothioate bonds are indicated with * below or beside the bond.

FIG. 29 is a violin plot with each point representing mean % methylation at individual CpG motifs. Median methylation is indicated by dashed lines, with upper and lower quartiles indicated by dotted lines. Transcriptional start site (TSS)-proximal DNA methylation was measured from homogenized liver-extracted gDNA by amplicon enzymatic methylation sequencing (EM-seq) from N=3 mice sacrificed at days 7, 14, and 42 post-treatment, as described in example 12.

FIG. 30 is a violin plot with each point representing mean % methylation at individual CpGs. Median methylation is indicated by dashed lines, with upper and lower quartiles indicated by dotted lines. Transcriptional start site (TSS)-proximal DNA methylation was measured from homogenized liver-extracted gDNA by amplicon enzymatic methylation sequencing (EM-seq) from N=3 mice sacrificed at day 7 post-treatment, as described in example 13.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

As used in this specification and the appended claims, the singular forms “a,” “an,” and “the” include plural referents unless the context clearly dictates otherwise. Thus, for example, reference to “a host cell” includes two or more such host cells, reference to “an engineered CasX protein” includes one or more engineered CasX protein(s), reference to “a nucleic acid sequence” includes one or more nucleic acid sequences, and the like.

As used herein, the term “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also include any and all possible subranges and combinations of subranges thereof. Furthermore, as will be understood by one skilled in the art, a range includes each individual member. Thus, for example, a group having 1-3 members refers to groups having 1, 2, or 3 members. Similarly, a group having 1-5 members refers to groups having 1, 2, 3, 4, or 5 members, and so forth.

The term “combinations thereof includes every possible combination of elements to which the term refers.

The term” exemplary” as used herein, refers to an example or illustration, and is not intended to imply any preference or value.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like). Thus, the skilled artisan will understand that while individual bases within a sequence may not be complementary to another sequence, the sequence as a whole is still considered to be complementary.

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include coding and non-coding sequences that enhance expression, trafficking of the nucleic acid, or the function of mRNA or protein and include, inter alia, polyadenylation signals (poly(A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art. All Pol III promoters are envisaged as within the scope of the instant disclosure.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PTRE),” such as a hepatitis PTRE, refers to a DNA sequence that, when transcribed creates a tertiary structure capable of exhibiting post-transcriptional activity to enhance or promote expression of an associated gene operably linked thereto.

“Operably linked” means with reference to a juxtaposition of two or more components (such as sequence elements), in which the components are arranged such that both components function normally and allow the possibility that at least one of the components can mediate a function that is exerted upon at least one of the other components; e.g., a promoter and an encoding sequence. The skilled artisan will appreciate that the two components need not be physically linked to be operably linked.

In the context of the present disclosure and with respect to a gene, “repress”, “repression”, “transcriptional repression”, “repressing”, “inhibition of gene expression”, “downregulation”, and “silencing” are used interchangeably herein to refer to the inhibition or blocking of transcription of a gene or a portion thereof. Accordingly, transcriptional repression of a gene can result in a decrease in production of a gene product. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Transcriptional repression includes both reversible and irreversible inactivation of gene transcription; the latter can result from epigenetic modification of the gene.

“Repressor” or “repressor domain” are used interchangeably herein to refer to polypeptide factors that act as regulatory elements on DNA to inhibit, repress, or block transcription of DNA, resulting in repression of gene expression. In the context of the present disclosure, the linking of a repressor domain to a DNA binding protein that can, when bound to the target nucleic acid, prevent transcription from a promoter or otherwise inhibit the expression of a gene. Without wishing to be bound by theory, it is thought that transcriptional repressors can function by a variety of mechanisms, including physically blocking RNA polymerase passage by steric hindrance, altering the polymerase's post-translational modification state, modifying the epigenetic state of the nascent RNA, changing the epigenetic state of the DNA through methylation, changing the epigenetic state of the DNA through histone deacetylation or modulating nucleosome remodeling, or preventing enhancer-promoter interactions, thereby leading to gene silencing or a reduction in the level of gene expression.

“Long-term repressor fusion protein” or “LTRP” is used interchangeably herein with “repressor fusion protein” and refers to a fusion protein comprising a DNA binding protein (or DNA binding domain of a protein) fused to one or more domains capable of repressing transcription of a target nucleic acid sequence. Optionally, the repressor fusion proteins of the disclosure may contain additional elements, such as linkers between any of the domains of the fusion protein, nuclear localization signals, nuclear export signals, as well as additional protein domains that confer additional activities upon the repressor fusion protein.

As used herein an “LTRP:gRNA system” is a system for transcriptional repression and comprises a long-term repressor fusion protein comprising a catalytically-dead CRISPR protein and one or more linked repressor domains, and a guide nucleic acid (gRNA) that binds to the catalytically-dead CRISPR protein. For clarity, the system also includes any encoding DNA, RNA or vectors and the like that can be used to produce the repressor fusion proteins and gRNA components of the system.

As used herein, a DNA binding protein refers to a protein, or domain of a protein, capable of binding to DNA. Exemplary DNA binding proteins include zinc finger (ZF) proteins, Transcription Activator-like Effectors (TALEs), and clustered regularly interspaced short palindromic repeats (CRISPR) proteins. The skilled artisan will appreciate that in multi-functional proteins that are capable of both binding DNA and carrying out another activity such as DNA cleavage, such as, e.g., CRISPR proteins, the DNA binding function can be separated from the other functions of the protein, leading to catalytically-dead DNA binding proteins.

As used herein a “catalytically-dead DNA binding protein” refers to a protein that is able to bind DNA but is unable to nick or cleave the DNA. As used herein a “catalytically-dead CRISPR protein” refers to a CRISPR protein that lacks endonuclease activity. The skilled artisan will appreciate that a CRISPR protein can be catalytically-dead, and still able to carry out additional protein functions, such as DNA binding. Similarly, a “catalytically-dead CasX” refers to a CasX protein that lacks endonuclease activity but is still able to carry out additional protein functions, such as DNA binding.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, “lipid nanoparticle” or “LNP” refers to a particle having at least one dimension on the order of nanometers (e.g., 1-1,000 nm) comprising one or more lipids (e.g., cationic lipids, non-cationic lipids, helper phospholipids, and PEG-modified lipids), as well as cholesterol. Specific components of LNP are described more fully, below. Lipid nanoparticles can be included in a formulation that can be used to deliver an active agent or therapeutic agent, such as a nucleic acid (e.g., mRNA) to a target site of interest (e.g., cell, tissue, organ, tumor, and the like). The lipid nanoparticles of the disclosure can comprise a nucleic acid. Such lipid nanoparticles typically comprise neutral lipids, charged lipids, steroids and polymer conjugated lipids. The active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells; e.g. an adverse immune response.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L][P]/[LP], where [P], [L] and [LP] represent molar concentrations of the protein, ligand and complex, respectively.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, or cosmid, which can include another DNA segment, i.e., an expression cassette, so as to bring about the replication or expression of the other DNA segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an AAV vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an AAV vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4^{th} Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Epigenetic Modification and Repression of Genes

The disclosure provides specifically configured systems that have utility in the transcriptional repression and/or epigenetic modification of a gene in a cell. As used herein, a “system” is used interchangeably with “composition”. In some cases, the system is designed to repress transcription of a gene in eukaryotic cells having a mutation. In other cases, the system is designed to repress or silence transcription of a wild-type gene in eukaryotic cells that nevertheless contributes to a disease or a condition. Generally, any portion of a gene can be targeted using the programable systems and methods of the disclosure, described more fully, herein.

The present disclosure provides the components of systems of long-term repressor fusion proteins comprising or encoding various configurations of a DNA binding protein and linked repressor domains capable of binding a target nucleic acid sequence of a gene targeted for transcriptional repression and/or epigenetic modification. Such fusion proteins, referred to herein as long-term repressor fusion proteins (“LTRP”), allow for long-term repression or silencing effects on the targeted gene. The disclosure also provides nucleic acids encoding the systems. Also provided herein are methods of making the systems, as well as methods of using the systems, including methods of gene repression and/or epigenetic modification and methods of treatment of diseases or disorders for which gene repression or silencing is sought.

In some embodiments, the DNA binding proteins comprise zinc finger (ZF) or TALE (transcription-activator-like effector) proteins that bind but do not cleave the target nucleic acid. The DNA-binding domain of a TALE is comprised of a tandem array of 33-34 amino acid (aa)-long customizable monomers that theoretically can be assembled to recognize any genetic sequence following a one-repeat-binds-one-base-pair recognition code (Jain, S., et al. TALEN outperforms Cas9 in editing heterochromatin target sites. Nat. Commun. 12:606 (2021)). The specificity of TALEs for binding DNA arises from two polymorphic amino acids, the so-called repeat variable diresidues (RVDs) located at positions 12 and 13 of a repeated unit. By re-arranging the repeats, the DNA binding specificities of TALE can be changed at will. Zinc finger proteins are transcription factors, where each finger recognizes 3-4 bases. By mixing and matching these finger modules, a ZR protein can be customized for the sequence to be targeted.

In some embodiments, the DNA binding proteins comprise a catalytically-dead Class 1 or Class 2 CRISPR protein. Catalytically-dead CRISPR proteins are also referred to in the art as “catalytically inactive” CRISPR proteins. In some embodiments, the Class 2, Type II protein is a catalytically-dead Cas9. In another embodiment, the Class 2 CRISPR protein is selected from the group consisting of a Type II, Type V, or Type VI protein. In some embodiments, the Class 2, Type V protein is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ, in each case rendered catalytically-dead by specific mutations, as described herein. The CRISPR-based systems further comprise a guide ribonucleic acid (gRNA) with a targeting sequence complementary to the target sequence of a gene to be bound and repressed by a complex of the fusion protein (the CRISPR protein and linked repressor domains) and the gRNA.

In some embodiments, the present disclosure provides systems comprising or encoding a long-term repressor fusion protein comprising a catalytically-dead CasX nuclease protein and linked repressor domains, and a guide ribonucleic acid (gRNA) comprising a targeting sequence complementary to a target nucleic acid sequence of a gene targeted for transcriptional repression, silencing, or epigenetic modification. In some embodiments, the system comprises a long-term repressor fusion protein and a gRNA of the disclosure as gene repressor pairs (“LTRP:gRNA systems”) that are capable of forming a ribonucleoprotein (RNP) complex and binding a target nucleic acid. In some embodiments, the target nucleic acid is in a eukaryotic cell. In other cases, the disclosure provides systems of nucleic acids encoding the long-term repressor fusion protein and gRNA. In still other cases, the disclosure provides systems of a gRNA and an mRNA encoding the long-term repressor fusion protein for use in certain particle formulations (e.g., an LNP) described herein.

Also provided herein are methods of making long-term repressor fusion protein and gRNA, as well as methods of using the LTRP:gRNA systems, including methods of gene repression and/or epigenetic modification and methods of treatment. The catalytically dead CRISPR protein (e.g., dCasX protein) and linked repressor domain(s) and gRNA components of the LTRP:gRNA systems and their features, as well as the delivery modalities and the methods of using the systems for the transcriptional repression, epigenetic modification, or silencing of a gene are described more fully, below.

### III. Catalytically-dead CRISPR Proteins for Use in the Long-term Repressor Fusion Protein Systems

In some embodiments, the DNA binding protein for use in the systems of the disclosure is a catalytically-dead Class 1 or Class 2 CRISPR protein. In some embodiments, the Class 2 CRISPR protein is a Class 2, Type II protein; for example a catalytically-dead Cas9. In other embodiments, the catalytically-dead Class 2 CRISPR protein is selected from the group consisting of a Type II, Type V, or Type VI protein. In one embodiment, the Class 2 CRISPR Type V protein is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or CasΦ, in each case rendered catalytically-dead by specific mutations, as described herein. In another embodiment, the Class 2 CRISPR Type V protein is a catalytically-dead CasX (dCasX) protein.

The term “CasX protein”, as used herein, refers to a family of proteins, and encompasses all naturally-occurring CasX proteins (“reference CasX”), as well as engineered CasX proteins with multiple sequence modifications (CasX variants), in addition to those rendering the CasX catalytically-dead (dCasX), that possess one or more improved characteristics relative to a catalytically-dead reference CasX protein, described more fully, below. CasX proteins of the disclosure comprise the following domains: a non-target strand binding (NTSB) domain, a target strand loading (TSL) domain, a helical I domain, a helical II domain, an oligonucleotide binding domain (OBD), and a RuvC domain, and, in some cases, domains can be further divided into subdomains, as listed in Table 1.

In the context of the present disclosure, the CasX for use in the systems are catalytically-dead (dCasX); achieved by mutations introduced at select locations in the RuvC sequence, described below.

a. Reference CasX Proteins

The disclosure provides naturally-occurring CasX proteins (referred to herein as a “reference CasX protein”), which were subsequently modified to create the engineered dCasX of the disclosure. For example, reference CasX proteins can be isolated from naturally occurring prokaryotes, such as Deltaproteobacteria, Planctomycetes, or Candidatus Sungbacteria species. A reference CasX protein (interchangeably referred to herein as a reference CasX polypeptide) is a Class 2, Type V CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter, and comprises a sequence of SEQ ID NO: 1.

In some cases, a reference CasX protein is isolated or derived from Planctomycetes, and comprises a sequence of SEQ ID NO: 2.

In some cases, a reference CasX protein is isolated or derived from Candidatus Sungbacteria, and comprises a sequence of SEQ ID NO: 3.

b. Catalytically-dead Class 1 or Class 2 CRISPR Proteins

In the long-term repressor protein systems of the disclosure, the catalytically-dead Class 1 or Class 2 CRISPR protein is catalytically-dead in that it is unable to cleave DNA, but retains the ability to bind a target nucleic acid when complexed with a guide RNA (gRNA). The present disclosure provides catalytically-dead variants of Class 1 or Class 2 CRISPR proteins, wherein the catalytically-dead variants comprise multiple modifications in select domains. In some embodiments, the present disclosure provides catalytically-dead CasX variants (interchangeably referred to herein as “dCasX variant” or “dCasX variant protein”), wherein the catalytically-dead CasX variants comprise multiple modifications in the RuvC domain relative to the sequences of SEQ ID NOS: 1-3 (described, supra). In some embodiments, a catalytically-dead reference CasX protein comprises substitutions at residues 672, 769 and/or 935 with reference to SEQ ID NO: 1. In one embodiment, a catalytically-dead reference CasX protein comprises substitutions of D672A, E769A and/or D935A with reference to SEQ ID NO: 1. In other embodiments, a catalytically-dead reference CasX protein comprises substitutions at amino acids 659, 756 and/or 922 with reference to SEQ ID NO: 2. In some embodiments, a catalytically-dead reference CasX protein comprises D659A, E756A and/or D922A substitutions with reference to of SEQ ID NO: 2. An exemplary RuvC domain of the dCasX variants of the disclosure comprises amino acids 661-824 and 935-986 of SEQ ID NO: 1, or amino acids 648-812 and 922-978 of SEQ ID NO: 2, with one or more amino acid modifications relative to said RuvC cleavage domain sequence, wherein the dCasX variant exhibits one or more improved characteristics compared to the reference dCasX. In further embodiments, a catalytically-dead CasX variant protein comprises deletions of all or part of the RuvC domain of the reference CasX protein. It will be understood that the same foregoing substitutions or deletions can similarly be introduced into CasX variants known in the art, resulting in a dCasX variant (see, e.g., WO2022120095A1 and U.S. Pat. No. 11,560,555, incorporated by reference herein, for exemplary sequences).

In some embodiments, the long-term repressor fusion protein comprising dCasX variant with linked repressor domains exhibits at least one improved characteristic compared to a long-term repressor fusion protein comprising a reference dCasX protein with comparable linked repressor domains. All dCasX variants that improve one or more functions or characteristics of the long-term repressor fusion protein comprising the dCasX variant protein compared to a comparable long-term repressor fusion protein comprising the reference dCasX protein are envisaged as being within the scope of the disclosure. In some embodiments, the modification is a mutation in one or more amino acids of the reference dCasX other than those rendering the dCasX catalytically-dead. For example, dCasX variants can comprise one or more amino acid substitutions, insertions, deletions, or swapped domains, or any combinations thereof, relative to a reference dCasX protein sequence. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference dCasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a dCasX variant protein of the disclosure. Exemplary improved characteristics of the dCasX variant embodiments include, but are not limited to improved folding of the variant, increased binding affinity to the target nucleic acid, improved ability to utilize a greater spectrum of PAM sequences in the transcriptional repression and/or binding of target nucleic acid, improved unwinding of the target DNA, increased target strand loading, increased binding of the non-target strand of DNA, improved protein stability, increased ability to complex with gRNA, increased binding affinity to the gRNA, improved protein:gRNA (RNP) complex stability, and, with linked repressor domains and when complexed as an RNP, increased repressor activity, improved repressor specificity for the target nucleic acid, decreased off-target repression, increased percentage of a eukaryotic genome that can be efficiently repressed and/or epigenetically modified. In some embodiments, an improved characteristic of the dCasX variant is at least about 1.1 to about 100,000-fold improved relative to the reference dCasX protein. In some embodiments, an improved characteristic of the dCasX variant is at least about 1.1 to about 10,000-fold improved, at least about 1.1 to about 1,000-fold improved, at least about 1.1 to about 500-fold improved, at least about 1.1 to about 400-fold improved, at least about 1.1 to about 300-fold improved, at least about 1.1 to about 200-fold improved, at least about 1.1 to about 100-fold improved, at least about 1.1 to about 50-fold improved, at least about 1.1 to about 40-fold improved, at least about 1.1 to about 30-fold improved, at least about 1.1 to about 20-fold improved, at least about 1.1 to about 10-fold improved, at least about 1.1 to about 9-fold improved, at least about 1.1 to about 8-fold improved, at least about 1.1 to about 7-fold improved, at least about 1.1 to about 6-fold improved, at least about 1.1 to about 5-fold improved, at least about 1.1 to about 4-fold improved, at least about 1.1 to about 3-fold improved, at least about 1.1 to about 2-fold improved, at least about 1.1 to about 1.5-fold improved, at least about 1.5 to about 3-fold improved, at least about 1.5 to about 4-fold improved, at least about 1.5 to about 5-fold improved, at least about 1.5 to about 10-fold improved, at least about 5 to about 10-fold improved, at least about 10 to about 20-fold improved, at least 10 to about 30-fold improved, at least 10 to about 50-fold improved or at least 10 to about 100-fold improved relative to the reference dCasX protein. In some embodiments, an improved characteristic of the dCasX variant is at least about 10 to about 1000-fold improved relative to the reference dCasX protein. Additional disclosure on improved characteristics is described herein, below.

In other embodiments, the modification is a substitution of one or more domains of the reference dCasX with one or more domains from a different CasX. In some embodiments, insertion includes the insertion of a part or all of a domain from a different CasX protein. Mutations can be placed in any one or more domains of the dCasX variant, and may include, for example, deletion of part or all of one or more domains, or one or more amino acid substitutions, deletions, or insertions in any domain. The domains of dCasX proteins include the non-target strand binding (NTSB) domain, the target strand loading (TSL) domain, the helical I domain, the helical II domain, the oligonucleotide binding domain (OBD), and the RuvC DNA domain, which can further comprise subdomains, described below.

In some embodiments, the dCasX variant protein comprises between 800 and 1100 amino acids or between 900 and 1000 amino acids.

The long-term repressor fusion proteins comprising dCasX and linked repressor domains of the disclosure have an enhanced ability to efficiently bind target nucleic acid, when complexed with a gRNA as an RNP, utilizing and binding to a PAM TC motif, including PAM sequences selected from TTC, ATC, GTC, or CTC, compared to an RNP of a fusion protein comprising a reference dCasX protein and comparable linked repressor domains and a gRNA in a comparable assay system. In the foregoing, the PAM sequence is located at least 1 nucleotide 5′ to the non-target strand of the protospacer having identity with the targeting sequence of the gRNA.

In some embodiments, an RNP comprising the long-term repressor fusion protein comprising the dCasX variant protein with linked repressor domains and a gRNA of the disclosure, at a concentration of 20 μM or less, is capable of binding a double stranded DNA target with an efficiency of at least 70%, at least 80%, at least 85%, at least 90% or at least 95%. In one embodiment, the RNP of the long-term repressor fusion protein comprising the dCasX variant with linked repressor domains and a gRNA variant exhibits greater binding affinity for a target sequence in the target nucleic acid compared to an RNP comprising a repressor fusion protein comprising a reference dCasX protein with linked repressor domains and a gRNA in a comparable assay system, wherein the PAM sequence of the target nucleic acid is TTC. In another embodiment, an RNP of a long-term repressor fusion protein comprising the dCasX variant with linked repressor domains and the gRNA variant exhibits greater binding affinity for a target sequence in the target nucleic acid compared to an RNP comprising a long-term repressor fusion protein comprising a reference dCasX protein with linked repressor domains and a reference gRNA in a comparable assay system, wherein the PAM sequence of the target nucleic acid is ATC. In another embodiment, the RNP of the long-term repressor fusion protein comprising the dCasX variant with linked repressor domains and a gRNA variant exhibits greater binding affinity for a target sequence in the target nucleic acid compared to an RNP comprising a long-term repressor fusion protein comprising the reference dCasX protein with linked repressor domains and a reference gRNA in a comparable assay system, wherein the PAM sequence of the target nucleic acid is CTC. In another embodiment, an RNP of a long-term repressor fusion protein comprising the dCasX variant with linked repressor domains and the gRNA variant exhibits greater binding affinity for a target sequence in the target nucleic acid compared to an RNP comprising a long-term repressor fusion protein comprising the reference dCasX protein with linked repressor domains and a reference gRNA in a comparable assay system, wherein the PAM sequence of the target nucleic acid is GTC. In other embodiments, an RNP of a long-term repressor fusion protein comprising the dCasX variant with linked repressor domains and a gRNA exhibits greater binding affinity for a target sequence in the target nucleic acid compared to an RNP comprising a comparable repressor fusion protein comprising the reference dCasX protein with linked repressor domains and the gRNA in a comparable assay system, wherein the PAM sequence of the target nucleic acid is GTC, TTC, ATC or CTC. In the foregoing embodiments, the increased binding affinity for the one or more PAM sequences is at least 1.5-fold greater or more compared to the binding affinity of an RNP of any one of the reference dCasX proteins (modified from SEQ ID NOS:1-3) with linked repressor domains and the gRNA of Table 8 for the PAM sequences.

c. dCasX Variant Proteins with Domains from Multiple Source Proteins

In certain embodiments, the disclosure provides a chimeric dCasX variant protein for use in the repressor fusion proteins.

As used herein, a “chimeric dCasX” protein refers to both a dCasX protein containing at least two domains from different sources, as well a dCasX protein containing at least one domain that itself is chimeric. Accordingly, in some embodiments, a chimeric dCasX protein is one that includes at least two domains isolated or derived from different sources, such as from two different naturally occurring CasX proteins, (e.g., from two different CasX reference proteins). In other embodiments, the chimeric dCasX protein is one that contains at least one domain that is a chimeric domain; e.g. in some embodiments, part of a domain comprises a substitution from a different CasX protein (from a reference CasX protein, or another CasX variant protein).

In some embodiments, the at least one chimeric domain can be any of the NTSB, TSL, helical I, helical II, OBD or RuvC domains as described herein. In the case of split or non-contiguous domains such as helical I, RuvC and OBD, a portion of the non-contiguous domain can be replaced with the corresponding portion from any other source. In some embodiments, the helical I-II domain of the dCasX variant derived from SEQ ID NO: 2 is replaced with the corresponding helical I-II sequence from SEQ ID NO: 1, resulting in a chimeric dCasX protein. In some embodiments, the helical I-II domain and NTSB domain of the dCasX variant derived from SEQ ID NO: 2 is replaced with the corresponding helical I-II and NTSB sequences from SEQ ID NO: 1, resulting in a chimeric dCasX protein.

A chimeric dCasX variant protein may comprise an NTSB, TSL, helical I-I, helical I-II, helical II, OBD-I, and OBD-II domains from a CasX protein of SEQ ID NO: 2, and a RuvC-I and/or RuvC-II domain from a CasX protein of SEQ ID NO: 1, or vice versa, in which mutations or other sequence alterations are introduced to create the catalytically-dead variant with improved properties of the variant, relative to the reference dCasX protein. As an example of the foregoing, the chimeric RuvC domain comprises amino acids 660 to 823 of SEQ ID NO: 1 and amino acids 921 to 978 of SEQ ID NO: 2. As an alternative example of the foregoing, a chimeric RuvC domain comprises amino acids 647 to 810 of SEQ ID NO: 2 and amino acids 934 to 986 of SEQ ID NO: 1. In a particular embodiment, a dCasX for use in the long-term repressor fusion protein comprises an NTSB domain and helical I-II domain from SEQ ID NO: 1 and a helical I-I domain from SEQ ID NO: 2; the latter being a chimeric domain, it being understood that the dCasX variants have additional amino acid changes at select locations (relative to the reference sequence), and the resulting chimeric dCasX proteins have improved characteristics relative to the reference dCasX proteins. Sequences of Table 2 having the NTSB domain and helical I-II domain from SEQ ID NO: 1 and a helical I-I domain from SEQ ID NO: 2 include dCasX 491 (SEQ ID NO: 4), 515 (SEQ ID NO: 6), 516 (SEQ ID NO: 7), 518-520 (SEQ ID NOS: 9-11), 522-527 (SEQ ID NOS: 12-17), 532 (SEQ ID NO: 22), 593 (SEQ ID NO: 25), 676 (with a L169K substitution in the NTSB domain, SEQ ID NO: 28), and 812 (SEQ ID NO: 29). Coordinates of CasX domains in the reference CasX proteins of SEQ ID NO: 1 and SEQ ID NO: 2 are provided in Table 1 below. The skilled artisan will understand that the domain boundaries indicated in Table 1 below are approximate, and that protein fragments whose boundaries differ from those given in the table below by 1, 2, or 3 amino acids may have the same activity as the domains described below.

TABLE 1
Domain coordinates in Reference CasX proteins

| | Coordinates in | Coordinates in |
|---|---|---|
| Domain Name | SEQ ID NO: 1* | SEQ ID NO: 2* |
| OBD-I | 1-55 | 1-57 |
| helical I-I | 56-99 | 58-101 |
| NTSB | 100-190 | 102-191 |
| helical I-II | 191-331 | 192-332 |
| helical II | 332-508 | 333-500 |
| OBD-II | 509-659 | 501-646 |
| RuvC-I | 660-823 | 647-810 |
| TSL | 824-933 | 811-920 |
| RuvC-II | 934-986 | 921-978 |

*amino acid position

In some embodiments, a dCasX variant protein utilized in the long-term repressor fusion proteins of the disclosure comprises a sequence selected from the group consisting of the sequences of SEQ ID NOS: 4-29 as set forth in Table 2, wherein the long-term repressor fusion protein comprising the dCasX retains the ability to form an RNP with a gRNA. In other embodiments, a dCasX variant protein utilized in the repressor fusion proteins of the disclosure comprises a sequence at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence selected from the group consisting of the sequences of SEQ ID NOS: 4-29 as set forth in Table 2, wherein the long-term repressor fusion protein comprising the dCasX retains the ability to form an RNP with a gRNA. In some embodiments, a dCasX variant protein utilized in the repressor fusion proteins of the disclosure comprises a sequence selected from the group consisting of the sequences of SEQ ID NOS: 4-29, wherein the long-term repressor fusion protein comprising the dCasX retains the ability to form an RNP with a gRNA. In a particular embodiment, the dCasX variant protein utilized in the long-term repressor fusion protein of the gene repressor systems of the disclosure comprises a sequence of SEQ ID NO: 4 (dCasX 491). In another particular embodiment, the dCasX variant protein utilized in the long-term repressor fusion protein of the gene repressor systems of the disclosure comprises a sequence of SEQ ID NO: 6 (dCasX 515). In another particular embodiment, the dCasX variant protein utilized in the long-term repressor fusion protein of the gene repressor systems of the disclosure comprises a sequence of SEQ ID NO: 28 (dCasX 676). In another particular embodiment, the dCasX variant protein utilized in the long-term repressor fusion protein of the gene repressor systems of the disclosure comprises a sequence of SEQ ID NO: 29 (dCasX 812).

TABLE 2
dCasX Variant Sequences

| SEQ | | |
|---|---|---|
| ID | | |
| NO | dCasX | Amino Acid Sequence |
| 4 | dCasX | QEIKRINKIRRRLVKDSNTKKAGKTGPMKTLLVRVMTPDLRERLENLRKKPENIPQPIS |
| | 491 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 5 | dCasX | QEIKRINKIRRRLVKDSNTKKAGKTGPMKTLLVRVMTPDLRERLENLRKKPENIPQPIS |
| | 514 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTIHTSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 6 | dCasX | QEIKRINKIRRRLVKDSNTKKAGKTGPMKTLLVRVMTPDLRERLENLRKKPENIPQPIS |
| | 515 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 7 | dCasX | QEIKRINKIRRRLVKDSNTKKAGKTGPMKTLLVRVMTPDLRERLENLRKKPENIPQPIS |
| | 516 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNHNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 8 | dCasX | QEIKRINKIRRRLVKDSNTKKAGKTGPMKTLLVRVMTPDLRERLENLRKKPENIPQPIS |
| | 517 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGAPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 9 | dCasX | RQEIKRINKIRRRLVKDSNTKKAGKTGPMKTLLVRVMTPDLRERLENLRKKPENIPQPI |
| | 518 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLILLAQLKPEK |
| | | DSDEAVTYSLGKFGQRALDFYSIHVTKESTHPVKPLAQIAGNRYASGPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 10 | dCasX | QEIKRINKIRRRLVKDSNTKKAGKTGPMKTLLVRVMTPDLRERLENLRKKPENIPQPIS |
| | 519 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHIQLRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 11 | dCasX | QEIKRINKIRRRLVKDSNTKKAGKTGPMKTLLVRVMTPDLRERLENLRKKPENIPQPIS |
| | 520 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTTQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 12 | dCasX | QEIKRINKIRRRLVKDSNTKKAGKTGPMKTLLVRVMTPDLRERLENLRKKPENIPQPIS |
| | 522 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKRSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 13 | dCasX | QEIKRINKIRRRLVKDSNTKKAGKTYPMKTLLVRVMTPDLRERLENLRKKPENIPQPIS |
| | 523 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 14 | dCasX | QEIKRINKIRRRLVKDSNTKKAGKTGPMKTLLVRVMTPDLRERLENLRKKPENIPQPIS |
| | 524 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTIHSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 15 | dCasX | QEIKRINKIRRRLVKDSNTKKAGKTGPMKTLLVRVMTPDLRERLENLRKKPENIPQPIS |
| | 525 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAATQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 16 | dCasX | QEIKRINKIRRRLVKDSNTKKAGKTGPMKTLLVRVMTPDLRERLENLRKKPENIPQPIS |
| | 526 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAA |
| | | KEVEQRRAGGYSRKYASKAKNLADDMVRNTARDLLYYAVTQDAMLIFANLSRGFGRQGK |
| | | RTFMAERQYTRMEDWLTAKLAYEGLSKTYLSKTLAQYTSKTCSNCGFTITSADYDRVLE |
| | | KLKKTATGWMTTINGKELKVEGQITYYNRYKRQNVVKDLSVELDRLSEESVNNDISSWT |
| | | KGRSGEALSLLKKRFSHRPVQEKFVCLNCGFETHAAEQAALNIARSWLFLRSQEYKKYQ |
| | | TNKTTGNTDKRAFVETWQSFYRKKLKEVWKPAV |
| 17 | dCasX | QEIKRINKIRRRLVKDSNTKKAGKTRGPMKTLLVRVMTPDLRERLENLRKKPENIPQPI |
| | 527 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLILLAQLKPEK |
| | | DSDEAVTYSLGKFGQRALDFYSIHVTKESTHPVKPLAQIAGNRYASGPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 18 | dCasX | QEIKRINKIRRRLVKDSNTKKAGKTGPMKTLLVRVMTPDLRERLENLRKKPENIPQPIS |
| | 528 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASYPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 19 | dCasX | QEIKRINKIRRRLVKDSNTKKAGKTGPMKTLLVRVMTPDLRERLENLRKKPENIPQPIS |
| | 529 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASNPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 20 | dCasX | QEIKRINKIRRRLVKDSNTKKAGKTGPMKTLLVRVMTPDLRERLENLRKKPENIPQPIS |
| | 530 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGWGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 21 | dCasX | QEIKRINKIRRRLVKDSNTKKAGKTGPMKTLLVRVMTPDLRERLENLRKKPENIPQPIS |
| | 531 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGYGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 22 | dCasX | QEIKRINKIRRRLVKDSNTKKAGKTRGPMKTLLVRVMTPDLRERLENLRKKPENIPQPI |
| | 532 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLILLAQLKPEK |
| | | DSDEAVTYSLGKFGQRALDFYSIHVTKESTHPVKPLAQIAGNRYASGPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 23 | dCasX | QEIKRINKIRRRLVKDSNTKKAGKTRGPMKTLLVRVMTPDLRERLENLRKKPENIPQPI |
| | 533 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLILLAQLKPEK |
| | | DSDEAVTYSLGKFGQRALDFYSIHVTKESTHPVKPLAQIAGNRYASYPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 24 | dCasX | QEIKRINKIRRRLVKDSNTKKAGKTGPMKTLLVRVMTPDLRERLENLRKKPENIPQPIS |
| | 535 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASSPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 25 | dCasX | QEIKRINKIRRRLVKDSNTKKAGKTGPMKTLLVRVMTPDLRERLENLRKKPENIPQPIS |
| | 593 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRWWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 26 | dCasX | QEIKRINKIRRRLVKDSNTKKAGKTRGPMKTLLVRVMTPDLRERLENLRKKPENIPQPI |
| | 668 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLILLAQLKPEK |
| | | DSDEAVTYSLGKFGQRALDFYSIHVTKESTHPVKPLAQIAGNRYASSPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 27 | dCasX | QEIKRINKIRRRLVKDSNTKKAGKTGPMKTLLVRVMTPDLRERLENLRKKPENIPQPIS |
| | 672 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLIKLAQLKPEKD |
| | | SDEAVTYSLGKFGQRALDFYSIHVTKESTHPVKPLAQIAGNRYASSPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKGFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |
| 28 | dCasX | QEIKRINKIRRRLVKDSNTKKAGKTRGPMKTLLVRVMTPDLRERLENLRKKPENIPQPI |
| | 676 | SNTSRANLNKLLTDYTEMKKAILHVYWEEFQKDPVGLMSRVAQPASKKIDQNKLKPEMD |
| | | EKGNLTTAGFACSQCGQPLFVYKLEQVSEKGKAYTNYFGRCNVAEHEKLIKLAQLKPEK |
| | | DSDEAVTYSLGKFGQRALDFYSIHVTKESTHPVKPLAQIAGNRYASSPVGKALSDACMG |
| | | TIASFLSKYQDIIIEHQKVVKGNQKRLESLRELAGKENLEYPSVTLPPQPHTKEGVDAY |
| | | NEVIARVRMWVNLNLWQKLKLSRDDAKPLLRLKGFPSFPLVERQANEVDWWDMVCNVKK |
| | | LINEKKEDGKVFWQNLAGYKRQEALRPYLSSEEDRKKGKKFARYQLGDLLLHLEKKHGE |
| | | DWGKVYDEAWERIDKKVEGLSKHIKLEEERRSEDAQSKAALTDWLRAKASFVIEGLKEA |
| | | DKDEFCRCELKLQKWYGDLRGKPFAIEAENSILDISGFSKQYNCAFIWQKDGVKKLNLY |
| | | LIINYFKGGKLRFKKIKPEAFEANRFYTVINKKSGEIVPMEVNFNFDDPNLIILPLAFG |
| | | KRQGREFIWNDLLSLETGSLKLANGRVIEKTLYNRRTRQDEPALFVALTFERREVLDSS |
| | | NIKPMNLIGVARGENIPAVIALTDPEGCPLSRFKDSLGNPTHILRIGESYKEKQRTIQA |
| | | KKEVEQRRAGGYSRKYASKAKNLADDMVRNTARDLLYYAVTQDAMLIFANLSRGFGRQG |
| | | KRTFMAERQYTRMEDWLTAKLAYEGLPSKTYLSKTLAQYTSKTCSNCGFTITSADYDRV |
| | | LEKLKKTATGWMTTINGKELKVEGQITYYNRYKRQNVVKDLSVELDRLSEESVNNDISS |
| | | WTKGRSGEALSLLKKRFSHRPVQEKFVCLNCGFETHAAEQAALNIARSWLFLRSQEYKK |
| | | YQTNKTTGNTDKRAFVETWQSFYRKKLKEVWKPAV |
| 29 | dCasX | QEIKRINKIRRRLVKDSNTKKAGKTGPMKTLLVRVMTPDLRERLENLRKKPENIPQPIS |
| | 812 | NTSRANLNKLLTDYTEMKKAILHVYWEEFQKDPVGLMSRVAQPASKKIDQNKLKPEMDE |
| | | KGNLTTAGFACSQCGQPLFVYKLEQVSEKGKAYTNYFGRCNVAEHEKLILLAQLKPEKD |
| | | SDEAVTYSLGKFGQRALDFYSIHVTKESTHPVKPLAQIAGNRYASGPVGKALSDACMGT |
| | | IASFLSKYQDIIIEHQKVVKGNQKRLESLRELAGKENLEYPSVTLPPQPHTKEGVDAYN |
| | | EVIARVRMWVNLNLWQKLKLSRDDAKPLLRLKKFPSFPLVERQANEVDWWDMVCNVKKL |
| | | INEKKEDGKVFWQNLAGYKRQEALRPYLSSEEDRKKGKKFARYQLGDLLLHLEKKHGED |
| | | WGKVYDEAWERIDKKVEGLSKHIKLEEERRSEDAQSKAALTDWLRAKASFVIEGLKEAD |
| | | KDEFCRCELKLQKWYGDLRGKPFAIEAENSILDISGFSKQYNCAFIWQKDGVKKLNLYL |
| | | IINYFKGGKLRFKKIKPEAFEANRFYTVINKKSGEIVPMEVNFNFDDPNLIILPLAFGK |
| | | RQGREFIWNDLLSLETGSLKLANGRVIEKTLYNRRTRQDEPALFVALTFERREVLDSSN |
| | | IKPMNLIGVARGENIPAVIALTDPEGCPLSRFKDSLGNPTHILRIGESYKEKQRTIQAK |
| | | KEVEQRRAGGYSRKYASKAKNLADDMVRNTARDLLYYAVTQDAMLIFANLSRGFGRQGK |
| | | RTFMAERQYTRMEDWLTAKLAYEGLPSKTYLSKTLAQYTSKTCSNCGFTITSADYDRVL |
| | | EKLKKTATGWMTTINGKELKVEGQITYYNRYKRQNVVKDLSVELDRLSEESVNNDISSW |
| | | TKGRSGEALSLLKKRFSHRPVQEKFVCLNCGFETHAAEQAALNIARSWLFLRSQEYKKY |
| | | QTNKTTGNTDKRAFVETWQSFYRKKLKEVWKPAV |

d. Affinity for the gRNA

In some embodiments, a long-term repressor fusion protein comprising a dCasX with linked repressor domains has improved affinity for the gRNA relative to a long-term repressor fusion protein comprising a reference dCasX protein with corresponding linked repressor domains, leading to the formation of the ribonucleoprotein complex (RNP). Increased affinity of the long-term repressor fusion protein for the gRNA may, for example, result in a lower K_{d} for the generation of a RNP complex, which can, in some cases, result in a more stable ribonucleoprotein complex formation. In some embodiments, the K_{d} of a long-term repressor fusion protein for a gRNA is increased relative to a reference dCasX protein and linked repressor domains by a factor of at least about 1. 1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the long-term repressor fusion protein comprising the dCasX variant and linked repressor domains has about 1.1 to about 10-fold increased binding affinity to the gRNA compared to the corresponding repressor fusion protein comprising the catalytically-dead variant of the reference CasX protein of SEQ ID NO: 2.

In some embodiments, increased affinity of the long-term repressor fusion protein for the gRNA results in increased stability of the ribonucleoprotein complex when delivered to mammalian cells, including in vivo delivery to a subject. This increased stability can affect the function and utility of the complex in the cells of a subject, as well as result in improved pharmacokinetic properties in blood, when delivered to a subject. In some embodiments, increased affinity of the repressor fusion protein, and the resulting increased stability of the ribonucleoprotein complex, allows for a lower dose of the long-term repressor fusion protein to be delivered to the subject or cells while still having the desired activity; for example in vivo or in vitro gene repression and/or epigenetic modification. The increased ability to form RNP and keep them in stable form can be assessed using in vitro assays known in the art.

In some embodiments, a higher affinity (tighter binding) of a long-term repressor fusion protein comprising a dCasX variant protein and linked repressor domains to a gRNA allows for a greater amount of transcriptional repression and/or epigenetic modification events when both the long-term repressor fusion protein and the gRNA remain in an RNP complex. Increased transcriptional repression events can be assessed using assays described herein.

Methods of measuring long-term repressor fusion protein binding affinity for a gRNA include in vitro methods using a purified long-term repressor fusion protein and a gRNA. The binding affinity for a long-term repressor fusion protein can be measured by fluorescence polarization if the gRNA or long-term repressor fusion protein is tagged with a fluorophore. Alternatively, or in addition, binding affinity can be measured by biolayer interferometry, electrophoretic mobility shift assays (EMSAs), or filter binding. Additional standard techniques to quantify absolute binding affinities of the repressor fusion proteins of the disclosure for specific gRNAs include, but are not limited to, isothermal calorimetry (ITC), and surface plasmon resonance (SPR).

e. Improved Specificity for a Target Nucleic Acid Sequence

In some embodiments, a long-term repressor fusion protein comprising a dCasX variant protein with linked repressor domains has improved specificity for a target nucleic acid sequence that is complementary to the targeting sequence of the gRNA relative to a reference dCasX protein with linked repressor domains. As used herein, “specificity,” sometimes referred to as “target specificity,” refers to the degree to which an RNP complex binds off-target sequences that are similar, but not identical to the target nucleic acid sequence; e.g., a long-term repressor fusion protein RNP with a higher degree of specificity would exhibit reduced off-target methylation of sequences relative to an RNP of a reference dCasX with linked repressor domains. The specificity, and the reduction of potentially deleterious off-target effects, of long-term repressor fusion proteins can be important in order to achieve an acceptable therapeutic index for use in mammalian subjects. Without wishing to be bound by theory, it is possible that amino acid changes in the helical I and II domains that increase the specificity of the dCasX for the target nucleic acid strand, and can thereby increase the specificity of the long-term repressor fusion protein for the target nucleic acid overall. In some embodiments, amino acid changes that increase specificity of repressor fusion proteins for target nucleic acid may also result in decreased affinity of repressor fusion proteins for DNA, but the overall benefit and safety of the composition is enhanced.

f. Repressor fusion proteins with heterologous proteins

Also contemplated within the scope of the disclosure are repressor fusion proteins comprising a heterologous protein fused to the long-term repressor fusion protein for use in the systems of the disclosure. This includes repressor fusion proteins comprising N-terminal and/or C-terminal fusions to a heterologous protein or a domain thereof. In some embodiments, the long-term repressor fusion protein is fused to one or more proteins or domains thereof that has a different activity of interest.

In some cases, a heterologous polypeptide (a fusion partner) for use with a long-term repressor fusion protein provides for subcellular localization, i.e., the heterologous polypeptide contains a subcellular localization sequence (e.g., a nuclear localization signal (NLS) for targeting to the nucleus, a sequence to keep the fusion protein out of the nucleus, a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like).

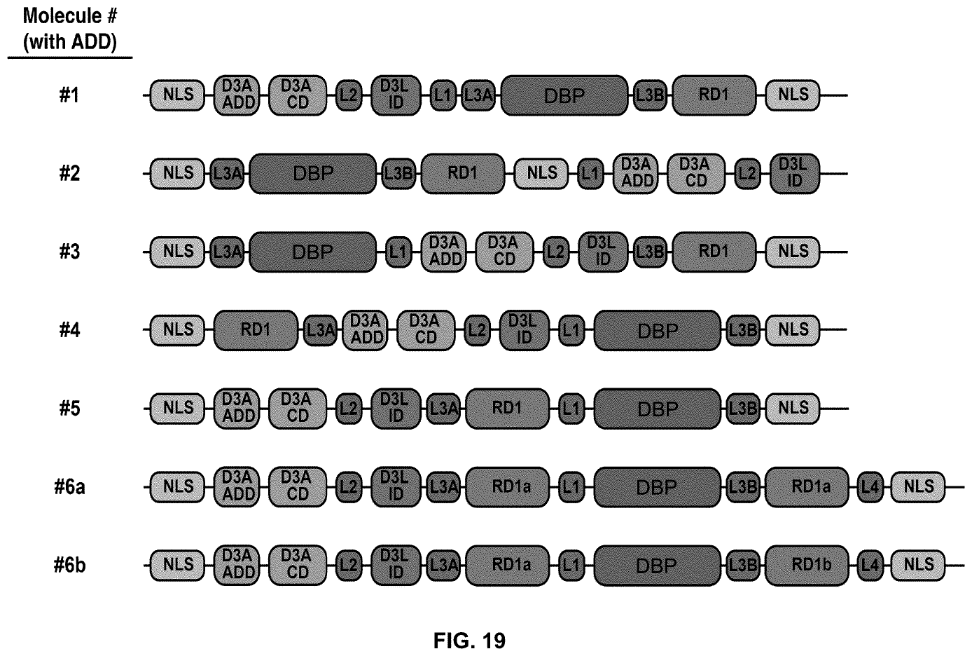

In some cases, a long-term repressor fusion protein includes (is fused to) a nuclear localization signal (NLS). In some cases, a long-term repressor fusion protein is fused to 2 or more, 3 or more, 4 or more, or 5 or more 6 or more, 7 or more, 8 or more NLSs. In some cases, one or more NLSs (2 or more, 3 or more, 4 or more, or 5 or more NLSs) are positioned at or near (e.g., within 20 amino acids of) the N-terminus and/or the C-terminus of the repressor fusion protein. In some cases, one or more NLSs (2 or more, 3 or more, 4 or more, or 5 or more NLSs) are positioned at or near (e.g., within 20 amino acids of) the N-terminus of the repressor fusion protein. In some cases, one or more NLSs (2 or more, 3 or more, 4 or more, or 5 or more NLSs) are positioned at or near (e.g., within 20 amino acids of) the C-terminus of the repressor fusion protein. In some cases, one or more NLSs (3 or more, 4 or more, or 5 or more NLSs) are positioned at or near (e.g., within 20 amino acids of) both the N-terminus and the C-terminus of the repressor fusion protein. In some cases, a single NLS is positioned at the N-terminus and a single NLS is positioned at the C-terminus of the repressor fusion protein. The person of ordinary skill in the art will understand that an NLS at or near the N- or C-terminus of a protein can be within 1, 2, 3, 4, 5, 6, 7, 8, 9, 10, 11, 12, 13, 14, 15, 16, 17, 18, 19 or 20 amino acids of the N- or C-terminus. In some embodiments, the NLS linked to the N-terminus of the long-term repressor fusion protein are identical to the NLS linked to the C-terminus. In other embodiments, the NLS linked to the N-terminus of the long-term repressor fusion protein are different to the NLS linked to the C-terminus. Representative configurations of repressor fusion proteins with NLS are shown in FIG. 19. In some embodiments, NLSs suitable for use with a long-term repressor fusion protein in the systems of the disclosure comprise sequences having at least about 85%, at least about 90%, or at least about 95% identity or are identical to sequences derived from: the NLS of the simian virus 40 (SV40) virus large T-antigen, having the amino acid sequence PKKKRKV (SEQ ID NO: 30); the NLS from nucleoplasmin (e.g., the nucleoplasmin bipartite NLS with the sequence KRPAATKKAGQAKKKK (SEQ ID NO: 31); the c-MYC NLS having the amino acid sequence PAAKRVKLD (SEQ ID NO: 32) or RQRRNELKRSP (SEQ ID NO: 33). In some embodiments, the NLS and a short peptide linker linked to the N-terminus of the long-term repressor fusion protein is the sequence PKKKRKVSR (SEQ ID NO: 34). In some embodiments, the NLS and a short peptide linker linked to the N-terminus of the long-term repressor fusion protein is the sequence PKKKRKVSRVNGSGSGGG (SEQ ID NO: 21840). In some embodiments, the NLS and a short peptide linker linked to the C-terminus of the long-term repressor fusion protein is the sequence TSPKKKRKV (SEQ ID NO: 21841). In some embodiments, the NLS linked to, or proximal to the N-terminus of the long-term repressor fusion protein is selected from the group consisting of SEQ ID NOS: 34-67. In some embodiments, the NLS linked to, or proximal to the C-terminus of the long-term repressor fusion protein is selected from the group consisting of SEQ ID NOS: 68-97. In some embodiments, NLSs suitable for use with a long-term repressor fusion protein in the systems of the disclosure include sequences having at least about 8000, at least about 9000, or at least about 9500 identity or are identical to one or more sequences of Table 3 or Table 4. The person of ordinary skill in the art will appreciate that any of the NLS sequences set forth in Table 3 and Table 4 can be fused to, or proximal to either the N- or C-terminus of the repressor fusion proteins described herein.

TABLE 3
N-terminal NLS Amino Acid Sequences

| | SEQ |
|---|---|
| | ID |
| NLS Amino Acid Sequence* | NO |
| PKKKRKVSR | 34 |
| PKKKRKVGGSPKKKRKVGGSPKKKRKVGGSPKKKRKVSR | 35 |
| PKKKRKVGGSPKKKRKVGGSPKKKRKVGGSPKKKRKVGGS | 36 |
| PKKKRKVGGSPKKKRKVSR | |
| PAAKRVKLDSR | 37 |
| PAAKRVKLDGGSPAAKRVKLDSR | 38 |
| PAAKRVKLDGGSPAAKRVKLDGGSPAAKRVKLDGGSPAAK | 39 |
| RVKLDSR | |
| PAAKRVKLDGGSPAAKRVKLDGGSPAAKRVKLDGGSPAAK | 40 |
| RVKLDGGSPAAKRVKLDGGSPAAKRVKLDSR | |
| KRPAATKKAGQAKKKKSR | 41 |
| KRPAATKKAGQAKKKKGGSKRPAATKKAGQAKKKKSR | 42 |
| PAAKRVKLDGGSPKKKRKVSR | 43 |
| PAAKKKKLDGGSPKKKRKVSR | 44 |
| PAAKKKKLDSR | 45 |
| PAAKKKKLDGGSPAAKKKKLDGGSPAAKKKKLDSR | 46 |
| PAAKKKKLDGGSPAAKKKKLDGGSPAAKKKKLDGGSPAAK | 47 |
| KKKLDSR | |
| PAKRARRGYKCSR | 48 |
| PAKRARRGYKCGSPAKRARRGYKCSR | 49 |
| PRRKREESR | 50 |
| PYRGRKESR | 51 |
| PLRKRPRRSR | 52 |
| PLRKRPRRGSPLRKRPRRSR | 53 |
| PAAKRVKLDGGKRTADGSEFESPKKKRKVGGS | 54 |
| PAAKRVKLDGGKRTADGSEFESPKKKRKVPPPPG | 55 |
| PAAKRVKLDGGKRTADGSEFESPKKKRKVGIHGVPAAPG | 56 |
| PAAKRVKLDGGKRTADGSEFESPKKKRKVGGGSGGGSPG | 57 |
| PAAKRVKLDGGKRTADGSEFESPKKKRKVPGGGSGGGSPG | 58 |
| PAAKRVKLDGGKRTADGSEFESPKKKRKVAEAAAKEAAAK | 59 |
| EAAAKAPG | |
| PAAKRVKLDGGSPKKKRKVGGS | 60 |
| PAAKRVKLDPPPPKKKRKVPG | 61 |
| PAAKRVKLDPG | 62 |
| PAAKRVKLDGGGSGGGSGGGS | 63 |
| PAAKRVKLDPPP | 64 |
| PAAKRVKLDGGGSGGGSGGGSPPP | 65 |
| PKKKRKVPPP | 66 |
| PKKKRKVGGS | 67 |

*Residues in bold are NLS residues, while unbolded residues are linkers.

TABLE 4
C-terminal NLS Amino Acid Sequences

| | SEQ |
|---|---|
| | ID |
| NLS Amino Acid Sequence | NO |
| GSPKKKRKVGGSPKKKRKVGGSPKKKRKVGGSPKKKRKV | 68 |
| GSPKKKRKVGGSPKKKRKVGGSPKKKRKVGGSPKKKRKV | 69 |
| GGSPKKKRKVGGSPKKKRKV | |
| GSPAAKRVKLDGGSPAAKRVKLD | 70 |
| GSPAAKRVKLDGGSPAAKRVKLDGGSPAAKRVKLDGGSP | 71 |
| AAKRVKLD | |
| GSKRPAATKKAGQAKKKK | 72 |
| KRPAATKKAGQAKKKKGGSKRPAATKKAGQAKKKK | 73 |
| GSKLGPRKATGRWGS | 74 |
| GSKRKGSPERGERKRHWGS | 75 |
| GSPKKKRKVGSGSKRPAATKKAGQAKKKKLE | 76 |
| GPKRTADSQHSTPPKTKRKVEFEPKKKRKV | 77 |
| GGGSGGGSKRTADSQHSTPPKTKRKVEFEPKKKRKV | 78 |
| AEAAAKEAAAKEAAAKAKRTADSQHSTPPKTKRKVE | 79 |
| FEPKKKRKV | |
| GPPKKKRKVGGSKRTADSQHSTPPKTKRKVEFEPKK | 80 |
| KRKV | |
| GPAEAAAKEAAAKEAAAKAPAAKRVKLD | 81 |
| GPGGGSGGGSGGGSPAAKRVKLD | 82 |
| GPPAAKRVKLD | 83 |
| VGSKRPAATKKAGQAKKKK | 84 |
| TGGGPGGGAAAGSGSPKKKRKVGSGSKRPAATKKAG | 85 |
| QAKKKKLE | |
| TGGGPGGGAAAGSGSPKKKRKVGSGS | 86 |
| PPPPKKKRKVPPP | 87 |
| GGSPKKKRKVPPP | 88 |
| PPPPKKKRKV | 89 |
| GGSPKKKRKV | 90 |
| GGSPKKKRKVGGSGGSGGS | 91 |
| GGSPKKKRKVGGSPKKKRKV | 92 |
| GGSGGSGGSPKKKRKVGGSPKKKRKV | 93 |
| VGGGSGGGSGGGSPAAKRVKLD | 94 |
| VPPPPAAKRVKLD | 95 |
| VPPPGGGSGGGSGGGSPAAKRVKLD | 96 |
| VGSPAAKRVKLD | 97 |

In some embodiments, the one or more NLSs are linked to the long-term repressor fusion protein or to adjacent NLS with a linker peptide. In some embodiments, the linker peptide is selected from the group consisting of SR, GS, GP, TS, VGS, GGS, (G)n (SEQ ID NO: 98), (GS)n (SEQ ID NO: 99), (GSGGS)n (SEQ ID NO: 100), (GGSGGS)n (SEQ ID NO: 101), (GGGS)n (SEQ ID NO: 102), GGSG (SEQ ID NO: 103), GGSGG (SEQ ID NO: 104), GSGSG (SEQ ID NO: 105), GSGGG (SEQ ID NO: 106), GGGSG (SEQ ID NO: 107), GSSSG (SEQ ID NO: 108), GPGP (SEQ ID NO: 109), GGP, PPP, VPPP, PPAPPA (SEQ ID NO: 110), PPPG (SEQ ID NO: 111), PPPGPPP (SEQ ID NO: 112), PPP(GGGS)n (SEQ ID NO: 113), (GGGS)nPPP (SEQ ID NO: 114), AEAAAKEAAAKEAAAKA (SEQ ID NO: 115), VPPPGGGSGGGSGGGS (SEQ ID NO: 116), TGGGPGGGAAAGSGS (SEQ ID NO: 117), GGGSGGGSGGGSPPP (SEQ ID NO: 118), TPPKTKRKVEFE (SEQ ID NO: 119), GGSGGGS (SEQ ID NO: 120), GSGSGGG (SEQ ID NO: 121), SSGNSNANSRGPSFSSGLVPLSLRGSH (SEQ ID NO: 122), GGPSSGAPPPSGGSPAGSPTSTEEGTSESATPESGPGTSTEPSEGSAPGSPAGSPTSTEEGTS TEPSEGSAPGTSTEPSE (SEQ ID NO: 123), GGSGGG (SEQ ID NO: 124), GSGS (SEQ ID NO: 1988), GSGSGSG (SEQ ID NO: 2130), GGSGGGSA (SEQ ID NO: 2131), where n is 1 to 5.

In general, NLS (or multiple NLSs) are of sufficient strength to drive accumulation of a long-term repressor fusion protein in the nucleus of a eukaryotic cell. Detection of accumulation in the nucleus may be performed by any suitable technique known in the art. For example, a detectable marker may be fused to a long-term repressor fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

### IV. Long-Term Repressor Domain Fusion Proteins

The disclosure provides systems comprising a long-term repressor fusion proteins (LTRP) comprising a DNA binding protein linked to multiple repressor domains in designed configurations, wherein the system is capable of binding to a target nucleic acid of a gene and repressing transcription, including by epigenetic modification of the target nucleic acid. Exemplary DNA binding proteins for use in the fusion proteins include zinc finger (ZF), TALE (transcription-activator-like effector) proteins, and catalytically-dead CRISPR proteins.

In some embodiments, the disclosure provides systems of repressor fusion proteins comprising a catalytically-dead CasX variant protein (dCasX) linked to multiple repressor domains that, when complexed with a guide ribonucleic acid (gRNA) comprising a targeting sequence complementary to a target nucleic acid sequence of a gene, is capable of binding to the target nucleic acid and repressing or silencing transcription and/or affecting epigenetic modification of the target nucleic acid. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Transcriptional repression includes both reversible and irreversible inactivation of gene transcription; the latter can result from epigenetic modification of the target nucleic acid.

Amongst repressor domains that have the ability to repress, or silence genes, the Kruppel-associated box (KRAB) repressor domain is amongst the most powerful in human genome systems (Alerasool, N., et al. An efficient KRAB domain for CRISPRi applications. Nat. Methods 17:1093 (2020)). KRAB-like domains are present in approximately 400 human zinc finger protein-based transcription factors that upon binding of the linked dCasX to the target nucleic acid, is capable of recruiting additional repressor domains such as, but not limited to, Trim28 (also known as Kap1 or Tifl-beta) that, in turn, assembles a protein complex with chromatin regulators such as CBX5/HP1ca and SETDB1 that induce repression of transcription of the gene, but do so in a limited, temporal fashion, including modification of the histones associated with DNA rather than modification of the DNA. By decreasing histone H3-acetylation and increasing H3 lysine 9 trimethylation at the cell level, KRAB/KAP1 mediates reversible and long-range transcriptional repression through heterochromatin spreading. Representative, non-limiting examples of KRAB domains include ZIM3 (SEQ ID NOS: 129 and 1892) and ZNF10 (SEQ ID NOS: 128 and 1891). The present disclosure provides repressor domains that are from human sources, as well as repressor domains that are from non-human sources with markedly distinct sequences (referred to herein as “RD1”) that have been found to result in enhanced transcriptional repression compared to ZIM3 and ZNF10 when incorporated in long-term repressor fusion protein construct embodiments, described more fully, below.

In some embodiments, the disclosure provides systems in which the modification to a gene imparted by use of the LTRP:gRNA system is epigenetic, and hence the silencing of a gene is heritable by mechanisms other than by DNA editing replication. As used herein “epigenetic modification” means a modification to either DNA or histones associated with DNA, other than a change in the DNA sequence itself (e.g., a substitution, deletion or rearrangement), wherein the modification is either a direct modification by a component of the system or is indirect by the recruitment of one or more additional cellular components, but in which the DNA target nucleic acid sequence itself is not edited to change the sequence. For example, DNA methyltransferase 3A (DNMT3A) (or its catalytic domain) directly modifies the DNA by methylating it, whereas KRAB recruits KAP-1/TIF1β corepressor complexes that act as potent transcriptional repressors and can further recruit factors associated with DNA methylation and formation of repressive chromatin, such as heterochromatin protein 1 (HP1), histone deacetylases and histone methyltransferases (Ying, Y., et al. The Kruppel-associated box repressor domain induces reversible and irreversible regulation of endogenous mouse genes by mediating different chromatin states. Nucleic Acids Res. 43(3): 1549 (2015)). Further, the catalytically inactive DNMT3 like (DNMT3L) cofactor helps establish a heritable methylation pattern after DNA replication, together with endogenous DNMT1 of the cell. The ATRX-DNMT3-DNMT3L (ADD) domain of DNMT3A is known to have two key functions: 1) it allosterically regulates the catalytic activity of DNMT3A by serving as a methyltransferase auto-inhibitory domain, and 2) it specifically interacts with histone H3 tails that are unmethylated at lysine (K) 4 (H3K4me0), leading to the preferential methylation of DNA bound to chromatin H3 tails that are unmethylated at K4 (Zhang, Y., et al. Chromatin methylation activity of Dnmt3a and Dnmt3a/3L is guided by interaction of the ADD domain with the histone H3 tail. Nucleic Acids Research 38:4246 (2010)). In some embodiments, the inclusion of the ADD domain enhances transcriptional repression of targeted genes when incorporated into designs of LTRP, when compared to otherwise equivalent LTRP lacking the ADD domain. In other embodiments, the inclusion of the ADD domain enhances the specificity of the transcriptional repression of targeted genes when incorporated into designs of LTRP, when compared to otherwise equivalent LTRP lacking the ADD domain. Supporting data for the foregoing are provided in the Examples, as well as in WO2023049742A2, incorporated by reference herein.

In some embodiments, the long-term repressor fusion protein comprises a DNA binding protein linked to a first, second, and third repressor domain, wherein each of the repressor domains are different. In some embodiments, the long-term repressor fusion protein comprises a DNA binding protein linked to a first, second, third, and fourth repressor domain, wherein each of the repressor domains are different. In any of the foregoing embodiments, the long-term fusion protein is capable of forming an RNP with a gRNA of the system that binds to the target nucleic acid.

In some embodiments, the DNA binding protein is a TALE that can bind but not cleave the target nucleic acid. In some embodiments, the DNA binding protein is a zinc-finger protein modified to bind but not cleave the target nucleic acid. In some embodiments, the DNA binding protein is a catalytically dead CRISPR protein that can bind but not cleave the target nucleic acid. In some embodiments, the long-term repressor fusion protein comprises a catalytically-dead CRISPR protein sequence, a first repressor domain (hereinafter referred to as “RD1”), a DNMT3A catalytic domain (herein after referred to as “DNMT3A”) from the DNMT3A protein as the second domain, and a DNMT3L interaction domain (herein after referred to as “DNMT3L”) from the DNMT3L protein as the third domain. In some embodiments, the long-term repressor fusion protein comprises a catalytically-dead CRISPR protein sequence, an RD1, a DNMT3A as the second domain, a DNMT3L as the third domain, and an ATRX-DNMT3-DNMT3L domain (herein after referred to as “ADD”) from the DNMT3A protein as the fourth domain. In some embodiments, the long-term repressor fusion protein further comprises a first and a second NLS and one or more linker peptides described herein. In some embodiments, the long-term repressor fusion protein is capable of forming an RNP with a gRNA that binds to the target nucleic acid. It has been discovered that the use of the foregoing domains, when configured in select orientations relative to the DNA binding protein in a long-term repressor fusion protein, can result in pronounced epigenetic modification of a target nucleic acid when bound to defined regions of a gene to be silenced, and that the combination of the repressor domains work in synchrony, resulting in an additive or synergistic effect on transcriptional silencing of the targeted gene, depending on the configuration.

Representative amino acid sequences of the components utilized in the long-term repressor fusion protein constructs are provided herein. In some embodiments, the DNA binding protein of the long-term repressor fusion protein is a dCasX comprising a sequence selected from the group consisting of SEQ ID NOS: 4-29, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the DNA binding protein of the long-term repressor fusion protein is a dCasX comprising a sequence of SEQ ID NO: 4, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the DNA binding protein of the long-term repressor fusion protein is a dCasX comprising a sequence of SEQ ID NO: 4. In some embodiments, the DNA binding protein of the long-term repressor fusion protein is a dCasX comprising a sequence of SEQ ID NO: 5, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the DNA binding protein of the long-term repressor fusion protein is a dCasX comprising a sequence of SEQ ID NO: 28, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the DNA binding protein of the long-term repressor fusion protein is a dCasX comprising a sequence of SEQ ID NO: 29, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto.

In some embodiments, the RD1 of the long-term repressor fusion protein comprises a sequence selected from the group consisting of SEQ ID NO: 1891 and SEQ ID NO: 1892, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the RD1 of the long-term repressor fusion protein comprises a sequence selected from the group consisting of SEQ ID NO: 1891 and SEQ ID NO: 1892. In another embodiment, the RD1 of the long-term repressor fusion protein comprises a sequence selected from the group consisting of SEQ ID NOS: 130-1726, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence selected from the group consisting of SEQ ID NOS: 130-1726. In another embodiment, the RD1 of the long-term repressor fusion protein comprises a sequence selected from the group consisting of SEQ ID NOS: 130-224 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence selected from the group consisting of SEQ ID NOS: 130-224. In another embodiment, the RD1 of the long-term repressor fusion protein comprises a sequence selected from the group consisting of SEQ ID NOS: 130-138 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence selected from the group consisting of SEQ ID NOS: 130-138. In another embodiment, the RD1 of the long-term repressor fusion protein comprises a sequence of SEQ ID NOS: 135 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence of SEQ ID NOS: 131 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence of SEQ ID NOS: 130 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the RD1 of the long-term repressor fusion protein comprises a sequence selected from the group consisting of SEQ ID NOS: 130, 131 and 135.

In some embodiments, the second repressor domain of the long-term repressor fusion protein is a DNMT3A, comprising a sequence of SEQ ID NO: 126, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the second repressor domain of the long-term repressor fusion protein comprises a sequence of SEQ ID NO: 126.

In some embodiments, the third repressor domain of the long-term repressor fusion protein is a DNMT3L, comprising a sequence of SEQ ID NO: 127, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the third repressor domain of the long-term repressor fusion protein comprises a sequence of SEQ ID NO: 127.

In some embodiments, the fourth repressor domain of the long-term repressor fusion protein is an ADD, comprising a sequence of SEQ ID NO: 125, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the fourth repressor domain of the long-term repressor fusion protein comprises a sequence of SEQ ID NO: 125. In some embodiments, the C terminal of the ADD is linked to the N-terminal of the DNMT3A. In a surprising finding, it has been discovered that the addition of the ADD to the constructs comprising the DNA binding protein, RD1, DNMT3A, and DNMT3L greatly enhances or increases the long-term repression and/or epigenetic modification of the target nucleic acid, as well as the specificity of the repression, in comparison to constructs lacking the ADD. Exemplary data for the improved transcriptional repression and specificity of constructs comprising the ADD are presented in the Examples, and is described in WO2023049742A2, incorporated by reference herein.

The present disclosure provides a system comprising a long-term repressor fusion protein comprising a first, a second, a third, and, optionally, a fourth repressor domain operably linked to a DNA binding protein, wherein the DNA binding protein is a dCasX comprising the sequence of SEQ ID NO: 4, or a sequence having at least about 70%, at least about 75%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the second repressor domain is a DNMT3A domain comprising the sequence of SEQ ID NO: 126, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the third repressor is a DNMT3L domain comprising the sequence of SEQ ID NO: 127, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, and the fourth repressor is an ADD domain comprising the sequence of SEQ ID NO: 125, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments, the long-term repressor fusion protein comprises a first, a second, a third, and, optionally, a fourth repressor domain operably linked to a DNA binding protein, wherein the DNA binding protein is a dCasX comprising the sequence selected from the group consisting of SEQ ID NOS: 4-29, the second repressor domain is a DNMT3A domain comprising the sequence of SEQ ID NO: 126, the third repressor is a DNMT3L domain comprising the sequence of SEQ ID NO: 127, and the fourth repressor is an ADD domain comprising the sequence of SEQ ID NO: 125. In some embodiments, the long-term repression fusion protein comprises one or more linker peptides selected from the group consisting of SEQ ID NOS: 98-124, 1823-1874, 1988, and 2130-2131 (Table 5). In some embodiments, the long-term repression fusion protein comprises one or more NLS comprising a sequence selected from the group consisting of SEQ ID NOS: 30, 32, 34, and 21841. In some embodiments, the first RD1 comprises a sequence selected from the group consisting of SEQ ID NOS: 130-1726, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments, the first RD1 comprises a sequence selected from the group consisting of SEQ ID NOS: 130-1726. In other embodiments of the long-term repressor protein, the first RD1 comprises a sequence selected from the group consisting of SEQ ID NOS: 130-224, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In other embodiments of the long-term repressor protein, the first RD1 comprises a sequence selected from the group consisting of SEQ ID NOS: 130-224. In other embodiments of the long-term repressor protein, the first RD1 comprises a sequence selected from the group consisting of SEQ ID NOS: 130-138, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In other embodiments of the long-term repressor protein, the first RD1 comprises a sequence selected from the group consisting of SEQ ID NOS: 130-138. In other embodiments of the long-term repressor protein, the first RD1 comprises the sequence of SEQ ID NO: 135, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In other embodiments of the long-term repressor protein, the first RD1 comprises the sequence of SEQ ID NO: 135. In other embodiments of the long-term repressor protein, the first RD1 comprises the sequence of SEQ ID NO: 131, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In other embodiments of the long-term repressor protein, the first RD1 comprises the sequence of SEQ ID NO: 131. In other embodiments of the long-term repressor protein, the first RD1 comprises the sequence of SEQ ID NO: 130, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In other embodiments of the long-term repressor protein, the first RD1 comprises the sequence of SEQ ID NO: 130. In other embodiments of the long-term repressor protein, the first RD1 comprises a sequence selected from the group consisting of SEQ ID NOS: 130, 131 and 135. In some embodiments, a long-term repressor protein embodiment of the paragraph is capable of forming an RNP with a gRNA of the disclosure that binds to and represses or silences the expression of a targeted gene.

In some embodiments, the long-term repressor fusion protein comprises, from N- to C-terminus, a DNMT3A, a DNMT3L, a DNA binding protein, and an RD1. In some embodiments, the long-term repressor fusion protein comprises, from N- to C-terminus, an ADD, a DNMT3A, a DNMT3L, a DNA binding protein, and an RD1. In some embodiments, the DNA binding protein may be a zinc finger, a TALE, or a catalytically-dead CRISPR protein. In some embodiments the long-term repressor fusion protein comprises an NLS at the N terminus, the C terminus, or both. In some embodiments, the long-term repressor fusion protein comprises one or more linkers between the DNMT3A and DNMT3L, between the DNMT3L and DNA binding protein, and/or between the DNA binding protein and the RD1.

In some embodiments, the long-term repressor fusion protein comprises, from N- to C-terminus, a DNA binding protein, an RD1, a DNMT3A, and a DNMT3L. In some embodiments, the long-term repressor fusion protein comprises, from N- to C-terminus, a DNA binding protein, an RD1, and ADD, a DNMT3A, and a DNMT3L. In some embodiments, the DNA binding protein may be a zinc finger, a TALE, or a catalytically-dead CRISPR protein. In some embodiments the long-term repressor fusion protein comprises an NLS at the N terminus. In some embodiments the long-term repressor fusion protein comprises an NLS between the RD1 and the DNMT3A. In some embodiments the long-term repressor fusion protein comprises an NLS at the N terminus, the C terminus, or both. In some embodiments, the long-term repressor fusion protein comprises one or more linkers between an N terminal NLS and the DNA binding protein, between the DNA binding protein and the RD1, between the RD1 and the DNMT3A, or optionally, the ADD, and/or between the DNMT3A and the DNMT3A.

In some embodiments, the long-term repressor fusion protein comprises, from N- to C-terminus, a DNA binding protein, a DNMT3A, a DNMT3L, and an RD1. In some embodiments, the long-term repressor fusion protein comprises, from N- to C-terminus, a DNA binding protein, an ADD, a DNMT3A, a DNMT3L, and an RD1. In some embodiments, the DNA binding protein may be a zinc finger, a TALE, or a catalytically-dead CRISPR protein. In some embodiments the long-term repressor fusion protein comprises an NLS at the N terminus, the C terminus, or both. In some embodiments, the long-term repressor fusion protein comprises one or more linkers between an N terminal NLS and the DNA binding protein, between the DNA binding protein and the DNMT3A, or optionally the ADD, between the DNT3A and the DNMT3L, and/or between the DNMT3L and the RD1.

In some embodiments, the long-term repressor fusion protein comprises, from N- to C-terminus, an RD1, a DNMT3A, a DNMT3L, and a DNA binding protein. In some embodiments, the long-term repressor fusion protein comprises, from N- to C-terminus, an RD1, an ADD, a DNMT3A, a DNMT3L, and a DNA binding protein. In some embodiments, the DNA binding protein may be a zinc finger, a TALE, or a catalytically-dead CRISPR protein. In some embodiments the long-term repressor fusion protein comprises an NLS at the N terminus, the C terminus, or both. In some embodiments, the long-term repressor fusion protein comprises one or more linkers between the RD1 and the DNMT3A, or optionally the ADD, between the DNMT3A and the DNMT3L, between the DNMT3L and the DNA binding protein, and/or between the DNA binding protein and a C terminal NLS.

In some embodiments, the long-term repressor fusion protein comprises, from N- to C-terminus, a DNMT3A, a DNMT3L, an RD1, and a DNA binding protein. In some embodiments, the long-term repressor fusion protein comprises, from N- to C-terminus, an ADD, a DNMT3A, a DNMT3L, an RD1, and a DNA binding protein. In some embodiments, the DNA binding protein may be a zinc finger, a TALE, or a catalytically-dead CRISPR protein. In some embodiments the long-term repressor fusion protein comprises an NLS at the N terminus, the C terminus, or both. In some embodiments, the long-term repressor fusion protein comprises one or more linkers between the DNMT3A and the DNMT3L, between the DNTM3L and the RD1, between the RD1 and the DNA binding protein, and/or between the DNA binding protein and a C terminal NLS.

In some embodiments, the long-term repressor fusion protein comprises, from N- to C-terminus, a DNMT3A, a DNMT3L, an RD1, a DNA binding protein, and a second RD1. In some embodiments, the long-term repressor fusion protein comprises, from N- to C-terminus, an ADD, a DNMT3A, a DNMT3L, an RD1, a DNA binding protein. and a second RD1. In one embodiment of the foregoing, the second RD1 may be identical in sequence to the first RD1. In another embodiment of the foregoing, the second RD1 may be different in sequence to the first RD1. In some embodiments, the DNA binding protein may be a zinc finger, a TALE, or a catalytically-dead CRISPR protein. In some embodiments the long-term repressor fusion protein comprises an NLS at the N terminus, the C terminus, or both. In some embodiments, the long-term repressor fusion protein comprises one or more linkers between the DNMT3A and DNMT3L, between the DNMT3L and the RD1, between he RD1 and the DNA binding protein, between the DNA binding protein and the second RD1, and/or between the second RD1 and a C terminal NLS.

In some cases, the long-term repressor fusion protein also comprises one or more NLS. In some embodiments, the long-term repressor fusion protein comprises a configuration of, N-terminal to C-terminal of NLS-ADD-DNMT3A-DNMT3L-DNA binding protein-RD1-NLS, NLS-DNA binding protein-RD1-NLS-ADD-DNMT3A-DNMT3L, NLS- DNA binding protein-ADD-DNMT3A-DNMT3L- RD1-NLS), NLS-RD1-ADD-DNMT3A-DNMT3L-DNA binding protein-NLS, NLS-ADD-DNMT3A-DNMT3L-RD1-DNA binding protein-NLS, or NLS-ADD-DNMT3A-DNMT3L-RD1-DNA binding protein-RD1-NLS. In some embodiments, the DNA binding protein may be a zinc finger, a TALE, or a catalytically-dead CRISPR protein. In some embodiments, the long-term repressor fusion protein is capable of forming an RNP with a gRNA embodiment of the disclosure and is capable of binding to and repressing or silencing the gene target nucleic acid.

In some embodiments, the long-term repressor fusion protein, one or more linker peptides may be inserted between any two adjacent domains of the long-term repressor fusion protein. In some embodiments, the long-term repressor fusion protein comprises a configuration of, N-terminal to C-terminal, of NLS-ADD-DNMT3A-Linker2-DNMT3L-Linker1-Linker3A-DNA binding protein-Linker3B-RD1-NLS (configuration 1). In some embodiments, the long-term repressor fusion protein comprises a configuration of, N-terminal to C-terminal, of NLS-Linker3A-DNA binding protein-Linker3B-RD1-NLS-Linker1-ADD-DNMT3A-Linker2-DNMT3L (configuration 2). In some embodiments, the long-term repressor fusion protein comprises a configuration of, N-terminal to C-terminal, of NLS-Linker3A-DNA binding protein-Linker1-ADD-DNMT3A-Linker2-DNMT3L-Linker3B-RD1-NLS (configuration 3). In some embodiments, the long-term repressor fusion protein comprises a configuration of, N-terminal to C-terminal, of NLS-RD1-Linker3A-ADD-DNMT3A-Linker2-DNMT3L-Linker1-DNA binding protein-Linker3B-NLS (configuration 4). In some embodiments, the long-term repressor fusion protein comprises a configuration of, N-terminal to C-terminal, of NLS-ADD-DNMT3A-Linker2-DNMT3L-Linker3A-RD1-Linker1-DNA binding protein-Linker3B-NLS (configuration 5). In some embodiments, the DNA binding protein of the foregoing configurations may be a zinc finger, a TALE, or a catalytically-dead CRISPR protein. Schematics of the configurations portrayed in FIG. 19. In some embodiments of the LTRP of configurations 1-5, the NLS may comprise a sequence selected from the group consisting of SEQ ID NOS: 30-97 (Tables 3 and 4), the linker sequences may comprise sequences independently selected from the group consisting of SEQ ID NOS: 98-124, 1823-1874, 1988, and 2130-2131 (representative linkers shown in Table 5). In some embodiments of the LTRP, the one or more NLS may comprise a sequence of SEQ ID NO: 30, the one or more linker sequences may independently comprise a sequence selected from the group consisting of SEQ ID NOS: 120, and 122-124. In some embodiments of the LTRP, the DNA binding protein may be a dCasX sequence selected from the group consisting of SEQ ID NOS: 4-29, or a sequence having or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments of the LTRP of configurations 1-5, the second repressor domain is a DNMT3A domain comprising the sequence of SEQ ID NO: 126, or sequence variants having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments of the LTRP, the third repressor is a DNMT3L domain comprising the sequence of SEQ ID NO: 127, or a sequence variant having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments of the LTRP, the fourth repressor is an ADD domain comprising the sequence of SEQ ID NO: 125, or a sequence variant having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto. In some embodiments, the long-term repressor fusion proteins are in one of configurations 1-5, as shown in FIG. 19. In some embodiments, the LTRP are capable of forming an RNP with a gRNA of the disclosure and the RNP is capable of binding to and repressing or silencing the gene target nucleic acid.

In some embodiments, the disclosure provides a system comprising a long-term repressor fusion protein comprising two copies of a first repressor domain (RD1), a second repressor domain, a third repressor domain, and a fourth repressor domain operably linked to a DNA binding protein; e.g., a zinc finger, TALE, or catalytically-dead CRISPR protein. In some embodiments, the DNA binding protein may be a catalytically-dead CasX selected from the group consisting of SEQ ID NOS: 4-29, or sequence having at least 70%, at least about 75%, at least about 80%, at least about 85%, at least about 90% at least about 91%, at least about 92%, at least about 93% at least about 94% at least about 95%, at least about 96%, at least about 97%, at least about 98%, or at least about 99% sequence identity thereto. In some embodiments, the DNA binding protein may be a catalytically-dead CasX sequence of SEQ ID NO: 4. In some embodiments, the sequences of the two copies of the RD1 are identical. In other embodiments, the two RD1 have different sequences. In some embodiments, the two copies of the RD1 are located N-terminal to the DNA binding protein. In some embodiments, the two copies of the RD1 are located C-terminal to the DNA binding protein. In some embodiments, one copy of the RD1 is located N-terminal to the DNA binding protein and one copy of the RD1 is located C-terminal to the DNA binding protein.

In some embodiments, the long-term repressor fusion protein comprises two RD1. In some embodiments, the long-term repressor fusion protein comprises a configuration of, N-terminal to C-terminal, of NLS-ADD-DNMT3A-Linker2-DNMT3L-Linker3A-a RDla-Linker1-DNA binding protein-Linker3B-RDla-Linker 4-NLS (configuration 6a), wherein the RDla sequences are identical (see FIG. 19 as a schematic of the fusion protein). In some embodiments, the long-term repressor fusion protein comprises a configuration of, N-terminal to C-terminal, of NLS-ADD-DNMT3A-Linker2-DNMT3L-Linker3A-a RDla-Linker1-DNA binding protein-Linker3B-RD1b-Linker 4-NLS (configuration 6b), wherein the RDla and RDlb sequences are different (see FIG. 19 as a schematic of the fusion protein). In some embodiments, the DNA binding protein may be a zinc finger, a TALE, or a catalytically-dead CRISPR protein. In some embodiments, a long-term repressor protein embodiment of the paragraph is capable of forming an RNP with a gRNA of the disclosure that binds to and represses or silences the expression of the target nucleic acid.

In some embodiments, the disclosure provides a long-term repressor fusion protein of configuration 6a, wherein the two RD1 sequences are identical. In some embodiments of the configuration 6a long-term repressor fusion protein, wherein the two copies of the RD1 are identical, the DNA binding protein comprises a dCasX of the sequence of SEQ ID NO: 4, or a sequence having at least about 70%, at least about 75%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the first and second copies of the first repressor domain (RDla) comprise a sequence selected from the group consisting of SEQ ID NOS: 130-1726, or a sequence having at least about 70%, at least about 80%, at least about 85%, at least about 90% at least about 91%, at least about 92%, at least about 93% at least about 94% or at least about 95% identity thereto, the second repressor domain is a DNMT3A comprising the sequence of SEQ ID NO: 126, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the third repressor is a DNMT3L comprising the sequence of SEQ ID NOS: 127, or a sequence variant having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the fourth repressor is an ADD comprising the sequence of SEQ ID NO: 125, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the NLSs comprise sequences independently selected from the group consisting of SEQ ID NOS: 30-97 (Tables 3 and 4), the L1 linker comprises a sequence of SEQ ID NO: 123, the L2 linker comprises a sequence of SEQ ID NO: 122, the L3A linker comprises a sequence of SEQ ID NO: 124, the L3B linker comprises a sequence of SEQ ID NO: 120, and the L4 linker comprises a sequence of SEQ ID NO: 1988 or SEQ ID NO: 2130. In some embodiments of the long-term repressor fusion protein of configuration 6a, the linker sequences are independently selected from the group consisting of SEQ ID NOS: 98-124, 1823-1874, 1988, and 2130-2131 (exemplary linkers shown in Table 5). In some embodiments of the long-term repressor fusion protein of configuration 6a, wherein the two copies of the RD1 are identical, each of the RD1 comprise a sequence independently selected from the group consisting of SEQ ID NOS: 130, 131, and 135, or a sequence having at least about 70%, at least about 80%, at least about 85%, at least about 90% at least about 91%, at least about 92%, at least about 93% at least about 94% at least about 95% identity thereto. In some embodiments, the DNA binding protein may be a catalytically-dead CasX sequence of SEQ ID NO: 4. A schematic of configuration 6a is shown in FIG. 19. In some embodiments, the long-term repressor fusion protein of configuration 6a is capable of forming an RNP with a gRNA of the disclosure and is capable of binding to and repressing or silencing the gene target nucleic acid.

In some embodiments of the configuration 6b long-term repressor fusion protein, wherein the two copies of the RD1 are different, the DNA binding protein comprises a dCasX of the sequence of SEQ ID NO: 4, or a sequence having at least about 65%, at least about 70%, at least about 75%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the first copy of the first repressor domain (RDla) comprises a sequence selected from the group consisting of SEQ ID NOS: 130-1726, or a sequence having at least about 70%, at least about 80%, at least about 85%, at least about 90% at least about 91%, at least about 92%, at least about 93% at least about 94% or at least about 95% identity thereto, the second copy of the first repressor domain (RDIb) comprises a sequence selected from the group consisting of SEQ ID NOS: 130-1726, or a sequence having at least about 70%, at least about 80%, at least about 85%, at least about 90% at least about 91%, at least about 92%, at least about 93% at least about 94% or at least about 95% identity thereto, the second repressor domain is a DNMT3A comprising the sequence of SEQ ID NO: 126, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the third repressor is a DNMT3L comprising the sequence of SEQ ID NOS: 127, or a sequence variant having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the fourth repressor is an ADD comprising the sequence of SEQ ID NO: 125, or a sequence having at least about 70%, at least about 80%, at least about 85%, at least about 90% at least about 91%, at least about 92%, at least about 93% at least about 94% at least about 95%, at least about 96%, at least about 97%, at least about 98%, or at least about 99% identity thereto, the NLSs comprise sequences independently selected from the group consisting of SEQ ID NOS: 30-97 of Tables 3 and 4, the L1 linker comprises a sequence of SEQ ID NO: 123, the L2 linker comprises a sequence of SEQ ID NO: 122, the L3A linker comprises a sequence of SEQ ID NO: 124, the L3B linker comprises a sequence of SEQ ID NO: 120, and the L4 linker comprises a sequence of SEQ ID NO: 1988 or SEQ ID NO: 2130. In some embodiments of the long-term repressor fusion protein of configuration 6b, the linker sequences are independently selected from the group consisting of SEQ ID NOS: 98-124, 1823-1874, 1988, and 2130-2131 (exemplary sequences show in Table 5). In some embodiments of the configuration 6b long-term repressor fusion protein, wherein the two copies of the RD1 are different, the RDla comprises a sequence of SEQ ID NO: 130, or a sequence having at least about 70%, at least about 80%, at least about 85%, at least about 90% at least about 91%, at least about 92%, at least about 93% at least about 94% at least about 95% identity thereto, and the RD1b comprises a sequence of SEQ ID NO: 131, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RDla comprises a sequence of SEQ ID NO: 130, or a sequence having at least about 70%, at least about 80%, at least about 85%, at least about 90%, at least about 91%, at least about 92%, at least about 93%, at least about 94%, or at least about 95% identity thereto, and the RD1b comprises a sequence of SEQ ID NO: 135, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RDla comprises a sequence of SEQ ID NO: 131, or a sequence having at least about 70%, at least about 80%, at least about 85%, at least about 90%, at least about 91%, at least about 92%, at least about 93%, at least about 94%, or at least about 95% identity thereto, and the RDlb comprises a sequence of SEQ ID NO: 130, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RDla comprises a sequence of SEQ ID NO: 131, or a sequence having at least about 70%, at least about 80%, at least about 85%, at least about 90%, at least about 91%, at least about 92%, at least about 93%, at least about 94%, or at least about 95% identity thereto, and the RD1b comprises a sequence of SEQ ID NO: 135, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RDla comprises a sequence of SEQ ID NO: 135, or a sequence having at least about 70%, at least about 80%, at least about 85%, at least about 90%, at least about 91%, at least about 92%, at least about 93%, at least about 94%, or at least about 95% identity thereto, and the RD1b comprises a sequence of SEQ ID NO: 130, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RD1a comprises a sequence of SEQ ID NO: 135, or a sequence having at least about 70%, at least about 80%, at least about 85%, at least about 90%, at least about 91%, at least about 92%, at least about 93%, at least about 94%, or at least about 95% identity thereto, and the RD1b comprises a sequence of SEQ ID NO: 131, or a sequence having at least about 70%, at least about 80%, at least about 85%, at least about 90%, at least about 91%, at least about 92%, at least about 93%, at least about 94%, or at least about 95% identity thereto. In some embodiments of the configuration 6b long-term repressor fusion protein, wherein the two copies of the RD1 are different, the RD1a and Rdlb are independently selected from the group consisting of the sequences of SEQ ID NOS: 132-134 and 136-138. In some embodiments of the configuration 6b long-term repressor fusion protein, wherein the two copies of the RD1 are different, the RD1a and Rdlb are independently selected from the group consisting of the sequences of SEQ ID NOS: 130, 131 and 135. A schematic of configuration 6b is shown in FIG. 19. In some embodiments of the configuration 6b long-term repressor fusion protein, the DNA binding protein may be a catalytically-dead CasX sequence of SEQ ID NO: 4. In some embodiments, the long-term repressor fusion protein is capable of forming an RNP with a gRNA of the disclosure and is capable of binding to and repressing or silencing the gene target nucleic acid.

TABLE 5
Exemplary combinations of linker amino acid sequences for repressor fusion
proteins

| Linker | | | |
|---|---|---|---|
| position | Linker Set # | Amino acid sequence | SEQ ID NO |
| L1 | Baseline/Original | GGPSSGAPPPSGGSPAGSPTSTEEGTSESATPESGPG | 123 |
| | | TSTEPSEGSAPGSPAGSPTSTEEGTSTEPSEGSAPGT | |
| | | STEPSE | |
| | Set 85 | ASPAAPAPASPAAPAPSAPAA | 1827 |
| | Set 43 | GSGGSGGSGGSPVPSTPPTPSPSTPPTP | 1826 |
| | Set 36 | PPTPSPSPVPSTPPTNSSSTPPTPS | 1825 |
| | Set 1 | ASAAAPAAASAAASAPSAAAA | 1823 |
| | Set 66 | AASPAAPSAPPAAASP | 1824 |
| L2 | Baseline/Original | SSGNSNANSRGPSFSSGLVPLSLRGSH | 122 |
| L3A | Baseline/Original | GGSGGG | 124 |
| | Set 85 | IRAHGD | 1832 |
| | Set 43 | AFPAAPAPA | 1831 |
| | Set 36 | GSGNSSGSGGS | 1829 |
| | Set 1 | ASAAAPAAA | 1830 |
| | Set 66 | PPTP | 1828 |
| L3B | Baseline/Original | GGSGGGS | 120 |
| L4v1 | Baseline/Original | GSGS | 1988 |
| L4v2 | Baseline/Original | GSGSGSG | 2130 |

In some embodiments, the long-term repressor fusion protein comprises a dCasX and, optionally, an ADD, and is configured as configuration 1. In some embodiments, the long-term repressor protein comprises a sequence selected from the group consisting of SEQ ID NOS: 21903-21922, or a sequence having at least about 70%, at least about 75%, at least abvout 80%, at least about 8500, at least about 90%, at least about 9500, or at least 9900 identity thereto. In some embodiments of the long-term repressor fusion protein comprising a dCasX and, optionally, an ADD, and which is configured as configuration 1, the long-term repressor protein comprises a sequence selected from the group consisting of SEQ ID NOS: 21903-21922. In some embodiments of the long-term repressor fusion protein comprising a dCasX and, optionally, an ADD, and is configured as configuration 1, the long-term repressor protein comprises a sequence selected from the group consisting of SEQ ID NOS: 21905 and 21914. In some embodiments of the long-term repressor fusion protein comprising a dCasX and, optionally, an ADD, and which is configured as configuration 1, the long-term repressor protein comprises a sequence selected from the group consisting of SEQ ID NOS: 21906 and 21915. In some embodiments of the long-term repressor fusion protein comprising a dCasX and, optionally, an ADD, and is configured as configuration 1, the long-term repressor protein comprises a sequence selected from the group consisting of SEQ ID NOS: 21907 and 21916.

The present disclosure provides long-term repressor fusion proteins of configuration 1, configuration 2, configuration 3, configuration 4, configuration 5, configuration 6a and configuration 6b, as shown in FIG. 19 and described above, wherein the long-term repressor fusion protein is capable of complexing with a gRNA with a targeting sequence complementary to a target nucleic acid of a gene in a cell to form an RNP. Upon binding of the RNP to a target nucleic acid of a targeted gene in a cell, the nucleic acid of the gene is epigenetically-modified and transcription of the gene is repressed. In some embodiments, transcription of the gene is repressed by at least about 10%, at least about 20%, at least about 30%, at least about 40%, at least about 50%, at least about 60%, at least about 70%, at least about 75%, at least about 80%, at least about 85%, at least about 90%, at least about 95%, or at least 99%. In some embodiments, transcription of the gene in at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% or more of cells in a population of cells is repressed. Most preferably, gene repression results in complete inhibition of gene expression, such that no gene product is detectable. However, the skilled artisan will appreciate that incomplete inhibition may still be useful and desired for various applications. In some embodiments, the repression of transcription of the gene is sustained for at least about 8 hours, at least about 1 day, at least about 7 days, at least 2 weeks, at least about 3 weeks, at least about 1 month, or at least about 2 months, when assayed in an in vitro assay, including cell-based assays. In some embodiments, the repression of transcription of the gene is sustained for at least about 7 days, at least 2 weeks, at least about 3 weeks, at least about 1 month, at least about 2 months, at least about 3 months, at least about 4 months, at least about 5 months, or at least about 6 months or more in targeted cells. In some embodiments, use of the long-term repressor fusion protein configurations 1, 4, 5, 6a and 6b, when used in an LTRP:gRNA system of the embodiments, results in off-target methylation or off-target activity that is less than about 10%, less than about 9%, less than about 8%, less than about 7%, less than about 6%, less than about 5%, less than about 4%, less than about 3%, less than about 2%, less than about 1%, less that 0.5%, or less than 0.1% in the cells. In some embodiments, repression of transcription in cells treated with an LTRP:gRNA system of the embodiments is heritable and is stable through one or more cell divisions. In some embodiments, repression of transcription is stable through 1, 10, 20, 30, 40, 50, 60, 70, 80, 90, or 100 cell divisions, or more. In some embodiments, the transcriptional repression is assayed in an in vitro assay, including cell-based assays, and transcriptional repression is compared to untreated cells or cells treated with a comparable system in which the gRNA comprises a non-targeting spacer. In some embodiments, the transcriptional repression is assayed in vivo, in cells obtained from a subject in which the long-term repressor fusion protein and the gRNA with a targeting sequence complementary to a target nucleic acid of a gene in a cell are administered; either as a protein and gRNA, or as nucleic acids (e.g., a gRNA and an mRNA encoding the long-term repressor fusion protein), wherein the subject is selected from the group consisting of mouse, rat, pig, non-human primate, and human.

V. mRNA compositions encoding long-term repressor fusion proteins

In another aspect, the disclosure relates to messenger RNA (mRNA) compositions comprising sequences of the individual components, as well as the full-length mRNA sequences of the long-term repressor domain fusion protein constructs of the disclosure. The mRNA compositions have utility in the transcriptional repression and epigenetic modification of a gene, when used to express the long-term repressor fusion protein. In some cases, the mRNA compositions are designed for use in certain delivery formulations; e.g., nanoparticles such as synthetic nanoparticles or lipid nanoparticles (LNP). The disclosure also provides methods utilized to design the mRNA sequences of the mRNAs used in the compositions and formulations to deliver the compositions. In some cases, an mRNA encoding the long-term repressor fusion protein may be co-formulated in a nanoparticle with a gRNA comprising a targeting sequence complementary to a target nucleic acid sequence of a gene to be transcriptionally repressed or silenced, wherein upon delivery of the nanoparticle to a target cell, the long-term repressor domain fusion protein is expressed from the mRNA and can complex with the gRNA as an RNP capable of binding the target nucleic acid. In other cases, the mRNA encoding the long-term repressor domain fusion protein and the gRNA may be formulated in separate nanoparticles and delivered either separately or as a mixture.

In some embodiments, the mRNA compositions have been modified to result in one or more improved characteristics relative to an unmodified mRNA encoding the same long-term repressor protein, and therefore can have a significant impact on the efficacy of mRNA-based delivery. Exemplary improved characteristics of the mRNA described herein include, but are not limited to, improved expression upon delivery to a cell, reduced immunogenicity, increased stability, and enhanced manufacturability compared to unmodified mRNA. In some cases, the modifications to the mRNA result in an improved characteristic of at least about 1.1 to about 100,000-fold improved relative to the unmodified mRNA. In some embodiments, an improved characteristic of the modified mRNA is at least about 1.1 to about 10,000-fold improved, at least about 1.1 to about 1,000-fold improved, at least about 1.1 to about 500-fold improved, at least about 1.1 to about 400-fold improved, at least about 1.1 to about 300-fold improved, at least about 1.1 to about 200-fold improved, at least about 1.1 to about 100-fold improved, at least about 1.1 to about 50-fold improved, at least about 1.1 to about 40-fold improved, at least about 1.1 to about 30-fold improved, at least about 1.1 to about 20-fold improved, at least about 1.1 to about 10-fold improved, at least about 1.1 to about 9-fold improved, at least about 1.1 to about 8-fold improved, at least about 1.1 to about 7-fold improved, at least about 1.1 to about 6-fold improved, at least about 1.1 to about 5-fold improved, at least about 1.1 to about 4-fold improved, at least about 1.1 to about 3-fold improved, at least about 1.1 to about 2-fold improved, at least about 1.1 to about 1.5-fold improved, at least about 1.5 to about 3-fold improved, at least about 1.5 to about 4-fold improved, at least about 1.5 to about 5-fold improved, at least about 1.5 to about 10-fold improved, at least about 5 to about 10-fold improved, at least about 10 to about 20-fold improved, at least 10 to about 30-fold improved, at least 10 to about 50-fold improved or at least 10 to about 100-fold improved relative to the unmodified mRNA. In some embodiments, an improved characteristic of the modified mRNA is at least about 10 to about 1000-fold improved relative to the unmodified mRNA.

Optimization of coding sequences and untranslated regions (UTRs) may be useful when delivering an mRNA encoding a protein of interest, as opposed to a DNA template that would be transcribed into an mRNA. DNA templates are long-lived, can replicate, and can produce many RNA transcripts over their lifetimes. For DNA templates, efficiency of transcription and pre-mRNA processing are major determinants of protein expression levels. In contrast, mRNAs generally have a much shorter half-life, on the order of hours, as they are vulnerable to degradation in the cytoplasm, and cannot produce more copies of themselves. As such, mRNA stability and translation efficiency can be key determinants of protein expression levels for mRNA-based delivery, and the specific sequences of UTRs and coding sequences that dictate mRNA stability and translation efficiency can therefore be enhanced to improve the efficacy of mRNA-based delivery.

### a. 5′ Cap

In some embodiments of the mRNA encoding the LTRP of the disclosure, the mRNA comprises a 5′ cap linked 5′ to the 5′ UTR of the mRNA sequence of any of the embodiments described herein. In some embodiments, the 5′ cap is a 7-methylguanylate cap. In some embodiments, the 5′ cap comprises m7G(5′)ppp(5′)mAG. In other embodiments, the 5′ cap comprises of m7G(5′)ppp (5′(A,G(5′)ppp(5′)A or G(5′)ppp(5′)G. Exemplary caps are known in the art, and described, for example, in WO 2017/053297, the contents of which are incorporated by reference herein.

### b. 5′ Untranslated Region (UTR)

The 5′ UTR of an mRNA molecule can be a determinant of both the stability of the mRNA and how efficiently it is translated into protein. Specifically, the 5′ UTR, in conjunction with the 5′ cap structure, serves as a binding site and recruitment platform for the translation pre-initiation complex as well as additional regulatory proteins that may positively or negatively affect translation. Structures within the 5′ UTR can enhance translation by recruiting initiation factors or other protein or RNA factors, reduce translation by physically blocking ribosome binding and scanning, and contribute to the stability of the mRNA by affecting both hydrolysis and nuclease digestion.

An exemplary 5′ UTR sequence for use in the mRNA of the disclosure is provided in Table 6A. Table 6A lists the RNA sequence, RNA sequence with N1-methylpseudouridine substituted in place of uridine, and DNA sequence of the 5′ UTR.

TABLE 6A
5′ UTR sequences

| SEQ ID | | |
|---|---|---|
| NO | Nucleic Acid Sequence* | Description |
| 21831 | AAAUAAGAGAGAAAAGAAGAGUAAGAAGAAAUAUAAGA | RNA sequence of 5′ UTR |
| 21842 | AAAmYAAGAGAGAAAAGAAGAGmYAAGAAGAAAmYAmYAAGA | N1-methylpseudouridine |
| | | substituted RNA |
| | | sequence of 5′ UTR |

*‘mψ’ = N1-methyl-pseudouridine

In some embodiments, the 5′ UTR comprises the sequence of SEQ ID NO: 21831, or a sequence having at least about 50%, at least about 60%, at least about 70%, at least about 80%, at least about 90%, or at least about 95% identity thereto. In some embodiments, the 5′ UTR comprises the sequence of SEQ ID NO: 21831. In some embodiments, the 5′ UTR consists of the sequence of SEQ ID NO: 21831. In some embodiments, the 5′ UTR comprises the sequence of SEQ ID NO: 21842, or a sequence having at least about 50%, at least about 60%, at least about 70%, at least about 80%, at least about 90%, or at least about 95%, identity thereto. In some embodiments, the 5′ UTR comprises the sequence of SEQ ID NO: 21842. In some embodiments, the 5′ UTR consists of the sequence of SEQ ID NO: 21842.

### c. 3′ UTR

3′ UTR sequences can have a significant impact on mRNA stability and translation efficiency, and can determine both subcellular localization and tissue-specific expression. Factors influencing these properties include microRNA binding sites, AU-rich elements that recruit an array of RNA-binding proteins, Pumilio binding elements, and other binding sites for RNA-binding proteins. While many of these interactions with the 3′ UTR are known to negatively impact stability or expression, some can enhance translation. The effects of a 3′ UTR sequence can be highly cell-type specific due to differential expression of microRNAs and RNA binding proteins, which provides opportunities for engineering tissue-specific expression into a therapeutic mRNA. In some embodiments, the 3′ UTR for use in the mRNA of the disclosure is a mouse 3′ UTR. In some embodiments, the 3′ UTR is a mouse HBA gene 3′ UTR of Table 6B.

Exemplary 3′ UTR sequences of the disclosure are provided in Table 6B. Table 6B lists the RNA sequence, and the RNA sequence with N1-methylpseudouridine substituted in place of uridine.

TABLE 6B
3′ UTR sequences

| SEQ ID | | |
|---|---|---|
| NO | Nucleic Acid Sequence* | Description |
| 21844 | GCUGCCUUCUGCGGGGCUUGCCUUCUGGCCAUGCCCUUCUUCUCU | RNA sequence of |
| | CCCUUGCACCUGUACCUCUUGGUCUUUGAAUAAAGCCUGAGUAGG | 3′ UTR |
| | AAG | |
| 21845 | GCmYGCCmmYCmWGCGGGGCmYmWGCCmYmYCmYGGCCAmyGCC | N1- |
| | CmYmYCmYmWCmYCmWCCCmYmWGCACCmVGmVACCmWCmYmYGG | methylpseudouridine |
| | myCmYmYmYGAAmWAAAGCCmWGAGmWAGGAAG | substituted |
| | | RNA sequence of |
| | | 3′ UTR |

*‘mψ’ = N1-methyl-pseudouridine

In some embodiments, the 3′ UTR comprises the sequence of SEQ ID NO: 21844, or a sequence having at least about 50%, at least about 60%, at least about 70%, at least about 80%, at least about 90%, or at least about 95% identity thereto. In some embodiments, the 3′ UTR comprises the sequence of SEQ ID NO: 21844. In some embodiments, the 3′ UTR consists of the sequence of SEQ ID NO: 21844. In some embodiments, the 3′ UTR comprises the sequence of SEQ ID NO: 21845, or a sequence having at least about 50%, at least about 60%, at least about 70%, at least about 80%, at least about 90%, or at least about 95% identity thereto. In some embodiments, the 3′ UTR comprises the sequence of SEQ ID NO: 21845. In some embodiments, the 3′ UTR consists of the sequence of SEQ ID NO: 21845.

d. Poly(A) Sequence

Inclusion of a 3′ poly(A) tail in an mRNA sequence can contribute to the stability and translation efficiency of the mRNA. Generally, longer poly(A) tails are associated with increased mRNA stability, thereby allowing their translation and promoting high protein expression.

In some embodiments, an mRNA of the disclosure comprises a poly(A) sequence having at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 185, or at least about 190 adenine nucleotides. In some embodiments, the poly(A) sequence of an mRNA of the disclosure comprises 80 adenine nucleotides. In some embodiments, the poly(A) sequence comprises the nucleic acid sequence of AAAAAAAAAAAAAAAAAAAAAAAAAAAAAAAAAAAAAAAAAAAAAAAAAAAAAA AAAAAAAAAAAAAAAAAAAAAAAAAA (SEQ ID NO: 1961). In some embodiments, the poly(A) sequence comprises the nucleic acid sequence of AAAAAAAAAAAAAAAAAAAAAAAAAAAAAAAAAAAAAAAAAAAAAAAAAAAAAA AAAAAAGCAUGCAAAAAAAAAAAAAAAAAAAAAAAAAAAAAAAAAAAAAAAAAA AAAAAAAAAAAAAAA (SEQ ID NO: 21851).

e. Sequence Modifications to the mRNA

In some embodiments, an mRNA sequence of the disclosure was modified by codon optimization of the sequence encoding the long-term repressor protein using one or more parameters to enhance expression in the target cell. Non-limiting examples of such parameters include the codon usage in human host cells (e.g., utilizing the codon adaptation index (CAI)), codon-usage tables derived from biologics intended for use as therapeutics, mRNA stability index, or GC content. Methods of codon-optimization, and codon usage in various organisms is known in the art. See, for example, www.genscript.com/tools/codon-frequency-table. In some embodiments, provided herein are mRNA sequences for the long-term repressor protein constructs that are codon-optimized for expression in a human cell. In other cases, various naturally-occurring or modified nucleosides may be used to produce the modified mRNA according to the present disclosure.

In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the mRNA comprises one or more nonstandard nucleotide residues. The nonstandard nucleotide residues may include, e.g., 5-methyl-cytidine (“5 mC”), N1-methyl-pseudouridine (“WU”), and/or 2-thio-uridine (“2sU”). In a particular embodiment, one or more or all of the uridine residues of the mRNA of the disclosure are replaced with N1-methyl-pseudouridine. In some embodiments, all of the uridine residues of the mRNA of the disclosure are replaced with 1-methyl-pseudouridine. See, e.g., U.S. Pat. No. 8,278,036 or WO2011012316, incorporated by reference herein, for a discussion of such residues and their incorporation into mRNA. In some embodiments, the modifications to the mRNA result in an improved characteristic of at least about 1.1 to about 100,000-fold improved relative to the unmodified mRNA. In some embodiments, an improved characteristic of the modified mRNA is at least about 1.1 to about 10,000-fold improved, at least about 1.1 to about 1,000-fold improved, at least about 1.1 to about 500-fold improved, at least about 1.1 to about 400-fold improved, at least about 1.1 to about 300-fold improved, at least about 1.1 to about 200-fold improved, at least about 1.1 to about 100-fold improved, at least about 1.1 to about 50-fold improved, at least about 1.1 to about 40-fold improved, at least about 1.1 to about 30-fold improved, at least about 1.1 to about 20-fold improved, at least about 1.1 to about 10-fold improved, at least about 1.1 to about 9-fold improved, at least about 1.1 to about 8-fold improved, at least about 1.1 to about 7-fold improved, at least about 1.1 to about 6-fold improved, at least about 1.1 to about 5-fold improved, at least about 1.1 to about 4-fold improved, at least about 1.1 to about 3-fold improved, at least about 1.1 to about 2-fold improved, at least about 1.1 to about 1.5-fold improved, at least about 1.5 to about 3-fold improved, at least about 1.5 to about 4-fold improved, at least about 1.5 to about 5-fold improved, at least about 1.5 to about 10-fold improved, at least about 5 to about 10-fold improved, at least about 10 to about 20-fold improved, at least 10 to about 30-fold improved, at least 10 to about 50-fold improved or at least 10 to about 100-fold improved relative to the unmodified mRNA. In some embodiments, an improved characteristic of the modified mRNA is at least about 10 to about 1000-fold improved relative to the unmodified mRNA.

f. LTRP mRNA Component Sequences

The disclosure provides mRNA comprising sequences encoding the components utilized in the long-term repressor fusion proteins described herein. In some embodiments, the mRNA comprises sequences encoding the DNA binding proteins, including TALE, ZF, and catalytically dead CRISPR proteins. In some embodiments, the mRNA comprises the sequence of SEQ ID NO: 2211, encoding dCasX 515 (SEQ ID NO: 6), or a sequence having at least about 70%, at least about 80%, at least about 85%, at least about 90%, at least about 95%, at least about 96%, at least about 97%, at least about 98%, or at least about 99% sequence identity thereto. In some embodiments, the mRNA comprises the sequence of SEQ ID NO: 2213 or SEQ ID NO: 2214 encoding dCasX 812 (SEQ ID NO: 29), or a sequence having at least about 70%, at least about 80%, at least about 85%, at least about 90%, at least about 95%, at least about 96%, at least about 97%, at least about 98%, or at least about 99% sequence identity thereto. In some embodiments, the mRNA comprises the sequence of SEQ ID NO: 1948 or SEQ ID NO: 2405, encoding dCasX 491 (SEQ ID NO: 4), or a sequence having at least about 70%, at least about 80%, at least about 85%, at least about 90%, at least about 95%, at least about 96%, at least about 97%, at least about 98%, or at least about 99% sequence identity thereto. In some embodiments, the mRNA comprises a DNA binding protein encoded by a sequence consisting essentially of the sequence of SEQ ID NO: 2405, encoding dCasX 491 (SEQ ID NO: 4). In some embodiments, the mRNA comprises a sequence of SEQ ID NO: 2407 or of SEQ ID NO: 2408 encoding dCasX 676 (SEQ ID NO: 28), or a sequence having at least about 70%, at least about 80%, at least about 85%, at least about 90%, at least about 95%, at least about 96%, at least about 97%, at least about 98%, or at least about 99% sequence identity thereto. The sequences encoding the dCasX, described supra, are incorporated into the mRNA sequence encoding the long-term repressor fusion protein.

In some embodiments, the sequence of the mRNA encoding dCasX 491 (SEQ ID NO: 4) has pseudouridine nucleosides replacing one or more, or all uridines in the sequence (SEQ ID NO: 2406). In some embodiments, the sequence of the mRNA encoding dCasX 515 (SEQ ID NO: 6) has pseudouridine nucleosides replacing one or more, or all uridines in the sequence. In some embodiments, the sequence of the mRNA encoding dCasX 676 (SEQ ID NO: 28) has pseudouridine nucleosides replacing one or more, or all uridines in the sequence. In some embodiments, the sequence of the mRNA encoding dCasX 812 (SEQ ID NO: 29) has pseudouridine nucleosides replacing one or more, or all uridines in the sequence.

In some embodiments, the sequence of the mRNA encoding the dCasX is selected from the group consisting of SEQ ID NOS: 1948, 2211, 2213-2214, 2405-2408. In some embodiments, the sequence of the mRNA encoding the dCasX has pseudouridine nucleosides replacing one or more uridines. In some embodiments, the sequence of the mRNA encoding the dCasX has pseudouridine nucleosides replacing all uridines.

The disclosure provides mRNA sequences encoding the RD1 domains. In some embodiments, the sequence encoding the RD1 comprises a sequence selected from the group consisting of SEQ ID NOS: 1946, 21846, and 18637-20233, or a sequence having at least about 70%, at least about 80%, at least about 85%, at least about 90%, at least about 91%, at least about 92%, at least about 93%, at least about 94%, or at least about 95% identity thereto. In another embodiment, the sequence encoding the RD1 comprises a sequence selected from the group consisting of SEQ ID NOS: 18637-18731, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the sequence encoding the RD1 comprises a sequence selected from the group consisting of SEQ ID NOS: 18637-18645, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the sequence encoding the RD1 comprises a sequence of SEQ ID NO: 18642 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the sequence encoding the RD1 comprises a sequence of SEQ ID NO: 18638 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In another embodiment, the sequence encoding the RD1 comprises a sequence of SEQ ID NO: 18637 or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the sequence encoding the RD1 comprises SEQ ID NOS: 18637, 18638 or 18642.

In some embodiments, the mRNA comprises a sequence encoding the second repressor domain. In some embodiments, the second repressor domain comprises DNMT3A. In some embodiments, the sequence encoding the DNMT3A comprises a sequence of SEQ ID NO: 1923, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the sequence encoding the DNMT3A comprises a sequence of SEQ ID NO: 1923.

In some embodiments, the mRNA comprises a sequence encoding the third repressor domain. In some embodiments, the third repressor domain comprises DNMT3L. In some embodiments, the sequence encoding the DNMT3L comprises a sequence SEQ ID NO: 1945, or a sequence having at least about 70%, at least about 80%, at least about 90%, at least about 91%, at least about 92%, at least about 93%, at least about 94%, at least about 95%, at least about 96%, at least about 97%, at least about 98%, at least about 99% identity thereto. In some embodiments, the sequence encoding the DNMT3L comprises a sequence SEQ ID NO: 1945.

In some embodiments, the mRNA comprises a sequence encoding the fourth repressor domain. In some embodiments, the fourth repressor domain comprises the ADD. In some embodiments, the sequence encoding the ADD comprises a sequence of SEQ ID NO: 1954, or a sequence having at least about 70%, at least about 80%, at least about 90%, at least about 91%, at least about 92%, at least about 93%, at least about 94%, at least about 95%, at least about 96%, at least about 97%, at least about 98%, at least about 99% identity thereto. In some embodiments, the fourth repressor domain comprises the ADD. In some embodiments, the sequence encoding the ADD comprises a sequence of SEQ ID NO: 1954.

In some embodiments, the mRNA of the disclosure comprises a Kozak sequence. In some embodiments, the Kozak sequence comprises GCCACCAUGG (SEQ ID NO: 21848). In some embodiments, an mRNA of the disclosure comprises a Kozak sequence and two bases upstream of the NLS (to produce the methionine and alanine upstream of the NLS). In some embodiments, the mRNA comprises the sequence GCCACCAUGGCC (SEQ ID NO: 21832) between the 5′ UTR and the sequence encoding the NLS.

In another embodiment, the mRNA comprises an NLS. In some embodiments, the sequence encoding the NLS comprises a sequence selected from the group consisting of SEQ ID NOS: 21833 and SEQ ID NO: 21849, or a sequence having at least about 70%, at least about 80%, or at least about 90% identity thereto. In another embodiment, the mRNA comprises a sequence selected from the group consisting of SEQ ID NOS: 21833 and SEQ ID NO: 21849. In another embodiment, for examples those embodiments wherein the long-term repressor protein comprises more than one NLS, the mRNA comprises two or more sequences independently selected from the group consisting of SEQ ID NOS: 21833 and SEQ ID NO: 21849.

In some embodiments, the mRNA comprises sequences encoding the linkers of the long-term repressor fusion protein. In some embodiments, the sequence encoding the linker comprises a sequence selected from the group consisting of SEQ ID NOS: 21857-21873, or a sequence having at least about 80%, at least about 90%, at least about 91%, at least about 92%, at least about 93%, at least about 94%, at least about 95% identity thereto. In some embodiments, the sequence encoding the linker comprises a sequence selected from the group consisting of SEQ ID NOS: 21857-21873. In some embodiments, for example those embodiments where the long-term repressor fusion protein comprises more than one linker, the sequences encoding the linkers are independently selected from the group consisting of SEQ ID NOS: 21857-21873, or a sequence having at least about 80%, at least about 90%, at least about 91%, at least about 92%, at least about 93%, at least about 94%, at least about 95% identity thereto.

In some embodiments, the mRNA comprises a sequence encoding the long-term repressor fusion protein of the configurations provided herein. In some embodiments, the mRNA encodes a long-term repressor fusion protein of configuration 1, and the mRNA comprises a sequence selected from the group consisting of SEQ ID NOS: 2521-7311, or a sequence having at least about 80%, at least about 90%, at least about 91%, at least about 92%, at least about 93%, at least about 94%, at least about 95%, at least about 96%, at least about 97%, at least about 98%, at least about 99% identity thereto. In some embodiments, the mRNA comprises a sequence selected from the group consisting of SEQ ID NOS: 2521-7311.

In some embodiments, the mRNA comprises a sequence encoding the long-term repressor fusion protein of configuration 1, and comprises a sequence encoding an RD1 comprising a sequence of SEQ ID NO: 130. In some embodiments, the mRNA comprises a sequence of SEQ ID NOS: 18637 or 20234 or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18637 and 20234. In some embodiments, the mRNA comprises a sequence encoding the long-term repressor fusion protein of configuration 1, and comprises a sequence encoding anRDl comprising a sequence of SEQ ID NO: 131. In some embodiments, the mRNA comprises a sequence of SEQ ID NOS: 18638 or 20235, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18638 and 20235. In some embodiments, the mRNA comprises a sequence encoding the long-term repressor fusion protein of configuration 1, and comprises a sequence encoding an RD1 comprising a of SEQ ID NO: 132. In some embodiments, the mRNA comprises a sequence of SEQ ID NOS: 18639 or 20236, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence encoding the long-term repressor fusion protein of configuration 1, and comprises a sequence encoding an RD1 comprising a sequence of SEQ ID NO: 133. In some embodiments, the mRNA comprises a sequence of SEQ ID NOS: 18640 or 20237, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18640 or 20237. In some embodiments, the mRNA comprises a sequence encoding the long-term repressor fusion protein of configuration 1, and comprises a sequence encoding an RD1 comprising a sequence of SEQ ID NO: 134. In some embodiments, the mRNA comprises a sequence of SEQ ID NOS: 18641 or 20238, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18641 or 20238. In some embodiments, the mRNA comprises a sequence encoding the long-term repressor fusion protein of configuration 1, and comprises a sequence encoding an RD1 comprising a sequence of SEQ ID NO: 135. In some embodiments, the mRNA comprises a sequence of SEQ ID NOS: 18642 or 20239, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18642 or 20239. In some embodiments, wherein the mRNA comprises a sequence encoding the long-term repressor fusion protein of configuration 1, and comprises a sequence encoding an RD1 comprising a sequence of SEQ ID NO: 136. In some embodiments, the mRNA comprises a sequence of SEQ ID NOS: 18643 or 20240, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18643 or 20240. In some embodiments, the mRNA comprises sequence encoding the long-term repressor fusion protein of configuration 1, and comprises a sequence encoding the RD1 comprising a sequence of SEQ ID NO: 137. In some embodiments, the mRNA comprises a sequence of SEQ ID NOS: 18644 or 20241, or a sequence having at least about 80%, at least about 90%, at least about 91%, at least about 92%, at least about 93%, at least about 94%, at least about 95%, at least about 96%, at least about 97%, at least about 98%, at least about 99% identity thereto. In some embodiments, the mRNA comprises a sequence of SEQ ID NOS: 18644 or 20241. In some embodiments, the mRNA comprises a sequence encoding the long-term repressor fusion protein of configuration 1, and comprises a sequence encoding an RD1 comprising a sequence of SEQ ID NO: 138. In some embodiments, the mRNA comprises a sequence of SEQ ID NOS: 18645 or 20242, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence of SEQ ID NOS: 18645 or 20242.

The disclosure provides an mRNA encoding a long-term repressor fusion protein of configuration 5. In some embodiments, the mRNA comprises a sequence is selected from the group consisting of SEQ ID NOS: 8909-12102, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence is selected from the group consisting of SEQ ID NOS: 8909-12102.

The disclosure provides an mRNA encoding a long-term repressor fusion protein of configuration 6a. In some embodiments, the mRNA comprises a sequence is selected from the group consisting of SEQ ID NOS: 15297-16893, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence is selected from the group consisting of SEQ ID NOS: 15297-16893.

The disclosure provides an mRNA encoding a long-term repressor fusion protein of configuration 6b. In some embodiments, the mRNA comprises a sequence is selected from the group consisting of SEQ ID NOS: 18491-18563, or a sequence having at least about 80%, at least about 90%, at least about 91%, at least about 92%, at least about 93%, at least about 94%, at least about 95%, at least about 96%, at least about 97%, at least about 98%, or at least about 99% identity thereto. In some embodiments, the mRNA comprises a sequence is selected from the group consisting of SEQ ID NOS: 18491-18563.

In some embodiments of the mRNA encoding a long-term repressor fusion protein of the LTRP:gRNA systems of the disclosure, upon delivery of the system and expression in a cell, the long-term repressor fusion protein is capable of complexing with a gRNA and binding the target DNA of the targeted gene of the cell, resulting in repression or silencing of transcription of the gene in the cell.

### VI. Guide Nucleic Acids of the Systems

In another aspect, the disclosure relates to specifically-designed guide ribonucleic acids (gRNA) comprising a scaffold and a linked targeting sequence complementary to (and are therefore able to hybridize with) a target nucleic acid sequence of a gene. The gRNA described herein can be used with the long-term repressor proteins, and systems comprising same, to repress transcription of a target nucleic acid in a eukaryotic cell. As used herein, the term “gRNA” covers naturally-occurring molecules and gRNA variants, including chimeric gRNA variants comprising domains from different gRNA. gRNAs of the disclosure comprise a scaffold and a targeting sequence complementary to a target nucleic acid of a cell linked to the 3′ end of the scaffold.

In some embodiments, the systems comprising an mRNA encoding a long-term repressor fusion protein comprising a dCasX protein, and one or more gRNA, upon expression of the long-term repressor fusion protein in a cell, form a ribonucleoprotein (RNP) complex comprising the LTRP and the gRNA, which can target and bind specific locations in the target nucleic acid sequence of a gene in the cell to be repressed or silenced. The gRNA provides target specificity to the complex by including a targeting sequence (or “spacer”) having a nucleotide sequence that is complementary to a sequence of the target nucleic acid sequence while the long-term repressor fusion protein of the system provides the site-specific activity such as binding and transcriptional repression of the target gene that is guided to a target site (e.g., stabilized at a target site) within a target nucleic acid sequence by virtue of its association with the gRNA.

Embodiments of gRNAs, and formulations of mRNAs and gRNAs, for use in the transcriptional repression and/or epigenetic modification of a target nucleic acid are described herein, below.

a. Reference gRNA and gRNA Variants

As used herein, a “reference gRNA” refers to a CRISPR guide ribonucleic acid comprising a wild-type sequence of a naturally-occurring gRNA. In some embodiments, a gRNA scaffold of the disclosure may be subjected to one or more mutagenesis methods, such as the mutagenesis methods described in WO2023235818A2, WO2022120095A1 and WO2020247882A1, incorporated by reference herein, which may include Deep Mutational Evolution (DME), deep mutational scanning (DMS), error prone PCR, cassette mutagenesis, random mutagenesis, staggered extension PCR, gene shuffling, domain swapping, or chemical modification to generate one or more gRNA variants with enhanced or varied properties relative to the gRNA scaffold that was modified. The activity of the gRNA scaffold from which a gRNA variant was derived may be used as a benchmark against which the activity of the gRNA variant is compared, thereby measuring improvements in function or other characteristics of the gRNA scaffold.

Table 7 provides the sequences of reference gRNA tracr and scaffold sequences. In some embodiments, the disclosure provides gRNA variants wherein the gRNA has a scaffold comprising a sequence having one or more nucleotide modifications relative to a reference gRNA sequence of any one of SEQ ID NOS: 1731-1743 of Table 7.

TABLE 7
Reference gRNA tracr and scaffold sequences

| SEQ ID NO. | Nucleotide Sequence |
|---|---|
| 1731 | ACAUCUGGCGCGUUUAUUCCAUUACUUUGGAGCCAGUCCCAGCGACUAUGUCGUAUGGAC |
| | GAAGCGCUUAUUUAUCGGAGAGAAACCGAUAAGUAAAACGCAUCAAAG |
| 1732 | UACUGGCGCUUUUAUCUCAUUACUUUGAGAGCCAUCACCAGCGACUAUGUCGUAUGGGUA |
| | AAGCGCUUAUUUAUCGGAGAGAAAUCCGAUAAAUAAGAAGCAUCAAAG |
| 1733 | ACAUCUGGCGCGUUUAUUCCAUUACUUUGGAGCCAGUCCCAGCGACUAUGUCGUAUGGAC |
| | GAAGCGCUUAUUUAUCGGAGA |
| 1734 | ACAUCUGGCGCGUUUAUUCCAUUACUUUGGAGCCAGUCCCAGCGACUAUGUCGUAUGGAC |
| | GAAGCGCUUAUUUAUCGG |
| 1735 | UACUGGCGCUUUUAUCUCAUUACUUUGAGAGCCAUCACCAGCGACUAUGUCGUAUGGGUA |
| | AAGCGCUUAUUUAUCGGAGA |
| 1736 | UACUGGCGCUUUUAUCUCAUUACUUUGAGAGCCAUCACCAGCGACUAUGUCGUAUGGGUA |
| | AAGCGCUUAUUUAUCGG |
| 1737 | GUUUACACACUCCCUCUCAUAGGGU |
| 1738 | GUUUACACACUCCCUCUCAUGAGGU |
| 1739 | UUUUACAUACCCCCUCUCAUGGGAU |
| 1740 | GUUUACACACUCCCUCUCAUGGGGG |
| 1741 | CCAGCGACUAUGUCGUAUGG |
| 1742 | GCGCUUAUUUAUCGGAGAGAAAUCCGAUAAAUAAGAAGC |
| 1743 | GGCGCUUUUAUCUCAUUACUUUGAGAGCCAUCACCAGCGACUAUGUCGUAUGGGUAAAGC |
| | GCUUAUUUAUCGGA |

b. GRNA Domains and their Function

The gRNAs of the systems of the disclosure comprise two segments: a targeting sequence and a protein-binding segment. The targeting segment of a gRNA includes a nucleotide sequence (referred to interchangeably as a spacer, a targeter, or a targeting sequence) that is complementary to (and therefore hybridizes with) a specific sequence (a target site) within the target nucleic acid sequence (e.g., a strand of a double stranded target DNA, a target ssRNA, a target ssDNA, etc.), described more fully below. The targeting sequence of a gRNA is capable of binding to a target nucleic acid sequence, including, in the context of the present disclosure, a coding sequence, a complement of a coding sequence, a non-coding sequence, and to accessory elements. The protein-binding segment (or “activator” or “protein-binding sequence”) of the gRNA interacts with (e.g., binds to) a CasX protein as a complex, forming an RNP (described more fully, below). As used herein, “scaffold” refers to all parts to the guide with the exception of the targeting sequence, which is comprised of several regions, described more fully, below. The properties and characteristics of CasX gRNA, both wild-type and variants, are described in WO2020247882A1, US20220220508A1, WO2022120095A1, and WO2023235818A2, incorporated by reference herein.

In the case of a reference gRNA, the gRNA occurs naturally as a dual guide RNA (dgRNA), wherein the targeter and the activator portions each have a duplex-forming segment that have complementarity with one another and hybridize to one another to form a double stranded duplex (dsRNA duplex for a gRNA). The term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. In the case of the gRNA for use in the systems of the disclosure, the scaffolds are designed such that the activator and targeter portions are covalently linked to one another (rather than hybridizing to one another) and comprise a single molecule, and can be referred to as a “single-molecule gRNA,” “single guide RNA”, a “single-molecule guide RNA,” a “one-molecule guide RNA”, or a “sgRNA”. The gRNA variants of the disclosure for use in the systems are all single molecule versions.

Collectively, the assembled gRNAs of the disclosure comprise distinct structured regions, or domains: the RNA triplex, the scaffold stem loop, the extended stem loop, the pseudoknot, and the targeting sequence that, in the embodiments of the disclosure is specific for a target nucleic acid and is located on the 3′ end of the gRNA. The RNA triplex, the scaffold stem loop, the pseudoknot and the extended stem loop, together with the unstructured triplex loop that bridges portions of the triplex, together, are referred to as the “scaffold” of the gRNA. In some cases, the scaffold stem further comprises a bubble. In other cases, the scaffold further comprises a triplex loop region. In still other cases, the scaffold further comprises a 5′ unstructured region. In some embodiments, the gRNA scaffolds of the disclosure for use in the LTRP:gRNA systems comprise a scaffold stem loop having the sequence of CCAGCGACUAUGUCGUAGUGG (SEQ ID NO: 1822), or a sequence with at least 1, 2, 3, 4 or 5 mismatches thereto.

Each of the structured domains contribute to the establishment of the global RNA fold of the guide and retain functionality of the guide, particularly the ability to properly complex with the dCasX protein. For example, the guide scaffold stem interacts with the helical I domain of dCasX protein, while residues within the triplex, triplex loop, and pseudoknot stem interact with the OBD of the dCasX protein. Together, these interactions confer the ability of the guide to bind and form an RNP with the dCasX that retains stability, while the spacer (or targeting sequence) directs and defines the specificity of the RNP for binding a specific sequence of DNA.

Site-specific binding of a target nucleic acid sequence (e.g., genomic DNA) by the dCasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC protospacer adjacent motif (PAM) motif or a PAM sequence, such as ATC, CTC, GTC, or TTC. Because the targeting sequence of a guide sequence hybridizes with a sequence of a target nucleic acid sequence, a targeting sequence can be modified by a user to hybridize with a specific target nucleic acid sequence, so long as the location of the PAM sequence is considered. In some embodiments, for design of a targeting sequence, the target nucleic acid comprises a PAM sequence located 5′ of the targeting sequence with at least a single nucleotide separating the PAM from the first nucleotide of the target nucleic acid complementary to that of the targeting sequence. This feature distinguishes the systems described herein from Cas9 systems, and results in the ability of the systems of the disclosure to modify different locations in a DNA sequence when compared to Cas9 systems. In some embodiments, the PAM is located on the non-targeted strand of the target region, i.e. the strand that is complementary to the target nucleic acid. In some embodiments, the targeting sequence of the gRNA is complementary to a target nucleic acid sequence one nucleotide from an ATC PAM sequence. In some embodiments, the targeting sequence of the gRNA is complementary to a target nucleic acid sequence one nucleotide from an CTC PAM sequence. In some embodiments, the targeting sequence of the gRNA is complementary to a target nucleic acid sequence one nucleotide from an GTC PAM sequence. In some embodiments, the targeting sequence of the gRNA is complementary to a target nucleic acid sequence one nucleotide from an TTC PAM sequence. By selection of the targeting sequences of the gRNA, defined regions of the target nucleic acid sequence or sequences bracketing a particular location within the target nucleic acid can be repressed using the LTRP:gRNA systems described herein.

In some embodiments, the targeting sequence of the gRNA has between 15 and 22 consecutive nucleotides. In some embodiments, the targeting sequence has 15, 16, 17, 18, 19, 20, 21, and 22 consecutive nucleotides. In some embodiments, the targeting sequence consists of 22 consecutive nucleotides. In some embodiments, the targeting sequence consists of 21 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. By selection of the targeting sequences of the gRNA, defined regions of the target nucleic acid sequence can be repressed and/or epigenetically modified using the LTRP:gRNA systems described herein.

The gene repressor systems of the present disclosure can be designed to target any region of, or proximal to, a gene or region of a gene for which repression of transcription is sought. When the entirety of the gene is to be repressed, designing a guide with a targeting sequence complementary to a sequence encompassing or proximal to the transcription start site (TSS) is contemplated by the disclosure. The TSS selection occurs at different positions within the promoter region, depending on promoter sequence and initiating-substrate concentration. The core promoter serves as a binding platform for the transcription machinery, which comprises Pol II and its associated general transcription factors (GTFs) (Haberle, V. et al. Eukaryotic core promoters and the functional basis of transcription initiation (Nat Rev Mol Cell Biol. 19(10):621 (2018)). Variability in TSS selection has been proposed to involve DNA ‘scrunching’ and ‘anti-scrunching,’ the hallmarks of which are: (i) forward and reverse movement of the RNA polymerase leading edge, but not trailing edge, relative to DNA, and (ii) expansion and contraction of the transcription bubble. In some embodiments, the target nucleic acid sequence bound by an RNP of the LTRP:gRNA system is within 1.5 kilobase (kb) of a transcription start site (TSS) in a gene. In some embodiments, the target nucleic acid sequence bound by an RNP of the system is within 20 basepairs (bp), 50 bp, 100 bp, 150 bp, 200 bp, 250 bp, 500 bps, 1 kb, or 1.5 kb upstream of a TSS of a gene. In some embodiments, the target nucleic acid sequence bound by an RNP of the system is within 20 bp, 50 bp, 100 bp, 150 bp, 200 bp, 250 bp, 500 bps, 1 kb, or 1.5 kb downstream of a TSS of a gene. In some embodiments, the target nucleic acid sequence bound by an RNP of the system is within 500 bp upstream to 500 bp downstream, or 300 bp upstream to 300 bp downstream, or 100 bp upstream to 100 bp downstream of a TSS of a gene. In some embodiments, the target nucleic acid sequence bound by an RNP of the system is within 20 bp, 50 bp, 100 bp, 150 bp, 200 bp, 250 bp, 500 bps, or 1 kb of an enhancer of a gene. In some embodiments, the target nucleic acid sequence bound by an RNP of the system of the disclosure is within 1 kb 3′ to a 5′ untranslated region of a gene. In other embodiments, the target nucleic acid sequence bound by an RNP of the system is within the open reading frame of a gene, inclusive of introns (if any). In some embodiments, the targeting sequence of a gRNA of the system of the disclosure is complementary to an exon of a gene. In a particular embodiment, the targeting sequence of a gRNA of the system of the disclosure is complementary to exon 1 of a gene. In other embodiments, the targeting sequence of a gRNA of the system of the disclosure is complementary to an intron of a gene. In other embodiments, the targeting sequence of the gRNA of the system of the disclosure is complementary to an intron-exon junction of a gene. In other embodiments, the targeting sequence of the gRNA of the system of the disclosure is complementary to a regulatory element of a gene. In other embodiments, the targeting sequence of the gRNA of the system of the disclosure is complementary to a sequence of an intergenic region of a gene. In other embodiments, the targeting sequence of a gRNA of the system of the disclosure is specific for a junction of the exon, an intron, and/or a regulatory element of a gene. In those cases where the targeting sequence is specific for a regulatory element, such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. The promoter region is intended to encompass nucleotides within 5 kb of the initiation point of the encoding sequence or, in the case of gene enhancer elements or conserved elements, can be thousands of bp, hundreds of thousands of bp, or even millions of bp away from the encoding sequence of a gene. In the foregoing, the targets are those in which the gene comprising the target nucleic acid is intended to be repressed such that a gene product is not expressed or is expressed at a lower level in a cell. In some embodiments, upon binding of the RNP of the system of the disclosure to the binding location of the target nucleic acid, the system is capable of repressing transcription of a gene 5′ to the binding location of the RNP. In other embodiments, upon binding of the RNP of the system to the binding location of the target nucleic acid, the system is capable of repressing transcription of a gene 3′ to the binding location of the RNP.

c. gRNA Modifications

In another aspect, the disclosure relates to gRNA variants for use in the systems of the disclosure, which comprise modifications relative to a reference gRNA from which the gRNAs were derived. In some embodiments, a gRNA variant for use in the systems of the disclosure comprises one or more nucleotide substitutions, insertions, deletions, or swapped or replaced domains relative to a gRNA sequence of the disclosure that improve a characteristic relative to the reference gRNA. Exemplary regions for modifications and swapped regions or domains include the RNA triplex, the pseudoknot, the scaffold stem loop, and the extended stem loop. In some embodiments, the gRNA variant of the disclosure comprises at least a first swapped region from a different gRNA, resulting in a chimeric gRNA. A representative example of such a chimeric gRNA is guide 316 (SEQ ID NO: 1746), in which the extended stem loop of gRNA scaffold 235 (SEQ ID NO: 1745) is replaced with the extended stem loop of gRNA scaffold 174 (SEQ ID NO: 1744), wherein the resulting 316 variant retains the ability to form an RNP with a dCasX and a long-term repressor fusion protein and exhibits an improved characteristic compared to the parent 235, when assessed in an in vitro or in vivo assay under comparable conditions.

All gRNAs that have one or more improved functions, characteristics, or add one or more new functions when the gRNA scaffold variant is compared to a gRNA scaffold from which it was derived, while retaining the functional properties of being able to complex with the long-term repressor fusion protein and guide the ribonucleoprotein holo RNP complex to the target nucleic acid are envisaged as within the scope of the disclosure. In some embodiments, the gRNA has an improved characteristic selected from the group consisting of increased pseudoknot stem stability, increased triplex region stability, increased scaffold stem stability, extended stem stability, reduced off-target folding intermediates, increased binding affinity to a repressor fusion protein, and increased transcriptional repression activity when complexed with a repressor fusion protein, or any combination thereof. In some cases of the foregoing, the improved characteristic is assessed in an in vitro assay, including the assays of the Examples. In other cases of the foregoing, the improved characteristic is assessed in vivo.

Table 8 provides exemplary gRNA variant scaffold sequences of the disclosure that are utilized as gRNA scaffolds or for the generation of the gRNAs for use in the LTRP:gRNA systems of the disclosure. In some embodiments, the gRNA variant scaffold for use in the systems comprises any one of the sequences of SEQ ID NOS: 1744-1746 as listed in Table 8, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto, wherein the gRNA variant retains the ability to form an RNP with a dCasX of the disclosure. In other embodiments, the gRNA variant scaffold for use in the LTRP:gRNA systems comprise any one of the sequences of SEQ ID NOS: 1744-1746, wherein the gRNA variant retains the ability to form an RNP with a long-term repressor fusion protein of the disclosure. It will be understood that in those embodiments wherein a vector comprises a DNA encoding sequence for a gRNA, that thymine (T) bases can be substituted for the uracil (U) bases of any of the gRNA sequence embodiments described herein. Similarly, any RNA sequence disclosed herein can be encoded by a DNA in which the uracil bases are substituted by thymine. In some embodiments, the disclosure provides gRNA variants that are chemically-modified, described below. In some embodiments, the gRNA comprises a scaffold comprising a sequence of SEQ ID NOS: 1744-1746, and is chemically modified.

TABLE 8
gRNA Scaffold Sequences

| SEQ ID | Scaffold | |
|---|---|---|
| NO: | variant ID | Nucleotide sequence |
| 1744 | 174 | ACUGGCGCUUUUAUCUGAUUACUUUG |
| | | AGAGCCAUCACCAGCGACUAUGUCGU |
| | | AGUGGGUAAAGCUCCCUCUUCGGAGG |
| | | GAGCAUCAAAG |
| 1745 | 235 | ACUGGCGCUUCUAUCUGAUUACUCUG |
| | | AGCGCCAUCACCAGCGACUAUGUCGU |
| | | AGUGGGUAAAGCCGCUUACGGACUUC |
| | | GGUCCGUAAGAGGCAUCAGAG |
| 1746 | 316 | ACUGGCGCUUCUAUCUGAUUACUCUG |
| | | AGCGCCAUCACCAGCGACUAUGUCGU |
| | | AGUGGGUAAAGCUCCCUCUUCGGAGG |
| | | GAGCAUCAGAG |

Additional gRNA variants contemplated for use in the systems of the disclosure are selected from the group consisting of SEQ ID NOS: 1747-1821. In some embodiments, the gRNA comprises a scaffold comprising a sequence of SEQ ID NOS: 1747-1821, and is chemically modified.

Guide scaffolds can be made by several methods, including recombinantly or by solid-phase RNA synthesis. However, the length of the scaffold can affect the manufacturability when using solid-phase RNA synthesis, with longer lengths resulting in increased manufacturing costs, decreased purity and yield, and higher rates of synthesis failures. For use in particle formulations, such as lipid nanoparticle (LNP) formulations, solid-phase RNA synthesis of the scaffold is preferred to generate the quantities needed for commercial development. While previous experiments had identified gRNA scaffold 235 (SEQ ID NO: 1745) as having enhanced properties relative to gRNA scaffold 174 (SEQ ID NO: 1744), its increased length (in nucleotides) potentially rendered its use for LNP formulations problematic due to synthetic manufacturing constraints. Accordingly, alternative sequences were sought. In some embodiments, the disclosure provides gRNA variant scaffolds having improved manufacturability compared to the gRNA scaffold from which it was derived. In some embodiments, the disclosure provides a gRNA wherein the gRNA scaffold and linked targeting sequence has a sequence that is less than about 115 nucleotides, less than about 110 nucleotides, or less than about 100 nucleotides. In a particular embodiment, the 316 gRNA scaffold (SEQ ID NO: 1746) has a shorter sequence compared to the 235 scaffold from which it was derived. The 316 gRNA scaffold was designed wherein the scaffold 235 sequence was modified by a domain swap in which the extended stem loop of scaffold 174 replaced the extended stem loop of the 235 scaffold, resulting in the chimeric gRNA scaffold 316, having the sequence ACUGGCGCUUCUAUCUGAUUACUCUGAGCGCCAUCACCAGCGACUAUGUCGUAGU GGGUAAAGCUCCCUCUUCGGAGGGAGCAUCAGAG (SEQ ID NO: 1746), having 89 nucleotides, compared with the 99 nucleotides of gRNA scaffold 235. The resulting 316 scaffold had the further advantage in that the extended stem loop does not contain CpG motifs; an enhanced property conferring reduced potential to elicit an immune response. In some embodiments, the shorter sequence length of the 316 scaffold confers the improvements of a higher fidelity in the ability to create the guide synthetically with the correct and complete sequence, as well as an enhanced ability to be successfully incorporated into an LNP. In some embodiments, the disclosure provides gRNA 316 variants that are chemically-modified, described below.

d. Chemically-Modified gRNAs

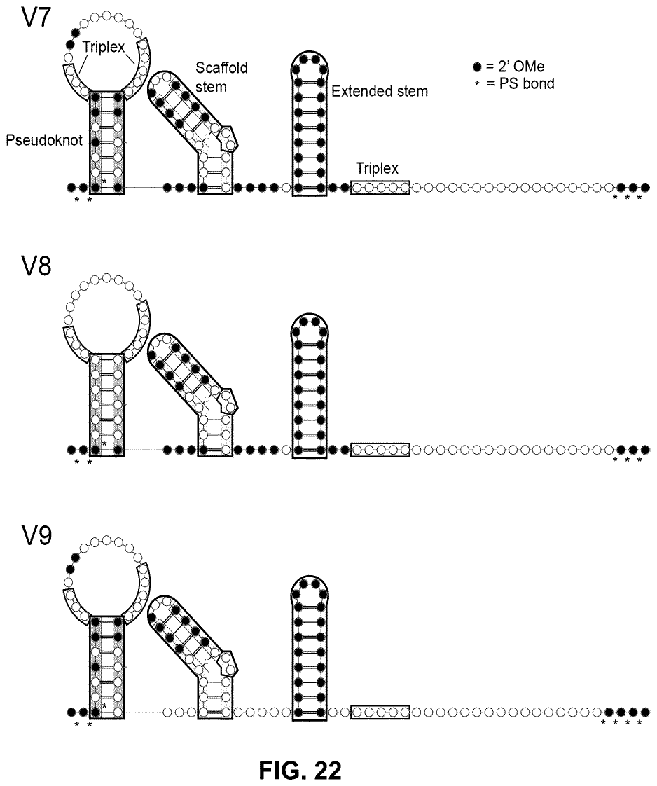

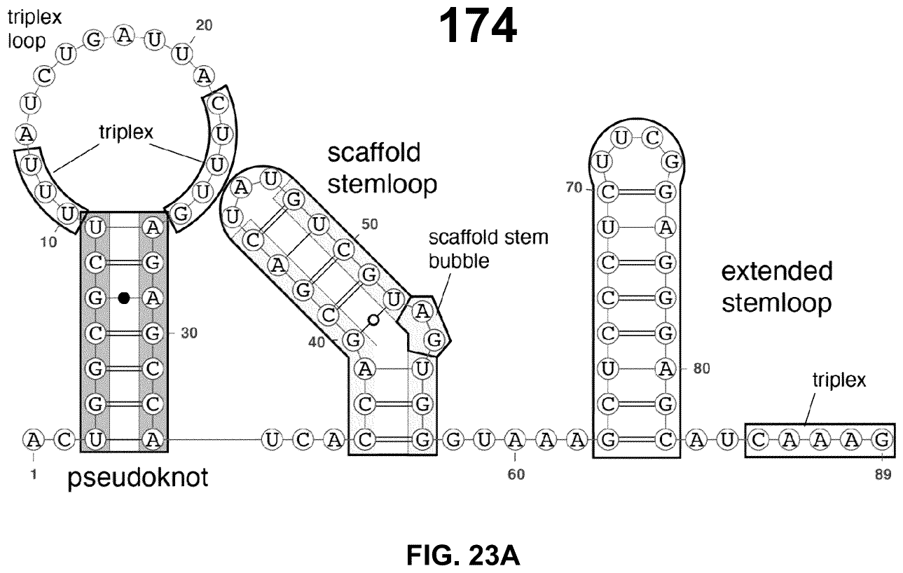

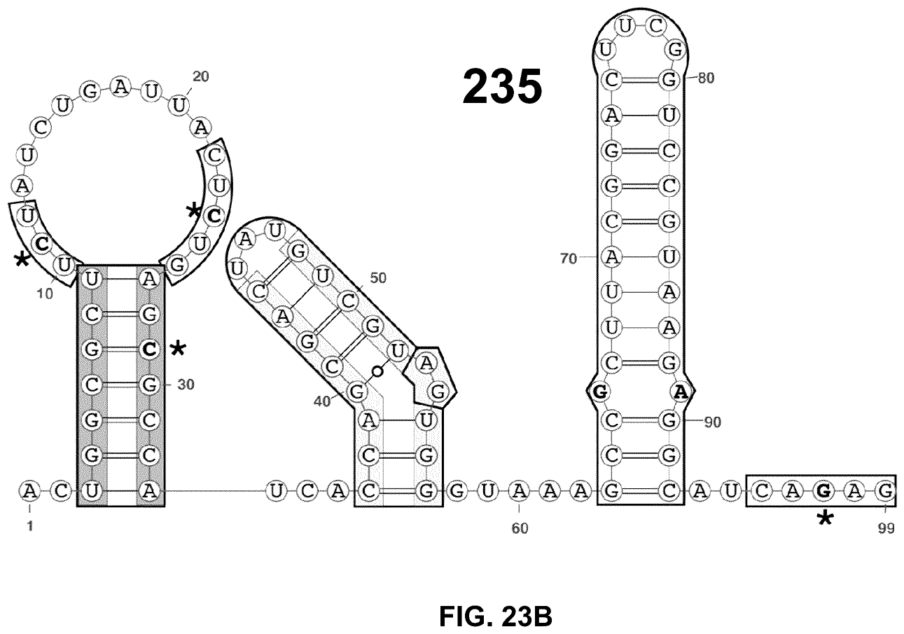

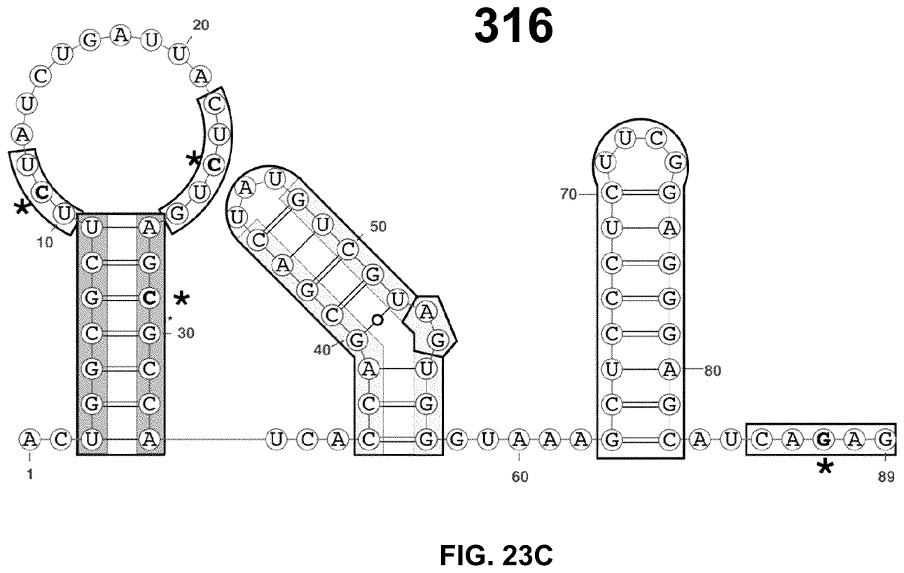

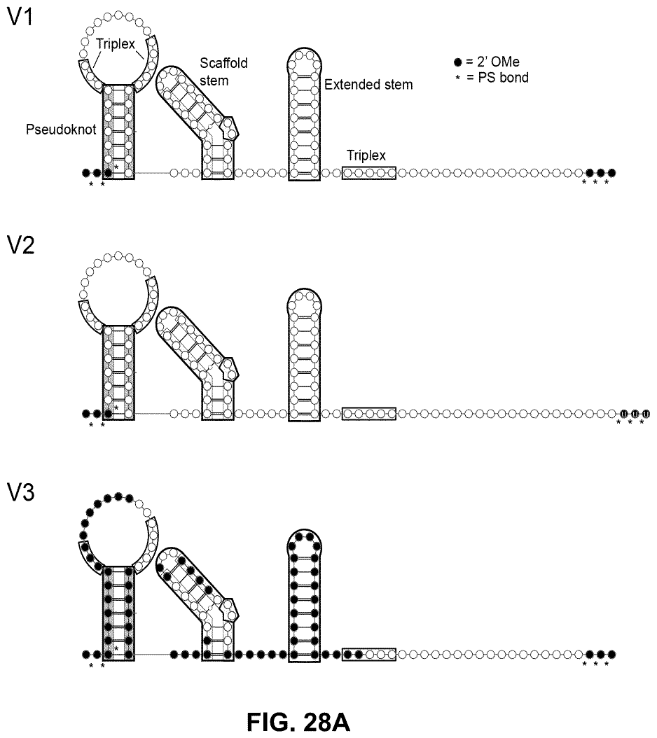

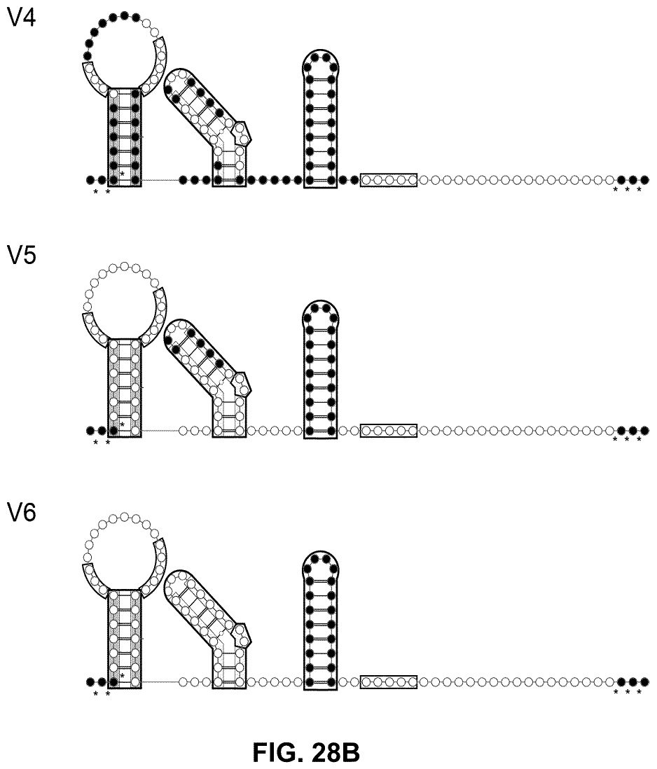

In some embodiments, the gRNAs have one or more chemical modifications. In some embodiments, the chemical modification is the addition of a 2′O-methyl group to one or more nucleotides of the sequence. In some embodiments, one or more nucleotides on each terminal end of the gRNA are modified by an addition of a 2′O-methyl group. In some embodiments, the chemical modification is substitution of a phosphorothioate bond between two or more nucleotides of the sequence. In some embodiments, the chemical modification is a substitution of phosphorothioate bonds between two or more nucleotides on each terminal end of the gRNA. In some embodiments, the gRNA comprises a substitution of phosphorothioate bonds between two or more nucleotides located 1, 2, 3 or 4 nucleotides the from the 5′ terminal end, the 3′ terminal, or both terminal ends of the gRNA. In some embodiments, the gRNA comprises an addition of a 2′O-methyl group to one or more nucleotides of the gRNA. In some embodiments, one or more nucleotides located 1, 2, 3, or 4 nucleotides from the 5′ terminal end, the 3′ terminal, or both terminal ends of the gRNA are modified by an addition of a 2′O-methyl group. In some embodiments, the first 1, 2, or 3 nucleotides of the 5′ end of the scaffold (i.e., A, C, and U in the case of gRNA 174, 235, and 316) are modified by the addition of a 2′O-methyl group and each of the modified nucleotides is linked to the adjoining nucleotide by a phosphorothioate bond. Similarly, the last 1, 2, or 3 nucleotides of the 3′ end of the targeting sequence linked to the 3′ end of the scaffold are similarly modified. In some embodiments, the gRNA comprising one or more chemical modifications comprises a sequence selected from the group consisting of the sequences of SEQ ID NOS: 2136-2144; 2146-2154; and 2156-2164, or a sequence having at least about 70%, at least about 80%, at least about 90%, at least about 95%, at least about 96%, at least about 97%, at least about 98%, at least about 99% sequence identity thereto. In some embodiments, the gRNA with chemical modifications comprises a scaffold of SEQ ID NOS: 2136-2144; 2146-2154; and 2156-2164; i.e., a sequence of SEQ ID NOS: 2136-2144; 2146-2154; and 2156-2164 without the 20 nucleotide spacer on the 3′ end represented in the foregoing sequences as undefined nucleotides. A schematic of the structure of gRNA variants 174, 235, and 316 are shown in FIGS. 23A-23C, respectively, and schematics of chemically-modified gRNA are show in FIGS. 22, 28A, and 28B. In some embodiments, the gRNA with chemical modifications exhibits improved stability compared to a gRNA without chemical modifications.

e. Complex Formation with Long-term Repressor Fusion Protein

Upon delivery or expression of the components of the system in a target cell, the gRNA variant is capable of complexing with the long-term repressor fusion protein as an RNP, and binding to the target nucleic acid targeted by the targeting sequence of the gRNA. In some embodiments, a gRNA variant has an improved ability to form an RNP complex with a long-term repressor fusion protein when compared to a reference gRNA or another gRNA variant from which it was derived. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene repression of a target nucleic acid.

### VII. Polynucleotides and Vectors

The disclosure provides polynucleotides encoding the long-term repressor fusion protein and/or gRNA. The disclosure provides polynucleotides encoding mRNA encoding the long-term repressor fusion protein, e.g. DNA polynucleotides encoding the corresponding mRNA.

A long-term repressor fusion protein or an mRNA encoding the long-term repressor fusion protein of the disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids or nucleotides (as applicable) may be substituted with unnatural amino acids or nucleotides. The particular sequence and the manner of preparation will be determined by convenience, economics, purity required, and the like. A gRNA can also be produced synthetically; for example, by use of a T7 RNA polymerase system known in the art.

The long-term repressor fusion protein and/or the gRNA may also be prepared by recombinantly producing a polynucleotide sequence coding for the long-term repressor fusion protein or gRNA of any of the embodiments described herein using standard recombinant techniques known in the art and incorporating the encoding gene into an expression vector appropriate for a host cell. For production of the encoded long-term repressor fusion protein and/or gRNA of any of the embodiments described herein, the methods include transforming an appropriate host cell with an expression vector comprising the encoding polynucleotide, and culturing the host cell under conditions causing or permitting the resulting long-term repressor fusion protein or gRNA of any of the embodiments described herein to be expressed or transcribed in the transformed host cell, which are recovered by methods described herein or by standard purification methods known in the art or as described in the Examples. Standard recombinant techniques in molecular biology are used to make the polynucleotides and expression vectors of the present disclosure.

A long-term repressor fusion protein and/or a gRNA of the disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 50% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases, a long-term repressor fusion protein or gRNA of the present disclosure is at least 80% pure, at least 85% pure, at least 90% pure, at least 95% pure, at least 98% pure, or at least 99% pure (e.g., free of contaminants or other macromolecules, etc.).

Additionally, the disclosure provides vectors comprising polynucleotides encoding the repressor fusion proteins and the gRNAs described herein. In some cases, the vectors are utilized for the expression and recovery of the CasX and gRNA components of the LTRP:gRNA system, when the system is delivered as a repressor fusion protein and gRNA, or an RNP. In other cases, the vectors are utilized for the delivery of the encoding polynucleotides to target cells for the transcriptional repression and/or epigenetic modification of the target nucleic acid, as described more fully, below. In some embodiments, sequences encoding the long-term repressor fusion protein and a gRNA are templated on the same vector. In some embodiments, sequences encoding the long-term repressor fusion protein and a gRNA are templated on different vectors. Suitable vectors are described, for example, in WO2023235818A2, WO2022120095A1 and WO2020247882A1, incorporated by reference herein. As described in WO2023235818A2, WO2022120095A1 and WO2020247882A1, depending on the host/vector system utilized, any of a number of suitable transcription and translation control elements, including constitutive and inducible promoters, transcription enhancer elements, transcription terminators, etc. may be used in the expression vector.

In some embodiments, the disclosure provides polynucleotide sequences encoding the long-term repressor fusion protein of any of the embodiments described herein, including the long-term repressor fusion proteins comprising a sequence of SEQ ID NOS: 1883-1903, 1909-1912, or 1915-1924, or sequences having at least about 50%, at least about 60%, at least about 70%, at least about 80%, at least about 90%, at least about 95%, at least about 96%, at least about 97%, at least about 98%, or at least about 99% sequence identity thereto. In some embodiments, the polynucleotide comprises a sequence encoding the long-term repressor fusion protein comprising a sequence of SEQ ID NOS: 1883-1903, 1909-1912, or 1915-1924. In some embodiments, the polynucleotide comprises a sequence of an mRNA encoding the long-term repressor fusion protein of any of the embodiments described herein for use in a particle system for delivery to a cell. In some embodiments, the mRNA sequences encoding the long-term repressor fusion protein for use in LNP particle formulations for delivery to a cell. In a particular embodiment, the disclosure provides gRNA and mRNA sequences encoding the long-term repressor fusion protein for use in LNP particle formulations for delivery to a cell, described more fully, below.

In some embodiments, the disclosure provides an isolated polynucleotide sequence encoding a gRNA variant of any of the embodiments described herein. In some embodiments, the disclosure provides polynucleotides encoding a gRNA comprising a scaffold sequence comprising SEQ ID NOS: 1744-1746, or a sequence having at least about 50%, at least about 60%, at least about 70%, at least about 80%, at least about 90%, at least about 95%, at least about 96%, at least about 97%, at least about 98%, or at least about 99% sequence identity thereto, wherein the expressed gRNA variant retains the ability to form an RNP with a repressor fusion protein. In the foregoing embodiment, the gRNA further comprises a targeting sequence complementary to a target nucleic acid of a gene to be repressed.

In some embodiments, the disclosure relates to methods to produce polynucleotide sequences encoding the long-term repressor fusion protein or the gRNA of any of the embodiments described herein, including variants thereof, as well as methods to express the proteins or RNA transcribed by the polynucleotide sequences. In general, the methods include producing a polynucleotide sequence coding for the long-term repressor fusion protein or the gRNA of any of the embodiments described herein and incorporating the encoding gene into an expression vector. In some embodiments, the vector is designed for transduction of cells for transcriptional repression and/or epigenetic modification of a target nucleic acid. Such vectors can include a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector. In other embodiments, the expression vector is designed for production of repressor fusion protein, and mRNA encoding the long-term repressor fusion protein, or gRNA in either a cell-free system or in a host cell. For production of the encoded long-term repressor fusion protein or the gRNA of any of the embodiments described herein in a host cell, the methods include transforming an appropriate host cell with an expression vector comprising the encoding polynucleotide, and culturing the host cell under conditions causing or permitting the resulting long-term repressor fusion protein or the gRNA of any of the embodiments described herein to be expressed or transcribed in the transformed host cell, thereby producing the long-term repressor fusion protein or the gRNA, which are recovered by methods described herein (e.g., in the Examples, below) or by standard purification methods known in the art. Standard recombinant techniques in molecular biology are used to make the polynucleotides and expression vectors of the present disclosure.

In accordance with the disclosure, nucleic acid sequences that encode the long-term repressor fusion protein or the gRNA of any of the embodiments described herein are used to generate recombinant nucleic acid molecules that direct the expression in appropriate host cells. Several cloning strategies are suitable for performing the present disclosure, many of which are used to generate a construct that comprises a gene coding for a long-term repressor fusion protein or gRNA of the present disclosure, or its complement. In some embodiments, the cloning strategy is used to create a gene that encodes a construct that comprises nucleotides encoding the long-term repressor fusion protein or the gRNA. In some embodiments, the gene, for example as part of a vector, is used to transform a host cell for expression of the gene, e.g. expression of the long-term repressor fusion protein or gRNA.

In one approach, a construct is first prepared containing the DNA sequence encoding a long-term repressor fusion protein or a gRNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the repressor fusion protein, or the gRNA. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), yeast cells, or other eukaryotic cells known in the art suitable for the production of recombinant products. Exemplary methods for the creation of expression vectors, the transformation of host cells and the expression and recovery of the long-term repressor fusion protein or the gRNA are described in the Examples.

The gene encoding the long-term repressor fusion protein or the gRNA construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components into a gene of a desired sequence. Genes encoding polypeptide compositions are assembled from oligonucleotides using standard techniques of gene synthesis.

In some embodiments, the nucleotide sequence encoding a long-term repressor fusion protein is codon optimized. This type of optimization can entail a mutation of an encoding nucleotide sequence to mimic the codon preferences of the intended host organism or cell while encoding the same protein, as well as other parameters, including the codon adaptation index (CAI), codon-usage tables derived from biologics intended for use as therapeutics, mRNA stability index, or GC content. Thus, the codons can be changed, but the encoded protein remains unchanged. For example, if the intended target cell of the long-term repressor fusion protein was a human cell, a human codon-optimized long-term repressor fusion protein-encoding nucleotide sequence could be used. As another non-limiting example, if the intended host cell were a mouse cell, then a mouse codon-optimized long-term repressor fusion protein-encoding nucleotide sequence could be generated. The gene design can be performed using algorithms that optimize codon usage and amino acid composition appropriate for the host cell utilized in the production of the long-term repressor fusion protein or the gRNA. In one method of the disclosure, a library of polynucleotides encoding the components of the constructs is created and then assembled, as described above. The resulting genes are then assembled and the resulting genes used to transform a host cell and produce and recover the long-term repressor fusion protein or the gRNA compositions for evaluation of its properties or for use in the modification of a target nucleic acid, as described herein.

In some embodiments, a nucleotide sequence encoding a long-term repressor fusion protein or a gRNA is operably linked to a control element, e.g., a transcriptional control element, such as a promoter. In some embodiments, a nucleotide sequence encoding a long-term repressor fusion protein is operably linked to a control element, e.g., a transcriptional control element, such as a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hepatocytes or a liver sinusoidal endothelial cell.

Non-limiting examples of Pol II promoters operably linked to the polynucleotide encoding the long-term repressor fusion protein of the disclosure include, but are not limited to EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken 3-actin promoter (CBA), CBA hybrid (CBh), chicken 3-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the Ulb2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POL1 promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the Pol II promoter is EF-1alpha, wherein the promoter enhances transfection efficiency, the transgene transcription or expression of the CRISPR nuclease, the proportion of expression-positive clones and the copy number of the episomal vector in long-term culture.

Non-limiting examples of Pol III promoters operably linked to the polynucleotide encoding the gRNA variants of the disclosure include, but are not limited to U6, mini U6, U6 truncated promoters,7SK, and H1 variants, BiHi (Bidrectional H1 promoter), BiU6, Bi7SK, BiH1 (Bidirectional U6, 7SK, and H1 promoters), gorilla U6, rhesus U6, human 7SK, human H1 promoters, and truncated versions and sequence variants thereof. In the foregoing embodiment, the pol III promoter enhances the transcription of the gRNA. In a particular embodiment, the Pol III promoter is U6, wherein the promoter enhances expression of the gRNA. Experimental details and data for the use of such promoters are provided in the Examples.

Selection of the appropriate vector and promoter is well within the level of ordinary skill in the art, as it related to controlling expression. The expression vector may also contain a ribosome binding site for translation initiation, and a transcription terminator. The expression vector may also include appropriate sequences for amplifying expression. The expression vector may also include nucleotide sequences encoding protein tags (e.g., 6xHis tag, hemagglutinin tag, fluorescent protein, etc.) that can be fused to the long-term repressor fusion protein, thus resulting in a chimeric protein that are used for purification or detection.

Recombinant expression vectors of the disclosure can also comprise elements that facilitate robust expression of the proteins and the gRNAs of the disclosure. For example, recombinant expression vectors can include one or more of a polyadenylation signal (poly(A)), an intronic sequence or a post-transcriptional regulatory element such as a woodchuck hepatitis post-transcriptional regulatory element (WPRE). Exemplary poly(A) sequences include hGH poly(A) signal (short), HSV TK poly(A) signal, synthetic polyadenylation signals, SV40 poly(A) signal, P-globin poly(A) signal, and a split poly(A) sequence with a SphI restriction site between two 60 A stretches (SEQ ID NO: 21851), and the like. A person of ordinary skill in the art will be able to select suitable elements to include in the recombinant expression vectors described herein.

The polynucleotides encoding the long-term repressor fusion protein or the gRNA sequences can be individually cloned into an expression vector. Selection of the appropriate vector and promoter is well within the level of ordinary skill in the art, as it relates to controlling expression, e.g., for repressing expression and/or epigenetic modification of a gene. The expression vector may also contain a ribosome binding site for translation initiation and a transcription terminator. The expression vector may also include appropriate sequences for amplifying expression.

The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the long-term repressor fusion protein or gRNA can be determined using any nucleic acid or protein assay known in the art. For example, the presence of transcribed mRNA of the long-term repressor fusion protein can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of CasX polynucleotide.

In some embodiments, a vector is created for the transcription of the long-term repressor fusion protein, and expression and recovery of the resulting encoding mRNA. In some embodiments, the mRNA is generated by in vitro transcription (IVT) using a PCR product or linearized plasmid DNA template and a T7 RNA polymerase, wherein the plasmid contains a T7 promoter. If using a PCR product, DNA sequences encoding candidate mRNAs will be cloned into a plasmid containing a T7 promoter, wherein the plasmid DNA template will be linearized and then used to perform IVT reactions for expression of the mRNA. Exemplary methods for the generation of such vectors and the production and recovery of the mRNA are provided in the Examples, below.

VIII. Particles for Delivery of LTRP:gRNA systems

In another aspect, the present disclosure provides particle compositions for delivery of gene repressor systems, such as the LTRP:gRNA systems described herein, to cells or to subjects for the transcriptional repression or silencing of a gene. Particles envisaged as within the scope of the instant disclosure include, but are not limited to, nanoparticles such as synthetic nanoparticles, polymeric nanoparticles, lipid nanoparticles, viral particles and virus-like particles. Particles of the disclosure may encapsulate payloads such as gRNA variants, as described herein, optionally in combination with mRNA encoding the repressor fusion proteins of any of the embodiments described herein. Alternatively, or in addition, particles of the disclosure may encapsulate payloads of gRNA variants and repressor fusion proteins, for example when associated as a ribonucleoprotein (RNP) complex. In some embodiments, the particles are synthetic nanoparticles that encapsulate payloads of gRNA variants and mRNA encoding repressor fusion proteins of any of the embodiments described herein. In some embodiments, the synthetic nanoparticles comprise biodegradable polymeric nanoparticles (PNP). In some embodiments, materials for the creation of biodegradable polymeric nanoparticles (PNP) include polylactide, poly (lactic-co-glycolic acid) (PLGA), poly(ethyl cyanoacrylate), poly(butyl cyanoacrylate), poly(isobutyl cyanoacrylate), and poly(isohexyl cyanoacrylate), polyglutamic acid (PGA), poly (e-caprolactone) (PCL), cyclodextrin, and natural polymers for instance chitosan, albumin, gelatin, and alginate, which are the most utilized polymers for the synthesis of PNP (Production and clinical development of nanoparticles for gene delivery. Molecular Therapy-Methods & Clinical Development 3:16023; doi:10.1038 (2016)). In other embodiments, the particles are lipid nanoparticles (LNPs) that encapsulate a gRNA variant and a mRNA encoding long-term repressor fusion protein of any of the embodiments described herein, described more fully, below. In other embodiments, the particles are lipid nanoparticles that separately encapsulate a gRNA variant and a mRNA encoding long-term repressor fusion protein of any of the embodiments described herein in different particles, that are the co-formulated as a mixture for administration, described more fully, below. In other embodiments, the particles are lipid nanoparticles that separately encapsulate a gRNA variant and a mRNA encoding long-term repressor fusion protein of any of the embodiments, and the two types of particles are administered separately.

a. Lipid Nanoparticles (LNP)

The present disclosure provides lipid nanoparticles (LNP) for delivery of the LTRP:gRNA systems described herein to cells or to subjects. In some embodiments, the LNPs of the disclosure are tissue-specific, have excellent biocompatibility, and can deliver the LTRP:gRNA systems with high efficiency, and thus can be used for the transcriptional repression of the targeted gene.

The disclosure further provides LNP compositions and pharmaceutical compositions comprising a plurality of the LNP described herein.

In their native forms, nucleic acid polymers are generally unstable in biological fluids and cannot penetrate into the cytoplasm of target cells, thus requiring delivery systems. Lipid nanoparticles (LNP) have proven useful for both the protection and delivery of nucleic acids to tissues and cells. Furthermore, the use of mRNA in LNPs to encode the long-term repressor fusion protein eliminates the possibility of undesirable genome integration, as compared to DNA vectors. Moreover, mRNA efficiently transfects both mitotic and non-mitotic cells, as it does not require entry into the nucleus since it exerts its function in the cytoplasmic compartment. LNPs as a delivery platform thus offer the additional advantage of being able to co-formulate both the mRNA encoding the long-term repressor fusion protein and a gRNA into single LNP particles.

Accordingly, in various embodiments, the disclosure encompasses lipid nanoparticles and compositions that may be used for a variety of purposes, including the delivery of encapsulated or associated (e.g., complexed) therapeutic agents such as nucleic acids to cells, both in vitro and in vivo. In certain embodiments, the disclosure encompasses methods of treating or preventing diseases or disorders in a subject in need thereof by contacting the subject with a lipid nanoparticle that encapsulates or is associated with a suitable therapeutic agent complexed through various physical, chemical or electrostatic interactions between one or more of the lipid components used in the compositions to make LNPs.

In some embodiments, the LNP comprises an LTRP:gRNA system as described herein for repression or silencing of a gene associated with the disease or disorder. In some embodiments, the present disclosure provides LNP in which the gRNA and mRNA encoding the long-term repressor fusion protein are incorporated into single LNP particles. In some embodiments, the LNP comprises an mRNA comprising a sequence selected from the group consisting of SEQ ID NOS: 2409-18636, or a sequence having at least about 70%, at least about 80%, at least about 90%, at least about 95%, at least about 96%, at least about 97%, at least about 98%, at least about 99% sequence identity thereto, and a gRNA comprising a sequence selected from the group consisting of SEQ ID NOS: 1744-1746, 2136-2144, 2146-2154, and 2156-2164, with a linked targeting sequence complementary to a sequence of a gene targeted for repression or silencing. In one embodiment, the LNP comprises an mRNA comprising a sequence selected from the group consisting of SEQ ID NOS: 2411, 2421, 2467, and 2477, and a gRNA comprising a sequence of SEQ ID NO: 2156 with a linked targeting sequence complementary to a sequence of a gene targeted for repression or silencing. In other embodiments, the present disclosure provides LNP in which the gRNA and mRNA encoding the long-term repressor fusion protein are incorporated into separate populations of LNPs, which can be formulated together in varying ratios for administration.

The lipid nanoparticles and lipid nanoparticle compositions of the disclosure may be used to repress expression of a desired protein both in vitro and in vivo by contacting cells with a lipid nanoparticle comprising one or more ionizable lipids described herein, wherein the lipid nanoparticle encapsulates or is associated with a nucleic acid that is expressed to produce the desired protein (e.g., a messenger RNA encoding the repressor fusion protein). In some embodiments, the lipid nanoparticles and compositions may be used to repress the expression of a target gene both in vitro and in vivo by contacting cells with a lipid nanoparticle comprising one or more novel cationic lipids described herein, wherein the lipid nanoparticle encapsulates or is associated with one or more nucleic acids of the LTRP:gRNA systems of the disclosure. The lipid nanoparticles and compositions of embodiments of the disclosure may also be used for co-delivery of different nucleic acids (e.g., mRNA and plasmid DNA) separately or in combination, such as may be useful to provide an effect requiring colocalization of different nucleic acids (e.g., mRNA encoding for a suitable gene repressing factor or enzyme and gRNA for targeting of the gene).

In some embodiments, LNPs and LNP compositions described herein include at least one cationic lipid, at least one conjugated lipid, at least one steroid or derivative thereof, at least one additional lipid, or any combination thereof. Alternatively, the lipid compositions of the disclosure can include an ionizable lipid, such as an ionizable cationic lipid, a helper lipid (usually a phospholipid), cholesterol, and a polyethylene glycol-lipid conjugate (PEG-lipid) to improve the colloidal stability in biological environments by, for example, reducing a specific absorption of plasma proteins and forming a hydration layer over the nanoparticles. Such lipid compositions can be formulated at typical mole ratios of 50:10:37-39:1.5-2.5 or 20-50:8-65:25-40:1-2.5, with variations made to adjust individual properties.

The LNPs and LNP compositions of the present disclosure are configured to protect and deliver an encapsulated payload of the systems of the disclosure to tissues and cells, both in vitro and in vivo. Various embodiments of the LNPs and LNP compositions of the present disclosure are described in further detail herein.

### Cationic Lipid

In some aspects, the LNPs and LNP compositions of the present disclosure include at least one cationic lipid. The term “cationic lipid,” refers to a lipid species that has a net positive charge. In some embodiments, the cationic lipid is an ionizable cationic lipid that has a net positive charge at a selected pH, such as physiological pH. In some embodiments, the ionizable cationic lipid has a pKa less than 7 such that the LNPs and LNP compositions achieve efficient encapsulation of the payload at a relatively low pH. In some embodiments, the cationic lipid has a pKa of 5 to 8, 5.5 to 7.5, 6 to 7, or 6.5 to 7. In some embodiments, the cationic lipid may be protonated at a pH below the pKa of the cationic lipid, and it may be substantially neutral at a pH over the pKa. The LNPs and LNP compositions may be safely delivered to a target organ (for example, the liver, lung, heart, spleen, as well as to tumors) and/or cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

Early formulations of LNP utilizing permanently cationic lipids resulted in LNPs with positive surface charge that proved toxic in vivo, plus were rapidly cleared by phagocytic cells. By changing to ionizable cationic lipids bearing tertiary amines, especially those with pKa <7, results in LNP achieving efficient encapsulation of nucleic acid polymers at low pH by interacting electrostatically with the negative charges of the phosphate backbone of mRNA, that also result in largely neutral systems at physiological pH values, thus alleviating problems associated with permanently-charged cationic lipids.

As used herein, “ionizable lipid” means an amine-containing lipid which can be easily protonated, and, for example, it may be a lipid of which charge state changes depending on the surrounding pH. The ionizable lipid may be protonated (positively charged) at a pH below the pKa of a cationic lipid, and it may be substantially neutral at a pH over the pKa. In one example, the LNP may comprise a protonated ionizable lipid and/or an ionizable lipid showing neutrality. In some embodiments, the LNP has a pKa of 5 to 8, 5.5 to 7.5, 6 to 7, or 6.5 to 7. The pKa of the LNP is important for in vivo stability and release of the nucleic acid payload of the LNP in the target cell or organ. In some embodiments, the LNP having the foregoing pKa ranges may be safely delivered to a target organ (for example, the liver, lung, heart, spleen, as well as to tumors) and/or target cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

The ionizable lipid is an ionizable compound having characteristics similar to lipids generally, and through electrostatic interaction with a nucleic acid (for example, an mRNA of the disclosure), may play a role of encapsulating the nucleic acid payloads within the LNP with high efficiency.

According to the type of the amine and the tail group comprised in the ionizable lipid, (i) the nucleic acid encapsulation efficiency, (ii) PDI (polydispersity index). and/or (iii) the nucleic acid delivery efficiency to tissue and/or cells constituting an organ (for example, hepatocytes or liver sinusoidal endothelial cells in the liver) of the LNP may be different. In certain embodiments, the ionizable lipid is an ionizable cationic lipid, and comprises from about 46 mol % to about 66 mol % of the total lipid present in the particle.

The LNP comprising an ionizable lipid comprising an amine may have one or more kinds of the following characteristics: (1) the ability to encapsulate a nucleic acid with high efficiency; (2) uniform size of prepared particles (or having a low PDI value); and/or (3) excellent nucleic acid delivery efficiency to organs such as liver, lung, heart, spleen, bone marrow, as well as to tumors, and/or cells constituting such organs (for example, hepatocytes, LSEC, cardiac cells, cancer cells, etc.).

In particular embodiments, the cationic lipid form plays a crucial role both in nucleic acid encapsulation through electrostatic interactions and intracellular release by disrupting endosomal membranes. The nucleic acid payloads are encapsulated within the LNP by the ionic interactions they form with the positively charged cationic lipid. Non-limiting examples of cationic lipid components utilized in the LNP of the disclosure are selected from DLin-MC3-DMA (heptatriaconta-6,9,28,31-tetraen-19-yl4-(dimethylamino)butanoate), DLin- KC2-DMA (2,2-dilinoleyl-4-(2-dimethylaminoethyl)-[1,3]-dioxolane), and TNT (1,3,5-triazinane-2,4,6-trione) and TT (N1,N3,N5-tris(2-aminoethyl)benzene-1,3,5-tricarboxamide). Non-limiting examples of helper lipids utilized in the LNP of the disclosure are selected from DSPC (1,2-distearoyl-sn-glycero-3-phosphocholine), POPC (2-Oleoyl-1-palmitoyl-sn-glycero-3-phosphocholine) and DOPE (1,2-Dioleoyl-sn-glycero-3-phosphoethanolamine), 1,2-dioleoyl-sn-glycero-3-phospho-(1′-rac-glycerol) DOPG, 1,2-Dimyristoyl-sn-glycero-3-phosphoethanolamine (DMPE), 1,2-dilauroyl-sn-glycero-3-phosphocholine (DLPC), sphingolipid, and ceramide. Cholesterol and PEG-DMG ((R)-2,3-bis(octadecyloxy)propyl-1-(methoxy polyethylene glycol 2000) carbamate), PEG-DSG (1,2-Distearoyl-rac-glycero-3-methylpolyoxyethylene glycol 2000), or DSPE-PEG2k (1,2-distearoyl-sn-glycero-3-phosphoethanolamine-N-[amino(polyethylene glycol)-2000]), are components utilized in the LNP of the disclosure for the stability, circulation, and size of the LNP.

In some embodiments, the cationic lipid in the LNP of the disclosure comprises a tertiary amine. In some embodiments, the tertiary amine includes alkyl chains connected to N of the tertiary amine with ether linkages. In some embodiments, the alkyl chains comprise C12-C30 alkyl chains having 0 to 3 double bonds. In some embodiments, the alkyl chains comprise C16-C22 alkyl chains. In some embodiments, the alkyl chains comprise C18 alkyl chains. A number of cationic lipids and related analogs have been described in U.S. Patent Publication Nos. 20060083780, 20060240554, 20110117125, 20190336608, 20190381180 and 20200121809; U.S. Pat. Nos. 5,208,036; 5,264,618; 5,279,833; 5,283,185; 5,753,613; 5,785,992; 9,738,593; 10,106,490; 10,166,298; 10,221,127; and 11,219,634; and PCT Publication No. WO 96/10390, the disclosures of which are herein incorporated by reference in their entirety.

In some embodiments, the cationic lipid in the LNP of the disclosure may comprise, for example, one or more ionizable cationic lipids wherein the ionizable cationic lipid is a dialkyl lipid. In other embodiments, the ionizable cationic lipid is a trialkyl lipid.

In some embodiments, the cationic lipid in the LNP of the disclosure is selected from 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N (N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), and any combination of the forgoing.

In some embodiments, the cationic lipid in the LNP of the disclosure is selected from heptatriaconta-6,9,28,31-tetraen-19-yl4-(dimethylamino)butanoate (DLin-MC3-DMA), 2,2-dilinoleyl-4-(2-dimethylaminoethyl)-[1,3]-dioxolane (DLin- KC2-DMA), (1,3,5-triazinane-2,4,6-trione) (TNT), N1,N3,N5-tris(2-aminoethyl)benzene-1,3,5-tricarboxamide (TT), and any combination of the forgoing.

In some embodiments, the N/P ratio (nitrogen from the cationic/ionizable lipid and phosphate from the nucleic acid) in the LNP of the disclosure is in the range of is about 3:1 to 7:1, or about 4:1 to 6:1, or is 3:1, or is 4:1, or is 5:1, or is 6:1, or is 7:1.

### Conjugated Lipid

In some embodiments, the LNPs and LNP compositions of the present disclosure include at least one conjugated lipid. In some embodiments, the conjugated lipid may be selected from a polyethyleneglycol (PEG)-lipid conjugate, a polyamide (ATTA)-lipid conjugate, a cationic-polymer-lipid conjugate (CPL), and any combination of the foregoing. In some cases, conjugated lipids can inhibit aggregation of the LNPs of the disclosure.

In some embodiments, the conjugated lipid of the LNP of the disclosure comprises a pegylated lipid. The terms “polyethyleneglycol (PEG)-lipid conjugate,” “pegylated lipid” “lipid-PEG conjugate”, “lipid-PEG”, “PEG-lipid”, “PEG-lipid”, or “lipid-PEG” are used interchangeably herein and refer to a lipid attached to a polyethylene glycol (PEG) polymer which is a hydrophilic polymer. The pegylated lipid contributes to the stability of the LNPs and LNP compositions and reduces aggregation of the LNPs.

As the PEG-lipid can form the surface lipid, the size of the LNP can be readily varied by varying the proportion of surface (PEG) lipid to the core (ionizable cationic) lipids. In some embodiments, the PEG-lipid of the LNP of the disclosure can be varied from −1 to 5 mol % to modify particle properties such as size, stability, and circulation time.

The lipid-PEG conjugate contributes to the particle stability in serum of the nanoparticle within the LNP, and plays a role of preventing aggregation between nanoparticles. In addition, the lipid-PEG conjugate may protect nucleic acids, such as mRNAs encoding the repressor fusion proteins of the disclosure, or gRNAs of the disclosure, from degrading enzymes during in vivo delivery of the nucleic acids and enhance the stability of the nucleic acids in vivo and increase the half-life of the delivered nucleic acids encapsulated in the nanoparticle. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof.

In some embodiments, the pegylated lipid of the LNP of the disclosure is selected from a PEG-ceramide, a PEG-diacylglycerol, a PEG-dialkyloxypropyl, a PEG-dialkoxypropylcarbamate, a PEG-phosphatidylethanoloamine, a PEG-phospholipid, a PEG-succinate diacylglycerol, and any combination of the foregoing.

In some embodiments, the pegylated lipid of the LNP of the disclosure is a PEG-dialkyloxypropyl. In some embodiments, the pegylated lipid is selected from PEG-didecyloxypropyl (C10), PEG-dilauryloxypropyl (C12), PEG-dimyristyloxypropyl (C14), PEG-dipalmityloxypropyl (C16), PEG-distearyloxypropyl (C18), and any combination of the foregoing.

In other embodiments, the lipid-PEG conjugate of the LNP of the disclosure may be PEG bound to phospholipid such as phosphatidylethanolamine (PEG-PE), PEG conjugated to ceramide (PEG-CER, ceramide-PEG conjugate, ceramide-PEG, cholesterol or PEG conjugated to derivative thereof, PEG-c-DOMG, PEG-DMG, PEG-DLPE, PEG-DMPE, PEG-DPPC, PEG-DSPE(DSPE-PEG), and a mixture thereof, and for example, may be C16-PEG2000 ceramide (N-palmitoyl-sphingosine-1-{succinyl[methoxy(polyethylene glycol)2000]}), DMG-PEG 2000, 14:0 PEG2000 PE.

In some embodiments, the pegylated lipid of the LNP of the disclosure is selected from 1-(monomethoxy-polyethyleneglycol)-2,3-dimyristoylglycerol, 4-O-(2′,3′-di(tetradecanoyloxy)propyl-1-O-(o-methoxy(polyethoxy)ethyl)butanedioate (PEG-S-DMG), ω-methoxy(polyethoxy)ethyl-N-(2,3-di(tetradecanoxy)propyl)carbamate, 2,3-di(tetradecanoxy)propyl-N-(o-methoxy(polyethoxy)ethyl)carbamate, and any combination of the foregoing.

In some embodiments, the pegylated lipid of the LNP of the disclosure is selected from mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-w-methyl-poly(ethylene glycol) (2 KPEG-DMG), and any combination of the foregoing.

In some embodiments, the PEG is directly attached to the lipid of the pegylated lipid. In other embodiments, the PEG is attached to the lipid of the pegylated lipid by a linker moiety selected from an ester-free linker moiety or an ester-containing linker moiety. Non-limiting examples of the ester-free linker moiety include amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide and combinations thereof. For example, the linker may contain a carbamate linker moiety and an amido linker moiety. Non-limiting examples of the ester-containing linker moiety include carbonate (—OC(O)O—), succinoyl, phosphate ester (—O—(O)POH—O—), sulfonate ester, and combinations thereof.

The PEG moiety of the pegylated lipid of the LNP of the disclosure described herein may have an average molecular weight ranging from about 550 daltons to about 10,000 daltons. In certain embodiments, the PEG moiety has an average molecular weight of from about 750 daltons to about 5,000 daltons, about 1,000 daltons to about 4,000 daltons, about 1,500 daltons to about 3,000 daltons, about 750 daltons to about 3,000 daltons, or about 1750 daltons to about 2,000 daltons.

In some embodiments, the conjugated lipid (e.g., pegylated lipid) comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In certain embodiments, the conjugated lipid comprises from about 0.5 mol % to about 3 mol % of the total lipid present in the particle.

In additional embodiments, the conjugated lipid (e.g., pegylated lipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

For the lipid in the lipid-PEG conjugate of the LNP of the disclosure, any lipid capable of binding to polyethyleneglycol may be used without limitation, and the phospholipid and/or cholesterol which are other elements of the LNP may be also used. In some embodiments, the lipid in the lipid-PEG conjugate may be ceramide, dimyristoylglycerol (DMG), succinoyl-diacylglycerol (s-DAG), distearoylphosphatidylcholine (DSPC), distearoylphosphatidylethanolamine (DSPE), or cholesterol, but not limited thereto.

In the lipid-PEG conjugate of the LNP of the disclosure, the PEG may be directly conjugated to the lipid or linked to the lipid via a linker moiety. Any linker moiety suitable for binding PEG to the lipid may be used, and for example, includes an ester-free linker moiety and an ester-containing linker moiety. The ester-free linker moiety includes not only amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide but also combinations thereof (for example, a linker containing both a carbamate linker moiety and an amido linker moiety), but not limited thereto. The ester-containing linker moiety includes for example, carbonate (—OC(O)O—), succinoyl, phosphate ester (—O—(O)POH—O—), sulfonate ester, and combinations thereof, but not limited thereto.

### Steroids

In some embodiments, the LNPs and LNP compositions of the present disclosure include at least one steroid or derivative thereof. In some embodiments, the steroid comprises cholesterol. In some embodiments, the LNPs and LNP compositions comprise a cholesterol derivative selected from cholestanol, cholestanone, cholestenone, coprostanol, cholesteryl-2′-hydroxyethyl ether, cholesteryl-4′-hydroxybutyl ether, and any combination of the foregoing.

In some embodiments, the steroid (e.g., cholesterol) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the steroid (e.g., cholesterol) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

### Additional Lipid

In some embodiments, the LNPs and LNP compositions of the present disclosure include at least one additional lipid. In some embodiments, the additional lipid is non-cationic lipid selected from an anionic lipid, a neutral lipid, or both. In some embodiments, the additional lipid comprises at least one phospholipid. In some embodiments, the phospholipid is selected from an anionic phospholipid, a neutral phospholipid, or both. The phospholipid of the elements of the LNPs and LNP compositions can play a role in covering and protecting a core of the LNP formed by interaction of the cationic lipid and nucleic acid in the LNP, and may facilitate cell membrane permeation and endosomal escape during intracellular delivery of the nucleic acid by binding to the phospholipid bilayer of a target cell. A phospholipid which can promote fusion of the LNP to a cell may include without limitation, any of the phospholipids selected from the group described below.

In some embodiments, the LNPs and LNP compositions comprise at least one phospholipid selected from, but not limited to, dipalmitoyl-phosphatidylcholine (DPPC), distearoyl-phosphatidylcholine (DSPC), dioleoyl-phosphatidylethanolamine (DOPE), dioleoyl-phosphatidylcholine (DOPC), dioleoyl-phosphatidylglycerol (DOPG), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dipalmitoyl-phosphatidylglycerol (DPPG), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), phosphatidylethanolamine (PE), 1,2-dioleoyl-sn-glycero-3-phosphoethanolamine, 1-palmitoyl-2-oleoyl-sn-glycero-3-phosphocholine (POPC), 1,2-dioleoyl-sn-glycero-3-[phospho-L-serine](DOPS), 1,2-dioleoyl-sn-glycero-3-[phospho-L-serine], and any combination of the foregoing. In one example, the LNP comprising DOPE may be effective in mRNA delivery.

In some embodiments, the additional lipid (e.g., phospholipid) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the additional lipid (e.g., phospholipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

It will be appreciated that the total lipid present in the LNPs and/or LNP compositions comprises the combination of the cationic lipid or ionizable cationic lipid, the conjugated lipid, (e.g., pegylated lipid), the steroid (e.g., cholesterol), and the additional lipid (e.g., phospholipid).

The LNPs and/or LNP compositions may be prepared by dissolving the total lipids (or a portion thereof) in an organic solvent (e.g., ethanol) followed by mixing through a micromixer with the payload (e.g., nucleic acids of the systems) dissolved in an acidic buffer (e.g., pH 4). At this pH the cationic lipid is positively charged and interacts with the negatively-charged nucleic acid polymers. The resulting nanostructures containing the nucleic acids are then converted to neutral LNPs when dialyzed against a neutral buffer which may then be followed by removal of the organic solvent (e.g., ethanol) and exchange the LNPs into physiologically relevant buffer. The LNPs and/or LNP compositions thus formed have a distinct electron-dense nanostructured core where the cationic lipids are organized into inverted micelles around the encapsulated payload, as opposed to traditional bilayer liposomal structures. In another embodiment, the LNP may form a bleb-like structure with nucleic acids in aqueous pockets along the non-electron dense lipid core.

b. Lipid Nanoparticle Properties

In some embodiments, the LNPs and/or LNP compositions comprise from about 50 mol % to about 85 mol % of the cationic lipid or ionizable cationic lipid, from about 0.5 mol % to about 10 mol % of the conjugated lipid, (e.g., pegylated lipid), from about 0.5 mol % to about 10 mol % of the steroid (e.g., cholesterol) and from about 5 mol % to about 50 mol % of the additional lipid (e.g., phospholipid). In some embodiments, the LNPs and/or LNP compositions comprise from about 50 mol % to about 85 mol % of the cationic lipid or ionizable cationic lipid, from about 0.5 mol % to about 5 mol % of the conjugated lipid, (e.g., pegylated lipid), from about 0.5 mol % to about 5 mol % of the steroid (e.g., cholesterol) and from about 5 mol % to about 20 mol % of the additional lipid (e.g., phospholipid).

In some embodiments, the LNPs and/or LNP compositions of the disclosure comprise cationic lipid:additional lipid (e.g., phospholipid): steroid (e.g., cholesterol): conjugated lipid, (e.g., pegylated lipid) at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5.

In some embodiments, the LNPs and/or LNP compositions of the disclosure have a total lipid:payload ratio (mass/mass) of from about 1 to about 100. In some embodiments, the total lipid:payload ratio is about 1 to about 50, from about 2 to about 25, from about 3 to about 20, from about 4 to about 15, or from about 5 to about 10. In some embodiments, the total lipid: payload ratio is about 5 to about 15, e.g., about 5, 6, 7, 8, 9, 10, 11, 12, 13, 14, 15, or an intermediate range of any of the foregoing.

In certain embodiments, the LNPs of the disclosure comprise a total lipid:nucleic acid mass ratio of from about 5:1 to about 15:1. In some embodiments, the weight ratio of the cationic lipid and nucleic acid comprised in the LNP may be 1 to 20:1, 1 to 15:1, 1 to 10:1, 5 to 20:1, 5 to 15:1, 5 to 10:1, 7.5 to 20:1, 7.5 to 15:1, or 7.5 to 10:1.

In some embodiments, the LNP of the disclosure may comprise the cationic lipid of 20 to 50 parts by weight, the phospholipid of 10 to 30 parts by weight, cholesterol of 20 to 60 parts by weight (or 20 to 60 parts by weight), and lipid-PEG conjugate of 0.1 to 10 parts by weight (or 0.25 to 10 parts by weight, 0.5 to 5 parts by weight). Alternatively, the LNP may comprise the cationic lipid of 20 to 50% by weight, phospholipid of 10 to 30% by weight, cholesterol of 20 to 60% by weight (or 30 to 60% by weight), and lipid-PEG conjugate of 0.1 to 10% by weight (or 0.25 to 10% by weight, 0.5 to 5% by weight) based on the total nanoparticle weight. As a further alternative, the LNP may comprise the cationic lipid of 25 to 50% by weight, phospholipid of 10 to 20% by weight, cholesterol of 35 to 55% by weight, and lipid-PEG conjugate of 0.1 to 10% by weight (or 0.25 to 10% by weight, 0.5 to 5% by weight), based on the total nanoparticle weight.

In some embodiments, the LNPs of the present disclosure have a mean diameter of from about 20 to 200 nm, 20 to 180 nm, 20 to 170 nm, 20 to 150 nm, 20 to 120 nm, 20 to 100 nm, 20 to 90 nm, 30 to 200 nm, 30 to 180 nm, 30 to 170 nm, 30 to 150 nm, 30 to 120 nm, 30 to 100 nm, 30 to 90 nm, 40 to 200 nm, 40 to 180 nm, 40 to 170 nm, 40 to 150 nm, 40 to 120 nm, 40 to 100 nm, 40 to 90 nm, 40 to 80 nm, 40 to 70 nm, 50 to 200 nm, 50 to 180 nm, 50 to 170 nm, 50 to 150 nm, 50 to 120 nm, 50 to 100 nm, 50 to 90 nm, 60 to 200 nm, 60 to 180 nm, 60 to 170 nm, 60 to 150 nm, 60 to 120 nm, 60 to 100 nm, 60 to 90 nm, 70 to 200 nm, 70 to 180 nm, 70 to 170 nm, 70 to 150 nm, 70 to 120 nm, 70 to 100 nm, 70 to 90 nm, 80 to 200 nm, 80 to 180 nm, 80 to 170 nm, 80 to 150 nm, 80 to 120 nm, 80 to 100 nm, 80 to 90 nm, 90 to 200 nm, 90 to 180 nm, 90 to 170 nm, 90 to 150 nm, 90 to 120 nm, or 90 to 100 nm, or an intermediate range of any of the foregoing.

In some embodiments, the LNPs and/or LNP compositions of the disclosure have a positive charge at acidic pH and may encapsulate the payload (e.g., a therapeutic agent such as the LTRP:gRNA systems, or polynucleotides encoding same) through electrostatic charges produced by negative charges of the payload (e.g., therapeutic agent). The term “encapsulation,” refers to the mixture of lipids surrounding and embedding the payload (e.g., therapeutic agent) at physiological conditions, forming the LNPs. The term “encapsulation efficiency,” as used herein is the amount of payload (e.g., therapeutic agent) encapsulated by the LNPs divided by the total amount of payload (e.g., therapeutic agent) used to load the payload (e.g., therapeutic agent) into the LNPs. The encapsulation efficiency of the LNPs and/or LNP compositions may be 70% or more, 75% or more, 80% or more, 85% or more, 90% or more, 91% or more, 92% or more, 94% or more, or 95% or more. In other embodiments, the encapsulation efficiency of the LNPs and/or LNP compositions is about 80% to 99%, about 85% to 98%, about 88% to 95%, about 90% to 95%, or the payload (e.g., nucleic acids of the systems) may be fully encapsulated within the lipid portion of the LNPs compositions, and thereby protected from enzymatic degradation. In some embodiments, the payload (e.g., therapeutic agent) is not substantially degraded after exposure of the LNPs and/or LNP compositions to a nuclease at 37° C. for at least about 20, 30, 45, or 60 minutes or at least about 2, 3, 4, 5, 6, 7, 8, 9, 10, 12, 14, 16, 18, 20, 22, 24, 26, 28, 30, 32, 34, or 36 hours. In some embodiments, the payload (e.g., nucleic acids of the systems) is complexed with the lipid portion of the LNPs and/or LNP compositions. The LNPs and/or LNP compositions of the present disclosure are non-toxic to mammals such as humans.

The term “fully encapsulated” indicates that the payload (e.g., the nucleic acids of the system) in the LNPs and/or LNP compositions is not significantly degraded after exposure to conditions that significantly degrade free DNA, RNA, or protein. In a fully encapsulated system, less than about 25%, more preferably less than about 10%, and most preferably less than about 5% of the payload (e.g., nucleic acids of the system) in the LNPs and/or LNP compositions is degraded by conditions that would degrade 100% of a non-encapsulated payload. “Fully encapsulated” also indicates that the LNPs and/or LNP compositions are serum-stable, and do not decompose into their component parts upon in vivo administration.

In some embodiments, the amount of the LNPs and/or LNP compositions having the payload (e.g., therapeutic agent), encapsulated therein is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%0, 70%, 75%, 80%, 85%, 90%, 91%, 92%, 93%, 94%, 95%, 96%, 97%, 98%, 99%, or an intermediate range of any of the foregoing.

In some embodiments, the amount of the payload (e.g., the nucleic acids), encapsulated within the LNPs and/or LNP compositions is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the nucleic acids of the disclosure, such as the mRNA encoding the repressor fusion protein, and/or the gRNA, may be provided in a solution to be mixed with a lipid solution such that the nucleic acids may be encapsulated in the lipid nanoparticles. A suitable nucleic acid solution may be any aqueous solution containing the nucleic acid to be encapsulated at various concentrations. For example, a suitable nucleic acid solution may contain the nucleic acid (or nucleic acids) at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.25 mg/ml, 1.5 mg/ml, 1.75 mg/ml, or 2.0 mg/ml. In some embodiments, the nucleic acid comprises an mRNA encoding a repressor fusion protein, and a suitable mRNA solution may contain the mRNA at a concentration ranging from about 0.01-2.0 mg/ml, 0.01-1.5 mg/ml, 0.01-1.25 mg/ml, 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml. In some embodiments, a suitable gRNA solution may contain an gRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml.

In some embodiments, the LNP may have an average diameter of 20 nm to 200 nm, 20 to 180 nm, 20 nm to 170 nm, 20 nm to 150 nm, 20 nm to 120 nm, 20 nm to 100 nm, 20 nm to 90 nm, 30 nm to 200 nm, 30 to 180 nm, 30 nm to 170 nm, 30 nm to 150 nm, 30 nm to 120 nm, 30 nm to 100 nm, 30 nm to 90 nm, 40 nm to 200 nm, 40 to 180 nm, 40 nm to 170 nm, 40 nm to 150 nm, 40 nm to 120 nm, 40 nm to 100 nm, 40 nm to 90 nm, 40 nm to 80 nm, 40 nm to 70 nm, 50 nm to 200 nm, 50 to 180 nm, 50 nm to 170 nm, 50 nm to 150 nm, 50 nm to 120 nm, 50 nm to 100 nm, 50 nm to 90 nm, 60 nm to 200 nm, 60 to 180 nm, 60 nm to 170 nm, 60 nm to 150 nm, 60 nm to 120 nm, 60 nm to 100 nm, 60 nm to 90 nm, 70 nm to 200 nm, 70 to 180 nm, 70 nm to 170 nm, 70 nm to 150 nm, 70 nm to 120 nm, 70 nm to 100 nm, 70 nm to 90 nm, 80 nm to 200 nm, 80 to 180 nm, 80 nm to 170 nm, 80 nm to 150 nm, 80 nm to 120 nm, 80 nm to 100 nm, 80 nm to 90 nm, 90 nm to 200 nm, 90 to 180 nm, 90 nm to 170 nm, 90 nm to 150 nm, 90 nm to 120 nm, or 90 nm to 100 nm for easy introduction into liver tissue, hepatocytes and/or LSEC (liver sinusoidal endothelial cells). The LNP may be sized for easy introduction into organs or tissues, including but not limited to liver, lung, heart, spleen, as well as to tumors. When the size of the LNP is smaller than the above range, it can be difficult to maintain stability as the surface area of the LNP is excessively increased, and thus delivery to the target tissue and/or drug effect may be reduced. The LNP may specifically target liver tissue. Without wishing to be bound by theory, it is thought that one mechanism by which LNP may be used to deliver therapeutic agents is through the imitation of the metabolic behaviors of natural lipoproteins, and so LNP may be usefully delivered to a subject through the lipid metabolism processes carried out by the liver. During the delivery of therapeutic agents to hepatocytes or and/or LSEC (liver sinusoidal endothelial cells), the diameter of the fenestrae leading from the sinusoidal lumen to the hepatocytes and LSEC is about 140 nm in mammals and about 100 nm in humans, so the LNP composition for therapeutic agent delivery having LNPs with a diameter in the above ranges may have excellent delivery efficiency to hepatocytes and LSEC when compared to LNP having the diameter outside the above range.

According to one example, the LNPs of the LNP composition may comprise the cationic lipid:phospholipid:cholesterol: lipid-PEG conjugate in the range described above or at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5. The LNP comprising components at a molar ratio in the above range may have excellent delivery efficiency of therapeutic agents specific to cells of target organs.

In certain aspects, the LNP exhibit a positive charge under the acidic pH condition by showing a pKa of 5 to 8, 5.5 to 7.5, 6 to 7, or 6.5 to 7, and may encapsulate a nucleic acid with high efficiency by easily forming a complex with a nucleic acid through electrostatic interaction with a therapeutic agent such as a nucleic acid showing a negative charge. In such cases, the LNP may be usefully used as a composition for intracellular or in vivo delivery of a therapeutic agent (for example, nucleic acid).

Herein, “encapsulate” or “encapsulation” refers to incorporation of a therapeutic agent efficient delivery, i.e., by surrounding it by the particle surface and/or embedding it within the particle interior. The encapsulation efficiency means the content of the therapeutic agent encapsulated in the LNP relative the total therapeutic agent content used for preparation of the LNP.

The encapsulation of the nucleic acids of the composition in the LNP may be 70% or more, 75% or more, 80% or more, 85% or more, 90% or more, 91% or more, 92% or more, 94% or more, or 95% or more of LNP in the composition encapsulate nucleic acids. In some embodiments, the encapsulation of the nucleic acids of the composition in the LNP is such that between 80% to 99%, between 80% to 97%, between 80% to 95%, between 85% to 95%, between 87% to 95%, between 90% to 95%, between 91% or more to 95% or less, 91% or more to 94% or less, over 91% to 95% or less, 92% to 99%, between 92% to 97%, or between 92% to 95% of the LNP in the composition encapsulate nucleic acids. In some embodiments, the mRNA encoding the LTRP and a gRNA of any of the embodiments of the disclosure are fully encapsulated in the LNP.

The target organs to which a nucleic acid is delivered by the LNP include, but are not limited to the liver, lung, heart, spleen, as well as to tumors. The LNP according to one example is liver tissue-specific and has excellent biocompatibility and can deliver the nucleic acids of a composition with high efficiency, and thus it can be usefully used in related technical fields such as lipid nanoparticle-mediated gene therapy. In a particular embodiment, the target cell to which the nucleic acids are delivered by the LNP according to one example may be a hepatocyte and/or LSEC in vivo. In other embodiments, the disclosure provides LNP formulated for delivery of the nucleic acids of the embodiments to cells ex vivo.

The disclosure provides a pharmaceutical composition comprising a plurality of LNPs comprising nucleic acids, such as mRNA encoding a long-term repressor fusion protein and/or a gRNA variant described herein, and a pharmaceutically acceptable carrier, diluent or excipient.

In certain embodiments, the LNP comprising the nucleic acid(s) has an electron dense core.

The disclosure provides LNP comprising one or more nucleic acids comprising: (a) an mRNA encoding the repressor fusion protein, and/or a gRNA variant described herein; (b) one or more cationic lipids or ionizable cationic lipids or salts thereof comprising from about 50 mol % to about 85 mol % of the total lipid present in the LNP; (c) one or more non-cationic lipids comprising from about 13 mol % to about 49.5 mol % of the total lipid present in the LNP; and (d) one or more conjugated lipids that inhibit aggregation of LNPs comprising from about 0.5 mol % to about 2 mol % of the total lipid present in the particle. In another embodiment, the disclosure provides LNP comprising one or more nucleic acids comprising: (a) an mRNA encoding the repressor fusion protein, and/or a gRNA variant described herein; (b) one or more cationic lipids or ionizable cationic lipids or salts thereof comprising from about 22 mol % to about 85 mol % of the total lipid present in the LNP; (c) one or more non-cationic/phospholipids comprising from about 10 mol % to about 70 mol % of the total lipid present in the LNP; (d) 15 mol % to about 50 mol % sterol, and (d) 1 mol % to about 5 mol % lipid-PEG or lipid-PEG-peptide in the particle. In certain embodiments the long-term repressor fusion protein mRNA and gRNA may be present in the same LNP, or they may be present in different LNPs.

The disclosure provides LNP comprising one or more nucleic acids comprising: (a) an mRNA encoding the long-term repressor fusion protein described herein; (b) a cationic lipid or a salt thereof comprising from about 52 mol % to about 62 mol % of the total lipid present in the LNP; (c) a mixture of a phospholipid and cholesterol or a derivative thereof comprising from about 36 mol % to about 47 mol % of the total lipid present in the LNP; and (d) a PEG-lipid conjugate comprising from about 1 mol % to about 2 mol % of the total lipid present in the LNP. In particular embodiments, the formulation is a four-component system comprising about 1.4 mol % PEG-lipid conjugate (e.g., PEG2000-C-DMA), about 57.1 mol % cationic lipid (e.g., DLin-K-C2-DMA) or a salt thereof, about 7.1 mol % DPPC (or DSPC), and about 34.3 mol % cholesterol (or derivative thereof). In some embodiments, the LNP comprises the mRNA encoding the CasX described herein and a gRNA.

In other embodiments, the LNP comprising one or more nucleic acids comprises: (a) an mRNA encoding the long-term repressor fusion protein and/or a gRNA of any of the embodiments described herein; (b) a cationic lipid or a salt thereof comprising from about 46.5 mol % to about 66.5 mol % of the total lipid present in the LNP; (c) cholesterol or a derivative thereof comprising from about 31.5 mol % to about 42.5 mol % of the total lipid present in the LNP; and (d) a PEG-lipid conjugate comprising from about 1 mol % to about 2 mol % of the total lipid present in the LNP. In particular embodiments, the formulation is a three-component system which is phospholipid-free and comprises about 1.5 mol % PEG-lipid conjugate (e.g., PEG2000-C-DMA), about 61.5 mol % cationic lipid (e.g., DLin-K-C2-DMA) or a salt thereof, and about 36.9 mol % cholesterol (or derivative thereof). In some embodiments, the LNP comprises the mRNA encoding the CasX described herein and a gRNA.

Additional formulations are described in PCT Publication No. WO 09/127060 and US patent publication numbers US 2011/0071208 A1 and US 2011/0076335 A1, the disclosures of which are herein incorporated by reference in their entirety.

In other embodiments, the LNP comprising one or more nucleic acids comprises: (a) an mRNA encoding the long-term repressor fusion protein and a gRNA of any of the embodiments described herein; (b) one or more cationic lipid or ionizable cationic lipids or salts thereof comprising from about 2 mol % to about 50 mol % of the total lipid present in the LNP; (c) one or more non-cationic lipid or ionizable cationic lipids comprising from about 5 mol % to about 90 mol % of the total lipid present in the LNP; and (d) one or more conjugated lipids that inhibit aggregation of particles comprising from about 0.5 mol % to about 20 mol % of the total lipid present in the LNP. In some embodiments, the LNP comprises the mRNA encoding the CasX described herein and a gRNA.

In other embodiments, the LNP comprising one or more nucleic acids comprises: (a) an mRNA encoding the long-term repressor fusion protein and a gRNA of any of the embodiments described herein; (b) a cationic lipid or a salt thereof comprising from about 30 mol % to about 50 mol % of the total lipid present in the LNP; (c) a mixture of a phospholipid and cholesterol or a derivative thereof comprising from about 47 mol % to about 69 mol % of the total lipid present in the LNP; and (d) a PEG-lipid conjugate comprising from about 1 mol % to about 3 mol % of the total lipid present in the LNP. In particular embodiments, the formulation is a four-component system which comprises about 2 mol % PEG-lipid conjugate (e.g., PEG2000-C-DMA), about 40 mol % cationic lipid (e.g., DLin-K-C2-DMA) or a salt thereof, about 10 mol % DPPC (or DSPC), and about 48 mol % cholesterol (or derivative thereof). In some embodiments, the LNP comprises the mRNA encoding the CasX described herein and a gRNA.

In other embodiments, the LNP comprising one or more nucleic acids comprises: (a) an mRNA encoding the long-term repressor fusion protein and a gRNA of any of the embodiments described herein; (b) one or more cationic lipid or ionizable cationic lipids or salts thereof comprising from about 50 mol % to about 65 mol % of the total lipid present in the LNP; (c) one or more non-cationic lipid or ionizable cationic lipids comprising from about 25 mol % to about 45 mol % of the total lipid present in the LNP; and (d) one or more conjugated lipids that inhibit aggregation of particles comprising from about 5 mol % to about 10 mol % of the total lipid present in the LNP. In some embodiments, the LNP comprises the mRNA encoding the CasX described herein and a gRNA.

In other embodiments, the LNP comprising one or more nucleic acids comprises: (a) an mRNA encoding the long-term repressor fusion protein and a gRNA of any of the embodiments described herein; (b) a cationic lipid or a salt thereof comprising from about 50 mol % to about 60 mol % of the total lipid present in the LNP; (c) a mixture of a phospholipid and cholesterol or a derivative thereof comprising from about 35 mol % to about 45 mol % of the total lipid present in the LNP; and (d) a PEG-lipid conjugate comprising from about 5 mol % to about 10 mol % of the total lipid present in the LNP.

In certain embodiments, the non-cationic lipid mixture in the formulation comprises: (i) a phospholipid of from about 10 mol % to about 70 mol % of the total lipid present in the LNP; (ii) cholesterol or a derivative thereof of from about 15 mol % to about 50 mol % of the total lipid present in the LNP; and 1-5% lipid-PEG or lipid-PEG-peptide. In particular embodiments, the formulation is a four-component system which comprises about 7 mol % PEG-lipid conjugate (e.g., PEG750-C-DMA), about 54 mol % cationic lipid (e.g., DLin-K-C2-DMA) or a salt thereof, about 7 mol % DPPC (or DSPC), and about 32 mol % cholesterol (or derivative thereof).

In other embodiments, the LNP comprising one or more nucleic acids comprises: (a) an mRNA encoding the long-term repressor fusion protein and/or a gRNA of any of the embodiments described herein; (b) a cationic lipid or a salt thereof comprising from about 55 mol % to about 65 mol % of the total lipid present in the LNP; (c) cholesterol or a derivative thereof comprising from about 30 mol % to about 40 mol % of the total lipid present in the LNP; and (d) a PEG-lipid conjugate comprising from about 5 mol % to about 10 mol % of the total lipid present in the LNP. In particular embodiments, the formulation is a three-component system which is phospholipid-free and comprises about 7 mol % PEG-lipid conjugate (e.g., PEG750-C-DMA), about 58 mol % cationic lipid (e.g., DLin-K-C2-DMA) or a salt thereof, and about 35 mol % cholesterol (or derivative thereof). In some embodiments, the LNP comprises the mRNA encoding the CasX described herein and a gRNA.

In other embodiments, the LNP comprising one or more nucleic acids comprises: (a) an mRNA encoding the long-term repressor fusion protein and/or a gRNA of any of the embodiments described herein; (b) a cationic lipid or a salt thereof comprising from about 48 mol % to about 62 mol % of the total lipid present in the LNP; (c) a mixture of a phospholipid and cholesterol or a derivative thereof, wherein the phospholipid comprises about 7 mol % to about 17 mol % of the total lipid present in the LNP, and wherein the cholesterol or derivative thereof comprises about 25 mol % to about 40 mol % of the total lipid present in the LNP; and (d) a PEG-lipid conjugate comprising from about 0.5 mol % to about 3.0 mol % of the total lipid present in the LNP. In some embodiments, the LNP comprises the mRNA encoding the CasX described herein and a gRNA.

### IX. Methods for Repression of Target Nucleic Acids

In another aspect, the present disclosure relates to methods of repressing or silencing transcription of a target nucleic acid sequence of a gene in a population of cells, using the LTRP:gRNA systems of the disclosure; either in vitro, ex vivo, or in vivo in a subject. The programmable nature of the systems provided herein allows for the precise targeting to achieve the desired effect at one or more regions of predetermined interest in a target nucleic acid of a gene. In some embodiments, it may be desirable to repress or silence a gene in the cells comprising mutations that result in a disease or disorder in a subject.

In some embodiments, the method comprises introducing into the cells a long-term repressor fusion protein of the disclosure and one or more gRNAs with a targeting sequence complementary to the target nucleic acid wherein the long-term repressor fusion protein is capable of complexing with the gRNA to form an RNP, and wherein the RNP is cable of binding the target nucleic acid and repressing or silencing the transcription of the gene in the cells (it being understood that additional cellular factors may be recruited and participate in the repression). In some embodiments, the method comprises introducing into the cells an mRNA encoding a long-term repressor fusion protein of the disclosure and one or more gRNAs with a targeting sequence complementary to the target nucleic acid, whereupon the long-term repressor fusion protein is expressed, and is capable of complexing with the gRNA to form an RNP, and wherein the RNP is capable of binding the target nucleic acid and repressing or silencing the transcription of the gene in the cells. In some embodiments, the mRNA encoding the long-term repressor fusion protein and gRNA may be co-formulated in a nanoparticle for delivery to the cells of the population. In some embodiments, the mRNA encoding the long-term repressor fusion protein and gRNA may be formulated in separate nanoparticles for delivery to the cells of the population. In some embodiments, the nanoparticle is a lipid nanoparticle (LNP), as described herein.

In some embodiments of the method of transcriptional repression of a gene, the LTRP:gRNA systems of the present disclosure can be designed to target any region of, or proximal to, a gene or region of a gene for which repression of transcription is sought. When the entirety of the gene is to be repressed, use of a guide with a targeting sequence complementary to a sequence encompassing or proximal to the transcription start site (TSS) is contemplated by the disclosure. The core promoter serves as a binding platform for the transcription machinery, which comprises Pol II and its associated general transcription factors (GTFs) (Haberle, V. et al. Eukaryotic core promoters and the functional basis of transcription initiation (Nat Rev Mol Cell Biol. 19(10):621 (2018)). Variability in TSS selection has been proposed to involve DNA ‘scrunching’ and ‘anti-scrunching,’ the hallmarks of which are: (i) forward and reverse movement of the RNA polymerase leading edge, but not trailing edge, relative to DNA, and (ii) expansion and contraction of the transcription bubble. In some embodiments, the targeting sequence of the gRNA of the LTRP:gRNA system is complementary to a target nucleic acid sequence located within 1 kb of a transcription start site (TSS) in the gene targeted for repression. In some embodiments of the method, the targeting sequence of the gRNA of the LTRP:gRNA system is complementary to a target nucleic acid sequence located within 20 bp, 50 bp, 100 bp, 150 bp, 200 bp, 250 bp, 500 bp, 1 kb, or 1.5 kb upstream of a TSS of the gene targeted for repression. In some embodiments of the method, the targeting sequence of the gRNA of the LTRP:gRNA system is complementary to a target nucleic acid sequence located within 20 bp, 50 bp, 100 bp, 150 bp, 200 bp, 250 bp, 500 bps, 1 kb, or 1.5 kb downstream of a TSS of a gene targeted for repression. In some embodiments of the method, the targeting sequence of the gRNA of the LTRP:gRNA system is complementary to a target nucleic acid sequence located within 700 bp upstream to 700 bp downstream, 500 bp upstream to 500 bp downstream, 300 bp upstream to 300 bp downstream, or 100 bp upstream to 100 bp downstream of a TSS of the gene. In some embodiments of the method, the targeting sequence of the gRNA of the LTRP:gRNA system is complementary to a target nucleic acid sequence located within 20 bp, 50 bp, 100 bp, 150 bp, 200 bp, 250 bp, 500 bp, or 1 kb of an enhancer of the gene targeted for repression. In some embodiments of the method, the targeting sequence of the gRNA of the LTRP:gRNA system is complementary to a target nucleic acid sequence located within 1 kb 3′ to a 5′ untranslated region of the gene targeted for repression. In some embodiments of the method, the targeting sequence of the gRNA of the LTRP:gRNA system is complementary to a target nucleic acid sequence located within the open reading frame of the gene targeted for repression. In some embodiments of the method, the targeting sequence of the gRNA of the LTRP:gRNA system is complementary to a target nucleic acid sequence of an exon of the gene targeted for repression. In a particular embodiment, the targeting sequence of a gRNA of the system of the disclosure is complementary to a target nucleic acid sequence for exon 1 of the gene targeted for repression. In other embodiments of the method, the targeting sequence of a gRNA of the system of the disclosure is complementary to a target nucleic acid sequence of an intron of the gene targeted for repression. In other embodiments of the method, the targeting sequence of the gRNA of the system of the disclosure is complementary to a target nucleic acid sequence of an intron-exon junction of the gene targeted for repression. In other embodiments of the method, the targeting sequence of the gRNA of the system of the disclosure is complementary to a target nucleic acid sequence of a regulatory element of the gene targeted for repression. In other embodiments of the method, the targeting sequence of the gRNA of the system of the disclosure is complementary to a sequence of an intergenic region of the gene targeted for repression. In other embodiments of the method, the targeting sequence of a gRNA of the system of the disclosure is complementary to a junction of the exon, an intron, or a regulatory element of the gene targeted for repression. In those cases where the targeting sequence is complementary to a regulatory element, such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. In some embodiments of the method, the targeting sequence of a gRNA of the system is complementary to a target nucleic acid sequence within 1 kb of an enhancer of the gene targeted for repression. In some embodiments of the method, the targeting sequence of a gRNA of the system of the disclosure is complementary to the target nucleic acid sequence within the 3′ untranslated region of the gene targeted for repression. The promoter region is intended to encompass nucleotides within 5 kb of the initiation point of the encoding sequence or, in the case of gene enhancer elements or conserved elements, can be thousands of bp, hundreds of thousands of bp, or even millions of bp away from the encoding sequence of the gene targeted for repression. In the foregoing, the targets are those in which the encoding gene of the target is intended to be repressed and/or epigenetically modified such that a gene product is not expressed or is expressed at a lower level in a cell. In some embodiments, upon binding of the RNP of the system of the disclosure to the binding location of the target nucleic acid, the system is capable of repressing transcription of the gene 5′ to the binding location of the RNP. In other embodiments, upon binding of the RNP of the system to the binding location of the target nucleic acid, the system is capable of repressing transcription of the gene 3′ to the binding location of the RNP.

The disclosure provides methods of transcriptional repression or silencing a target gene in a population of cells. In some embodiments, the method comprises contacting the population of cells with an LTRP:gRNA system comprising an mRNA comprising a sequence selected from the group consisting of SEQ ID NOS: 2409-18636, or a sequence having at least about 70%, at least about 80%, at least about 85%, at least about 90%, at least about 95%, at least about 96%, at least about 97%, at least about 98%, or at least about 99% sequence identity thereto, the gRNA comprising a scaffold comprising a sequence selected from the group consisting of SEQ ID NOS: 1744-1746, 2136-2144, 2146-2154, and 2156-2164, or a sequence having at least about 70%, at least about 80%, at least about 85%, at least about 90%, at least about 95%, at least about 96%, at least about 97%, at least about 98%, or at least about 99% sequence identity thereto, and wherein the gRNA comprises a linked targeting sequence complementary to a target nucleic acid of a gene to be repressed. In some embodiments, the LTRP:gRNA system comprises a gRNA variant comprising a sequence of SEQ ID NO: 1744. In some embodiments, the LTRP:gRNA system comprises a gRNA variant comprising a sequence of SEQ ID NO: 1745. In some embodiments, the LTRP:gRNA system comprises gRNA variant comprising a sequence of SEQ ID NO: 1746. In some embodiments, the LTRP:gRNA system comprises a gRNA variant comprising one or more chemical modifications, including gRNA variants comprising the sequences of SEQ ID NOS: 2136-2144; 2146-2154; or 2156-2164, with a targeting sequence complementary to a target nucleic acid substituted for the 20 nucleotides on the 3′ end of the gRNA of the listed sequences. In a particular embodiment, the LTRP:gRNA system comprises an mRNA encoding an LTRP comprising a sequence selected from the group consisting of SEQ ID NOS: 2411, 2421, 2467, and 2477, and the gRNA comprises a sequence of SEQ ID NO: 2156 and a linked targeting sequence complementary to a sequence of a gene targeted for repression or silencing substituted for the 20 nucleotides on the 3′ end of SEQ ID NO: 2156.

In some embodiments, the method for transcriptional repression or silencing of a target nucleic acid comprises contacting the population of cells with an LNP comprising an LTRP:gRNA system comprising an mRNA comprising a sequence selected from the group consisting of the sequences of SEQ ID NOS: 2409-18636, or a sequence having at least about 70%, at least about 80%, at least about 85%, at least about 90%, at least about 95%, at least about 96%, at least about 97%, at least about 98%, or at least about 99% sequence identity thereto, and a gRNA comprising a scaffold comprising a sequence selected from the group consisting of SEQ ID NOS: 1744-1746, or alternatively the gRNA comprises a sequence selected from the group consisting of 2136-2144, 2146-2154, and 2156-2164 wherein a targeting sequence complementary to a target nucleic acid is substituted for the 20 nucleotides on the 3′ end of the gRNA of the listed sequences, or a sequence having at least about 70%, at least about 80%, at least about 85%, at least about 90%, at least about 95%, at least about 96%, at least about 97%, at least about 98%, or at least about 99% sequence identity thereto, and the gRNA comprises a targeting sequence complementary to a target nucleic acid of a gene to be repressed. In some embodiments of the method, the LNP comprising an LTRP:gRNA system comprising a gRNA variant comprising a scaffold comprising a sequence of SEQ ID NO: 1744. In some embodiments of the method, the LNP comprising an LTRP:gRNA system comprises a gRNA variant comprising a scaffold comprising a sequence of SEQ ID NO: 1745. In some embodiments of the method, the LNP comprising an LTRP:gRNA system comprises a gRNA variant comprising a scaffold comprising a sequence of SEQ ID NO: 1746. In some embodiments of the method, the LNP comprising an LTRP:gRNA system comprises a gRNA variant with chemical modifications, including the sequences of SEQ ID NOS: 2136-2144; 2146-2154; and 2156-2164, with a targeting sequence complementary to a target nucleic acid substituted for the 20 nucleotides on the 3′ end of the gRNA of the listed sequences. In a particular embodiment of the method, the LNP comprising an LTRP:gRNA system comprises an mRNA encoding an LTRP comprising a sequence selected from the group consisting of SEQ ID NOS: 2411, 2421, 2467, and 2477, and a gRNA comprising the scaffold portion of SEQ ID NO: 2156 with a linked targeting sequence complementary to a sequence of a gene targeted for repression or silencing substituted for the 20 nucleotides on the 3′ end of SEQ ID NO: 2156.

In some embodiments of the method, contacting cells with an LTRP:gRNA system of the disclosure results in transcriptional repression of the targeted gene in at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60%, at least about 70%, at least about 80% or more of the cells of the population targeted by the LTRP:gRNA system. In some embodiments of the method, a gene in the cells targeted by the LTRP:gRNA system is repressed or silenced such that expression of the protein encoded by the gene is decreased by at least about 10%, at least about 20%, at least about 30%, at least about 40%, at least about 50%, at least about 60%, at least about 70%, at least about 80%, or at least about 90% in comparison to a cell where the gene has not been targeted. In some embodiments, the repression of transcription of the gene in the cells is sustained for at least about 8 hours, at least about 1 day, at least about 7 days, at least about 1 month, at least about 2 months, at least about 3 months, at least about 4 months, at least about 5 months, or at least about 6 months or more. In some embodiments, repression of transcription in cells treated with an LTRP:gRNA system of the embodiments is heritable and is stable through one or more cell divisions. In some embodiments, repression of transcription is stable through 1, 10, 20, 30, 40, 50, 60, 70, 80, 90, or 100 cell divisions, or more. In some embodiments, the repression is determined in an in vitro assay. In some embodiments, the repression is determined in a subject in assays of cells removed from the subject or by assays of a protein or marker in samples obtained from the subject.

The systems and methods described herein can be used in a variety of cells associated with disease, e.g., cells of the liver, the intestine, the lung, the heart, bone, the kidney, the eye, the central nervous system, smooth muscle cells, macrophages or cells of arterial walls, in which the gene product that is contributory to the disease or condition is to be repressed or silenced. In some embodiments, the cell targeted for transcriptional repression is a eukaryotic cell. In some embodiments, the eukaryotic cell is selected from the group consisting of a rodent cell, a mouse cell, a rat cell, a primate cell, and a non-human primate cell. In some embodiments, the eukaryotic cell is a human cell. In some embodiments of the method, the cell is an embryonic stem cell, an induced pluripotent stem cell, a germ cell, a fibroblast, an oligodendrocyte, a glial cell, a hematopoietic stem cell, a neuron progenitor cell, a neuron, an astrocyte, a muscle cell, a bone cell, a hepatocyte, a pancreatic cell, a lung cell, a renal cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, a bone marrow cell, a mesenchymal cell, a parenchymal cell, an epithelial cell, an endothelial cell, a mesothelial cell, a fibroblasts, an osteoblast, a chondrocyte, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogeneic cell, an allogenic cell, and a post-natal stem cell.

The disclosure provides a method for reversing the transcriptional repression resulting from the LTRP:gRNA systems. In some embodiments, the transcriptional repression is reversible by use of an inhibitor of DNMT. In some embodiments of the method, the transcriptional repression is reversible by treating the cells with a cytidine analog inhibitor of DNMT. In some embodiments, the transcriptional repression is reversible by treating the cells with an inhibitor selected from the group consisting of azacytidine, decitabine, clofarabine, and zebularine. In some embodiments of the method, wherein reversal of the transcriptional repression is in a subject treated with a system of the disclosure, the method comprises administrations of a therapeutically effective dose of the inhibitor of DNMT.

### X. Therapeutic Methods

The present disclosure provides methods of treating a disease or disorder in a subject in need thereof using the LTRP:gRNA systems of the disclosure. In some embodiments, the methods of the disclosure can prevent, treat and/or ameliorate a disease or disorder of a subject by the administering of a therapeutically effective dose of a composition of the disclosure to the subject. This approach, therefore, could be used for applications in a subject with a disease or disorder such as, but not limited to, autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, or age-related macular degeneration (AMD).

In some cases, one or both alleles of a gene of the subject with a disease or disorder comprises a mutation. In some cases, the mutation is a gain of function mutation. In other cases, the disorder mutation is a loss of function mutation.

In some embodiments, the disclosure provides methods of treating a disease or disorder in a subject in need thereof comprising repressing or silencing a gene in cells of the subject, the method comprising administering a therapeutically effective dose of: i) an LTRP:gRNA system comprising a long-term repressor fusion protein and a gRNA of any one of the embodiments described herein; ii) a gRNA of any of the embodiments described herein and an mRNA encoding the long-term repressor fusion protein; iii) an LNP or a synthetic nanoparticle comprising a gRNA and a mRNA encoding the long-term repressor fusion protein of any one of the embodiments described herein; or iv) combinations of two or more of (i) to (iv), wherein the target nucleic acid sequence of the cells targeted by the gRNA is repressed or silenced by the long-term repressor fusion protein of the LTRP:gRNA system.

In some embodiments of the method of treatment, the method comprises administering a therapeutically effective dose of an LTRP:gRNA system comprising an mRNA comprising a sequence selected from the group consisting of SEQ ID NOS: 2409-18636, or a sequence having at least about 70%, at least about 80%, at least about 85%, at least about 90%, at least about 95%, at least about 96%, at least about 97%, at least about 98%, or at least about 99% sequence identity thereto, a gRNA comprising a scaffold comprising a sequence selected from the group consisting of SEQ ID NOS: 1744-1746, or a sequence having at least about 70%, at least about 80%, at least about 85%, at least about 90%, at least about 95%, at least about 96%, at least about 97%, at least about 98%, or at least about 99% sequence identity thereto, and the gRNA comprises a targeting sequence complementary to a target nucleic acid of a gene to be repressed. In some embodiments, the LTRP:gRNA system comprises gRNA variant 174 (scaffold sequence of SEQ ID NO: 1744). In some embodiments of the method, the LTRP:gRNA system comprises gRNA variant 235 (scaffold sequence of SEQ ID NO: 1745). In some embodiments of the method, the LTRP:gRNA system comprises gRNA variant 316 (scaffold sequence of SEQ ID NO: 1746). In some embodiments of the method, the LTRP:gRNA system comprises a gRNA variant with chemical modifications, including the sequences of SEQ ID NOS: 2136-2144; 2146-2154; and 2156-2164 (with a targeting sequence complementary to a target nucleic acid substituted for the 20 nucleotides on the 3′ end of the gRNA). In a particular embodiment of the method, the LTRP:gRNA system comprises gRNA variant 316 with chemical modifications, the gRNA comprising a sequence selected from the group consisting of SEQ ID NOS: 2156-2164 (with a targeting sequence complementary to a target nucleic acid substituted for the 20 nucleotides on the 3′ end of the gRNA). In some embodiments of the method, the LTRP:gRNA system comprises an LTRP encoded by an mRNA sequence selected from the group consisting of SEQ ID NOS: 2411, 2421, 2467, and 2477, and gRNA variant 316 with chemical modifications, the gRNA comprising a sequence of SEQ ID NO: 2156, with a targeting sequence complementary to a target nucleic acid substituted for the 20 nucleotides on the 3′ end of the gRNA.

In some embodiments of the method, administering a therapeutically effective dose to a subject with an LTRP:gRNA system of the embodiments results in transcriptional repression of the targeted gene in at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60%, at least about 70%, at least about 80% or more of the cells of the targeted organ. In some embodiments of the method, the gene in the cells of the targeted organ in the subject are repressed such that expression of the encoded protein is decreased by at least about 10%, at least about 20%, at least about 30%, at least about 40%, at least about 50%, at least about 60%, at least about 70%, at least about 80%, or at least about 90% in comparison to an untreated cell. In some embodiments, the transcriptional repression of the targeted gene in the subject is sustained for at least about 1 day, at least about 7 days, at least about 2 weeks, at least about 3 weeks, at least about 1 month, at least about 2 months, at least about 3 months, at least about 4 months, at least about 5 months, or at least about 6 months or more. In some embodiments of the method of treating a disease or disorder in a subject, the subject is selected from the group consisting of mouse, rat, pig, non-human primate, and human.

A number of therapeutic strategies have been used to design the systems for use in the methods of treatment of a subject with a disease or disorder. In some embodiments, the disclosure provides a method of treatment of a subject having a disease or disorder, the method comprising administering to the subject an LTRP:gRNA system composition according to a treatment regimen comprising one or more consecutive doses using a therapeutically effective dose. In some embodiments of the treatment regimen, the therapeutically effective dose of the composition is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months or more. In some embodiments of the treatment regimen, the effective doses are administered by a route selected from the group consisting of intravenous, intraportal vein injection, intraperitoneal, intramuscular, subcutaneous, intraocular, and oral routes.

In some embodiments, the administering of the therapeutically effective amount of an LTRP:gRNA modality, including an LNP comprising a gRNA and an mRNA encoding a long-term repressor fusion protein disclosed herein, to repress or silence expression of a gene in a subject with a disease or disorder leads to the prevention or amelioration of the underlying disease or disorder such that an improvement is observed in the subject, notwithstanding that the subject may still be afflicted with the underlying disorder. In some embodiments, the administration of the therapeutically effective amount of the LTRP:gRNA modality leads to an improvement in at least one clinically-relevant endpoint. In some embodiments, the administration of the therapeutically effective amount of the LTRP:gRNA modality leads to an improvement in at least two clinically-relevant endpoints. In some embodiments, the improvement in the at least one or two clinically-relevant endpoints in the subject is sustained for at least about 7 days, at least about 2 weeks, at least about 3 weeks, at least about 1 month, at least about 2 months, at least about 3 months, at least about 4 months, at least about 5 months, or at least about 6 months or longer. In some embodiments, the subject is selected from mouse, rat, pig, dog, non-human primate, and human.

Methods of obtaining samples from treated subjects for analysis to determine the effectiveness of the treatment, such as body fluids or tissues, and methods of preparation of the samples to allow for analysis are well known to those skilled in the art. Methods for analysis of RNA and protein levels are discussed above and are well known to those skilled in the art. The effects of treatment can also be assessed by measuring protein or biomarkers associated with the target gene expression in the aforementioned fluids, tissues or organs, collected from an animal contacted with one or more compounds of the disclosure, by routine clinical methods known in the art.

### XI. Pharmaceutical Compositions, Kits, and Articles of Manufacture

In some embodiments, the disclosure provides pharmaceutical compositions comprising: i) a long-term repressor fusion protein and a gRNA comprising a targeting sequence specific for the targeted gene; ii) the gRNA of (i) and an mRNA encoding the long-term repressor fusion protein; iii) an LNP or synthetic nanoparticle comprising a gRNA and/or mRNA of (ii), together with one or more pharmaceutically suitable excipients, buffers, diluent or carriers. In some embodiments, the pharmaceutical composition is formulated for a route of administration selected from the group consisting of intravenous, intraportal vein injection, intraperitoneal, intramuscular, subcutaneous, intraocular, and oral routes. In one embodiment, the pharmaceutical composition is in a liquid form or a frozen form. In another embodiment, the pharmaceutical composition is in a pre-filled syringe for a single injection. In another embodiment, the pharmaceutical composition is in solid form, for example, the pharmaceutical composition is lyophilized.

Excipients can include a salt, an isotonic agent, a serum protein, a buffer or other pH-controlling agent, an anti-oxidant, a thickener, an uncharged polymer, a preservative or a cryoprotectant. Excipients used in compositions of the disclosure may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. In some embodiments the isotonic agent may be glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples of preservatives include polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

Additional pharmaceutical formulations appropriate for administration are applicable in the methods and compositions disclosed herein (see, e.g., Remington's Pharmaceutical Sciences (1990) 18th ed., Mack Publishing Co., Easton, Pa.; Remington's Pharmaceutical Sciences (2023) 23rd ed., Elsevier Publishing; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993).

In some embodiments, the disclosure provides compositions of a long-term repressor fusion protein and gRNA for use in the manufacture of a medicament for use in the treatment of a disease in a subject. In some embodiments, the disclosure provides compositions of LNP comprising a long-term repressor fusion protein and gRNA for use in the manufacture of a medicament for use in the treatment of a disease in a subject.

In other embodiments, provided herein are kits comprising the LTRP:gRNA systems, polynucleotides, vectors, and LNP formulations described herein. In some embodiments, the LNP formulation comprises LNPs encapsulating an mRNA encoding a long-term repressor fusion protein and one or a plurality of gRNA comprising a targeting sequence complementary to a target nucleic acid sequence for a gene for repression or silencing. In some embodiments, the kits comprise a suitable container (for example a tube, vial or plate). In exemplary embodiments, a kit of the disclosure comprises an LNP formulation encapsulating an mRNA comprising a sequence selected from the group consisting of SEQ ID NOS: 2409-18636, or a sequence having at least about 70%, at least about 80%, at least about 85%, at least about 90%, at least about 95%, at least about 96%, at least about 97%, at least about 98%, or at least about 99% sequence identity thereto, the gRNA comprises a scaffold comprising a sequence selected from the group consisting of SEQ ID NOS: 1744-1746, or alternatively, the gRNA comprises a sequence selected from the group consisting of SEQ ID NOS: 2136-2144, 2146-2154, and 2156-2164 wherein a targeting sequence complementary to a target nucleic acid is substituted for the 20 nucleotides on the 3′ end of the gRNA of the listed sequences, or a sequence having at least about 70%, at least about 80%, at least about 85%, at least about 90%, at least about 95%, at least about 96%, at least about 97%, at least about 98%, or at least about 99% sequence identity thereto, and the gRNA comprises a targeting sequence complementary to a target nucleic acid of a gene to be repressed. In some embodiments, the gRNA is chemically modified, as described herein.

In some embodiments, the kit further comprises a buffer, a nuclease inhibitor, a protease inhibitor, a liposome, a therapeutic agent, a label, a label visualization reagent, instructions for use, or any combination of the foregoing. In some embodiments, the kit further comprises a pharmaceutically acceptable carrier, buffer, diluent or excipient.

In some embodiments, the kit comprises appropriate control compositions for gene repression applications, and instructions for use.

### Enumerated Embodiments

The disclosure can be understood with reference to the following illustrative, enumerated embodiments:

1. A system for transcriptional repression of a gene, comprising an mRNA encoding a long-term repressor fusion protein (LTRP), wherein the LTRP comprises:
(a) a DNA binding protein;
(b) a DNA methyltransferase (DNMT) 3A catalytic domain (DNMT3A);
(c) a DNMT3 like interaction domain (DNMT3L); and
(d) a first repressor domain (RD1).

2. The system of embodiment 1, wherein the LTRP comprises, from N- to C-terminus:
(a) the DNMT3A;
(b) the DNMT3L;
(c) the DNA-binding domain; and
(d) the RD1.

3. The system of embodiment 1, wherein the LTRP comprises, from N- to C-terminus:
(a) the DNMT3A;
(b) the DNMT3L;
(c) the RD1; and
(d) the DNA binding protein.

4. The system of embodiment 1, wherein the LTRP comprises, from N- to C-terminus:
(a) the DNMT3A;
(b) the DNMT3L;
(c) the RD1; and
(d) the DNA-binding domain;
(e) a second RD1.

5. The system of embodiment 4, wherein the RD1 sequences are identical.

6. The system of embodiment 4, wherein the RD1 sequences are different.

7. The system of any one of embodiments 1-6, wherein the LTRP further comprises a DNMT3A ATRX-DNMT3-DNMT3L domain (ADD) linked N-terminal to the DNMT3A.

8. The system of any one of embodiments 1-7, wherein the DNA binding protein is a catalytically-dead Class 1 CRISPR protein or a catalytically-dead Class 2 CRISPR protein.

9. The system of embodiment 8, wherein the catalytically-dead Class 2 CRISPR is a catalytically-dead CasX (dCasX).

10. The system of any one of embodiments 1-9, wherein the sequence encoding the DNMT3A comprises SEQ ID NO: 1955 or SEQ ID NO: 21878, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

11. The system of any one of embodiments 1-9, wherein the sequence encoding the DNMT3L comprises SEQ ID NO: 1945 or SEQ ID NO: 21879, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

12. The system of any one of embodiments 1-9, wherein the sequence encoding the RD1 comprises a sequence selected from the group consisting of SEQ ID NOS: 1946, 18637-20233-21830, and 21846 or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

13. The system of embodiment 12, wherein the sequence encoding the RD1 comprises a sequence selected from the group consisting of SEQ ID NOS: 18637-18646 and 20234-20242, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

14. The system of embodiment 13, wherein the sequence encoding the RD1 comprises a sequence selected from the group consisting of SEQ ID NOS: 18642 and 20239, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

15. The system of embodiment 13, wherein the sequence encoding the RD1 comprises a sequence selected from the group consisting of SEQ ID NO: 18637 and 20234, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto

16. The system of embodiment 13, wherein the sequence encoding the RD1 comprises a sequence selected from the group consisting of SEQ ID NOS:18638 and 20235, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

17. The system of any one of embodiments 9-16, wherein the sequence encoding the dCasX comprises a sequence selected from the group consisting of SEQ ID NOS: 1948, 2211, 2212, 2213, 2214, and 2405-2408, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto

18. The system of embodiment 17, wherein the sequence encoding the dCasX comprises SEQ ID NO: 2405 or SEQ ID NO: 2406, or a sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity thereto.

19. The system of any one of embodiments 1-18, wherein the mRNA comprises one or more sequences encoding a nuclear localization sequence (NLS), optionally wherein the NLS comprises an amino acid sequence selected from the group consisting of SEQ ID NOS: 30-97.

20. The system of embodiment 19, wherein the sequence encoding the NLS of SEQ ID NO: 30 comprises SEQ ID NO: 21833 or SEQ ID NO: 21875.

21. The system of any one of embodiments 1-20, wherein the mRNA comprises one or more sequences encoding one or more linker peptides, optionally wherein the linker peptides comprise an amino acid sequence selected from the group consisting of SEQ ID NOS: 1823-1874.

22. The system of embodiment 21, wherein the one or more linker peptides are located between any one or more of the DNA binding protein, the DNMT3A, the DNMT3L and the RD1.

23. The system of embodiment 21 or 22, wherein each of the DNA binding protein, the DNMT3A, the DNMT3L and the RD1 are separated by one or more linker peptides.

24. The system of any one of embodiments 1-23, wherein the mRNA comprises a sequence encoding the LTRP selected from the group consisting of SEQ ID NOS: 2409-18636, or a sequence having at least about 70%, at least about 80%, at least about 90%, at least about 95%, at least about 95%, or at least about 96%, at least about 97%, at least about 98%, or at least about 99% sequence identity thereto.

25. The system of any one of embodiments 1-24, wherein the mRNA comprises a sequence encoding the LTRP selected from the group consisting of SEQ ID NOS: 2409-2428, 2465-2484, and 2521-8908, or a sequence having at least about 70%, at least about 80%, at least about 90%, at least about 95%, at least about 95%, at least about 96%, at least about 97%, at least about 98%, or at least about 99% sequence identity thereto.

26. The system of any one of embodiments 1-24, wherein the mRNA comprises a sequence encoding the LTRP selected from the group consisting of SEQ ID NOS: 2409-2428, and 2465-2484, or a sequence having at least about 70%, at least about 80%, at least about 90%, at least about 95%, at least about 95%, at least about 96%, at least about 97%, at least about 98%, or at least about 99% sequence identity thereto.

27. The system of any one of embodiments 1-24, wherein the mRNA comprises a sequence encoding the LTRP selected from the group consisting of SEQ ID NOS: 2411, 2421, 2467, and 2477, or a sequence having at least about 70%, at least about 80%, at least about 90%, at least about 95%, at least about 95%, at least about 96%, at least about 97%, at least about 98%, at least about 99% sequence identity thereto.

28. The system of any one of embodiments 1-24, wherein the mRNA comprises a sequence encoding the LTRP selected from the group consisting of SEQ ID NOS: 2410, 2420, 2466, and 2476, or a sequence having at least about 70%, at least about 80%, at least about 90%, at least about 95%, at least about 95%, at least about 96%, at least about 97%, at least about 98%, or at least about 99% sequence identity thereto.

29. The system of any one of embodiments 1-24, wherein the mRNA comprises a sequence encoding the LTRP selected from the group consisting of SEQ ID NOS: 2412, 2422, 2468, and 2478, or a sequence having at least about 70%, at least about 80%, at least about 90%, at least about 95%, at least about 96%, at least about 97%, at least about 98%, or at least about 99% sequence identity thereto.

30. The system of any one of embodiments 24-29, wherein the mRNA sequence encoding the LTRP is codon-optimized, optionally wherein the mRNA sequence encoding the LTRP is codon-optimized for expression in a human cell.

31. The system of any one of embodiments, 24-30, wherein the mRNA comprises a 5′ UTR, a 3′ UTR, a poly(A) sequence, and/or a 5′cap.

32. The system of any one of embodiments 24-31, wherein:
(a) the DNA binding protein comprises an amino acid sequence of SEQ ID NOS: 4-29, or a sequence having at least about 70%, at least about 80%, at least about 90%, at least about 95%, or at least about 99% sequence identity thereto;
(b) the RD1 comprises an amino acid sequence selected from the group consisting of SEQ ID NOS: 130-224, or a sequence having at least about 70%, at least about 80%, at least about 90%, at least about 95%, or at least about 99% sequence identity thereto;
(c) the DNMT3A comprises an amino acid sequence of SEQ ID NO: 126, or a sequence having at least about 70%, at least about 80%, at least about 90%, at least about 95%, or at least about 99% sequence identity thereto; and/or
(d) the DNMT3L comprises an amino acid sequence of SEQ ID NO: 127, or a sequence having at least about 70%, at least about 80%, at least about 90%, at least about 95%, or at least about 99% sequence identity thereto.

33. The system of any one of embodiments 24-31, wherein:
(a) the DNA binding protein comprises an amino acid sequence of SEQ ID NOS: 4-29;
(b) the RD1 comprises an amino acid sequence selected from the group consisting of SEQ ID NOS: 130-224, optionally wherein the RD1 comprises an amino acid sequence selected from the group consisting of SEQ ID NOS: 130, 131 and 135;
(c) the DNMT3A comprises an amino acid sequence of SEQ ID NO: 126; and/or
(d) the DNMT3L comprises an amino acid sequence of SEQ ID NO: 127.

34. The system of any one of embodiments 1-33, comprising a guide ribonucleic acid (gRNA) comprising a targeting sequence complementary to a target nucleic acid sequence of a gene in a cell.

35. The system of embodiment 34, wherein the targeting sequence of the gRNA is complementary to a target nucleic acid sequence within 1.5 kb of a transcription start site (TSS) in the gene.

36. The system of embodiment 34, wherein the targeting sequence of the gRNA is complementary to a target nucleic acid sequence within 500 bps upstream to 500 bps downstream of a TSS of the gene.

37. The system of embodiment 34, wherein the targeting sequence of the gRNA is complementary to a target nucleic acid sequence within 300 bps upstream to 300 bps downstream or is within 100 bps upstream to 100 bps downstream of a TSS of the gene.

38. The system of embodiment 34, wherein the targeting sequence of the gRNA is complementary to a gene target nucleic acid sequence within 100 bps upstream to 100 bps downstream of a TSS of the gene.

39. The system of embodiment 34, wherein the targeting sequence of the gRNA is complementary to a target nucleic acid sequence within 1 kb of an enhancer of the gene.

40. The system of embodiment 34, wherein the targeting sequence of the gRNA is complementary to a target nucleic acid sequence within the 3′ untranslated region of the gene.

41. The system of any one of embodiments 34-40, wherein the gRNA is a single-molecule gRNA (sgRNA).

42. The system of any one of embodiments 34-41, wherein the gRNA comprises a scaffold stem loop comprising the sequence of CCAGCGACUAUGUCGUAGUGG (SEQ ID NO: 1822), or a sequence having 1, 2, 3, 4, or 5 mismatches thereto.

43. The system of any one of embodiments 34-42, wherein the gRNA comprises a scaffold comprising a sequence selected from the group consisting of SEQ ID NOS: 1744-1821, or a sequence having at least about 70%, at least about 80%, at least about 90%, at least about 95%, at least about 96%, at least about 97%, at least about 98%, at least about 99% sequence identity thereto.

44. The system of any one of embodiments 34-42, wherein the gRNA comprises a scaffold comprising a sequence selected from the group consisting of SEQ ID NOS: 1744-1821.

45. The system of any one of embodiments 34-42, wherein the gRNA comprises a scaffold comprising a sequence of SEQ ID NOS: 1746, or a sequence having at least about 70%, at least about 80%, at least about 90%, at least about 95%, at least about 96%, at least about 97%, at least about 98%, at least about 99% sequence identity thereto.

46. The system of any one of embodiments 34-45, wherein the gRNA is chemically modified.

47. The system of embodiment 46, wherein the chemical modification comprises an addition of a 2′O-methyl group to one or more nucleotides of the gRNA.

48. The system of embodiment 47, wherein one or more nucleotides located 1, 2, 3, or 4 nucleotides from the 5′terminal end, the 3′ terminal end, or both terminal ends of the gRNA are modified by an addition of a 2′O-methyl group.

49. The system of embodiment 46, wherein the chemical modification to the gRNA comprises a substitution of a phosphorothioate bond between two or more nucleotides of the gRNA.

50. The system of embodiment 49, wherein the chemical modification comprises a substitution of phosphorothioate bonds between two or more nucleotides located 1, 2, 3, or 4 nucleotides from the 5′terminal end, the 3′ terminal end, or both terminal ends of the gRNA.

51. The system of any one of embodiments 46-50, wherein the gRNA comprises a sequence selected from the group consisting of SEQ ID NOS: 2136-2144, 2146-2154, and 2156-2164, or a sequence having at least about 70%, at least about 80%, at least about 90%, at least about 95%, at least about 96%, at least about 97%, at least about 98%, at least about 99% sequence identity thereto.

52. The system of embodiment 51, wherein the gRNA comprises a sequence selected from SEQ ID NOS: 2136-2144, 2146-2154, and 2156-2164, and comprises a targeting sequence complementary to a target nucleic acid substituted for the 20 nucleotides on the 3′ end of SEQ ID NOS: 2136-2144, 2146-2154, or 2156-2164.

53. The system of embodiment 51, wherein the gRNA comprises a sequence of SEQ ID NO: 2156, and comprises a targeting sequence complementary to a target nucleic acid substituted for the 20 nucleotides on the 3′ end of SEQ ID NO: 2156.

54. The system of any one of embodiments 34-53, wherein upon expression of the LTRP in a cell, the LTRP is capable of complexing with the gRNA to form a ribonucleoprotein (RNP) complex.

55. The system of embodiment 54, wherein the RNP is capable of binding to the target nucleic acid of the gene and repressing or silencing transcription of the gene.

56. A lipid nanoparticle (LNP) comprising the system of any one of embodiments 34-55.

57. The LNP of embodiment 56, wherein the LNP comprises one or more components selected from the group consisting of ionizable lipids, one or more helper phospholipids, one or more polyethylene glycol (PEG)-modified lipids, and cholesterol or a derivative thereof.

58. The LNP of embodiment 56 or embodiment 57, wherein the LNP comprises an ionizable lipid, a helper phospholipid, a polyethylene glycol (PEG)-modified lipid, and cholesterol or a derivative thereof.

59. The LNP of any one of embodiments 56-57, comprising a cationic lipid comprising a pKa of 5 to 8.

60. A plurality of the LNP of any one of embodiments 56-59, wherein individual LNP in the plurality comprise the mRNA, the gRNA, or a combination thereof.

61. A vector comprising the system of any one of embodiments 1-33, or one or more nucleic acids encoding same.

62. A vector comprising or encoding:
(a) the mRNA of any one of embodiments 1-33;
(b) the gRNA of any one of embodiments 3455; or a combination of the mRNA and gRNA.

63. A host cell comprising the vector of embodiment 61 or 62.

64. The host cell of embodiment 63, wherein the host cell is selected from the group consisting of a Baby Hamster Kidney fibroblast (BHK), a human embryonic kidney 293 (HEK293) cell, a human embryonic kidney 293T (HEK293T) cell, a NS0 cell, a SP2/0 cell, a YO myeloma cell, a P3X63 mouse myeloma cell, a PER cell, a PER.C6 cell, a hybridoma cell, a NIH3T3 cell, a CV-1 (simian) in Origin with SV40 genetic material (COS) cell, a HeLa cell, a Chinese hamster ovary (CHO) cell, and a yeast cell.

65. A pharmaceutical composition comprising:
(a) the system of any one of embodiments 1-33;
(b) the LNP of any one of embodiments56-59; or
(c) the vector of embodiment 61 or 62; and a pharmaceutically acceptable carrier, diluent or excipient.

66. The pharmaceutical composition of embodiment 65, wherein the pharmaceutical composition comprises the LNP.

67. The pharmaceutical composition of embodiment 66, wherein an average diameter of the LNP is between about 20 nm and about 200 nm.

68. The pharmaceutical composition of any one of embodiments 65-67, wherein the pharmaceutical composition is formulated for administration by intravenous, intraarterial, intraportal vein injection, intraperitoneal, intramuscular, intracerebroventricular, intracisternal, intrathecal, intracranial, intralumbar, intraocular, subcutaneous, or oral routes.

69. A method of repressing transcription of a gene in a population of cells, the method comprising, contacting cells of the population with:
(a) the system of any one of embodiments1-55;
(b) the LNP of any one of embodiments 56-59;
(c) the vector of embodiment 61 or 62;
(d) the pharmaceutical composition of any one of embodiments 65-68; or
(e) combinations of two or more of (a)-(e), thereby repressing transcription of the gene in the population of cells.

70. The method of embodiment 69, wherein the targeting sequence of the gRNA is complementary to a target nucleic acid sequence located:
(a) within 300 to 1,500 base pairs 5′ to a transcription start site (TSS) in the gene;
(b) within 300 to 1,500 base pairs 3′ to a TSS in the gene;
(c) within 300 to 1,000 base pairs to an enhancer of the gene;
(d) within the open reading frame of the gene;
(e) within an exon of the gene; or
(f) in the 5′ or 3′ untranslated region (UTR) of the gene.

71. The method of embodiment 69 or embodiment 70, wherein the cells of the population are eukaryotic cells.

72. The method of embodiment 71, wherein the eukaryotic cells are selected from the group consisting of rodent cells, mouse cells, rat cells, and non-human primate cells.

73. The method of embodiment 71, wherein the eukaryotic cells are human cells.

74. The method of any one of embodiments 69-73, wherein the cells are selected from the group consisting of an embryonic stem cell, an induced pluripotent stem cell, a germ cell, a fibroblast, an oligodendrocyte, a glial cell, a hematopoietic stem cell, a neuron progenitor cell, a neuron, an astrocyte, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, a bone marrow cell, a mesenchymal cell, a parenchymal cell, an epithelial cell, an endothelial cell, a mesothelial cell, a fibroblast cell, an osteoblast cell, a chondrocyte cell, a hematopoietic stem cell, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogeneic cell, an allogenic cell, and a post-natal stem cell.

75. The method of any one of embodiments 69-74, wherein transcription of the gene in at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60%, at least about 70%, at least about 80% or more of the cells of the population is repressed.

76. The method of any one of embodiments 69-75, wherein transcription of the gene in the population of cells is repressed by at least about 10%, at least about 20%, at least about 30%, at least about 40%, at least about 70%, at least about 75%, at least about 80%, at least about 85%, at least about 90%, at least about 95%, or at least 99% greater compared to untreated cells, when assessed in an in vitro assay.

77. The method of any one of embodiments 69-76, wherein off-target methylation or off-target transcription repression is less than about 10%, less than about 9%, less than about 8%, less than about 7%, less than about 6%, less than about 5%, less than about 4%, less than about 3%, less than about 2%, or less than about 1% in the cells, when compared to untreated, cells, when assessed in an in vitro assay.

78. The method of any one of embodiments 69-77, wherein the repression of transcription in the cells is sustained for at least about 8 hours, at least about 1 day, at least about 7 days, at least about 2 weeks, at least about 3 weeks, at least about 1 month, at least about 2 months, or at least about 6 months.

79. The method of any one of embodiments 69-78, wherein the repression of transcription is stable through one or more cell divisions.

80. The method of any one of embodiments 69-79, wherein epigenetic modification in the cells by the LTRP is heritable.

81. The method of any one of embodiment 69-80, wherein the repression of the gene of the population of cells occurs in vitro or ex vivo.

82. The method of embodiments 69-81, wherein the repression of the gene of the population of cells occurs in vivo in a subject.

83. The method of embodiment 82, wherein the subject is selected from the group consisting of a rodent, a mouse, a rat, and a non-human primate.

84. The method of embodiment 82, wherein the subject is a human.

85. The method of any one of embodiments 69-84, wherein the repression is reversible.

86. The method of embodiment 85, wherein the repression is reversible by use of an inhibitor of DNMT.

87. The method of embodiment 85 or embodiment 86, wherein the inhibitor is a cytidine analog.

88. The method of embodiment 86 or embodiment 87, wherein the inhibitor is selected from the group consisting of azacytidine, decitabine, clofarabine, and zebularine.

89. A method of treating a disease in a subject in need thereof, the method comprising administering a therapeutically effective dose of:
(a) the system of any one of embodiments 1-55;
(b) the LNP of any one of embodiments 56-60;
(c) the vector of embodiment 61 or 62;
(d) the pharmaceutical composition of any one of embodiments 65-68; or
(e) combinations of two or more of (a)-(d), wherein transcription of the target gene in the subject is repressed by the LTRP, thereby treating the disease.

90. The method of embodiment 89, comprising administering a therapeutically effective dose of the LNP.

91. The method of embodiment 90, wherein the LNP is administered by a route of administration selected from the group consisting of intravenous, intraarterial, intraportal vein injection, intraperitoneal, intramuscular, intracerebroventricular, intracisternal, intrathecal, intracranial, intralumbar, intraocular, subcutaneous, and oral routes.

92. A kit comprising the system of any one of embodiments 1-55, the LNP of any one of embodiments 56-60, the vector of embodiment 61 or 62, the pharmaceutical composition of any one of embodiments 65-68, or combinations thereof, and a suitable container.

93. The kit of embodiment 92, comprising a buffer, an excipient, a nuclease inhibitor, a protease inhibitor, a liposome, a therapeutic agent, a label, a label visualization reagent, instructions for use, or any combination of the foregoing.

94. The system of any one of embodiments 1-55, the LNP of any one of embodiments 56-60, the vector of embodiment 61 or 62, or the pharmaceutical composition of any one of embodiments 65-68, for use in the manufacture of a medicament for the treatment of a disease in a subject in need thereof.

The following Examples are merely illustrative and are not meant to limit any aspects of the present disclosure in any way.

### EXAMPLES

### Example 1: Demonstration that Inclusion of the ADD Domain into an LTRP Molecule Enhances Repression of an Endogenous Locus in Mouse Hepa1-6 Cells

Experiments were performed to demonstrate that incorporation of the ADD domain into an LTRP molecule enhances the ability of LTRPs to induce durable repression of an endogenous locus in mouse Hepa1-6 liver cells, when the LTRPs were delivered as encoding mRNA and co-transfected with a targeting gRNA.

### Materials and Methods

Generation of LTRP Configuration 5 mRNA:

mRNA encoding two variants of the LTRP configuration 5 molecule were generated by in vitro transcription (IVT): 1) an LTRP configuration 5 molecule containing the ZIM3-KRAB domain (hereafter known as LTRP5-ZIM3) and 2) a LTRP5-ZIM3 containing the DNMT3A-ADD domain (hereafter known as LTRP5-ZIM3-ADD). Briefly, constructs encoding for a 5′UTR region, LTRP5-ZIM3 or LTRP5-ZIM3-ADD with flanking SV40 NLSes, and a 3′UTR region were generated and cloned into a plasmid containing a T7 promoter and 79-nucleotide poly(A) tail. N1-methyl-pseudouridine residues were substituted for uridine in the sequences. Sequences encoding the LTRP5-ZIM3 or LTRP5-ZIM3-ADD molecules were codon-optimized using a codon utilization table, in addition to using a publicly available codon optimization tool and adjusting parameters such as GC content of the encoding LTRP sequence. The DNA sequences encoding the LTRP5-ZIM3 and LTRP5-ADD-ZIM3 mRNAs are listed in Table 9. The corresponding mRNA sequences and protein sequences are listed in Table 10.

TABLE 9
Encoding DNA and RNA sequences of the LTRP5-ZIM3 and LTRP5-
ZIM3-ADD mRNA molecules assessed in this example*

| LTRP | | DNA sequence | RNA sequence |
|---|---|---|---|
| molecule | Component | SEQ ID NO | SEQ ID NO |
| LTRP5-ZIM3 | 5′UTR | 1925 | 1941 |
| | START codon + NLS + linker | 1926 | 1942 |
| | START codon + DNMT3A catalytic | 1927 | 1943 |
| | domain | | |
| | Linker | 1928 | 1944 |
| | DNMT3L interaction domain | 1929 | 1945 |
| | Linker | 1930 | 1930 |
| | ZIM3-KRAB | 1931 | 1946 |
| | Linker | 1932 | 1947 |
| | dCasX491 | 1933 | 1948 |
| | Buffer + linker | 1934 | 1949 |
| | NLS + STOP codon + buffer sequence | 1935 | 1950 |
| | 3′UTR | 1936 | 1951 |
| | Buffer sequence | 1937 | 1952 |
| | Poly(A) tail | 1938 | 1938 |
| LTRP5-ADD- | 5′UTR | 1925 | 1941 |
| ZIM3 | START codon + NLS + linker | 1926 | 1942 |
| | START codon + DNMT3A ADD domain | 1939 | 1954 |
| | DNMT3A catalytic domain | 1940 | 1955 |
| | Linker | 1928 | 1944 |
| | DNMT3L interaction domain | 1929 | 1945 |
| | Linker | 1930 | 1930 |
| | ZIM3-KRAB | 1931 | 1946 |
| | Linker | 1932 | 1947 |
| | dCasX491 | 1933 | 1948 |
| | Buffer + linker | 1934 | 1949 |
| | NLS + STOP codon + buffer sequence | 1935 | 1950 |
| | 3′UTR | 1936 | 1951 |
| | Buffer sequence | 1937 | 1952 |
| | Poly(A) tail | 1938 | 1938 |

*Components are listed in a 5′ to 3′ order within the constructs

TABLE 10
Full-length RNA and protein sequences of LTRP5-ZIM3
and LTRP5-ZIM3-ADD molecules assessed in this example.
Modification ‘mψ’ = N1-methyl-pseudouridine

| | RNA Sequence | |
|---|---|---|
| LTRP molecule | SEQ ID NO | AA SEQ ID NO |
| LTRP5-ZIM3 | 1956 | 1909 |
| LTRP5-ADD-ZIM3 | 1957 | 1883 |

Synthesis of gRNAs:

Two gRNAs targeting the mouse PCSK9 locus were designed using gRNA scaffold 316 (SEQ ID NO: 1746) and chemically synthesized using the v1 modification profile (as described in Example 11; SEQ ID NO: 2156). PCSK9-targeting spacers 27.88 and 27.94 (sequences listed in Table 37) were assessed in these experiments. As shown in Example 6, use of spacer 27.88 was less effective in achieving PCSK9 knockdown than use of spacer 27.94.

Transfection of mRNA and gRNA into Hepa1-6 cells and intracellular PCSK9 staining were performed as described in Example 6. Briefly, each well of seeded Hepa1-6 cells was transfected with 300 ng of mRNA encoding LTRP5-ZIM3 or LTRP5-ADD-ZIM3 and 150 ng of PCSK9-targeting gRNA with spacer 27.88 or 27.94. Intracellular levels of PCSK9 protein were measured at various timepoints, up to day 53 post-transfection using an intracellular staining protocol as described earlier in Example 6. A non-targeting gRNA was used as an experimental control.

### Results:

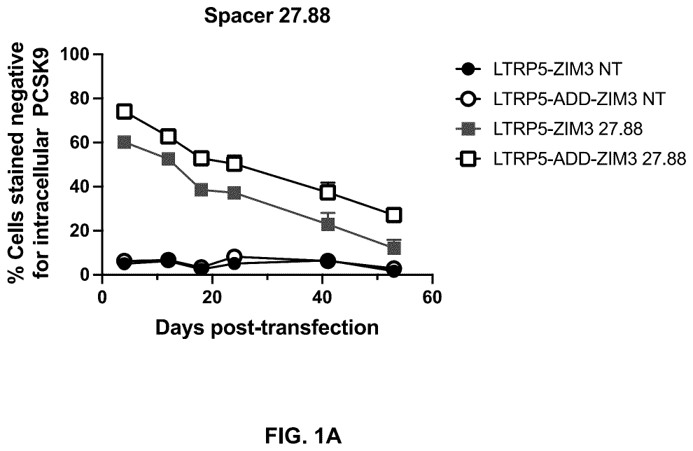

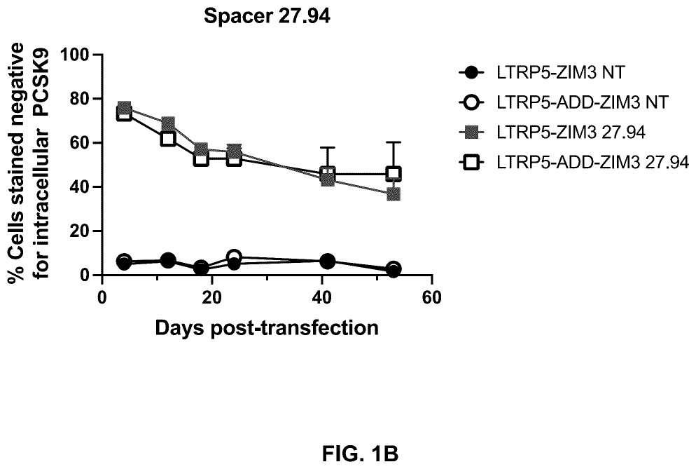

To determine the effects of incorporating the ADD domain into an LTRP molecule on activity, i.e., inducing more durable repression of an endogenous locus in vitro, mRNAs encoding LTRP5-ZIM3 or LTRP5-ADD-ZIM3 were co-transfected with a PCSK9-targeting gRNA into Hepa1-6 cells. The quantification of the resulting PCSK9 knockdown is shown in FIGS. 23A-23B. The data demonstrate a noticeable improvement in achieving PCSK9 knockdown when the cells were treated with LTRP5-ADD-ZIM3 than with LTRP5-ZIM3 without the ADD domain, and this improvement was more pronounced when using a PCSK9-targeting gRNA containing the weaker spacer 27.88. Further supporting the data discussed in Example 6, use of spacer 27.94 resulted in more durable repression than use of spacer 27.88 by day 53 when paired with either the LTRP5-ZIM3 or LTRP5-ADD-ZIM3 molecule (FIG. 1). As expected, use of the non-targeting spacer did not result in PCSK9 knockdown.

These experiments demonstrate that use of LTRP constructs with the ADD domain can result in increased durable repression of an endogenous locus in cells compared to LTRP constructs without the ADD domain. Furthermore, these findings show that LTRP molecules with the ADD domain can be delivered as mRNA and co-transfected with a targeting gRNA to cells to induce effective silencing.

### Example 2: Demonstration that LTRP Molecules Containing the ADD Domain can Induce Repression of an Endogenous Locus in Multiple Human Cell Lines

Experiments were performed to demonstrate that LTRP molecules containing the ADD domain can induce long-term repression of an endogenous target locus in various human cell lines, when delivered as mRNA co-transfected with a targeting gRNA.

### Materials and Methods

Generation of mRNA:

mRNA encoding the following molecules were generated by IVT following similar methods as described in Example 6:1) a catalytically-active CasX 676 (SEQ ID NO: 1962), 2) dXR1 (as described in Example 6), and 3) LTRP5-ADD-ZIM3 (as described in Example 1). Sequences encoding these molecules were codon-optimized using a codon utilization table, in addition to using a publicly available codon optimization tool and adjusting parameters such as GC content. The DNA sequences encoding the catalytically-active CasX 676 are listed in Table 11. The DNA and mRNA sequences encoding for dXR1 are listed in Table 35 and Table 36 respectively. The DNA and mRNA sequences encoding for LTRP5-ADD-ZIM3 are listed in Table 9 and Table 10 respectively.

TABLE 11
Encoding sequences of the catalytically-active
CasX 676 mRNA molecule assessed in this example*

| CasX | | | | SEQ |
|---|---|---|---|---|
| mRNA | Component | | | ID |
| ID | (ID) | Description | DNA sequence | NO: |
| CasX 676 | 5′ UTR | TriLink | AAATAAGAGAGAAAAGAAGAGTAAGAAGAAA | 1925 |
| mRNA | | | TATAAGAGCCACC | |
| | START codon + | | ATGGCCCCTGCTGCCAAGAGAGTGAAGCTGG | 1958 |
| | c-MYC NLS | | ATAGCAGA | |
| | CasX 676 | | CAGGAGATCAAGCGGATTAATAAAATTCGGA | 1959 |
| | | | GAAGACTGGTGAAGGATTCTAACACAAAGAA | |
| | | | GGCTGGCAAGACACGGGGCCCTATGAAGACA | |
| | | | CTGCTGGTGAGAGTGATGACACCCGACCTGA | |
| | | | GAGAAAGACTGGAAAACCTGAGAAAGAAGCC | |
| | | | TGAGAATATCCCCCAGCCCATCAGCAACACA | |
| | | | AGCCGGGCCAACCTGAATAAGCTGCTGACCG | |
| | | | ACTACACCGAAATGAAGAAGGCCATCCTGCA | |
| | | | CGTGTATTGGGAAGAGTTCCAGAAAGACCCA | |
| | | | GTCGGCCTGATGAGCAGAGTGGCTCAGCCTG | |
| | | | CCAGCAAGAAGATCGATCAGAACAAGCTGAA | |
| | | | GCCCGAAATGGACGAGAAGGGGAACCTGACA | |
| | | | ACCGCCGGCTTTGCCTGTAGCCAGTGCGGCC | |
| | | | AGCCCCTGTTTGTGTACAAACTGGAACAGGT | |
| | | | GAGCGAAAAGGGCAAGGCTTACACGAATTAC | |
| | | | TTCGGCAGATGCAACGTGGCCGAGCACGAGA | |
| | | | AGCTGATCAAGCTGGCCCAGCTGAAGCCTGA | |
| | | | GAAGGATAGCGATGAGGCAGTGACATATTCC | |
| | | | CTGGGCAAGTTCGGACAGCGGGCCCTGGATT | |
| | | | TTTATTCCATTCATGTGACCAAGGAATCCAC | |
| | | | CCACCCCGTCAAGCCTCTTGCCCAAATTGCC | |
| | | | GGCAACAGATACGCCTCCAGCCCCGTGGGCA | |
| | | | AGGCCCTGAGCGACGCCTGTATGGGCACCAT | |
| | | | CGCCAGCTTCCTGTCTAAGTACCAGGACATT | |
| | | | ATCATCGAGCACCAGAAGGTGGTGAAGGGCA | |
| | | | ACCAGAAGAGACTGGAGAGCCTGCGCGAGCT | |
| | | | GGCCGGCAAGGAAAACCTGGAGTATCCTAGC | |
| | | | GTGACCCTGCCTCCTCAGCCTCATACAAAGG | |
| | | | AGGGCGTGGATGCCTACAACGAAGTGATCGC | |
| | | | CCGGGTGCGGATGTGGGTGAACCTGAATCTG | |
| | | | TGGCAGAAGCTGAAGCTGTCTAGAGACGACG | |
| | | | CCAAGCCCCTGCTGAGACTGAAGGGCTTCCC | |
| | | | CAGCTTCCCTCTGGTGGAGAGACAGGCAAAT | |
| | | | GAAGTGGACTGGTGGGACATGGTGTGTAACG | |
| | | | TGAAGAAGCTGATCAATGAGAAGAAGGAGGA | |
| | | | CGGCAAAGTGTTCTGGCAGAATCTGGCCGGC | |
| | | | TACAAGCGTCAGGAGGCCCTGCGGCCCTACC | |
| | | | TGAGCAGCGAGGAAGACAGAAAGAAGGGCAA | |
| | | | GAAGTTCGCCCGGTATCAGCTGGGGGACCTG | |
| | | | CTGCTGCACCTCGAGAAGAAGCACGGCGAAG | |
| | | | ACTGGGGGAAGGTGTACGATGAGGCCTGGGA | |
| | | | GCGGATCGATAAGAAGGTGGAGGGCCTGAGC | |
| | | | AAGCACATCAAGCTGGAGGAGGAACGGAGAT | |
| | | | CTGAGGACGCCCAGAGCAAGGCCGCCCTGAC | |
| | | | CGACTGGCTGAGAGCCAAGGCCAGCTTCGTC | |
| | | | ATCGAGGGGCTGAAGGAGGCCGACAAGGACG | |
| | | | AGTTCTGCCGGTGCGAACTGAAGCTGCAGAA | |
| | | | GTGGTACGGAGATCTGAGAGGCAAACCTTTC | |
| | | | GCCATCGAGGCCGAGAACAGCATCCTGGACA | |
| | | | TCAGCGGCTTCAGCAAGCAGTACAACTGCGC | |
| | | | CTTTATTTGGCAGAAGGACGGAGTGAAGAAG | |
| | | | CTGAACCTGTACCTGATCATCAACTATTTCA | |
| | | | AGGGCGGCAAGCTGAGATTCAAGAAGATCAA | |
| | | | GCCTGAAGCCTTCGAGGCCAACAGATTCTAC | |
| | | | ACCGTGATTAACAAGAAAAGCGGAGAGATCG | |
| | | | TGCCAATGGAAGTGAACTTCAACTTCGACGA | |
| | | | CCCTAACCTGATCATCCTGCCCCTGGCATTT | |
| | | | GGCAAGCGGCAGGGCAGAGAGTTCATCTGGA | |
| | | | ACGACCTGCTGTCTCTGGAGACCGGCAGCCT | |
| | | | GAAGCTGGCCAACGGCAGAGTGATCGAGAAG | |
| | | | ACACTGTACAACAGACGAACCAGACAAGACG | |
| | | | AGCCCGCCCTGTTTGTGGCCCTGACCTTCGA | |
| | | | GAGAAGAGAGGTGCTGGACAGCAGCAATATC | |
| | | | AAGCCTATGAACCTGATCGGCGTGGACCGGG | |
| | | | GCGAGAACATCCCTGCCGTGATCGCCCTTAC | |
| | | | CGACCCCGAGGGATGCCCTCTGAGCCGGTTT | |
| | | | AAAGACAGCCTGGGCAACCCTACCCACATCC | |
| | | | TGAGAATTGGCGAGTCCTACAAGGAGAAGCA | |
| | | | GAGAACCATCCAGGCCAAGAAGGAGGTGGAG | |
| | | | CAGCGGCGGGCTGGCGGCTACTCCCGGAAGT | |
| | | | ACGCCAGCAAGGCCAAGAACCTGGCCGACGA | |
| | | | CATGGTTAGAAATACCGCCAGAGACCTCCTG | |
| | | | TACTACGCTGTGACCCAGGACGCCATGCTGA | |
| | | | TCTTCGAGAACCTGAGCAGAGGCTTCGGCAG | |
| | | | ACAGGGCAAGAGAACCTTCATGGCCGAGAGA | |
| | | | CAGTACACCCGGATGGAGGACTGGCTGACCG | |
| | | | CCAAGCTGGCCTACGAGGGCCTGCCCTCTAA | |
| | | | GACCTACCTGTCCAAGACCTTGGCACAGTAC | |
| | | | ACCAGCAAGACATGCTCTAACTGCGGCTTCA | |
| | | | CAATCACGAGCGCCGACTACGACCGGGTGCT | |
| | | | GGAGAAACTGAAGAAGACCGCCACAGGCTGG | |
| | | | ATGACCACCATTAACGGCAAGGAGCTGAAGG | |
| | | | TGGAGGGCCAGATCACCTACTACAACAGGTA | |
| | | | CAAACGGCAGAACGTGGTGAAGGACCTGAGC | |
| | | | GTGGAACTGGATAGACTGAGCGAGGAAAGCG | |
| | | | TAAACAATGACATCAGCAGCTGGACCAAGGG | |
| | | | CCGGAGCGGCGAGGCCCTGAGCCTGCTGAAG | |
| | | | AAGAGATTCTCCCACAGACCAGTGCAGGAGA | |
| | | | AGTTCGTGTGTCTGAACTGCGGCTTCGAGAC | |
| | | | CCACGCCGACGAGCAAGCCGCCCTGAACATC | |
| | | | GCCCGGTCTTGGCTTTTCCTGCGGAGCCAGG | |
| | | | AGTACAAGAAGTACCAGACAAACAAGACCAC | |
| | | | AGGCAACACAGACAAGAGAGCCTTCGTCGAG | |
| | | | ACCTGGCAGAGCTTCTACAGAAAGAAGCTGA | |
| | | | AGGAGGTGTGGAAGCCTGCCGTG | |
| | c-MYC NLS + | | GGAAGCCCCGCTGCCAAGAGAGTGAAGCTGG | 1960 |
| | STOP codons | | ACTAATAGATAA | |
| | 3′UTR | Mouse HBA | GCTGCCTTCTGCGGGGCTTGCCTTCTGGCCA | 1936 |
| | | | TGCCCTTCTTCTCTCCCTTGCACCTGTACCT | |
| | | | CTTGGTCTTTGAATAAAGCCTGAGTAGGAAG | |
| | XbaI | | TCTAG | 1937 |
| | restriction | | | |
| | site (partial) | | | |
| | Poly(A) tail | | AAAAAAAAAAAAAAAAAAAAAAAAAAAAAAA | 1961 |
| | | | AAAAAAAAAAAAAAAAAAAAAAAAAAAAAAA | |
| | | | AAAAAAAAAAAAAAAAAA | |

*Components are listed in a 5′ to 3′ order within the constructs

Synthesis of gRNAs:

gRNAs targeting the human PCSK9 locus were designed using gRNA scaffold 316 (SEQ ID NO: 1746) and chemically synthesized using the v1 modification profile (as described in Example 11; SEQ ID NO: 2156). Furthermore, a B2M-targeting gRNA was used as an experimental control. Sequences of targeting spacers as assessed in this example as listed in Table 12.

TABLE 12
Sequences of spacers assessed in this example

| Spacer | | Targeting spacer | SEQ |
|---|---|---|---|
| ID | Target | sequence (RNA) | ID NO |
| 7.37 | human B2M | GGCCGAGAUGUCUCGCUCCG | 1904 |
| 6.1 | human PCSK9 | GAGGAGGACGGCCUGGCCGA | 1963 |
| 6.125 | human PCSK9 | AGAAAGAGCAAGCCUCAUGU | 1964 |
| 6.138 | human PCSK9 | AGGGAUUUAUACUACAAAGA | 1965 |
| 6.153 | human PCSK9 | GUUAAUGUUUAAUCAGAUAG | 1966 |
| 6.157 | human PCSK9 | GGGUCUGAGCCUGGAGGAGU | 1967 |
| 6.172 | human PCSK9 | GCUGAAACAGAUGGAAUACU | 1968 |
| 6.181 | human PCSK9 | UCAUCUGCACUCGUGGCCAC | 1969 |

Transfection of mRNA and gRNA into HepG2 Cells, Hep3B Cells, and Huh7 Cells and ELISA to Assess PCSK9 Secretion:

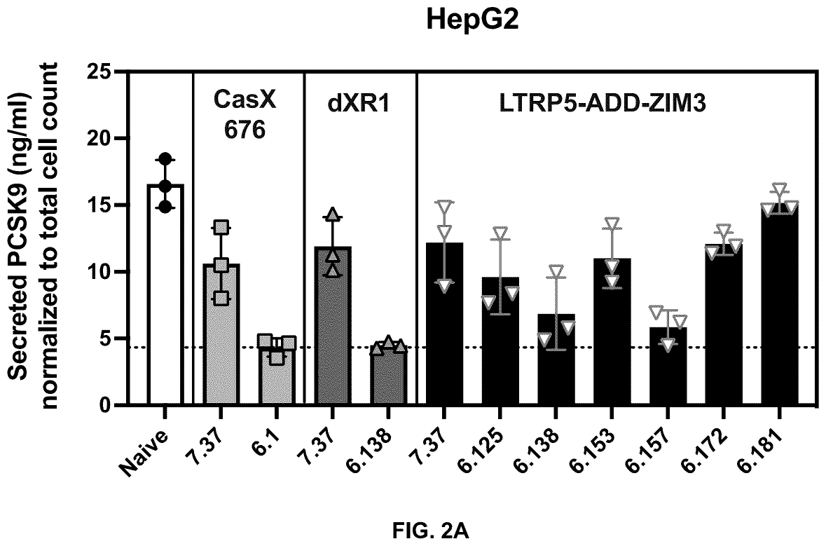

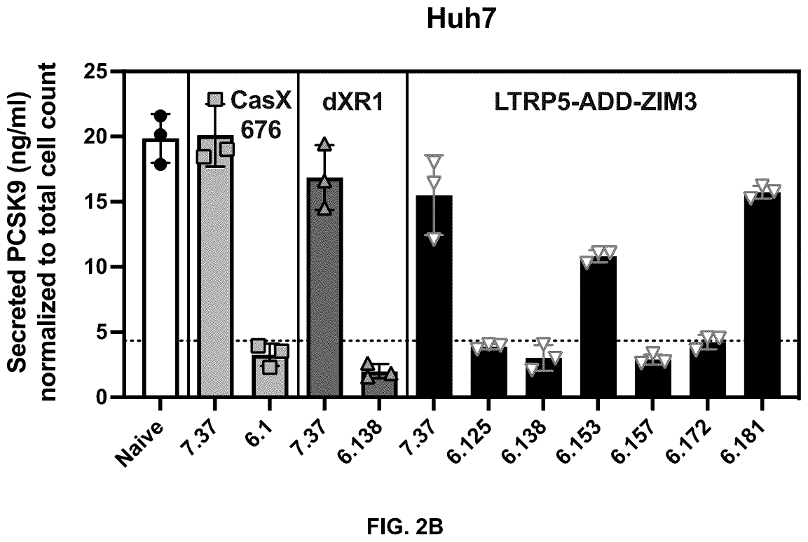

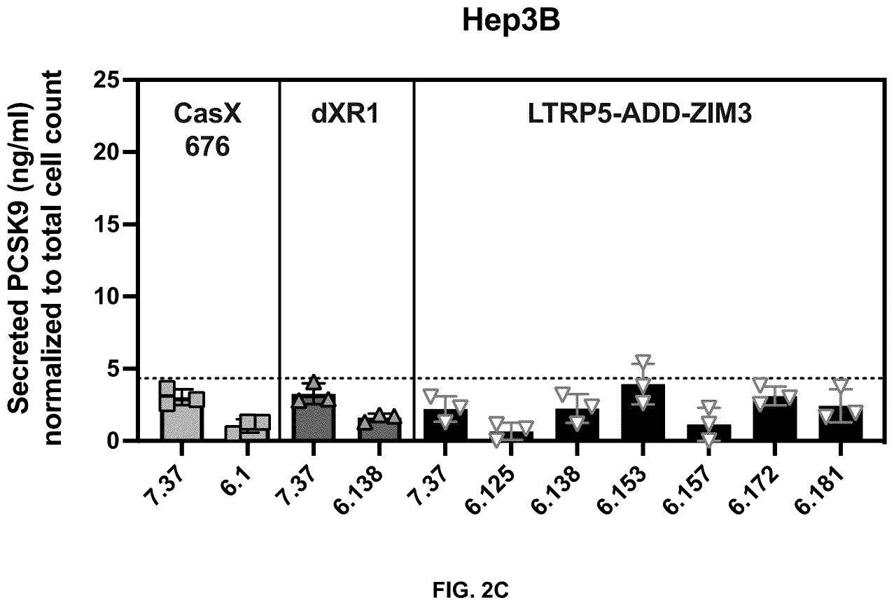

The following three human hepatocyte cancer cell lines were used in this experiment: HepG2 cells, Hep3B cells, and Huh7 cells. ~15,000 cells of each cell line were seeded in each well of a 96-well plate. The next day, seeded cells were transfected with mRNA encoding a catalytically-active CasX 676, dXR1, or LTRP5-ADD-ZIM3 and a gRNA with scaffold 316 and spacer targeting either the B2M or PCSK9 locus (see Table 12 for specific spacers and sequences). Media supernatant was harvested at 4 days post-transfection to assess level of PCSK9 secretion by ELISA, and levels of PCSK9 secretion were normalized to total cell count, as illustrated in FIGS. 2A-2C. Culturing of treated Huh7 cells continued, and media supernatant was harvested at 14 and 27 days post-transfection for measuring PCSK9 secretion by ELISA. As an additional experimental control, PCSK9 secretion was also measured in the media supernatant harvested from wells containing untreated, naïve cells.

### Results:

HepG2 cells, Hep3B cells, and Huh7 cells were transfected with mRNA encoding catalytically-active CasX 676, dXR1, or LTRP5-ADD-ZIM3 with a gRNA targeting either B2M or the PCSK9 locus, and secreted PCSK9 levels were measured. Quantification of normalized PCSK9 secretion levels for each condition at 4 days post-transfection is depicted in FIGS. 2A-2C. The data demonstrate that the most efficient knockdown of PCSK9 secretion by CasX 676, dXR1, or LTRP5-ADD-ZIM3 was observed in Huh7 cells, while HepG2 cells did not exhibit as efficient of a knockdown of secreted PCSK9 levels (FIG. 2A-2C). Meanwhile, Hep3B cells demonstrated low PCSK9 secretion levels overall, illustrating that of the cell lines used, the Hep3B cell line is the least amenable to treatment to induce and demonstrate PCSK9 repression (FIG. 2A-2C).

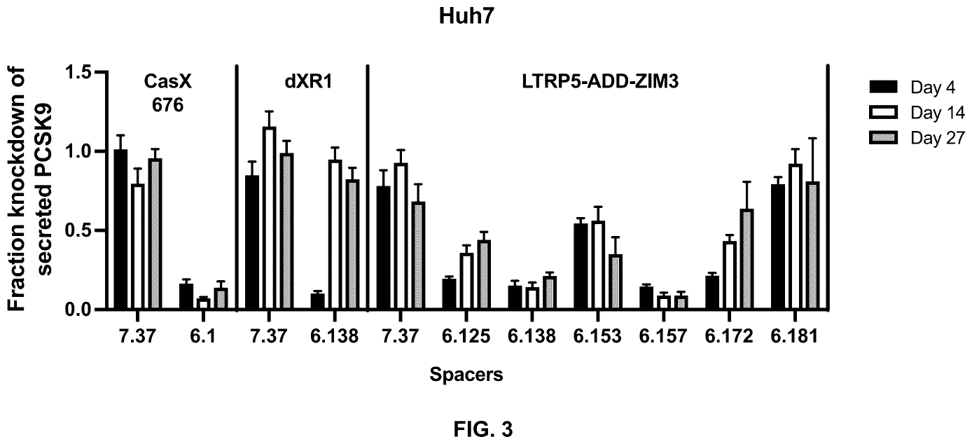

Culturing of treated Huh7 cells continued up to day 27 post-transfection, and PCSK9 secretion was measured at day 14 and day 27. The bar plot in FIG. 3 shows the quantification results of PCSK9 repression at day 4, day 14, and day 27 timepoints displayed as PCSK9 knockdown relative to the levels detected for the naïve control at the day 4 timepoint. The data demonstrate that treatment of Huh7 cells with LTRP5-ADD-ZIM3 with gRNAs having spacers 6.138 and 6.157 resulted in the most effective repression of PCSK9 secretion, and this repression was sustained through day 27 post-transfection (FIG. 3). Similarly, sustained knockdown was observed when Huh7 cells were treated with catalytically-active CasX 676 with spacer 6.1. While treatment with dXR1 and spacer 6.138 resulted in an initial strong repression at day 4, this repressive effect was transient as PCSK9 secretion levels returned to baseline levels at day 14 and day 27 post-transfection (FIG. 3). As anticipated, treatment with any of the three mRNA molecules with spacer 7.37 targeting the B2M locus did not affect PCSK9 secretion levels during this time-course experiment.

These experiments demonstrate that use of LTRP molecules with the ADD domain with the appropriate targeting spacer can result in long-term silencing of an endogenous target locus in various human cell lines. These findings also show that LTRP molecules with the ADD domain can be co-delivered as mRNA with a targeting gRNA to cells to induce repression.

### Example 3: Demonstration that Inclusion of a Second Repressor Domain Enhances Activity of LTRP-ADD Molecules while Maintaining their Specificity

Experiments were performed to determine whether incorporation of a second repressor domain would enhance the activity and specificity of LTRP molecules containing the ADD domain. This second repressor domain was positioned on the C-terminus of the LTRP5-ADD molecule, resulting in the generation of an LTRP molecule with a new configuration (hereafter described as an LTRP6 molecule). The schematic in FIG. 19 shows an LTRP6 molecule having either configuration #6a or #6b. A molecule with configuration #6a utilizes the same repressor domain at both the N-terminus and C-terminus positions relative to dCasX, and a molecule with configuration #6b uses two different repressor domains at the two positions.

Experiments were performed to assess whether incorporating a second repressor domain on the C-terminus of an LTRP5-ADD-ZIM3 molecule, which resulted in the generation of an LTRP6 molecule having a #6b configuration, would induce long-term repression of the target locus.

### Materials and Methods

### Generation of LTRP Constructs and Plasmid Cloning:

Plasmid constructs encoding for variants of the LTRP #5 construct with the ZIM3-KRAB domain (LTRP #5.A; see FIG. 19 for LTRP #5 configuration) were built using standard molecular cloning techniques. The resulting constructs comprised of sequences encoding for one of the following four alternative variations of LTRP5-ZIM3, where the additional DNMT3A domains were incorporated: 1) LTRP5-ZIM3+ADD; 2) LTRP5-ZIM3+ADD+PWWP; 3) LTRP5-ZIM3+ADD without the DNMT3A catalytic domain; and 4) LTRP5-ZIM3+ADD+PWWP without the DNMT3A catalytic domain. The sequences of key elements within the LTRP5-ZIM3 molecule and its variants are listed in Table 14, with the full-length protein sequence for each LTRP5-ZIM3 and its variants listed in Table 15. Table 14 and Table 15 are also described in PCT application WO/2023/049742. Sequences encoding the LTRP molecules also contained a 2× FLAG tag.

### Generation of LTRP6 Constructs and Plasmid Cloning:

Plasmid constructs coding for an LTRP6 (configuration 6) molecule using the ZIM3-KRAB domain as the first repressor domain were built using standard molecular cloning techniques as described. Briefly, representative members of the top 95 repressor domains described in Table 13, also described in WO/2023/049742, were cloned as the second repressor domain onto the C-terminus of an LTRP5-ADD-ZIM3 construct (with protein sequences provided in Table 15 see also WO/2023/049742). An LTRP5-ADD-ZIM3 molecule served as an experimental control. SEQ ID NOs corresponding to the domain IDs in the tables below are provided in Table 14 (the domains are also as disclosed in WO/2023/049742). Plasmids also harbored constructs encoding for the gRNA scaffold variant 174 (with an RNA sequence of SEQ ID NO: 1474) having either a spacer targeting the endogenous B2M locus (spacer 7.165; UCCCUAUGUCCUUGCUGUUU; SEQ ID NO: 1914) or a non-targeting control. These constructs were all cloned upstream of a P2A-puromycin element on the lentiviral plasmid.

### Transfection of HEK293T Cells:

Seeded HEK293T cells were transiently transfected with 100 ng of LTRP plasmids, each containing an LTRP:gRNA construct encoding for an LTRP6 variant containing a ZIM3-KRAB domain as the first repressor domain and an enhanced repressor domain as the second repressor domain, with the gRNA having either non-targeting spacer or a B2M-targeting spacer. 24 hours post-transfection, cells were selected with 1 g/mL puromycin for three days. Cells were harvested for repression analysis at day 4, day 8, and day 13 post-transfection. Briefly, repression analysis was conducted by analyzing B2M protein expression via HLA immunostaining followed by flow cytometry, as described in Example 1.

### Results:

The effect of incorporating a second repressor domain into the LTRP5-ADD-ZIM3 molecule, thereby generating an LTRP6 construct, on increasing long-term repression of the target B2M locus was assessed. The results from this time-course experiment are depicted in Tables 16-18, which shows the average percentage of cells characterized as HLA-negative (indicative of B2M repression) for each condition at 4, 8, and 13 days post-transfection.

TABLE 13
List of 95 repressor domain candidates identified from the high-throughput
screen assessing dXR repression of the HBEGF gene and subsequent application of
the following criteria: p-value < 0.01 and log₂(fold change) > 2

| | | | | |
|---|---|---|---|---|
| | | SEQ | Log2 (fold | |
| Domain ID | Species | ID NO | change) | P-value |

Top 95 most effective repressor domains

| | | | | |
|---|---|---|---|---|
| DOMAIN_7694 | Columba livia | 130 | 3.7111 | 1.13E−04 |
| DOMAIN_10123 | Rattus norvegicus | 131 | 3.6356 | 8.11E−06 |
| DOMAIN_15507 | Cebus imitator | 132 | 3.8531 | 1.53E−07 |
| DOMAIN_17905 | Chimp | 133 | 2.5038 | 5.60E−04 |
| DOMAIN_20505 | Chlorocebus sabaeus | 134 | 3.4989 | 2.91E−06 |
| DOMAIN_26749 | Ophiophagus hannah | 135 | 5.4323 | 1.53E−07 |
| DOMAIN_27604 | Ailuropoda melanoleuca | 136 | 2.8198 | 6.05E−05 |
| DOMAIN_29304 | Peromyscus maniculatus bairdii | 137 | 4.0496 | 1.53E−07 |
| DOMAIN_30173 | Phyllostomus discolor | 138 | 2.2538 | 5.41E−04 |
| DOMAIN_737 | Bonobo | 139 | 4.544 | 1.53E−07 |
| DOMAIN_10331 | Colobus angolensis palliatus | 140 | 3.6796 | 1.53E−07 |
| DOMAIN_10948 | Colobus angolensis palliatus | 141 | 3.2959 | 2.30E−06 |
| DOMAIN_11029 | Mandrillus leucophaeus | 142 | 3.5748 | 1.53E−07 |
| DOMAIN_17358 | Bos indicus × Bos taurus | 143 | 4.9878 | 1.53E−07 |
| DOMAIN_17759 | Felis catus | 144 | 3.3159 | 1.38E−06 |
| DOMAIN_18258 | Physeter macrocephalus | 145 | 3.75 | 3.42E−04 |
| DOMAIN_19804 | Callorhinus ursinus | 146 | 3.8217 | 1.53E−07 |
| DOMAIN_221 | Bonobo | 147 | 3.5533 | 3.06E−06 |
| DOMAIN_881 | Bonobo | 148 | 4.3546 | 4.59E−07 |
| DOMAIN_2380 | Orangutan | 149 | 3.2024 | 1.74E−04 |
| DOMAIN_2942 | Gibbon | 150 | 3.3658 | 1.38E−06 |
| DOMAIN_4687 | Marmoset | 151 | 5.2288 | 3.22E−06 |
| DOMAIN_4806 | Marmoset | 152 | 3.3896 | 1.58E−04 |
| DOMAIN_4968 | Marmoset | 153 | 3.0315 | 0.0022262 |
| DOMAIN_5066 | Marmoset | 154 | 2.9062 | 0.0067409 |
| DOMAIN_5290 | Owl Monkey | 155 | 3.0993 | 5.16E−05 |
| DOMAIN_5463 | Owl Monkey | 156 | 3.2102 | 0.0022788 |
| DOMAIN_6248 | Saimiri boliviensis | 157 | 2.4415 | 0.0056883 |
| DOMAIN_6445 | Alligator sinensis | 158 | 3.1151 | 4.51E−04 |
| DOMAIN_6802 | Pantherophis guttatus | 159 | 3.0403 | 5.18E−04 |
| DOMAIN_6807 | Xenopus laevis | 160 | 3.1615 | 5.16E−05 |
| DOMAIN_7255 | Microcaecilia unicolor | 161 | 4.5265 | 1.38E−06 |
| DOMAIN_8503 | Mus caroli | 162 | 2.8193 | 0.003503 |
| DOMAIN_8790 | Marmota monax | 163 | 2.7436 | 2.06E−04 |
| DOMAIN_8853 | Mesocricetus auratus | 164 | 4.6199 | 1.53E−07 |
| DOMAIN_9114 | Peromyscus maniculatus bairdii | 165 | 2.2058 | 0.0048423 |
| DOMAIN_9331 | Peromyscus maniculatus bairdii | 166 | 4.1063 | 4.59E−07 |
| DOMAIN_9538 | Mus musculus | 167 | 3.5443 | 1.20E−04 |
| DOMAIN_9960 | Octodon degus | 168 | 3.4751 | 1.07E−06 |
| DOMAIN_10277 | Dipodomys ordii | 169 | 2.8257 | 4.16E−04 |
| DOMAIN_10577 | Colobus angolensis palliatus | 170 | 4.1248 | 1.53E−07 |
| DOMAIN_11348 | Chlorocebus sabaeus | 171 | 3.3651 | 2.95E−05 |
| DOMAIN_11386 | Capra hircus | 172 | 3.7637 | 4.75E−06 |
| DOMAIN_11486 | Bos mutus | 173 | 4.8326 | 1.53E−07 |
| DOMAIN_11683 | Nomascus leucogenys | 174 | 2.9249 | 0.0015672 |
| DOMAIN_12292 | Sus scrofa | 175 | 4.3194 | 1.53E−07 |
| DOMAIN_12452 | Neophocaena asiaeorientalis | 176 | 3.8774 | 5.05E−06 |
| DOMAIN_12631 | Macaca fascicularis | 177 | 3.6926 | 1.53E−07 |
| DOMAIN_13331 | Macaca fascicularis | 178 | 3.5154 | 2.15E−04 |
| DOMAIN_13468 | Phascolarctos cinereus | 179 | 4.1548 | 1.38E−06 |
| DOMAIN_13539 | Gorilla | 180 | 3.4924 | 1.79E−05 |
| DOMAIN_14659 | Acinonyx jubatus | 181 | 4.0495 | 1.06E−05 |
| DOMAIN_14755 | Cebus imitator | 182 | 3.1667 | 1.88E−04 |
| DOMAIN_15126 | Callithrix jacchus | 183 | 2.9781 | 4.08E−04 |
| DOMAIN_16444 | Acinonyx jubatus | 184 | 3.2246 | 2.30E−06 |
| DOMAIN_16688 | Lipotes vexillifer | 185 | 3.5601 | 4.26E−05 |
| DOMAIN_16806 | Sapajus apella | 186 | 3.9386 | 1.53E−07 |
| DOMAIN_17317 | Otolemur garnettii | 187 | 3.4551 | 1.81E−04 |
| DOMAIN_17432 | Otolemur garnettii | 188 | 3.11 | 1.36E−05 |
| DOMAIN_18137 | Monodelphis domestica | 189 | 3.292 | 3.51E−05 |
| DOMAIN_18216 | Physeter macrocephalus | 190 | 3.0602 | 9.40E−04 |
| DOMAIN_18563 | OwlMonkey | 191 | 3.0406 | 0.0034849 |
| DOMAIN_19229 | Enhydra lutris kenyoni | 192 | 4.0294 | 5.01E−05 |
| DOMAIN_19460 | Monodelphis domestica | 193 | 3.995 | 1.97E−05 |
| DOMAIN_19476 | OwlMonkey | 194 | 4.1343 | 1.53E−07 |
| DOMAIN_19821 | Rhinopithecus roxellana | 195 | 3.583 | 1.53E−07 |
| DOMAIN_19892 | Ursus maritimus | 196 | 3.1396 | 5.21E−04 |
| DOMAIN_19896 | Ovis aries | 197 | 2.2228 | 1.58E−04 |
| DOMAIN_19949 | Callorhinus ursinus | 198 | 3.2903 | 2.62E−04 |
| DOMAIN_21247 | Neovison vison | 199 | 2.741 | 0.0043129 |
| DOMAIN_21317 | Pteropus vampyrus | 200 | 4.0893 | 1.18E−05 |
| DOMAIN_21336 | Equus caballus | 201 | 2.738 | 0.005135 |
| DOMAIN_21603 | Lipotes vexillifer | 202 | 2.8535 | 4.35E−04 |
| DOMAIN_21755 | Equus caballus | 203 | 3.1889 | 0.0028238 |
| DOMAIN_22153 | Zalophus californianus | 204 | 3.6967 | 3.52E−06 |
| DOMAIN_22270 | Bonobo | 205 | 2.3813 | 0.0030391 |
| DOMAIN_23394 | Vicugna pacos | 206 | 4.0769 | 3.06E−07 |
| DOMAIN_23723 | Carlito syrichta | 207 | 3.5301 | 8.71E−05 |
| DOMAIN_24125 | Saimiri boliviensis | 208 | 3.9692 | 1.53E−07 |
| DOMAIN_24458 | Lynx pardinus | 209 | 3.4012 | 9.66E−05 |
| DOMAIN_24663 | Myotis brandtii | 210 | 2.9806 | 1.49E−04 |
| DOMAIN_25289 | Ursus maritimus | 211 | 3.4113 | 7.70E−05 |
| DOMAIN_25379 | Sapajus apella | 212 | 3.5892 | 1.53E−07 |
| DOMAIN_25405 | Desmodus rotundus | 213 | 3.8846 | 3.20E−05 |
| DOMAIN_26070 | Geotrypetes seraphini | 214 | 3.7958 | 1.53E−07 |
| DOMAIN_26322 | Geotrypetes seraphini | 215 | 2.9265 | 7.13E−04 |
| DOMAIN_26732 | Meleagris gallopavo | 216 | 2.7548 | 0.0057183 |
| DOMAIN_27060 | Gopherus agassizii | 217 | 2.7943 | 0.0029172 |
| DOMAIN_27385 | Octodon degus | 218 | 4.1339 | 2.77E−05 |
| DOMAIN_27506 | Bos mutus | 219 | 3.8121 | 4.29E−06 |
| DOMAIN_27811 | Callithrix jacchus | 220 | 2.9728 | 8.34E−05 |
| DOMAIN_28640 | Colinus virginianus | 221 | 3.624 | 4.13E−06 |
| DOMAIN_28803 | Monodelphis domestica | 222 | 3.0697 | 2.07E−05 |
| DOMAIN_30661 | Physeter macrocephalus | 223 | 2.15 | 4.76E−05 |
| DOMAIN_31643 | Micrurus lemniscatus | 224 | 3.8782 | 3.57E−04 |

Log2 fold change and P-values for the full set of 1,597 domains repressor domains is disclosed in Table 19 of WO 2023/049742, the contents of which are incorporated by reference herein.

TABLE 14
Sequences of LTRP components
(e.g., additional domains fused to dCasX)
to generate LTRP5 variant plasmids

| | | SEQ ID |
|---|---|---|
| Component | Protein sequence | NO |
| ZIM3 KRAB domain | MNNSQGRVTFEDVTVNFTQGEWQRLNPEQRNLYRDVMLENYSNLV | 1892 |
| | SVGQGETTKPDVILRLEQGKEPWLEEEEVLGSGRAEKNGDIGGQI | |
| | WKPKDVKESL | |
| DNMT3A catalytic | NHDQEFDPPKVYPPVPAEKRKPIRVLSLFDGIATGLLVLKDLGIQ | 126 |
| domain (CD) | VDRYIASEVCEDSITVGMVRHQGKIMYVGDVRSVTQKHIQEWGPF | |
| | DLVIGGSPCNDLSIVNPARKGLYEGTGRLFFEFYRLLHDARPKEG | |
| | DDRPFFWLFENVVAMGVSDKRDISRFLESNPVMIDAKEVSAAHRA | |
| | RYFWGNLPGMNRPLASTVNDKLELQECLEHGRIAKFSKVRTITTR | |
| | SNSIKQGKDQHFPVFMNEKEDILWCTEMERVFGFPVHYTDVSNMS | |
| | RLARQRLLGRSWSVPVIRHLFAPLKEYFACV | |
| DNMT3L interaction | MGPMEIYKTVSAWKRQPVRVLSLFRNIDKVLKSLGFLESGSGSGG | 127 |
| domain | GTLKYVEDVTNVVRRDVEKWGPFDLVYGSTQPLGSSCDRCPGWYM | |
| | FQFHRILQYALPRQESQRPFFWIFMDNLLLTEDDQETTTRFLQTE | |
| | AVTLQDVRGRDYQNAMRVWSNIPGLKSKHAPLTPKEEEYLQAQVR | |
| | SRSKLDAPKVDLLVKNCLLPLREYFKYFSQNSLPL | |
| dCasX491 | QEIKRINKIRRRLVKDSNTKKAGKTGPMKTLLVRVMTPDLRERLE | 4 |
| | NLRKKPENIPQPISNTSRANLNKLLTDYTEMKKAILHVYWEEFQK | |
| | DPVGLMSRVAQPASKKIDQNKLKPEMDEKGNLTTAGFACSQCGQP | |
| | LFVYKLEQVSEKGKAYTNYFGRCNVAEHEKLILLAQLKPEKDSDE | |
| | AVTYSLGKFGQRALDFYSIHVTKESTHPVKPLAQIAGNRYASGPV | |
| | GKALSDACMGTIASFLSKYQDIIIEHQKVVKGNQKRLESLRELAG | |
| | KENLEYPSVTLPPQPHTKEGVDAYNEVIARVRMWVNLNLWQKLKL | |
| | SRDDAKPLLRLKGFPSFPLVERQANEVDWWDMVCNVKKLINEKKE | |
| | DGKVFWQNLAGYKRQEALRPYLSSEEDRKKGKKFARYQLGDLLLH | |
| | LEKKHGEDWGKVYDEAWERIDKKVEGLSKHIKLEEERRSEDAQSK | |
| | AALTDWLRAKASFVIEGLKEADKDEFCRCELKLQKWYGDLRGKPF | |
| | AIEAENSILDISGFSKQYNCAFIWQKDGVKKLNLYLIINYFKGGK | |
| | LRFKKIKPEAFEANRFYTVINKKSGEIVPMEVNFNFDDPNLIILP | |
| | LAFGKRQGREFIWNDLLSLETGSLKLANGRVIEKTLYNRRTRQDE | |
| | PALFVALTFERREVLDSSNIKPMNLIGVARGENIPAVIALTDPEG | |
| | CPLSRFKDSLGNPTHILRIGESYKEKQRTIQAKKEVEQRRAGGYS | |
| | RKYASKAKNLADDMVRNTARDLLYYAVTQDAMLIFANLSRGFGRQ | |
| | GKRTFMAERQYTRMEDWLTAKLAYEGLSKTYLSKTLAQYTSKTCS | |
| | NCGFTITSADYDRVLEKLKKTATGWMTTINGKELKVEGQITYYNR | |
| | YKRQNVVKDLSVELDRLSEESVNNDISSWTKGRSGEALSLLKKRF | |
| | SHRPVQEKFVCLNCGFETHAAEQAALNIARSWLFLRSQEYKKYQT | |
| | NKTTGNTDKRAFVETWQSFYRKKLKEVWKPAV | |
| Linker 1 | GGPSSGAPPPSGGSPAGSPTSTEEGTSESATPESGPGTSTEPSEG | 123 |
| | SAPGSPAGSPTSTEEGTSTEPSEGSAPGTSTEPSE | |
| Linker 2 | SSGNSNANSRGPSFSSGLVPLSLRGSH | 122 |
| Linker 3A | GGSGGG | 124 |
| Linker 3B | GGSGGGS | 120 |
| Linker 4 | GSGSGGG | 121 |
| NLS A | PKKKRKV | 30 |
| NLS B | | |
| DNMT3A ADD | ERLVYEVRQKCRNIEDICISCGSLNVTLEHPLFIGGMCQNCKNCF | 125 |
| domain | LECAYQYDDDGYQSYCTICCGGREVLMCGNNNCCRCFCVECVDLL | |
| | VGPGAAQAAIKEDPWNCYMCGHKGTYGLLRRREDWPSRLQMFFAN | |
| DNMT3A PWWP | TKAADDEPEYEDGRGFGIGELVWGKLRGFSWWPGRIVSWWMTGRS | 1907 |
| domain | RAAEGTRWVMWFGDGKFSVVCVEKLMPLSSFCSAFHQATYNKQPM | |
| | YRKAIYEVLQVASSRAGKLFPACHDSDESDSGKAVEVQNKQMIEW | |
| | ALGGFQPSGPKGLEPPEEEKNPYKEV | |
| Endogenous sequence | YTDMWVEPEAAAYAPPPPAKKPRKSTTEKPKVKEIIDERTR | 1908 |
| between DNMT3A | | |
| PWWP and ADD | | |
| domains (endo) | | |

TABLE 15
Protein sequences of LTRP5 variants assayed in this example

| LTRP ID | SEQ ID NO |
|---|---|
| LTRP5-ZIM3 | 1909 |
| LTRP5-ZIM3 + ADD | 1883 |
| LTRP5-ZIM3 + ADD + PWWP | 1910 |
| LTRP5-ZIM3 + ADD − CD | 1911 |
| LTRP5-ZIM3 + ADD + PWWP − CD | 1912 |

TABLE 16
Level of B2M repression mediated by LTRP constructs with
various repressor domains quantified at 4 days post-transfection.
The domain ID shown for each LTRP6 construct designates
the second repressor domain used and assessed

| | | | | Sam- |
|---|---|---|---|---|
| | | Mean % HLA- | Standard | ple |
| Repressor construct | Spacer | negative cells | deviation | Size |
| LTRP5-ADD-ZIM3 | NT | 6.827 | 0.359 | 3 |
| LTRP5-ADD-ZIM3 | 7.165 | 53.833 | 1.159 | 3 |
| LTRP6_DOMAIN_20505 | 7.165 | 69.333 | 0.493 | 3 |
| LTRP6_DOMAIN_30173 | 7.165 | 70.267 | 1.115 | 3 |
| LTRP6_DOMAIN_15507 | 7.165 | 71.800 | 0.436 | 3 |
| LTRP6_DOMAIN_18137 | 7.165 | 71.900 | 0.600 | 3 |
| LTRP6_DOMAIN_24458 | 7.165 | 73.333 | 0.961 | 3 |
| LTRP6_DOMAIN_6445 | 7.165 | 73.567 | 0.666 | 3 |
| LTRP6_DOMAIN_17432 | 7.165 | 74.667 | 0.603 | 3 |
| LTRP6_DOMAIN_9960 | 7.165 | 76.000 | 0.100 | 3 |
| LTRP6_DOMAIN_13468 | 7.165 | 76.167 | 0.493 | 3 |
| LTRP6_DOMAIN_8853 | 7.165 | 77.400 | 0.173 | 3 |
| LTRP6_DOMAIN_6802 | 7.165 | 77.700 | 0.361 | 3 |
| LTRP6_DOMAIN_7694 | 7.165 | 78.300 | 0.361 | 3 |
| LTRP6_DOMAIN_29304 | 7.165 | 78.933 | 0.814 | 3 |
| LTRP6_DOMAIN_25379 | 7.165 | 80.033 | 0.569 | 3 |
| LTRP6_DOMAIN_22153 | 7.165 | 80.200 | 1.493 | 3 |
| LTRP6_DOMAIN_7255 | 7.165 | 80.567 | 0.551 | 3 |
| LTRP6_DOMAIN_26749 | 7.165 | 81.467 | 0.503 | 3 |
| LTRP6_DOMAIN_10123 | 7.165 | 81.833 | 0.289 | 3 |

TABLE 17
Level of B2M repression mediated by LTRP constructs with
various repressor domains quantified at 8 days post-transfection.
The domain ID shown for each LTRP6 construct designates
the second repressor domain used and assessed

| | | | | Sam- |
|---|---|---|---|---|
| | | Mean % HLA- | Standard | ple |
| Repressor construct | Spacer | negative cells | deviation | Size |
| LTRP5-ADD-ZIM3 | NT | 2.100 | 0.311 | 2 |
| LTRP5-ADD-ZIM3 | 7.165 | 55.000 | 0.300 | 3 |
| LTRP6_DOMAIN_20505 | 7.165 | 72.367 | 1.686 | 3 |
| LTRP6_DOMAIN_30173 | 7.165 | 72.733 | 1.069 | 3 |
| LTRP6_DOMAIN_15507 | 7.165 | 74.033 | 0.351 | 3 |
| LTRP6_DOMAIN_18137 | 7.165 | 75.100 | 1.253 | 3 |
| LTRP6_DOMAIN_24458 | 7.165 | 76.167 | 0.737 | 3 |
| LTRP6_DOMAIN_6445 | 7.165 | 77.600 | 1.249 | 3 |
| LTRP6_DOMAIN_17432 | 7.165 | 78.233 | 0.153 | 3 |
| LTRP6_DOMAIN_9960 | 7.165 | 77.600 | 0.346 | 3 |
| LTRP6_DOMAIN_13468 | 7.165 | 79.067 | 0.651 | 3 |
| LTRP6_DOMAIN_8853 | 7.165 | 79.333 | 1.150 | 3 |
| LTRP6_DOMAIN_6802 | 7.165 | 78.400 | 0.265 | 3 |
| LTRP6_DOMAIN_7694 | 7.165 | 81.433 | 0.416 | 3 |
| LTRP6_DOMAIN_29304 | 7.165 | 80.433 | 0.569 | 3 |
| LTRP6_DOMAIN_25379 | 7.165 | 82.167 | 0.351 | 3 |
| LTRP6_DOMAIN_22153 | 7.165 | 81.333 | 1.210 | 3 |
| LTRP6_DOMAIN_7255 | 7.165 | 80.933 | 0.404 | 3 |
| LTRP6_DOMAIN_26749 | 7.165 | 85.767 | 0.777 | 3 |
| LTRP6_DOMAIN_10123 | 7.165 | 83.000 | 0.625 | 3 |

TABLE 18
Level of B2M repression mediated by LTRP constructs with
various repressor domains quantified at 13 days post-transfection.
The domain ID shown for each LTRP6 construct designates
the second repressor domain used and assessed

| | | | | Sam- |
|---|---|---|---|---|
| | | Mean % HLA- | Standard | ple |
| Repressor construct | Spacer | negative cells | deviation | Size |
| LTRP5-ADD-ZIM3 | NT | 1.587 | 0.670 | 3 |
| LTRP5-ADD-ZIM3 | 7.165 | 50.267 | 0.987 | 3 |
| LTRP6_DOMAIN_20505 | 7.165 | 70.133 | 0.945 | 3 |
| LTRP6_DOMAIN_30173 | 7.165 | 70.233 | 0.666 | 3 |
| LTRP6_DOMAIN_15507 | 7.165 | 72.067 | 0.252 | 3 |
| LTRP6_DOMAIN_18137 | 7.165 | 72.767 | 1.514 | 3 |
| LTRP6_DOMAIN_24458 | 7.165 | 73.967 | 0.404 | 3 |
| LTRP6_DOMAIN_6445 | 7.165 | 75.967 | 0.723 | 3 |
| LTRP6_DOMAIN_17432 | 7.165 | 75.667 | 0.351 | 3 |
| LTRP6_DOMAIN_9960 | 7.165 | 74.233 | 0.814 | 3 |
| LTRP6_DOMAIN_13468 | 7.165 | 76.767 | 0.764 | 3 |
| LTRP6_DOMAIN_8853 | 7.165 | 75.133 | 1.457 | 3 |
| LTRP6_DOMAIN_6802 | 7.165 | 74.700 | 1.136 | 3 |
| LTRP6_DOMAIN_7694 | 7.165 | 78.967 | 0.321 | 3 |
| LTRP6_DOMAIN_29304 | 7.165 | 78.233 | 1.332 | 3 |
| LTRP6_DOMAIN_25379 | 7.165 | 78.600 | 0.985 | 3 |
| LTRP6_DOMAIN_22153 | 7.165 | 77.533 | 0.839 | 3 |
| LTRP6_DOMAIN_7255 | 7.165 | 77.700 | 0.265 | 3 |
| LTRP6_DOMAIN_26749 | 7.165 | 83.800 | 1.127 | 3 |
| LTRP6_DOMAIN_10123 | 7.165 | 79.367 | 0.702 | 3 |

The data in Tables 16-18 show that all LTRP6 constructs with a B2M-targeting gRNA were able to induce higher levels of B12M repression compared to LTRP5 constructs through at least 13 days post-transfection, with a slight variation in the potency of repression depending on the repressor domain utilized as a second repressor domain. Notably, use of DOMAIN_26749 (from Ophiophagus hannah) as the second repressor domain resulted in ~83.800 of B12M repression, the highest level of repression amongst the other repressor domains assessed. In comparison, the LTRP5-ADD-ZIM3 construct only achieved a level of repression at ~50.3%, which is ~40% lower than that achieved by an LT-P6 construct with the ZIM3 and DOMAIN_26749 repressor domains (Tables 16-18). The observed improvement of repression from these experiments may be explained by the synergistic effects of using a more potent repressor domain, the presence of the ADD domain, and incorporation of the second repressor domain in the LTRP6 construct configuration. Example 4 below further investigates the effects of using two enhanced repressor domains on the activity and specificity of an LTRP6 construct.

The results from these experiments demonstrate that incorporation of a second repressor domain can enhance the activity of an LTRP molecule containing the ADD domain. The results described herein show that incorporating an enhanced repressor domain on the C-terminus of an LTRP5-ADD-ZIM3 molecule, resulting in the generation of an LTRP6 construct, improves repression activity at an endogenous locus in human cells.

### Example 4: Assessing the Effects of Using Two Repressor Domains and their Relative Positions within the LTRP6 Molecule on Activity and Specificity

Experiments were performed to determine whether variations of the LTRP configuration #6 (LTRP6) molecule, described previously in Example 3, would result in improved long-term repression of the target locus and reduced off-target methylation. Here, the effects of using an enhanced repressor domain as both a first repressor domain and a second repressor domain within an LTRP6 molecule, as well as the relative placements of these repressor domains, were evaluated. Two variations of the LTRP6 configuration (shown in FIG. 19) were tested in this example. A molecule with configuration 6a utilizes the same repressor domain at both the N-terminus and C-terminus positions relative to dCasX, and a molecule with configuration 6b uses two different repressor domains at the two positions.

### Materials and Methods

### Generation of LTRP6 Constructs and Plasmid Cloning:

Plasmid constructs encoding for variants of an LTRP6 molecule, having either configuration 6a or 6b shown in the schematic in FIG. 19, were built using standard molecular cloning techniques. In this example, the following four repressor domains were used to generate variants of the LTRP6 molecule: DOMAIN_22153, DOMAIN_7255, DOMAIN_26749, and DOMAIN_10123. These four domains were selected because they induced the highest level of B2M repression at 4 days post-transfection as shown in Example 3, when incorporated as the second repressor domain in an LTRP6 configuration (Tables 16-18). Each of the four repressor domains were tested in combination at the N-terminus and/or C-terminus positions relative to dCasX, resulting in a total of 16 combinations assessed. The encoding sequences of the 16 LTRP6 variants are shown in Table 19, with the corresponding protein sequences shown in Table 20. Plasmids also harbored constructs encoding for the gRNA scaffold variant 174 having either a spacer targeting the endogenous B2M locus (spacer 7.165; UCCCUAUGUCCUUJGCUGUUU; SEQ ID NO: 1914) or a non-targeting control. These constructs were all cloned upstream of a P2A-puromycin element on the lentiviral plasmid.

TABLE 19
Encoding sequences of the 16 LTRP6 variants assessed in this example*

| Component | Domain Combination # | Domains | SEQ ID NO |
|---|---|---|---|
| START codon + | | | 1970 |
| NLS + buffer sequence | | | |
| START codon + | | | 1971 |
| DNMT3A ADD | | | |
| domain | | | |
| DNMT3A catalytic | | | 1972 |
| domain | | | |
| Linker 2 (L2) | | | 1973 |
| DNMT3L interaction | | | 1974 |
| domain | | | |
| Linker 3A (L3A) | | | 1975 |
| N-terminus repressor | Combinations 1, 5, 9, and 13 | DOMAIN_22153 | 1976 |
| domain | | | |
| | Combinations 2, 6, 10, and 14 | DOMAIN_7255 | 1977 |
| | Combinations, 3, 7, 11, and 15 | DOMAIN_26749 | 1978 |
| | Combinations 4, 8, 12, and 16 | DOMAIN_10123 | 1979 |
| Linker 1 (L1) | | | 1980 |
| dCasX | | dCasX491 | 1981 |
| Buffer sequence + | | | 1982 |
| Linker 3B (L3B) + | | | |
| buffer sequence | | | |
| C-terminus repressor | Combinations 1-4 | DOMAIN_7255 | 1977 |
| domain | | | |
| | Combinations 5-8 | DOMAIN_22153 | 1976 |
| | Combinations 9-12 | DOMAIN_10123 | 1979 |
| | Combinations 13-16 | DOMAIN_26749 | 1978 |
| Linker 4v1 (L4v1) | | | 1983 |
| NLS | | | 1984 |

*Components are listed in a 5′ to 3′ order within the constructs

TABLE 20
Full-length protein sequences of the 16
LTRP6 variants assessed in this example

| | Domain Combination | | AA sequence |
|---|---|---|---|
| Component | # | Domains | SEQ ID NO |
| START codon + | | | 1985 |
| NLS + buffer | | | |
| sequence | | | |
| START codon + | | | 1986 |
| DNMT3A | | | |
| ADD domain | | | |
| DNMT3A | | | 126 |
| catalytic | | | |
| domain | | | |
| Linker 2 (L2) | | | 122 |
| DNMT3L | | | 127 |
| interaction | | | |
| domain | | | |
| Linker 3A | | | 124 |
| (L3A) | | | |
| N-terminus | Combinations 1, 5, 9, | DOMAIN_{—} | 204 |
| repressor | and 13 | 22153 | |
| domain | Combinations 2, 6, 10, | DOMAIN_{—} | 161 |
| | and 14 | 7255 | |
| | Combinations, 3, 7, 11, | DOMAIN_{—} | 135 |
| | and 15 | 26749 | |
| | Combinations 4, 8, 12, | DOMAIN_{—} | 131 |
| | and 16 | 10123 | |
| Linker 1 (L1) | | | 123 |
| dCasX | | dCasX491 | 4 |
| Buffer | | | 1987 |
| sequence + | | | |
| Linker 3B | | | |
| (L3B) + buffer | | | |
| sequence | | | |
| C-terminus | Combinations 1-4 | DOMAIN_{—} | 161 |
| repressor | | 7255 | |
| domain | Combinations 5-8 | DOMAIN_{—} | 204 |
| | | 22153 | |
| | Combinations 9-12 | DOMAIN_{—} | 131 |
| | | 10123 | |
| | Combinations 13-16 | DOMAIN_{—} | 135 |
| | | 26749 | |
| Linker 4v1 | | | 1988 |
| (L4v1) | | | |
| NLS | | | 30 |

Transfection of HEK293T cells was performed following similar methods as described in Example 3. Briefly, ITEK293T cells were transiently transfected with a plasmid encoding an LTRP6 variant (listed in Table 19) with a gRNA having a B2M-targeting spacer. 24 hours post-transfection, cells were selected with puromycin for five days. Cells were harvested for determining the level of B2M repression at 8, 12, 20, 27, 44, 59, 74, and 104 days post-transfection. An LTRP5-ZIM3 molecule and an LTRP5-ADD-ZIM3 molecule served as experimental controls. Furthermore, an LTRP6 variant using a ZIM3 domain as a first and second repressor domain, also known as an LTRP6 with dual ZIM3, was also included in this experiment for comparison.

Bisulfite sequencing was also performed to assess off-target methylation at the VEGFA locus, which was performed as described in Example 1. Briefly, HEK293T cells transiently transfected with LTRP6 variant plasmids, with a B2M-targeting gRNA or non-targeting gRNA, were harvested at five days post-transfection for gDNA extraction for bisulfite sequencing. The following controls were also included: 1) CasX 491 paired with B2M spacer 7.37 (GGCCGAGAUGUCUCGCUCCG; SEQ ID NO: 1904); 2) a dCas9-ZNF10-DNMT3A/3L with its corresponding B2M spacer; 3) LTRP1 (described in Example 1) with either a non-targeting or B2M spacer 7.165; 4) LTRP5-ZIM5 with B2M spacer 7.165; and 5) LTRP5-ADD-ZIM3 with either a non-targeting or B2M spacer 7.165.

### Results:

The effects of using an enhanced LTRP configuration having both a first repressor domain and a second repressor domain, as well as their relative placements within an LTRP6 molecule, were assessed. The results from a time-course experiment evaluating the effects on long-term repression of the target B2M locus are depicted in Tables 21-28. Overall, the data demonstrate that the incorporation of a second repressor domain to the C-terminus of an LTRP5-ADD molecule (resulting in the LTRP6 configuration) substantially improved durable B2M repression, recapitulating the findings observed in Example 3. The addition of a ZIM3 domain to the C-terminus of an LTRP5-ADD-ZIM3, resulting in the generation of an LTRP6-dual-ZIM3, enhanced long-term repression when compared to the level of B2M repression achieved by the LTRP5-ADD-ZIM3 construct, at least through 59 days post-transfection, although this improvement in long-term repression was not sustained by day 104 (Tables 21-28). The enhanced repression observed with the use of a second ZIM3 domain further supports that the observed improvement in activity in the LTRP6 configuration is due to the presence of a second repressor domain linked C-terminal to the dCasX. In comparison to LTRP6-dual-ZIM3, most of the other LTRP6 constructs harboring the two enhanced repressor domains further improved repressive activity even through 104 days post-transfection. Notably, LTRP6 molecules containing DOMAIN_26749 (either at the N-terminus and/or C-terminus) were the best performing molecules in inducing durable n2M repression (>900) through day 104, with LTRP6-dual-26749 (configuration #6a) demonstrating the highest level of repression and LTRP6-10123-26749 (configuration #6b) exhibiting the second highest repressive activity (Tables 21-28). Interestingly, in some instances, the placement of the repressor domains on the N-terminus of the dCasX compared to the C-terminus also affected activity of the molecule. For instance, placing DOMAIN_7255 on the N-terminus of the dCasX in combination with DOMAIN-22153 on the C-terminus resulted in ~58 repression; however, the level of repression increased to 9123 when DOMAIN_22153 was on the N-terminus and DOMAIN_7255 was on the C-terminus (Tables 21-28).

TABLE 21
Level of B2M repression mediated by LTRP constructs with various
repressor domains quantified at 8 days post-transfection. The
order of the domain IDs shown for each LTRP6 construct designates
the first repressor domain and second repressor domain at the
N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 8.437 | 2.483 | 3 |
| LTRP5-ZIM3 | 7.165 | 61.133 | 2.003 | 3 |
| LTRP5-ADD-ZIM3 | 7.165 | 74.967 | 1.443 | 3 |
| LTRP6-dual-ZIM3 | 7.165 | 89.833 | 0.416 | 3 |
| LTRP6-22153-7255 | 7.165 | 92.200 | 0.361 | 3 |
| LTRP6-dual-7255 | 7.165 | 91.267 | 0.321 | 3 |
| LTRP6-26749-7255 | 7.165 | 92.567 | 0.208 | 3 |
| LTRP6-10123-7255 | 7.165 | 92.100 | 0.529 | 3 |
| LTRP6-dual-22153 | 7.165 | 88.700 | 0.600 | 3 |
| LTRP6-7255-22153 | 7.165 | 91.500 | 0.100 | 3 |
| LTRP6-26749-22153 | 7.165 | 93.033 | 0.737 | 3 |
| LTRP6-10123-22153 | 7.165 | 91.967 | 0.702 | 3 |
| LTRP6-22153-10123 | 7.165 | 91.767 | 1.405 | 3 |
| LTRP6-7255-10123 | 7.165 | 90.800 | 2.128 | 3 |
| LTRP6-26749-10123 | 7.165 | 94.000 | 1.082 | 3 |
| LTRP6-dual-10123 | 7.165 | 93.100 | 1.136 | 3 |
| LTRP6-22153-26749 | 7.165 | 92.167 | 1.474 | 3 |
| LTRP6-7255-26749 | 7.165 | 91.567 | 1.365 | 3 |
| LTRP6-dual-26749 | 7.165 | 92.100 | 1.044 | 3 |
| LTRP6-10123-26749 | 7.165 | 92.367 | 0.802 | 3 |

TABLE 22
Level of B2M repression mediated by LTRP constructs with various
repressor domains quantified at 12 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 1.970 | 0.442 | 3 |
| LTRP5-ZIM3 | 7.165 | 50.433 | 1.250 | 3 |
| LTRP5-ADD-ZIM3 | 7.165 | 75.300 | 1.997 | 3 |
| LTRP6-dual-ZIM3 | 7.165 | 90.067 | 0.404 | 3 |
| LTRP6-22153-7255 | 7.165 | 93.567 | 0.493 | 3 |
| LTRP6-dual-7255 | 7.165 | 90.400 | 0.173 | 3 |
| LTRP6-26749-7255 | 7.165 | 94.367 | 0.306 | 3 |
| LTRP6-10123-7255 | 7.165 | 93.300 | 0.608 | 3 |
| LTRP6-dual-22153 | 7.165 | 89.067 | 0.924 | 3 |
| LTRP6-7255-22153 | 7.165 | 90.733 | 0.058 | 3 |
| LTRP6-26749-22153 | 7.165 | 95.267 | 0.709 | 3 |
| LTRP6-10123-22153 | 7.165 | 94.500 | 0.608 | 3 |
| LTRP6-22153-10123 | 7.165 | 93.400 | 0.100 | 3 |
| LTRP6-7255-10123 | 7.165 | 89.667 | 1.210 | 3 |
| LTRP6-26749-10123 | 7.165 | 95.700 | 0.200 | 3 |
| LTRP6-dual-10123 | 7.165 | 94.033 | 0.289 | 3 |
| LTRP6-22153-26749 | 7.165 | 95.067 | 0.651 | 3 |
| LTRP6-7255-26749 | 7.165 | 95.300 | 0.866 | 3 |
| LTRP6-dual-26749 | 7.165 | 95.567 | 0.404 | 3 |
| LTRP6-10123-26749 | 7.165 | 95.400 | 0.608 | 3 |

TABLE 23
Level of B2M repression mediated by LTRP constructs with various
repressor domains quantified at 20 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 6.060 | 3.841 | 3 |
| LTRP5-ZIM3 | 7.165 | 45.233 | 3.958 | 3 |
| LTRP5-ADD-ZIM3 | 7.165 | 71.700 | 2.107 | 3 |
| LTRP6-dual-ZIM3 | 7.165 | 89.767 | 1.124 | 3 |
| LTRP6-22153-7255 | 7.165 | 92.433 | 0.987 | 3 |
| LTRP6-dual-7255 | 7.165 | 89.233 | 1.528 | 3 |
| LTRP6-26749-7255 | 7.165 | 93.200 | 0.400 | 3 |
| LTRP6-10123-7255 | 7.165 | 92.133 | 0.929 | 3 |
| LTRP6-dual-22153 | 7.165 | 87.700 | 0.872 | 3 |
| LTRP6-7255-22153 | 7.165 | 88.633 | 0.814 | 3 |
| LTRP6-26749-22153 | 7.165 | 94.767 | 0.850 | 3 |
| LTRP6-10123-22153 | 7.165 | 93.400 | 0.794 | 3 |
| LTRP6-22153-10123 | 7.165 | 91.967 | 1.274 | 3 |
| LTRP6-7255-10123 | 7.165 | 87.233 | 1.960 | 3 |
| LTRP6-26749-10123 | 7.165 | 94.767 | 0.208 | 3 |
| LTRP6-dual-10123 | 7.165 | 92.367 | 0.404 | 3 |
| LTRP6-22153-26749 | 7.165 | 94.400 | 1.015 | 3 |
| LTRP6-7255-26749 | 7.165 | 93.667 | 1.069 | 3 |
| LTRP6-dual-26749 | 7.165 | 95.367 | 0.681 | 3 |
| LTRP6-10123-26749 | 7.165 | 94.867 | 0.907 | 3 |

TABLE 24
Level of B2M repression mediated by LTRP constructs with various
repressor domains quantified at 27 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 2.600 | 1.006 | 3 |
| LTRP5-ZIM3 | 7.165 | 36.733 | 2.608 | 3 |
| LTRP5-ADD-ZIM3 | 7.165 | 69.100 | 1.900 | 3 |
| LTRP6-dual-ZIM3 | 7.165 | 87.200 | 1.300 | 3 |
| LTRP6-22153-7255 | 7.165 | 91.300 | 1.179 | 3 |
| LTRP6-dual-7255 | 7.165 | 87.233 | 1.250 | 3 |
| LTRP6-26749-7255 | 7.165 | 92.367 | 0.551 | 3 |
| LTRP6-10123-7255 | 7.165 | 90.733 | 1.518 | 3 |
| LTRP6-dual-22153 | 7.165 | 85.567 | 1.007 | 3 |
| LTRP6-7255-22153 | 7.165 | 86.433 | 1.290 | 3 |
| LTRP6-26749-22153 | 7.165 | 94.067 | 0.681 | 3 |
| LTRP6-10123-22153 | 7.165 | 92.467 | 1.002 | 3 |
| LTRP6-22153-10123 | 7.165 | 90.900 | 1.646 | 3 |
| LTRP6-7255-10123 | 7.165 | 85.200 | 1.411 | 3 |
| LTRP6-26749-10123 | 7.165 | 93.567 | 0.643 | 3 |
| LTRP6-dual-10123 | 7.165 | 91.033 | 0.058 | 3 |
| LTRP6-22153-26749 | 7.165 | 93.900 | 0.693 | 3 |
| LTRP6-7255-26749 | 7.165 | 92.933 | 1.474 | 3 |
| LTRP6-dual-26749 | 7.165 | 95.300 | 0.557 | 3 |
| LTRP6-10123-26749 | 7.165 | 94.133 | 1.528 | 3 |

TABLE 25
Level of B2M repression mediated by LTRP constructs with various
repressor domains quantified at 44 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 1.307 | 0.070 | 3 |
| LTRP5-ZIM3 | 7.165 | 26.533 | 3.232 | 3 |
| LTRP5-ADD-ZIM3 | 7.165 | 63.700 | 1.345 | 3 |
| LTRP6-dual-ZIM3 | 7.165 | 82.600 | 1.929 | 3 |
| LTRP6-22153-7255 | 7.165 | 88.933 | 4.110 | 3 |
| LTRP6-dual-7255 | 7.165 | 83.233 | 2.610 | 3 |
| LTRP6-26749-7255 | 7.165 | 88.833 | 1.604 | 3 |
| LTRP6-10123-7255 | 7.165 | 86.567 | 1.909 | 3 |
| LTRP6-dual-22153 | 7.165 | 79.867 | 0.153 | 3 |
| LTRP6-7255-22153 | 7.165 | 80.033 | 3.807 | 3 |
| LTRP6-26749-22153 | 7.165 | 91.933 | 2.159 | 3 |
| LTRP6-10123-22153 | 7.165 | 89.567 | 0.929 | 3 |
| LTRP6-22153-10123 | 7.165 | 86.900 | 3.119 | 3 |
| LTRP6-7255-10123 | 7.165 | 77.433 | 2.031 | 3 |
| LTRP6-26749-10123 | 7.165 | 91.133 | 0.611 | 3 |
| LTRP6-dual-10123 | 7.165 | 87.033 | 0.231 | 3 |
| LTRP6-22153-26749 | 7.165 | 93.000 | 2.100 | 3 |
| LTRP6-7255-26749 | 7.165 | 89.367 | 3.758 | 3 |
| LTRP6-dual-26749 | 7.165 | 96.167 | 0.737 | 3 |
| LTRP6-10123-26749 | 7.165 | 94.050 | 0.919 | 2 |

TABLE 26
Level of B2M repression mediated by LTRP constructs with various
repressor domains quantified at 59 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 0.947 | 0.012 | 3 |
| LTRP5-ZIM3 | 7.165 | 19.667 | 2.627 | 3 |
| LTRP5-ADD-ZIM3 | 7.165 | 60.533 | 1.801 | 3 |
| LTRP6-dual-ZIM3 | 7.165 | 76.567 | 4.274 | 3 |
| LTRP6-22153-7255 | 7.165 | 87.933 | 6.385 | 3 |
| LTRP6-dual-7255 | 7.165 | 84.100 | 2.211 | 3 |
| LTRP6-26749-7255 | 7.165 | 85.733 | 1.704 | 3 |
| LTRP6-10123-7255 | 7.165 | 83.267 | 3.868 | 3 |
| LTRP6-dual-22153 | 7.165 | 74.500 | 0.608 | 3 |
| LTRP6-7255-22153 | 7.165 | 74.133 | 5.463 | 3 |
| LTRP6-26749-22153 | 7.165 | 89.833 | 3.691 | 3 |
| LTRP6-10123-22153 | 7.165 | 89.200 | 1.825 | 3 |
| LTRP6-22153-10123 | 7.165 | 84.733 | 3.581 | 3 |
| LTRP6-7255-10123 | 7.165 | 71.667 | 5.980 | 3 |
| LTRP6-26749-10123 | 7.165 | 90.567 | 0.808 | 3 |
| LTRP6-dual-10123 | 7.165 | 83.167 | 1.026 | 3 |
| LTRP6-22153-26749 | 7.165 | 93.467 | 2.301 | 3 |
| LTRP6-7255-26749 | 7.165 | 86.900 | 6.077 | 3 |
| LTRP6-dual-26749 | 7.165 | 96.833 | 0.874 | 3 |
| LTRP6-10123-26749 | 7.165 | 93.450 | 1.202 | 2 |

TABLE 27
Level of B2M repression mediated by LTRP constructs with various
repressor domains quantified at 74 days post-transfection.
The order of the domain IDs shown for each LTRP6 construct
designates the first repressor domain and second repressor
domain at the N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 2.407 | 1.086 | 3 |
| LTRP5-ZIM3 | 7.165 | 21.033 | 9.890 | 3 |
| LTRP5-ADD-ZIM3 | 7.165 | 61.167 | 5.807 | 3 |
| LTRP6-dual-ZIM3 | 7.165 | 68.133 | 7.305 | 3 |
| LTRP6-22153-7255 | 7.165 | 90.033 | 6.144 | 3 |
| LTRP6-dual-7255 | 7.165 | 87.833 | 2.219 | 3 |
| LTRP6-26749-7255 | 7.165 | 84.700 | 1.539 | 3 |
| LTRP6-10123-7255 | 7.165 | 83.833 | 3.859 | 3 |
| LTRP6-dual-22153 | 7.165 | 73.000 | 0.721 | 3 |
| LTRP6-7255-22153 | 7.165 | 71.267 | 7.343 | 3 |
| LTRP6-26749-22153 | 7.165 | 92.533 | 3.062 | 3 |
| LTRP6-10123-22153 | 7.165 | 92.133 | 2.542 | 3 |
| LTRP6-22153-10123 | 7.165 | 87.533 | 1.069 | 3 |
| LTRP6-7255-10123 | 7.165 | 69.533 | 5.265 | 3 |
| LTRP6-26749-10123 | 7.165 | 92.667 | 0.833 | 3 |
| LTRP6-dual-10123 | 7.165 | 80.133 | 2.458 | 3 |
| LTRP6-22153-26749 | 7.165 | 92.433 | 2.804 | 3 |
| LTRP6-7255-26749 | 7.165 | 87.800 | 8.143 | 3 |
| LTRP6-dual-26749 | 7.165 | 97.133 | 0.643 | 3 |
| LTRP6-10123-26749 | 7.165 | 94.067 | 1.686 | 3 |

TABLE 28
Level of B2M repression mediated by LTRP constructs with various
repressor domains quantified at 104 days post-transfection.
The order of the domain IDs shown for each LTRP6 construct designates
the first repressor domain and second repressor domain at the
N-terminus and C-terminus positions respectively.

| | | Mean % HLA- | Standard | Sample |
|---|---|---|---|---|
| Repressor construct | Spacer | negative cells | deviation | Size |
| LTRP5-ADD-ZIM3 | NT | 0.610 | 0.314 | 3 |
| LTRP5-ZIM3 | 7.165 | 14.270 | 12.499 | 3 |
| LTRP5-ADD-ZIM3 | 7.165 | 57.533 | 7.419 | 3 |
| LTRP6-dual-ZIM3 | 7.165 | 43.467 | 9.843 | 3 |
| LTRP6-22153-7255 | 7.165 | 90.900 | 1.929 | 3 |
| LTRP6-dual-7255 | 7.165 | 93.367 | 0.603 | 3 |
| LTRP6-26749-7255 | 7.165 | 84.000 | 1.044 | 3 |
| LTRP6-10123-7255 | 7.165 | 77.700 | 16.664 | 3 |
| LTRP6-dual-22153 | 7.165 | 69.567 | 6.336 | 3 |
| LTRP6-7255-22153 | 7.165 | 57.567 | 12.667 | 3 |
| LTRP6-26749-22153 | 7.165 | 87.867 | 4.277 | 3 |
| LTRP6-10123-22153 | 7.165 | 88.600 | 7.134 | 3 |
| LTRP6-22153-10123 | 7.165 | 84.600 | 0.624 | 3 |
| LTRP6-7255-10123 | 7.165 | 78.033 | 4.922 | 3 |
| LTRP6-26749-10123 | 7.165 | 94.400 | 1.375 | 3 |
| LTRP6-dual-10123 | 7.165 | 79.100 | 5.216 | 3 |
| LTRP6-22153-26749 | 7.165 | 92.067 | 5.595 | 3 |
| LTRP6-7255-26749 | 7.165 | 90.067 | 6.274 | 3 |
| LTRP6-dual-26749 | 7.165 | 98.267 | 0.929 | 3 |
| LTRP6-10123-26749 | 7.165 | 94.633 | 1.779 | 3 |

Bisulfite sequencing was performed to evaluate the degree of off-target CpG methylation at the VEGFA locus mediated by the 16 LTRP6 molecules at 5 days post-transfection, and the results are shown in Table 29. The data demonstrate that the majority of the LTRP6 constructs demonstrated relatively low off-target methylation levels, especially when compared with the levels achieved with constructs encoding for the dCas9-ZNF10-DNMT3A/3L, LTRP1, and LTRP5-ZIM3 controls. In fact, LTRP6 constructs containing DOMAIN_26749 at either or both positions (configuration #6a or #6b) exhibited comparable off-target methylation as the LTRP5-ADD-ZIM3 and LTRP6-dual-ZIM3 molecules (Table 29). LTRP6-dual-26749 and LTRP6-10123-26749, which exhibited the highest and second highest repressive activity, respectively (Tables 21-28), induced levels of off-target methylation of ~5.15% and ~3.84% respectively.

TABLE 29
Level of off-target CpG methylation at the VEGFA locus
mediated by the LTRP6 constructs with various repressor
domains quantified at day 5 post-transfection. The
order of the domain IDs shown for each LTRP6 construct
designates the first repressor domain and second repressor
domain at the N-terminus and C-terminus positions
respectively. Constructs encoding for CasX, dCas9-
ZNF10-DNMT3A/3L, LTRP1, LTRP5-ZIM3, and LTRP5-ADD-ZIM3
were also included as controls.

| | | Mean % CpG | Standard |
|---|---|---|---|
| Repressor construct | Spacer | methylation | error |
| CasX 491 | 7.37 | 0.851 | 0.160 |
| dCas9-ZNF10-DNMT3A/3L | 7.148 | 34.220 | 2.356 |
| LTRP1 | NT | 16.900 | 1.346 |
| LTRP1 | 7.165 | 30.880 | 1.829 |
| LTRP5-ADD-ZIM3 | NT | 6.887 | 0.403 |
| LTRP5-ZIM5 | 7.165 | 14.690 | 1.338 |
| LTRP5-ADD-ZIM3 | 7.165 | 4.841 | 0.508 |
| LTRP6-dual-ZIM3 | 7.165 | 5.782 | 0.565 |
| LTRP6-7255-22153 | 7.165 | 3.988 | 0.479 |
| LTRP6-dual-7255 | 7.165 | 11.330 | 0.993 |
| LTRP6-7255-26749 | 7.165 | 5.719 | 0.737 |
| LTRP6-7255-10123 | 7.165 | 5.796 | 0.635 |
| LTRP6-22153-26749 | 7.165 | 4.789 | 0.492 |
| LTRP6-22153-10123 | 7.165 | 1.244 | 0.422 |
| LTRP6-10123-22153 | 7.165 | 6.607 | 0.757 |
| LTRP6-10123-7255 | 7.165 | 16.530 | 1.417 |
| LTRP6-10123-26749 | 7.165 | 4.810 | 0.523 |
| LTRP6-dual-10123 | 7.165 | 5.729 | 0.699 |
| LTRP6-26749-22153 | 7.165 | 5.401 | 0.565 |
| LTRP6-26749-7255 | 7.165 | 4.608 | 0.582 |
| LTRP6-dual-22153 | 7.165 | 3.522 | 0.537 |
| LTRP6-22153-7255 | 7.165 | 4.388 | 0.669 |
| LTRP6-dual-26749 | 7.165 | 5.153 | 0.457 |
| LTRP6-26749-10123 | 7.165 | 3.836 | 0.482 |

The results of the experiments demonstrate that incorporation of a second repressor domain into an LTRP molecule, resulting in an LTRP6 configuration, improves durable repression, and this improvement is further enhanced with the use of an enhanced repressor domain at both the N-terminus and C-terminus positions relative to dCasX. Furthermore, the data demonstrate that use of some, but not all, novel repressor domains can induce highly durable repression of the target locus, and that their relative placement within the LTRP molecule could also affect activity. The findings of these experiments also show that incorporation of a second repressor domain into an LTRP-ADD molecule maintains molecule specificity while improving activity.

### Example 5: Optimization of Linker Sequences to Increase Overall LTRP Activity

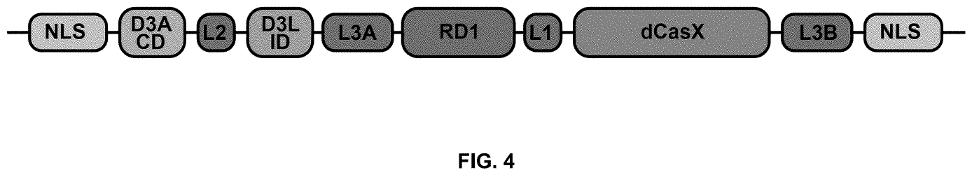

Experiments were performed to demonstrate that the repressive activity of an LTRP5 construct can be increased by optimizing the linker sequence at the following two positions: 1) between the DNMT3L interaction domain and the repressor domain and 2) between the repressor domain and the catalytically-dead CasX. These linker positions correspond to “L3A” and “L1” respectively in the schematic in FIG. 4 for the LTRP5 configuration. Here, experiments were performed to screen and identify new linker combinations that would improve LTRP activity.

### Materials and Methods

An entry vector was created to harbor a restriction enzyme site between the DNMT3L interaction domain and the repressor domain and between the repressor domain and the catalytically-dead CasX oriented in the LTRP5 configuration (FIG. 4). Furthermore, a list of 52 linkers (SEQ ID NO: 1823-1874, see Table 30) was generated by compiling linker sequences from multiple studies. The library of linkers was synthesized as an oligonucleotide pool that was subsequently amplified and randomly cloned into the entry vector via Golden Gate assembly, resulting in a library of LTRP5 variant plasmids covering ~2700 possible linker sets. ~90 LTRP5 variant plasmids containing the linker sets were randomly selected and subjected to arrayed screening, such that each LTRP5 variant plasmid was co-transfected with a plasmid encoding for a gRNA using scaffold 316 and a targeting spacer (see Table 31 for spacer sequences) into HEK293T cells seeded in 96-well plates. Following transfection, cells were selected with puromycin and hygromycin for three days and then harvested to evaluate repression at the target locus via immunostaining of the target cell surface marker, followed by flow cytometry using the Attune™ NxT flow cytometer. For variant plasmids containing linker sets 1-28 (see Table 30 for sequences of linker combinations), three different target loci (B2M, target 1, and target 2) were evaluated for LTRP repression activity. For linker sets 1-11, repression was measured at 8, 15, and 45 days post-transfection. For linker sets 12-28, repression was measured at 7 and 17 days post-transfection. For plasmids containing linker sets 31-89, along with linker sets 1, 10, and 25 (Table 30), the B2M locus was evaluated for LTRP repression activity at 6, 13, and 24 days post-transfection. A plasmid encoding for the LTRP5 molecule (without the ADD domain) with the original set of linkers (SEQ ID NO: 123 for L1; SEQ ID NO: 124 for L3A) was used as an experimental control. A non-targeting spacer was also included as a negative control.

TABLE 30
Amino acid sequences of the ~90 linker sets assessed in this example

| | AA sequence of | | AA sequence of | |
|---|---|---|---|---|
| Linker | first linker within | AA SEQ | second linker within | AA SEQ |
| Set ID | set (position L3A) | ID NO: | set (position L1) | ID NO: |
| Set 1 | ASAAAPAAASAAASAPSAAAA | 1823 | ASAAAPAAA | 1830 |
| Set 36 | PPTPSPSPVPSTPPTNSSSTPPTPS | 1825 | GSGNSSGSGGS | 1829 |
| Set 43 | GSGGSGGSGGSPVPSTPPTPSPSTP | 1826 | AFPAAPAPA | 1831 |
| | PTP | | | |
| Set 66 | AASPAAPSAPPAAASP | 1824 | PPTP | 1828 |
| Set 85 | ASPAAPAPASPAAPAPSAPAA | 1827 | IRAHGD | 1832 |
| Set 2 | LQPVPPQALKREQVSQQ | 1833 | HIGGINS | 1836 |
| Set 3 | GGGSGGGS | 1834 | GGGSGGGS | 1834 |
| Set 4 | AASPAAPSAPPAAASP | 1824 | SPAGSPTSTEEGTSESAT | 1861 |
| Set 5 | PGTSTEPSEGSAPGSP | 1835 | PPTP | 1828 |
| Set 6 | HIGGINS | 1836 | GGGS | 1851 |
| Set 7 | AASPAAPSAPPAAASP | 1824 | PPTPSPSPVP | 1856 |
| Set 8 | ASPAAPSP | 1837 | ILGELT | 1862 |
| Set 9 | GSGNSSGSGG | 1838 | HIGGINS | 1836 |
| Set 10 | PPTPSPSPVPSTPPTNSSSTPPTPS | 1825 | SGSETPGTSESATPES | 1842 |
| Set 11 | HIGGINS | 1836 | PPTPSPSPVP | 1856 |
| Set 12 | EAAKEAAK | 1839 | PPTP | 1828 |
| Set 13 | PPTP | 1828 | SWRVRYMVA | 1863 |
| Set 14 | LRTRYEADLA | 1840 | GSGNSSGSGGS | 1829 |
| Set 15 | EAAKEAAKEAAKEAAK | 1841 | PPTP | 1828 |
| Set 17 | SGSETPGTSESATPES | 1842 | GGGSGGGS | 1834 |
| Set 19 | GSGNSSGSGGS | 1829 | STEEGTSTEPSEGSAP | 1844 |
| Set 20 | PPTPSPSPVSSTPPTPPTPS | 1843 | EAAKEAAK | 1839 |
| Set 21 | STEEGTSTEPSEGSAP | 1844 | PPTPSPSPVP | 1856 |
| Set 23 | GGGSGGGS | 1834 | YGAHLM | 1864 |
| Set 24 | STEEGTSTEPSEGSAP | 1844 | GSGNSSGSGGSGGSGNSSGSGG | 1865 |
| | | | S | |
| Set 25 | PPTPSPSPVPSTPPTPPTPS | 1845 | GGGSGGGSGGGS | 1850 |
| Set 26 | GGGSGGGSGGGSEAAKEAAKEAAK | 1846 | VQQKYKVSDTAATVTG | 1853 |
| Set 27 | ASAAAPAAA | 1830 | GSGNSSGSGGSGGSGNSSGSGG | 1865 |
| | | | S | |
| Set 28 | PPTP | 1828 | GGGSGGGS | 1834 |
| Set 31 | SGSETPGTSESATPES | 1842 | STEEGTSTEPSEGSAP | 1844 |
| Set 32 | GSGGSGGSGGSPVPSTPPTPSPSTP | 1826 | SPAGSPTSTEEGTSESAT | 1861 |
| | PTP | | | |
| Set 33 | PPTP | 1828 | GGGS | 1851 |
| Set 34 | ASPAAPAPA | 1847 | GGGS | 1851 |
| Set 37 | GGGSGGGS | 1834 | GGGS | 1851 |
| Set 38 | GGGSGGGS | 1834 | SGSETPGTSESATPES | 1842 |
| Set 39 | GGGSGGGSEAAKEAAK | 1848 | STEEGTSTEPSEGSAP | 1844 |
| Set 40 | PPTPSPSPVPSTPPTPPTPS | 1845 | GGGSGGGSGGGS | 1850 |
| Set 41 | GGGSGGGSGGGSGGGS | 1849 | SPAGSPTSTEEGTSESAT | 1861 |
| Set 42 | GSGNSSGSGGS | 1829 | AYVVSADEREGG | 1866 |
| Set 44 | SGSETPGTSESATPES | 1842 | GSGNSSGSGGS | 1829 |
| Set 45 | GGGSGGGSGGGS | 1850 | PGTSTEPSEGSAPGSP | 1835 |
| Set 46 | GGGSGGGSGGGSGGGS | 1849 | EAAKEAAK | 1839 |
| Set 47 | GSGNSSGSGGS | 1829 | PPTPSPSPVPSTPPTPPTPS | 1845 |
| Set 48 | GGGSGGGSEAAKEAAK | 1848 | EAAKEAAKGGGSGGGS | 1859 |
| Set 49 | GGGS | 1851 | GGGSGGGS | 1834 |
| Set 50 | EAAK | 1852 | GSGGSGGSGGSPVPSTPPTPSP | 1826 |
| | | | STPPTP | |
| Set 51 | EAAKEAAK | 1839 | EAAKEAAK | 1839 |
| Set 52 | SGSETPGTSESATPES | 1842 | PPTP | 1828 |
| Set 53 | GGGSGGGS | 1834 | PPTP | 1828 |
| Set 55 | GGGSGGGSGGGS | 1850 | MIGP | 1867 |
| Set 56 | VQQKYKVSDTAATVTG | 1853 | SGSETPGTSESATPES | 1842 |
| Set 57 | GGGSGGGSGGVS | 1854 | GSGGSGGSGGSPVPSTPPTPSP | 1826 |
| | | | STPPTP | |
| Set 58 | EAAKEAAKEAAKEAAK | 1841 | STEEGTSTEPSEGSAP | 1844 |
| Set 59 | GGGSGGGS | 1834 | EAAKEAVKGGGSGGGS | 1868 |
| Set 60 | GGGSGGGSGGGSGGGS | 1849 | PPTP | 1828 |
| Set 61 | EAAK | 1852 | EAAKEAAKGGGSGGGS | 1859 |
| Set 62 | GGGS | 1851 | PPTPSPSPVP | 1856 |
| Set 63 | EAAKEAVKEAAKEAAK | 1855 | HIGGINS | 1836 |
| Set 64 | PPTPSPSPVPSTPPTPPTPS | 1845 | GGGSGGGS | 1834 |
| Set 65 | PPTPSPSPVP | 1856 | PPTP | 1828 |
| Set 67 | STEEGTSTEPSEGSAP | 1844 | PPTP | 1828 |
| Set 68 | GGGSGGGSGGGS | 1850 | PPTPSPSPVPSTPPTNSSSTPP | 1825 |
| | | | TPS | |
| Set 69 | PPTPSPSPVP | 1856 | PGTSTEPSEGSAPGSP | 1835 |
| Set 70 | ASPAAPAPA | 1847 | LMPYEL | 1869 |
| Set 71 | PPTPSPSPVP | 1856 | PPTP | 1828 |
| Set 72 | GGGSGGGSGGGS | 1850 | ASAAAPAAA | 1830 |
| Set 73 | GGGSGGGS | 1834 | STEEGTSTEPSEGSAP | 1844 |
| Set 74 | PPTPSPSPVP | 1856 | HIGGINS | 1836 |
| Set 75 | EAAKEAAK | 1839 | HIGGINS | 1836 |
| Set 76 | PPTPSPSPVPSTPPTPPTPS | 1845 | SPTP | 1870 |
| Set 77 | EAAKEAAKEAAKEAAK | 1841 | GGGSGGGSGGGSGGGSGGGS | 1871 |
| Set 78 | EAAKEAAK | 1839 | PPTPSPSPVPSTPPTPPTPS | 1845 |
| Set 79 | PPTPSPSPVP | 1856 | GGGSGGGSGGGSGGGS | 1849 |
| Set 80 | ITHKASSRMTFA | 1857 | GGGSGGGSGGGS | 1850 |
| Set 81 | STEEGTSTEPSEGSAP | 1844 | GCGS | 1872 |
| Set 82 | ASSAAPAPA | 1858 | GGGS | 1851 |
| Set 83 | STEEGTSTEPSEGSAP | 1844 | GGGSGGGS | 1834 |
| Set 84 | SGSETPGTSESATPES | 1842 | GGGSGGGSGGGSGGGSGGGS | 1871 |
| Set 86 | EAAKEAAKGGGSGGGS | 1859 | EAAKEAAKGGGSGGGS | 1859 |
| Set 87 | EAAKEAAKEAAKEAAK | 1841 | NKQNIWQA | 1873 |
| Set 88 | GGGSGGGSGGGS | 1850 | ASAAAPAAA | 1830 |
| Set 89 | AASPAAPSAPPASASP | 1860 | PPMPSPSPVP | 1874 |

TABLE 31
Sequences of spacers targeting the B2M locus

| Spacer | | Targeting spacer | SEQ |
|---|---|---|---|
| ID | Target | sequence (RNA) | ID NO |
| 7.165 | B2M | UCCCUAUGUCCUUGCUGUUU | 1914 |

### Results:

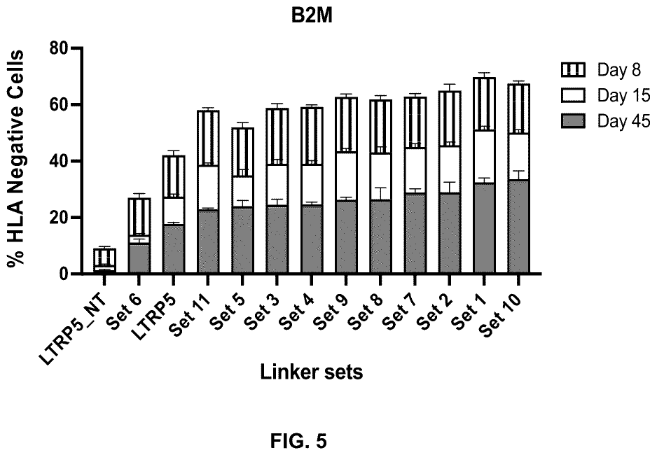

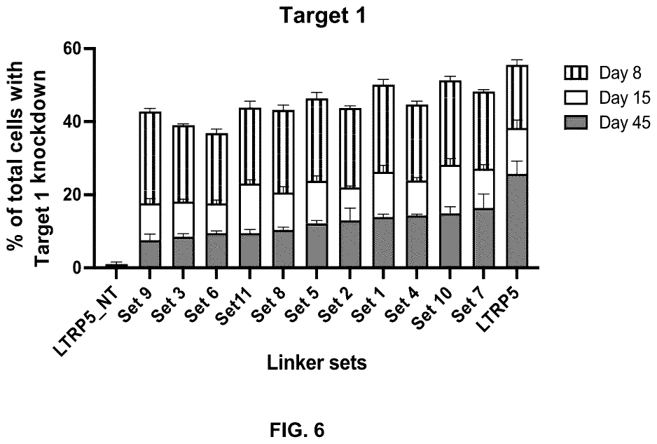

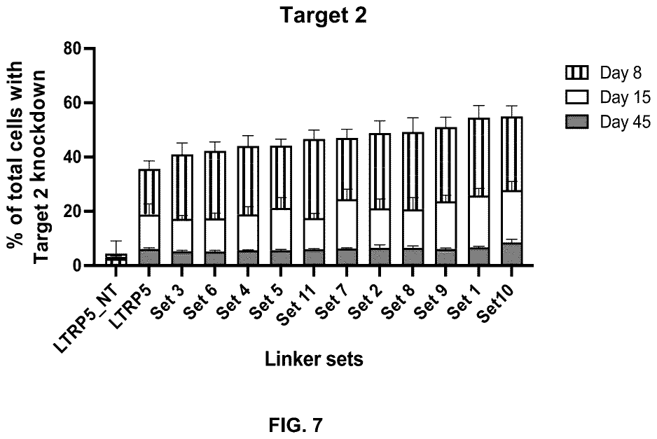

Arrayed screening was performed to evaluate the level of repressive activity at endogenous target loci in HEK293T cells transfected with LTRP5 variant plasmids harboring different combinations of linkers. Repression at the B2M, target 1, and target 2 loci was measured at 8, 15, and 45 days post-transfection for LTRP5 plasmids containing linker sets 1-11, and the results are illustrated in FIGS. 5-7. The data demonstrate that the top performing linker combinations varied by the specific target. Of the 11 linker combinations analyzed in this experiment, use of linker sets 1 and 10 in the LTRP5 constructs overall consistently induced the highest level of repression at both the B2M and target 2 loci, with constructs with linker set 10 exhibiting the second highest tested linker set at the target 1 locus. Linker set 1 was subsequently used as a benchmark in the subsequent two experiments analyzing linker sets 12-28 and linker sets 31-89.

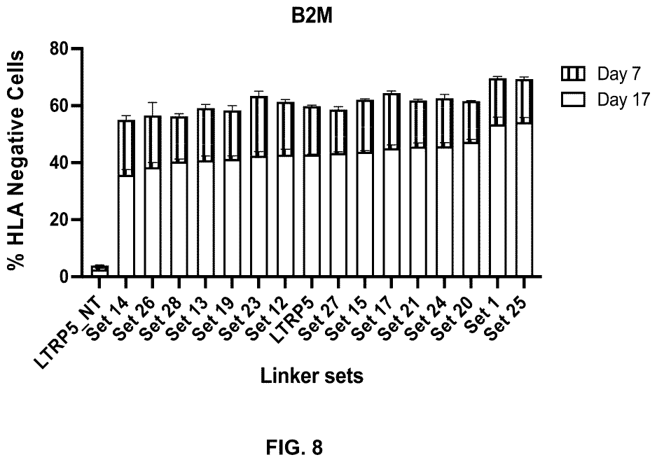

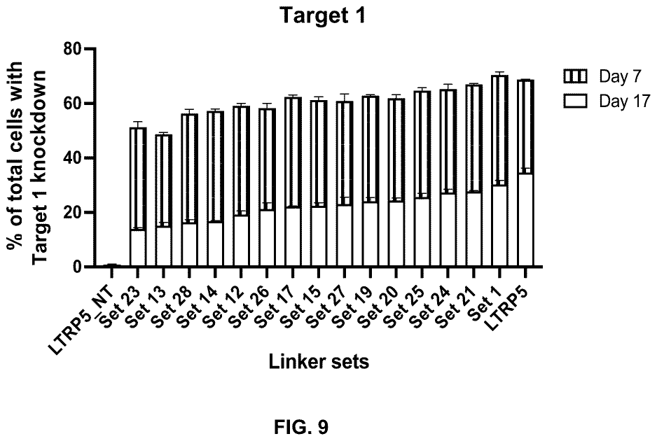

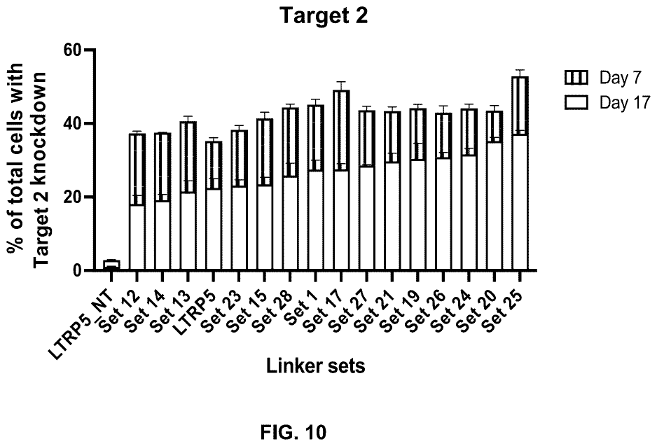

Repression at the B2M, target 1, and target 2 loci was measured at 7 and 17 days post-transfection for LTRP5 plasmids containing linker sets 12-28, with linker set 1 from the previous experiment as a benchmark, and the results are portrayed in FIGS. 8-10. The data show that as similarly observed in the previous experiment, the top performing linker sets varied by target. Of the 17 linker combinations analyzed in this second experiment, use of linker sets 1 and 25 in LTRP5 constructs resulted in the highest level of repression at two of the three target loci (B2M and target 1 for set 1; B2M and target 2 for set 25).

Repression at the B2M locus was measured at 6, 13, and 24 days post-transfection for LTRP5 plasmids containing linker sets 31-89, with linker sets 1, 10, and 25 from the previous two experiments as benchmarks, and the results are portrayed in Tables 32-34. The data show that use of linker sets 85, 43, 36, 10, 64, 1, 40, 25, 66, 76, and 52 resulted in at least a 50% increase in repressive activity relative to the activity level of the LTRP5 control when paired with the B2M spacer at 24 days post-transfection (Tables 32-34). Five of these top 11 performing linker sets were then selected for future validation based on having a sequence distinct from other top candidate combinations and from the original linker set of linkers (SEQ ID NO: 123 for L1; SEQ ID NO: 124 for L3A).

Table 32: Levels of B2M repression mediated by LTRP5 constructs with linker sets 1, 10, 25, and 31-39 quantified at 6 days post-transfection 177

TABLE 32
Levels of B2M repression mediated by LTRP5 constructs with linker
sets 1, 10, 25, and 31-39 quantified at 6 days post-transfection

| | | | Mean % | | |
|---|---|---|---|---|---|
| | | | HLA-negative | Standard | Sample |
| Construct | Linker set | Spacer | cells | deviation | size |
| LTRP5 | Original/ | NT | 4.607 | 0.289 | 3 |
| | Baseline | | | | |
| LTRP5 | Set 54 | 7.165 | 31.000 | 2.955 | 3 |
| LTRP5 | Set 69 | 7.165 | 33.400 | 1.015 | 3 |
| LTRP5 | Set 42 | 7.165 | 35.167 | 1.258 | 3 |
| LTRP5 | Set 89 | 7.165 | 34.733 | 0.603 | 3 |
| LTRP5 | Original/ | 7.165 | 38.667 | 0.379 | 3 |
| | Baseline | | | | |
| LTRP5 | Set 61 | 7.165 | 48.300 | 1.931 | 3 |
| LTRP5 | Set 59 | 7.165 | 49.467 | 0.709 | 3 |
| LTRP5 | Set 80 | 7.165 | 48.633 | 0.643 | 3 |
| LTRP5 | Set 33 | 7.165 | 53.800 | 1.200 | 3 |
| LTRP5 | Set 37 | 7.165 | 55.167 | 0.416 | 3 |
| LTRP5 | Set 51 | 7.165 | 51.900 | 0.819 | 3 |
| LTRP5 | Set 49 | 7.165 | 52.267 | 0.723 | 3 |
| LTRP5 | Set 48 | 7.165 | 50.500 | 1.082 | 3 |
| LTRP5 | Set 62 | 7.165 | 53.367 | 0.493 | 3 |
| LTRP5 | Set 41 | 7.165 | 53.067 | 0.473 | 3 |
| LTRP5 | Set 50 | 7.165 | 53.400 | 0.458 | 3 |
| LTRP5 | Set 38 | 7.165 | 53.833 | 0.231 | 3 |
| LTRP5 | Set 78 | 7.165 | 52.633 | 0.379 | 3 |
| LTRP5 | Set 86 | 7.165 | 53.833 | 1.484 | 3 |
| LTRP5 | Set 53 | 7.165 | 55.167 | 0.764 | 3 |
| LTRP5 | Set 87 | 7.165 | 55.533 | 0.833 | 3 |
| LTRP5 | Set 46 | 7.165 | 53.033 | 0.153 | 3 |
| LTRP5 | Set 55 | 7.165 | 50.633 | 1.650 | 3 |
| LTRP5 | Set 34 | 7.165 | 57.800 | 0.794 | 3 |
| LTRP5 | Set 75 | 7.165 | 53.400 | 0.794 | 3 |
| LTRP5 | Set 39 | 7.165 | 55.467 | 1.002 | 3 |
| LTRP5 | Set 56 | 7.165 | 54.000 | 1.114 | 3 |
| LTRP5 | Set 60 | 7.165 | 57.933 | 1.328 | 3 |
| LTRP5 | Set 82 | 7.165 | 57.500 | 0.889 | 3 |
| LTRP5 | Set 73 | 7.165 | 53.433 | 0.907 | 3 |
| LTRP5 | Set 83 | 7.165 | 59.333 | 1.069 | 3 |
| LTRP5 | Set 63 | 7.165 | 55.400 | 0.872 | 3 |
| LTRP5 | Set 88 | 7.165 | 58.567 | 1.436 | 3 |
| LTRP5 | Set 68 | 7.165 | 55.633 | 0.231 | 3 |
| LTRP5 | Set 58 | 7.165 | 54.433 | 0.723 | 3 |
| LTRP5 | Set 79 | 7.165 | 56.700 | 0.520 | 3 |
| LTRP5 | Set 70 | 7.165 | 55.467 | 1.350 | 3 |
| LTRP5 | Set 32 | 7.165 | 58.467 | 0.723 | 3 |
| LTRP5 | Set 77 | 7.165 | 56.133 | 0.651 | 3 |
| LTRP5 | Set 81 | 7.165 | 60.400 | 0.500 | 3 |
| LTRP5 | Set 57 | 7.165 | 57.800 | 1.153 | 3 |
| LTRP5 | Set 44 | 7.165 | 57.933 | 1.617 | 3 |
| LTRP5 | Set 47 | 7.165 | 55.400 | 0.173 | 3 |
| LTRP5 | Set 31 | 7.165 | 59.767 | 0.751 | 3 |
| LTRP5 | Set 74 | 7.165 | 58.067 | 0.569 | 3 |
| LTRP5 | Set 65 | 7.165 | 60.300 | 0.954 | 3 |
| LTRP5 | Set 84 | 7.165 | 59.333 | 0.902 | 3 |
| LTRP5 | Set 67 | 7.165 | 60.767 | 1.779 | 3 |
| LTRP5 | Set 72 | 7.165 | 57.433 | 0.723 | 3 |
| LTRP5 | Set 71 | 7.165 | 59.467 | 0.379 | 3 |
| LTRP5 | Set 52 | 7.165 | 61.533 | 0.153 | 3 |
| LTRP5 | Set 76 | 7.165 | 62.900 | 0.400 | 3 |
| LTRP5 | Set 66 | 7.165 | 61.933 | 0.379 | 3 |
| LTRP5 | Set 25 | 7.165 | 61.033 | 0.416 | 3 |
| LTRP5 | Set 40 | 7.165 | 62.800 | 0.889 | 3 |
| LTRP5 | Set 1 | 7.165 | 58.133 | 0.058 | 3 |
| LTRP5 | Set 64 | 7.165 | 57.400 | 1.229 | 3 |
| LTRP5 | Set 10 | 7.165 | 59.967 | 1.222 | 3 |
| LTRP5 | Set 36 | 7.165 | 65.333 | 0.902 | 3 |
| LTRP5 | Set 43 | 7.165 | 64.800 | 1.253 | 3 |
| LTRP5 | Set 85 | 7.165 | 64.333 | 1.274 | 3 |

TABLE 33
Levels of B2M repression mediated by LTRP5 constructs with linker
sets 1, 10, 25, and 31-39 quantified at 13 days post-transfection

| | | | Mean % | | |
|---|---|---|---|---|---|
| | | | HLA-negative | Standard | Sample |
| Construct | Linker set | Spacer | cells | deviation | size |
| LTRP5 | Original/ | NT | 1.877 | 0.315 | 3 |
| | Baseline | | | | |
| LTRP5 | Set 54 | 7.165 | 2.670 | 0.866 | 3 |
| LTRP5 | Set 69 | 7.165 | 1.200 | 0.190 | 3 |
| LTRP5 | Set 42 | 7.165 | 24.000 | 0.794 | 3 |
| LTRP5 | Set 89 | 7.165 | 25.500 | 0.755 | 3 |
| LTRP5 | Original/ | 7.165 | 30.800 | 0.917 | 3 |
| | Baseline | | | | |
| LTRP5 | Set 61 | 7.165 | 31.600 | 0.700 | 3 |
| LTRP5 | Set 59 | 7.165 | 32.267 | 0.473 | 3 |
| LTRP5 | Set 80 | 7.165 | 32.767 | 0.231 | 3 |
| LTRP5 | Set 33 | 7.165 | 36.833 | 1.137 | 3 |
| LTRP5 | Set 37 | 7.165 | 35.900 | 1.179 | 3 |
| LTRP5 | Set 51 | 7.165 | 34.767 | 0.723 | 3 |
| LTRP5 | Set 49 | 7.165 | 36.167 | 0.929 | 3 |
| LTRP5 | Set 48 | 7.165 | 36.267 | 1.274 | 3 |
| LTRP5 | Set 62 | 7.165 | 34.433 | 0.551 | 3 |
| LTRP5 | Set 41 | 7.165 | 35.067 | 1.484 | 3 |
| LTRP5 | Set 50 | 7.165 | 38.067 | 0.874 | 3 |
| LTRP5 | Set 38 | 7.165 | 36.567 | 0.351 | 3 |
| LTRP5 | Set 78 | 7.165 | 35.000 | 0.700 | 3 |
| LTRP5 | Set 86 | 7.165 | 34.933 | 1.747 | 3 |
| LTRP5 | Set 53 | 7.165 | 35.833 | 1.234 | 3 |
| LTRP5 | Set 87 | 7.165 | 35.800 | 1.015 | 3 |
| LTRP5 | Set 46 | 7.165 | 36.200 | 0.361 | 3 |
| LTRP5 | Set 55 | 7.165 | 36.033 | 1.950 | 3 |
| LTRP5 | Set 34 | 7.165 | 40.733 | 1.168 | 3 |
| LTRP5 | Set 75 | 7.165 | 36.033 | 1.604 | 3 |
| LTRP5 | Set 39 | 7.165 | 38.867 | 1.266 | 3 |
| LTRP5 | Set 56 | 7.165 | 37.467 | 0.764 | 3 |
| LTRP5 | Set 60 | 7.165 | 40.467 | 1.750 | 3 |
| LTRP5 | Set 82 | 7.165 | 38.567 | 1.069 | 3 |
| LTRP5 | Set 73 | 7.165 | 35.433 | 1.845 | 3 |
| LTRP5 | Set 83 | 7.165 | 39.100 | 0.889 | 3 |
| LTRP5 | Set 63 | 7.165 | 37.433 | 1.002 | 3 |
| LTRP5 | Set 88 | 7.165 | 39.033 | 1.930 | 3 |
| LTRP5 | Set 68 | 7.165 | 37.833 | 0.493 | 3 |
| LTRP5 | Set 58 | 7.165 | 38.700 | 0.866 | 3 |
| LTRP5 | Set 79 | 7.165 | 39.367 | 0.153 | 3 |
| LTRP5 | Set 70 | 7.165 | 37.467 | 1.701 | 3 |
| LTRP5 | Set 32 | 7.165 | 42.833 | 0.289 | 3 |
| LTRP5 | Set 77 | 7.165 | 38.767 | 0.907 | 3 |
| LTRP5 | Set 81 | 7.165 | 39.300 | 1.039 | 3 |
| LTRP5 | Set 57 | 7.165 | 41.233 | 1.779 | 3 |
| LTRP5 | Set 44 | 7.165 | 40.667 | 0.814 | 3 |
| LTRP5 | Set 47 | 7.165 | 39.667 | 0.850 | 3 |
| LTRP5 | Set 31 | 7.165 | 43.433 | 1.650 | 3 |
| LTRP5 | Set 74 | 7.165 | 40.467 | 0.802 | 3 |
| LTRP5 | Set 65 | 7.165 | 42.000 | 0.608 | 3 |
| LTRP5 | Set 84 | 7.165 | 41.700 | 0.200 | 3 |
| LTRP5 | Set 67 | 7.165 | 41.600 | 1.277 | 3 |
| LTRP5 | Set 72 | 7.165 | 39.400 | 0.781 | 3 |
| LTRP5 | Set 71 | 7.165 | 42.533 | 0.153 | 3 |
| LTRP5 | Set 52 | 7.165 | 44.333 | 0.635 | 3 |
| LTRP5 | Set 76 | 7.165 | 45.033 | 1.721 | 3 |
| LTRP5 | Set 66 | 7.165 | 43.233 | 0.833 | 3 |
| LTRP5 | Set 25 | 7.165 | 49.233 | 0.231 | 3 |
| LTRP5 | Set 40 | 7.165 | 47.767 | 1.002 | 3 |
| LTRP5 | Set 1 | 7.165 | 48.100 | 0.917 | 3 |
| LTRP5 | Set 64 | 7.165 | 44.133 | 1.097 | 3 |
| LTRP5 | Set 10 | 7.165 | 48.733 | 1.069 | 3 |
| LTRP5 | Set 36 | 7.165 | 48.833 | 1.358 | 3 |
| LTRP5 | Set 43 | 7.165 | 49.267 | 1.222 | 3 |
| LTRP5 | Set 85 | 7.165 | 46.933 | 2.312 | 3 |

TABLE 34
Levels of B2M repression mediated by LTRP5 constructs with linker
sets 1, 10, 25, and 31-39 quantified at 24 days post-transfection

| | | | Mean % | | |
|---|---|---|---|---|---|
| | | | HLA-negative | Standard | Sample |
| Construct | Linker set | Spacer | cells | deviation | size |
| LTRP5 | Original/ | NT | 1.290 | 0.098 | 3 |
| | Baseline | | | | |
| LTRP5 | Set 54 | 7.165 | 3.670 | 1.105 | 3 |
| LTRP5 | Set 69 | 7.165 | 4.270 | 0.282 | 3 |
| LTRP5 | Set 42 | 7.165 | 22.600 | 1.153 | 3 |
| LTRP5 | Set 89 | 7.165 | 24.500 | 0.781 | 3 |
| LTRP5 | Original/ | 7.165 | 25.067 | 0.907 | 3 |
| | Baseline | | | | |
| LTRP5 | Set 61 | 7.165 | 27.100 | 0.781 | 3 |
| LTRP5 | Set 59 | 7.165 | 27.167 | 0.351 | 3 |
| LTRP5 | Set 80 | 7.165 | 28.367 | 1.185 | 3 |
| LTRP5 | Set 33 | 7.165 | 28.367 | 0.208 | 3 |
| LTRP5 | Set 37 | 7.165 | 28.833 | 0.751 | 3 |
| LTRP5 | Set 51 | 7.165 | 28.933 | 0.551 | 3 |
| LTRP5 | Set 49 | 7.165 | 28.967 | 1.950 | 3 |
| LTRP5 | Set 48 | 7.165 | 29.467 | 0.757 | 3 |
| LTRP5 | Set 62 | 7.165 | 29.633 | 0.551 | 3 |
| LTRP5 | Set 41 | 7.165 | 29.767 | 1.457 | 3 |
| LTRP5 | Set 50 | 7.165 | 30.000 | 0.436 | 3 |
| LTRP5 | Set 38 | 7.165 | 30.167 | 1.050 | 3 |
| LTRP5 | Set 78 | 7.165 | 30.200 | 1.480 | 3 |
| LTRP5 | Set 86 | 7.165 | 30.367 | 1.041 | 3 |
| LTRP5 | Set 53 | 7.165 | 30.567 | 2.055 | 3 |
| LTRP5 | Set 87 | 7.165 | 30.733 | 1.258 | 3 |
| LTRP5 | Set 46 | 7.165 | 31.033 | 0.723 | 3 |
| LTRP5 | Set 55 | 7.165 | 31.167 | 2.413 | 3 |
| LTRP5 | Set 34 | 7.165 | 31.833 | 0.802 | 3 |
| LTRP5 | Set 75 | 7.165 | 31.867 | 2.892 | 3 |
| LTRP5 | Set 39 | 7.165 | 31.867 | 0.874 | 3 |
| LTRP5 | Set 56 | 7.165 | 32.267 | 0.681 | 3 |
| LTRP5 | Set 60 | 7.165 | 32.733 | 1.206 | 3 |
| LTRP5 | Set 82 | 7.165 | 32.967 | 1.274 | 3 |
| LTRP5 | Set 73 | 7.165 | 33.133 | 0.351 | 3 |
| LTRP5 | Set 83 | 7.165 | 33.333 | 0.569 | 3 |
| LTRP5 | Set 63 | 7.165 | 33.400 | 2.551 | 3 |
| LTRP5 | Set 88 | 7.165 | 33.500 | 1.803 | 3 |
| LTRP5 | Set 68 | 7.165 | 33.533 | 0.666 | 3 |
| LTRP5 | Set 58 | 7.165 | 33.633 | 1.266 | 3 |
| LTRP5 | Set 79 | 7.165 | 34.000 | 0.100 | 3 |
| LTRP5 | Set 70 | 7.165 | 34.167 | 2.836 | 3 |
| LTRP5 | Set 32 | 7.165 | 34.167 | 1.193 | 3 |
| LTRP5 | Set 77 | 7.165 | 34.467 | 0.814 | 3 |
| LTRP5 | Set 81 | 7.165 | 34.733 | 0.404 | 3 |
| LTRP5 | Set 57 | 7.165 | 34.833 | 2.250 | 3 |
| LTRP5 | Set 44 | 7.165 | 34.933 | 1.012 | 3 |
| LTRP5 | Set 47 | 7.165 | 35.167 | 0.802 | 3 |
| LTRP5 | Set 31 | 7.165 | 35.433 | 2.060 | 3 |
| LTRP5 | Set 74 | 7.165 | 35.833 | 0.651 | 3 |
| LTRP5 | Set 65 | 7.165 | 36.467 | 0.681 | 3 |
| LTRP5 | Set 84 | 7.165 | 37.167 | 1.069 | 3 |
| LTRP5 | Set 67 | 7.165 | 37.300 | 1.311 | 3 |
| LTRP5 | Set 72 | 7.165 | 37.667 | 0.850 | 3 |
| LTRP5 | Set 71 | 7.165 | 37.733 | 0.681 | 3 |
| LTRP5 | Set 52 | 7.165 | 38.767 | 0.929 | 3 |
| LTRP5 | Set 76 | 7.165 | 39.167 | 1.858 | 3 |
| LTRP5 | Set 66 | 7.165 | 39.367 | 0.503 | 3 |
| LTRP5 | Set 25 | 7.165 | 40.400 | 1.808 | 3 |
| LTRP5 | Set 40 | 7.165 | 40.633 | 1.629 | 3 |
| LTRP5 | Set 1 | 7.165 | 41.000 | 1.127 | 3 |
| LTRP5 | Set 64 | 7.165 | 41.367 | 1.877 | 3 |
| LTRP5 | Set 10 | 7.165 | 42.167 | 2.113 | 3 |
| LTRP5 | Set 36 | 7.165 | 42.333 | 1.250 | 3 |
| LTRP5 | Set 43 | 7.165 | 42.433 | 0.666 | 3 |
| LTRP5 | Set 85 | 7.165 | 42.500 | 2.272 | 3 |

The experiments show that the repressive activity of the LTRP molecule can be enhanced by altering the linker sequences positioned in between key domains of the LTRP molecule. Here, optimizing the linker sequences at two positions, i.e., between the DNM/T3L interaction domain and the repressor domain and between the repressor domain and the catalytically-dead CasX, resulted in substantial improvement in repressive activity of the LTRP molecule.

### Example 6: Demonstration that Use of a Long-Term Repressor Protein (LTRP) Molecule can Induce Silencing of an Endogenous Locus in Mouse Hepa1-6 Cells

Experiments were performed to demonstrate the ability of LTRPs to induce durable repression of an alternative endogenous locus in mouse Hepa1-6 liver cells, when delivered as mRNA co-transfected with a targeting gRNA.

### Materials and Methods

Experiment #1: DXR1 vs. LTRP #1 in Hepa1-6 Cells when Delivered as mRNA Generation of dXR1 and LTRP #1 mRNA:

mRNA encoding dXR1 or LTRP configuration 1 constructs containing the ZIM3-KRAB (hereafter known as dXR1 and LTRP1-ZIM3 respectively) domain was generated by in vitro transcription (IVT). Briefly, constructs encoding for a 5′UTR region, dXR1 or LTRP1-ZIM3 harboring the ZIM3-KRAB domain with flanking SV40 NLSes, and a 3′UTR region were generated and cloned into a plasmid containing a T7 promoter and 80-nucleotide poly(A) tail. These constructs also contained a 2× FLAG sequence. Sequences encoding the dXR1 and LTRP1-ZIM3 molecules were codon-optimized using a codon utilization table, in addition to using a publicly available codon optimization tool and adjusting parameters such as GC content. For IVT, the resulting plasmid was linearized prior to use for in vitro transcription (IVT) reactions, which were carried out with CleanCap® AG and N1-methyl-pseudouridine. IVT reactions were then subjected to DNase digestion and oligodT purification on-column. For experiment #1, the DNA sequences encoding the dXR1 and LTRP1-ZIM3 molecules are listed in Table 35. The corresponding mRNA sequences encoding the dXR1 and LTRP1-ZIM3 mRNAs are listed in Table 36. The protein sequences of the dXR1 and LTRP1-ZIM3 are listed in Table 36.

TABLE 35
Encoding sequences of the dXR1 and LTRP1-ZIM3 mRNA
molecules assessed in experiment #1 of this example*

| dXR or | | DNA sequence |
|---|---|---|
| LTRP ID | Component | SEQ ID NO |
| dXR1 | 5′UTR | 1925 |
| | START codon + NLS + linker | 1999 |
| | dCasX491 | 1933 |
| | Linker + buffer sequence | 2000 |
| | ZIM3-KRAB | 1931 |
| | Buffer sequence + NLS | 2001 |
| | Tag | 2002 |
| | STOP codon + buffer sequence | 2003 |
| | 3′UTR | 1936 |
| | Buffer sequence | 1937 |
| | Poly(A) tail | 1961 |
| LTRP1- | 5′UTR | 1925 |
| ZIM3 | START codon + NLS + buffer sequence + | 2004 |
| | linker | |
| | START codon + DNMT3A catalytic domain | 1927 |
| | Linker | 1928 |
| | DNMT3L interaction domain | 1929 |
| | Linker | 2005 |
| | dCasX491 | 1933 |
| | Linker | 2000 |
| | ZIM3-KRAB | 1931 |
| | Buffer sequence + NLS | 2001 |
| | Tag | 2002 |
| | STOP codons + buffer sequence | 2003 |
| | 3′UTR | 1936 |
| | Buffer sequence | 1937 |
| | Poly(A) tail | 1961 |

*Components are listed in a 5′ to 3′ order within the constructs

TABLE 36
Full-length RNA sequences of dXR1 and LTRP1-ZIM3 mRNA
molecules assessed in experiment #1 of this example.

| dXR or LTRP | RNA sequence | AA sequence |
|---|---|---|
| ID | SEQ ID NO | SEQ ID NO |
| dXR1 | 2006 | 2008 |
| LTRP1-ZIM3 | 2007 | 1918 |

Modification ‘mψ’ = N1-methyl-pseudouridine

Synthesis of gRNAs:

In experiment #1, gRNAs targeting the PCSK9 locus were designed using gRNA scaffold 174 and chemically synthesized using the v1 modification profile (as described in Example 11; SEQ ID NO: 2136). The sequences of the PCSK9-targeting spacers are listed in Table 37.

TABLE 37
Sequences of spacers targeting
the PCSK9 locus used in this example

| gRNA ID | | | |
|---|---|---|---|
| (scaffold- | | | SEQ |
| variant | | Targeting spacer | ID |
| spacer) | Target | sequence (RNA) | NO |
| 174-6.7 | human PCSK9 | UCCUGGCUUCCUGGUGAAGA | 2009 |
| 174-27.1 | mouse PCSK9 | GCCUCGCCCUCCCCAGACAG | 2010 |
| 174-27.88 | mouse PCSK9 | CGCUACCUGCCUAAACUUUG | 2011 |
| 174-27.92 | mouse PCSK9 | CCCUCCAACAAUAUUAACUA | 2012 |
| 174-27.93 | mouse PCSK9 | GGGGUCUCCCAGCCACCCCU | 2013 |
| 174-27.94 | mouse PCSK9 | CCCCUCUUAAUCCCCACUCC | 2014 |
| 174-27.100 | mouse PCSK9 | CUCUCUCUUUCUGAGGCUAG | 2015 |
| 174-27.103 | mouse PCSK9 | UAAUCUCCAUCCUCGUCCUG | 2016 |

Transfection of mRNA and gRNA into Hepa1-6 Cells and Intracellular PCSK9 Staining:

Seeded Hepa1-6 cells treated with the NATE™ inhibitor were lipofected with 300 ng of mRNA encoding dXR1 or LTRP1-ZIM3 (Table 36) and 150 ng of a PCSK9-targeting gRNA (Table 37). Seven different gRNAs with spacers spanning the promoter region of the mouse PCSK9 locus were tested, in addition to a sequence complementary to the human PCSK9 gene that served as the non-targeting control (Table 37). Cells were harvested at 6, 13, and 25 days after transfection to measure intracellular levels of the PCSK9 protein using an intracellular flow cytometry staining protocol. Briefly, cells were fixed using 4% paraformaldehyde in PBS, permeabilized, and stained using a mouse anti-PCSK9 primary antibody (R&D Systems), followed by a fluorescent goat anti-mouse IgG secondary antibody (Thermo Fisher). Fluorescence levels were measured using the Attune™ NxT flow cytometer, and data were analyzed using the FlowJo™ software. Cell populations were gated using the non-targeting gRNA as a negative control.

Experiment #2: LTRP #1 vs. LTRP #5 in Hepa1-6 Cells when Delivered as mRNA Generation of mRNA:

mRNA encoding LTRP #1 or LTRP #5 containing the ZIM3-KRAB domain (hereafter known as LTRP1-ZIM3 or LTRP5-ZIM3 respectively) was generated by IVT using plasmid-based PCR templates. Briefly, PCR was performed on plasmids encoding LTRP #1 or LTRP #5 harboring the ZIM3-KRAB domain with flanking NLSes with a forward primer containing a T7 promoter and reverse primer encoding a 120-nucleotide poly(A) tail. These constructs also contained a 2× FLAG sequence. DNA sequences encoding these molecules are listed in Table 38. The resulting PCR templates were used for IVT reactions, which were carried out with CleanCap® AG and N1-methyl-pseudouridine modifications of the mRNA. IVT reactions were then subjected to DNase digestion and on-column oligo dT purification. Full-length RNA sequences encoding the LTRP mRNAs are listed in Table 39.

As experimental controls, mRNA encoding catalytically-active CasX 491 was also similarly generated by IVT using a PCR template as described. Generation of mRNAs encoding LTRP1-ZIM3 and dCas9-ZNF10-DNMT3A/3L, a catalytically-dead Cas9 fused to both the ZNF10-KRAB domain and DNMT3A/L domains was performed as described above for experiment #1.

TABLE 38
Encoding sequences of the LTRP1-ZIM3 and LTRP5-ZIM3 mRNA
molecules assessed in experiment #2 of this example*

| LTRP | | DNA sequence |
|---|---|---|
| ID | Component | SEQ ID NO |
| LTRP1- | 5′UTR | 2017 |
| ZIM3 | START codon + NLS + linker | 2018 |
| | START codon + DNMT3A catalytic domain | 2019 |
| | Linker | 1973 |
| | DNMT3L interaction domain | 2020 |
| | Linker | 1980 |
| | Linker + buffer | 2021 |
| | dCasX491 | 1981 |
| | Linker + buffer | 1982 |
| | ZIM3-KRAB | 2022 |
| | Buffer + NLS | 2023 |
| | Tag | 2024 |
| | Buffer | 2025 |
| | Poly(A) tail | 2026 |
| LTRP5- | 5′UTR | 2017 |
| ZIM3 | START codon + NLS + buffer | 1970 |
| | START codon + DNMT3A catalytic domain | 2019 |
| | Linker | 1973 |
| | DNMT3L interaction domain | 2020 |
| | Linker | 1975 |
| | ZIM3-KRAB | 2022 |
| | Linker | 1980 |
| | dCasX491 | 1981 |
| | Linker + buffer | 1982 |
| | NLS | 2027 |
| | Tag | 2024 |
| | Buffer | 2025 |
| | Poly(A) tail | 2026 |

*Components are listed in a 5′ to 3′ order within the constructs

TABLE 39
Full-length RNA sequences of LTRP1-ZIM3 and LTRP5-ZIM3
mRNA molecules assessed in experiment #2 of this
example. Modification ‘mψ‘ = N1-methyl-pseudouridine

| | RNA sequence |
|---|---|
| LTRP ID | SEQ ID NO |
| LTRP1-ZIM3 | 2028 |
| LTRP5-ZIM3 | 2029 |

For experiment #2, synthesis of PCSK9-targeting gRNAs was performed as described above for experiment #1, and the sequences of the targeting spacers are listed in Table 37. For pairing with dCas9-ZNF10-DNMT3A/3L, targeting spacers were as follows: 1) 7.148 (B2M, as non-targeting control; SEQ ID NO: 1905), 27.126 (PCSK9; CACGCCACCCCGAGCCCCAU; SEQ ID NO: 2030), and 27.128 (PCSK9; CAGCCUGCGCGUCCACGUGA; SEQ ID NO: 2031).

Transfection of mRNA and gRNA into Hepa1-6 Cells and Intracellular PCSK9 Staining:

Seeded Hepa1-6 cells treated with the NATE™ inhibitor were lipofected with 300 ng of mRNA encoding LTRP1-ZIM3, LTRP5-ZIM3, catalytically-active CasX 491, or dCas9-ZNF10-DNMT3A/3L, and 150 ng of PCSK9-targeting gRNA (Table 37). Intracellular levels of PCSK9 protein were measured at day 7, 14, 21, 36, and 71 days post-transfection using an intracellular staining protocol as described for experiment #1.

### Results:

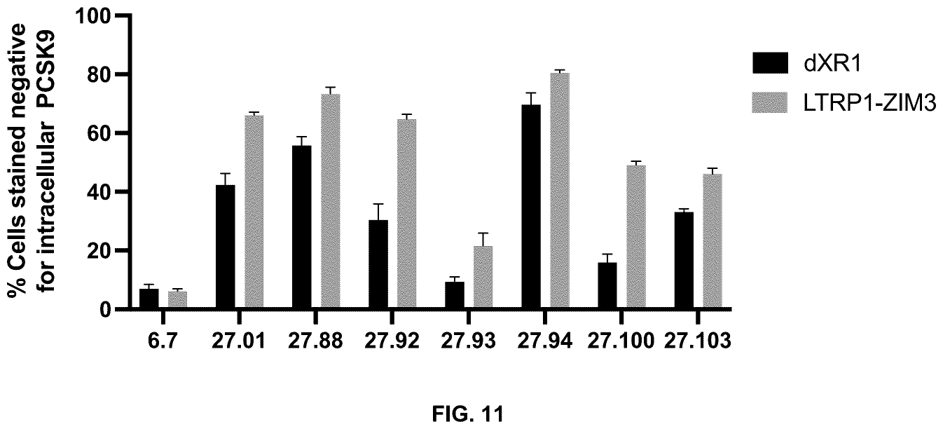

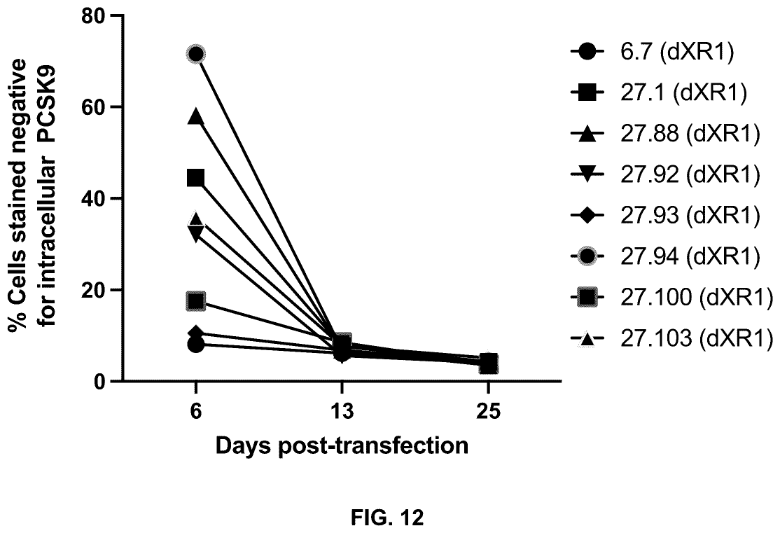

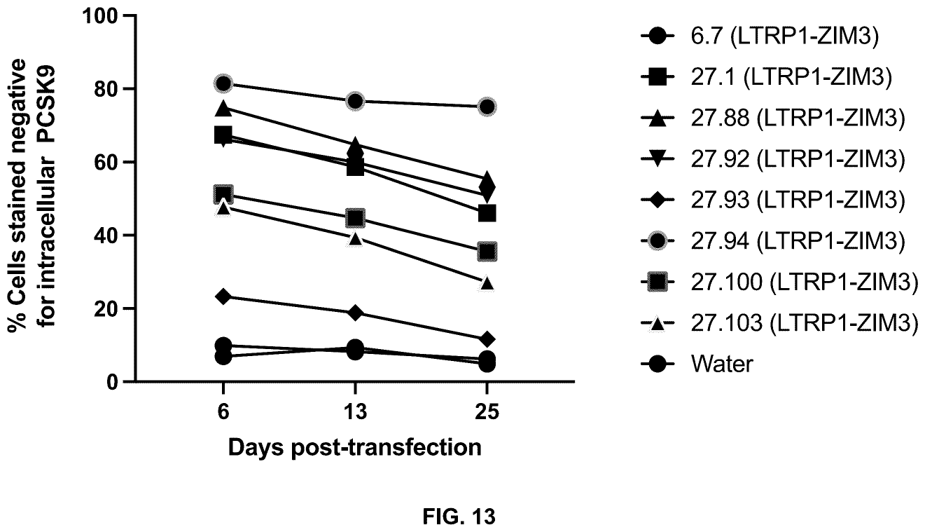

In experiment #1, mRNAs encoding dXR1 or LTRP1-ZIM3 were co-transfected with a PCSK9-targeting gRNA into mouse Hepa1-6 cells to assess their ability to induce PCSK9 knockdown by silencing the mouse PCSK9 locus. The quantification of the resulting PCSK9 knockdown is shown in FIGS. 11-13. The data demonstrate that at day 6, use of six out of seven gRNAs targeting the mouse PCSK9 locus with LTRP1-ZIM3 mRNA resulted in >50% knockdown of intracellular PCSK9, with the leading spacer 27.94 achieving >80% repression level (FIG. 11). A similar trend was observed with use of dXR1 mRNA at day 6, although the degree of repression was less substantial when paired with certain spacers, such as spacer 27.92 and 27.100 (FIG. 11). The results also demonstrate that use of LTRP1-ZIM3 mRNA led to sustained repression of the PCSK9 locus through at least 25 days, with use of the top two spacers 27.94 and 27.88 showing the strongest persistence of silencing PCSK9 (FIG. 13). However, the PCSK9 repression mediated by dXR1 that was observed at day 6 reverted to similar levels of PCSK9 expression as detected with the non-targeting control (spacer 6.7) by day 13; such transient repression with the dXR1 was noticeable for all gRNAs assayed that targeted the PCSK9 gene (FIG. 12).

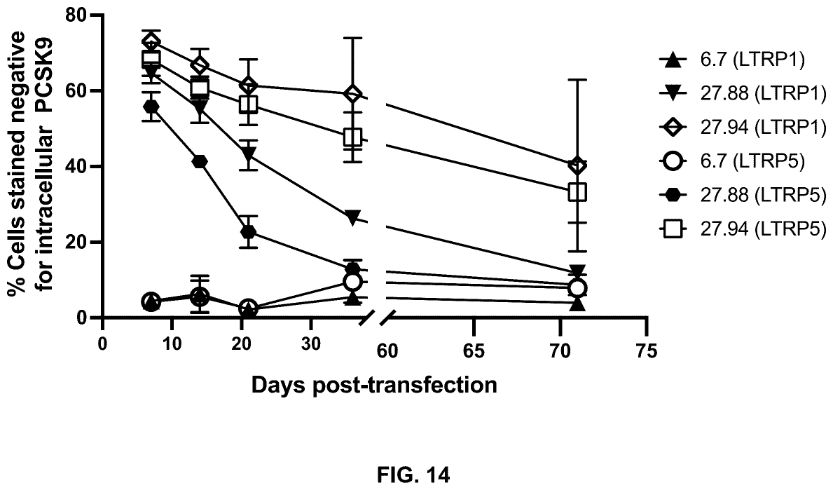

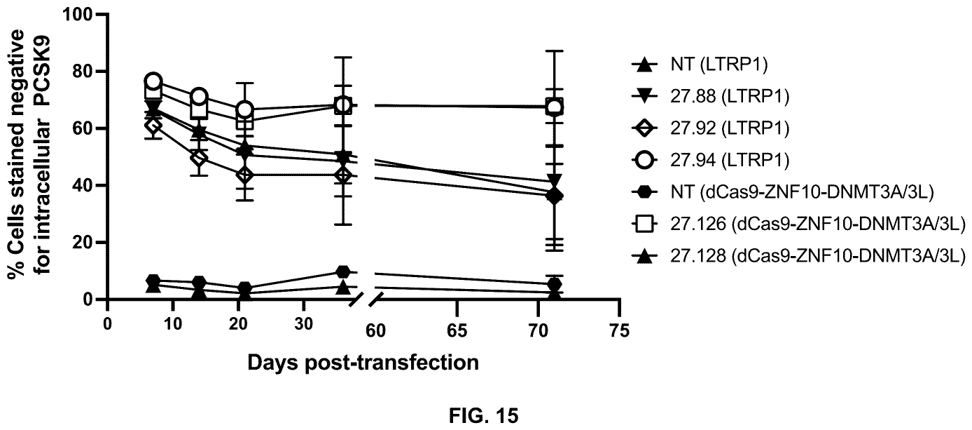

In experiment #2, mRNAs encoding LTRP1-ZIM3 or LTRP5-ZIM3, dCas9-ZNF10-DNMT3A/3L, or catalytically active CasX 491 were co-transfected with a PCSK9-targeting gRNA into mouse Hepa1-6 cells to assess their ability to induce PCSK9 knockdown by silencing the mouse PCSK9 locus. The quantification of the resulting PCSK9 repression is shown in FIGS. 14-15. The data demonstrate that delivery of IVT-produced LTRP1-ZIM3 or LTRP5-ZIM3 mRNA resulted in comparable levels of sustained PCSK9 knockdown when paired with a targeting gRNA with the top spacer 27.94 (~40% knockdown by day 71), while use of spacer 27.88 did not result in as effective of a sustained PCSK9 knockdown by day 71 (~12%) (FIG. 14). Furthermore, mRNA encoding LTRP1-ZIM3 and dCas9-ZNF10-DNMT3A/3L led to similar levels of durable PCSK9 knockdown when paired with gRNAs containing various spacers, with use of spacer 27.94 still resulting in the highest level of PCSK9 repression (FIG. 15).

These experiments demonstrate that LTRP molecules, having different configurations, can induce heritable silencing of an endogenous locus in a mouse liver cell line. Meanwhile, as anticipated, use of dXR constructs result in efficient repression of the target locus at early timepoints, but their use does not lead to durable silencing. These findings also show that dXR and LTRP molecules of different configurations can be delivered as mRNA and co-transfected with a targeting gRNA to cells, indicating that the transient nature of the delivered payload is still sufficient to induce silencing.

### Example 7: Demonstration that Silencing of a Target Locus Mediated by LTRP Molecules is Reversible Using a DNMT1 Inhibitor

Experiments were performed to demonstrate that durable repression of a target locus mediated by LTRP molecules is reversible, such that treatment with a DNMT1 inhibitor would remove methyl marks to reactivate expression of the target gene.

### Materials and Methods

LTRP configuration 5 containing the ZIM3-KRAB domain, which was generated as described in Example 1, and CasX variant 491 were used in this experiment. A B2M-targeting gRNA with scaffold 174 containing spacer 7.37 (SEQ ID NO: 1904) or a non-targeting gRNA containing spacer 0.0 (SEQ ID NO: 1906) were used in this experiment.

### Transfection of HEK293T Cells:

HEK293T cells were transfected with 100 ng of a plasmid containing a construct encoding for either CasX 491 or LTRP #5 containing the ZIM3-KRAB domain with a B2M-targeting gRNA or non-targeting gRNA and cultured for 58 days. These transfected HEK293T cells were subsequently re-seeded at ~30,000 cells well of a 96-well plate and were treated with 5-aza-2′-deoxycytidine (5-azadC), a DNMT1 inhibitor, at concentrations ranging from 0 μM to 20 μM. Six days post-treatment with 5-azadC, cells were harvested for B2M silencing analysis at day 5, day 12, and day 21 post-transfection. Briefly, repression analysis was conducted by analyzing B2M protein expression via HLA immunostaining followed by flow cytometry, as described in Example 1. Treatments for each dose of 5-azadC for each experimental condition were performed in triplicates.

### Results:

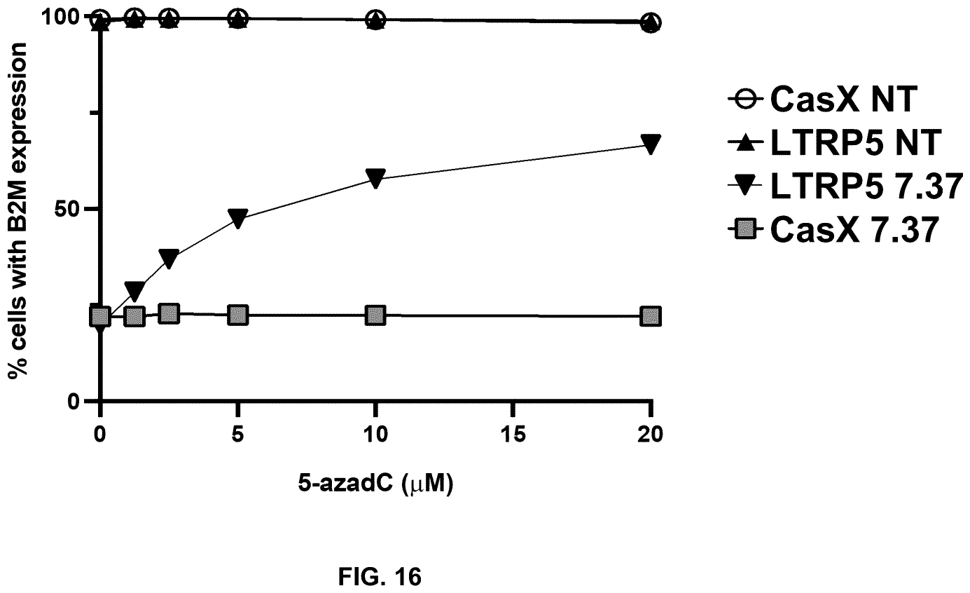

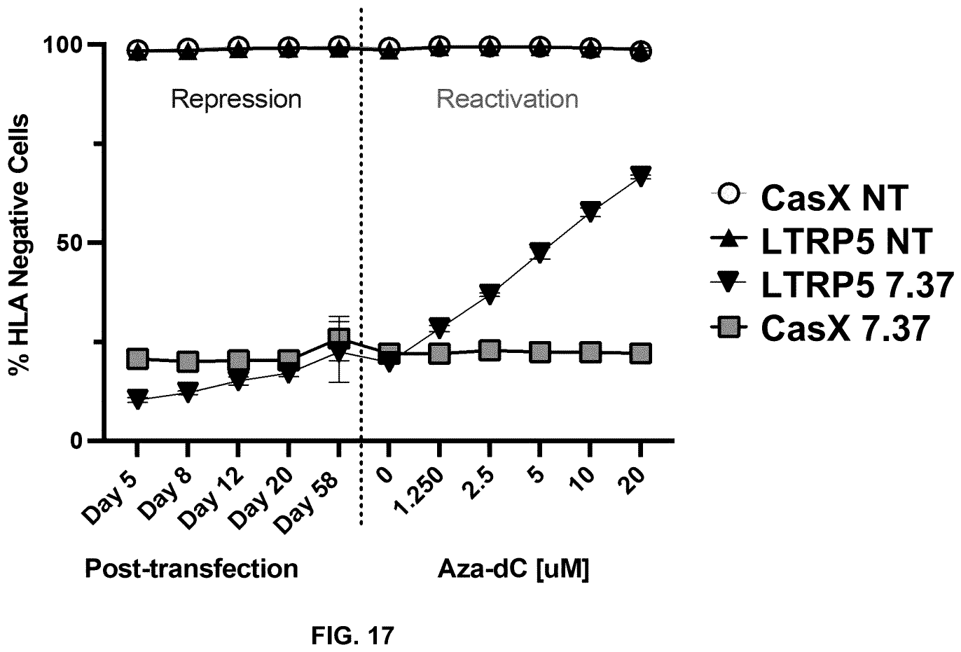

The plot in FIG. 16 shows the percentage of transfected HEK293T cells treated with the indicated concentrations of 5-azadC that expressed the B2M protein. The data demonstrate that 5-azadC treatment of cells transfected with a plasmid encoding LTRP5-ZIM3 with the B2M-targeting gRNA resulted in a reactivation of the B2Mgene (FIG. 16). Specifically, ~75% of treated cells exhibited B2M expression with 20 μM 5-azadC by day 20, compared to the 25% of cells with B2M expression at 0 μM concentration (FIG. 16). Furthermore, 5-azadC treatment of cells transfected with a plasmid encoding CasX 491 with the B2M-targeting gRNA did not exhibit reactivation of the B2M gene. FIG. 17 is a plot that juxtaposes B2M repression activity with gene reactivation upon 5-azadC treatment. The data show B2M repression post-transfection with either CasX 491 or LTRP5-ZIM3 with the B2M-targeting gRNA, resulting in ~75% repression of B2M expression by day 58; however, B2M expression is increased upon 5-azadC treatment (FIG. 17). As anticipated, 5-azadC treatment of cells transfected with either CasX 491 or LTRP5-ZIM3 with the non-targeting gRNA did not demonstrate repression or reactivation (FIGS. 16-17).

The experiments demonstrate reversibility of LTRP-mediated repression of a target locus. By using a DNMT1 inhibitor to remove methyl marks implemented by LTRP molecules, the silenced target gene was reactivated to induce expression of the target protein.

### Example 8: Assessment of Spacers in Achieving Repression of a Therapeutically-Relevant Locus in Human Hepatocyte Cells when Paired with an LTRP5-ADD Molecule

Experiments were performed to demonstrate that multiple gRNA having spacers with the TTC PAM motif, when paired with an LTRP molecule in configuration 5 (LTRP5) containing the ADD domain, can induce durable repression of a therapeutically-relevant endogenous locus (the PCSK9 gene) in human cells. Specifically, an initial proof-of-concept experiment was performed in human Huh7 cells to evaluate a subset of spacers that exhibit sequence conservation to the non-human primate (NHP) genome to identify leading spacers for testing in future in vivo NHP studies.

### Materials and Methods

Computational Selection of PCSK9-Targeting Spacers for Experimental Testing with an LTRP5 Molecule Containing the ADD Domain:

To determine potential LTRP-specific spacers throughout the human PCSK9 locus, a target search region was defined as starting at 5 KB upstream of the transcription start site (TSS) through 5 KB downstream of the transcription stop site. Spacers were determined based on the availability of TTC PAMs in the PCSK9 locus; consequently, a total of 1,121 TTC spacers were identified throughout the target PCSK9 locus. These spacers were then functionally annotated by overlaying key genomic features based on their positioning; i.e., determining whether the putative spacer targeted an exon, an intron, or a candidate cis-regulatory element (cCRE), within the promoter region, and/or overlapped with a common site of genetic variation (e.g., SNPs). To narrow down and determine an initial group of spacers for experimental screening, the extracted spacers were subjected to a set of filtering criteria. Firstly, non-specific spacers were excluded by removing spacers with off-target sites that contained up to one base pair mismatch with the on-target site. Furthermore, spacers containing the following mononucleotide repeats were excluded: thymine nucleotide repeats greater than four base pairs (bp) in length or adenine, guanine, or cytosine nucleotide repeats greater than 5 bp in length. Next, from this filtered set, spacers with more than one off-target site containing mismatches in the last four nucleotides of the spacer were excluded. Lastly, spacers that targeted >2 KB upstream of the TSS and >2 KB downstream of the transcription stop site were excluded. This resulted in a filtered set of 722 TTC spacers. From this filtered set of 722 spacers, spacers that were TSS-proximal (within 1100 bp upstream and downstream of the TSS) were selected for experimental assessment, resulting in the identification of 67 TTC spacers. Two additional spacers, TG-06-354 and TG-06-352, positioned beyond the 1100 bp threshold window, were also selected for inclusion. The sequences of the resulting 69 TTC spacers are shown in Table 40.

TABLE 40
Sequences of the 69 TTC spacers
targeting the human PCSK9 locus. Bolded
spacers were spacers having sequence consensus
between human and non-human primate
genomes and were assessed in this example.

| | | RNA SEQ |
|---|---|---|
| Spacer ID | Spacer RNA sequence | ID NO: |
| TG-06-342 | AAUUACAGGCAACAGGAAGG | 2067 |
| TG-06-343 | CCCCAUGUAAGAGAGGAAGU | 2068 |
| TG-06-344 | CAGUUUCUGCCUCGCCGCGG | 2069 |
| TG-06-345 | GCCUCGCCGCGGCACAGGUG | 2070 |
| TG-06-346 | CCCACCUGUGCCGCGGCGAG | 2071 |
| TG-06-347 | CUCCUUCACCCACCUGUGCC | 2072 |
| TG-06-348 | AGGCAUUCACUCCUUCACCC | 2073 |
| TG-06-349 | CUGUGCCUGGUGCAGUUCCC | 2074 |
| TG-06-350 | GUGUCAUAAAGAAAUUGCCU | 2075 |
| TG-06-351 | UUAUGACACAGAACUCAUGC | 2076 |
| TG-06-001 | GAGGAGGACGGCCUGGCCGA | 1963 |
| TG-06-002 | ACCGCUGCGCCAAGGUGCGG | 2077 |
| TG-06-004 | GCCAGGCCGUCCUCCUCGGA | 2078 |
| TG-06-005 | GUGCUCGGGUGCUUCGGCCA | 2079 |
| TG-06-117 | ACUUUGUUUGCAAAGACCUC | 2080 |
| TG-06-118 | GAGUGAAAUGGCCUGCUCUG | 2081 |
| TG-06-119 | GAGCAGGCCAUUUCACUCGG | 2082 |
| TG-06-120 | CUCGGAAUCUGCUGUGCAUC | 2083 |
| TG-06-121 | GGAAGGGCUGUCGAUACUGG | 2084 |
| TG-06-123 | UCCCAGUAUCGACAGCCCUU | 2085 |
| TG-06-124 | CAGUAUCGACAGCCCUUCCA | 2086 |
| TG-06-125 | AGAAAGAGCAAGCCUCAUGU | 1964 |
| TG-06-128 | AGAAAUCAACUGGACAAGCA | 2087 |
| TG-06-131 | UGAACAUGGUGUGUAAAAGG | 2088 |
| TG-06-132 | AGAAGAUUCAAUUUGCAAAG | 2089 |
| TG-06-133 | AUGGUAGGCACAAGCUCAGC | 2090 |
| TG-06-134 | GAAUUCUAUGGUAGGCACAA | 2091 |
| TG-06-135 | GGAAAGCUGAGCUUGUGCCU | 2092 |
| TG-06-138 | AGGGAUUUAUACUACAAAGA | 1965 |
| TG-06-139 | AGGAGCAGCUAGUUGGUAAG | 2093 |
| TG-06-140 | AAACUUAGCCUGGACCCCCU | 2094 |
| TG-06-141 | ACUGGCCUUAACCUGGCAGC | 2095 |
| TG-06-142 | UUCCACUGGCCUUAACCUGG | 2096 |
| TG-06-143 | GAAUCAAUCCUACUGUGGAC | 2097 |
| TG-06-144 | GUGGGCAGCGAGGAGUCCAC | 2098 |
| TG-06-145 | UGGGUCCACCUUGUCUCCUG | 2099 |
| TG-06-146 | GAAGUCUCACUGGUCAGCAG | 2100 |
| TG-06-147 | GUGUUUCCUGGGUCCACCUU | 2101 |
| TG-06-149 | AGCCCAGUUAGGAUUUGGGA | 2102 |
| TG-06-150 | UCCCUCUGCGCGUAAUCUGA | 2103 |
| TG-06-151 | CUCUGCGCGUAAUCUGACGC | 2104 |
| TG-06-152 | GCCUCGCCCUCCCCAAACAG | 2105 |
| TG-06-153 | GUUAAUGUUUAAUCAGAUAG | 1966 |
| TG-06-154 | AGGGUGUGGGUGCUUGACGC | 2106 |
| TG-06-155 | GCAGCGACGUCGAGGCGCUC | 2107 |
| TG-06-157 | GGGUCUGAGCCUGGAGGAGU | 1967 |
| TG-06-158 | GGAGCAGGGCGCGUGAAGGG | 2108 |
| TG-06-159 | GCGCGCCCCUUCACGCGCCC | 2109 |
| TG-06-160 | CGCGCCCUGCUCCUGAACUU | 2110 |
| TG-06-161 | GCUCCUGCACAGUCCUCCCC | 2111 |
| TG-06-167 | CACUGAAUAGCGCAGCCGCA | 2112 |
| TG-06-168 | GUGGGAAGGUUCGCGGGGUU | 2113 |
| TG-06-169 | CGGGGUUGGGAGACCCGGAG | 2114 |
| TG-06-170 | UCGGCCUCCGGGUCUCCCAA | 2115 |
| TG-06-171 | CAGUACGUUCCAGGCAUUCA | 2116 |
| TG-06-172 | GCUGAAACAGAUGGAAUACU | 1968 |
| TG-06-249 | AAACCAAAUCGGAACCCACU | 2117 |
| HS-6-147 | UGUUGCCUGUAAUUGGAAUU | 2118 |
| HS-6-149 | CCUUCCUGUUGCCUGUAAUU | 2119 |
| TG-06-122 | CCUUUGUUUCUUCCCAGUAU | 2120 |
| TG-06-127 | UCCUCCUGCCUGGUACACAA | 2121 |
| TG-06-137 | GAAUGUACCUAUAUGACGUC | 2122 |
| TG-06-188 | CCCCGGCCUCCCAUCCCUAC | 2123 |
| TG-06-243 | CUUGGCACGAUCUUGGGGAC | 2124 |
| TG-06-250 | GAUUUGGUUUGGAAAACAUG | 2125 |
| TG-06-251 | CUCCAGGCCCUCCACCCUCC | 2126 |
| HS-6-159 | CACCCCGCCCCUGUCUCGGG | 2127 |
| TG-06-354 | CCCCUGCCCCUUCAGCUGGU | 2128 |
| TG-06-352 | UCCCUCACCAAUUACCCCUC | 2129 |

Assessment of PCSK9 Secretion Levels for Select PCSK9-Targeting Spacers Having Sequence Conservation with the Non-Human Primate Genome:

Of the 69 TTCN spacers identified, 15 spacers that exhibited sequence conservation between human and non-human primate genomes (bolded spacers in Table 40) were initially tested to assess their effect on PCSK9 secretion levels.

mRNA encoding the following molecules were generated by IVT following similar methods as described in Example 6: 1) a catalytically-active CasX 676 (as described in Example 2), 2) dXR1 (as described in Example 6), and 3) LTRP5-ADD-ZIM3 (as described in Example 6). The DNA sequences for CasX 676 are shown in Table 11; the DNA and mRNA sequences for dXR1 are shown in Tables 35 and 36; the DNA and mRNA sequences for LTRP5-ZIM3-ADD are shown in Tables 9 and 10.

gRNAs containing NHP-conserved spacers targeting the PCSK9 locus (bolded spacers in Table 40) were designed using gRNA scaffold 316 and chemically synthesized using the v1 modification profile (as described in Example 11; SEQ ID NO: 2156). Furthermore, a B2M-targeting gRNA was used as a non-targeting control, while spacer TG-06-138 (also known as spacer 6.138; SEQ ID NO: 1965) was used to pair with dXR1, and spacer TG-06-001 (also known as spacer 6.1; SEQ ID NO: 1963) was used to pair with CasX 676. Spacer TG-06-157 (also known as spacer 6.157; SEQ ID NO: 1967), which is not an NHP-conserved spacer, was included as a positive control given its demonstrated efficacy in sustaining repression of the PCSK9 locus, which was shown in Example 2.

To assess PCSK9 secretion, seeded Huh7 cells were transfected with mRNA encoding a catalytically-active CasX 676, dXR1, or LTRP5-ADD-ZIM3 and a gRNA with scaffold 316 and a spacer targeting either the B2M or PCSK9 locus. Media supernatant was harvested at 6, 18, and 36 days post-transfection to assess level of PCSK9 secretion by ELISA. Levels of PCSK9 secretion were normalized to total cell count. As an additional control, PCSK9 secretion was also measured in the media supernatant harvested from wells containing untreated, naïve cells.

### Results:

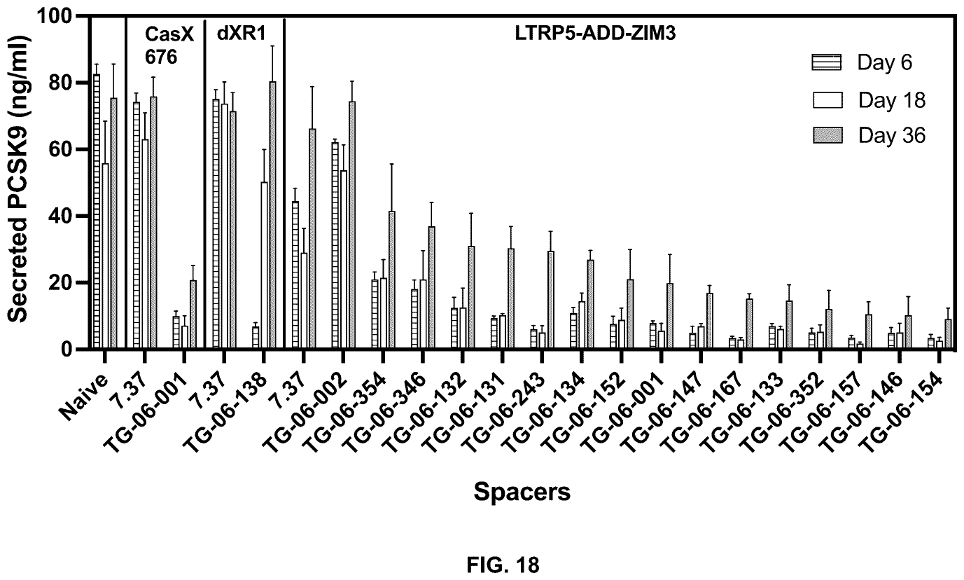

Quantification of normalized PCSK9 secretion level for Huh7 cells transfected with mRNA encoding catalytically-active CasX 676, dXR1, or LTRP5-ADD-ZIM3 with an NHP-conserved gRNA targeting the PCSK9 locus at the three timepoints is shown FIG. 18. The data demonstrate that use of most NHP-conserved spacers with LTRP5-ADD-ZIM3 resulted in sustained repression through 36 days post-transfection when compared with control conditions, i.e., naïve, untreated cells, cells treated with dXR1, and cells treated with the non-targeting control (using spacer 7.37 targeting the B2M locus). Specifically, in comparison to the PCSK9 secretion level observed with use of spacer 6.1 paired with CasX 676, use of TSS-proximal spacers TG-06-147, TG-06-167, TG-06-133, TG-06-146, and TG-06-154 paired with LTRP5-ADD-ZIM3 resulted in similar or further reduced level of sustained repression (FIG. 18). Interestingly, use of TG-06-352, which is positioned beyond the 1100 bp threshold window (designated here as “TSS-proximal”), also resulted in effective repression (FIG. 18). Similar to the findings observed in Example 2, treatment with LTRP5-ADD-ZIM3 with spacer 6.157 resulted in sustained repression of secreted PCSK9 levels, while treatment with dXR1 and spacer 6.138 resulted in transient repression, with PCSK9 levels increasing in the latter two intervals. Furthermore, treatment with any of the three mRNA molecules with spacer 7.37 targeting the B2M locus did not affect PCSK9 secretion (FIG. 18).

These results demonstrate that delivery of mRNA encoding an LTRP molecule with the ADD domain with the appropriate PCSK9-targeting gRNA can result in sustained repression of an endogenous target locus in human cells. Furthermore, these experiments revealed that several human spacers having consensus sequence with the non-human primate species achieved strong phenotypic effects from targeting a therapeutically-relevant locus, supporting the potential use of these select spacers in preclinical efficacy studies utilizing non-human primate models.

### Example 9: Exemplary Sequences of LTRP mRNA Molecules

Table 41 provides exemplary full-length mRNA and amino acid sequences of LTRP molecules. In Table 41, the mRNA sequences are shown without the N1-methyl-pseudouridine modification.

TABLE 41
Exemplary mRNA and amino acid sequences of LTRP mRNA molecules

| | | | mRNA | |
|---|---|---|---|---|
| | | | sequence | AA sequence |
| Molecule | Repressor domain | Linker Set | SEQ ID NO | SEQ ID NO |
| LTRP5-ADD-ZIM3 | Human Zim3 | Baseline/Original | 1875 | 1883 |
| LTRP5-ADD- | DOMAIN_26749 | Baseline/Original | 1876 | 1884 |
| Ophiophagus_hannah | | | | |
| LTRP5-ADD- | DOMAIN_10123 | Baseline/Original | 1877 | 1885 |
| Rattus_norvegicus | | | | |
| LTRP5-ADD- | DOMAIN_26749 | Set 85 | 1878 | 1886 |
| Ophiophagus_hannah_Link85 | | | | |
| LTRP5-ADD- | DOMAIN_26749 | Set 43 | 1879 | 1887 |
| Ophiophagus_hannah_Link43 | | | | |
| LTRP6-ADD- | DOMAIN_26749 | Baseline/Original | 1880 | 1888 |
| 2x_Ophiophagus_hannah | | | | |
| LTRP6-ADD- | DOMAIN_10123 | Baseline/Original | 1881 | 1889 |
| Rattus_Ophiophagus | and | | | |
| | DOMAIN_26749 | | | |
| LTRP6-ADD- | DOMAIN_26749 | Set 85 | 1882 | 1890 |
| 2x_Ophiophagus_hannah_{—} | | | | |
| Link85 | | | | |

Table 42 provides exemplary full-length LTRP constructs in configurations 1, 4, 5, or 6 (FIG. 19), with the ADD domain, with each of the top nine KRAB domains: DOMAIN_7694 DOMAIN_10123, DOMAIN_15507, DOMAIN_17905, DOMAIN_20505, DOMAIN_26749, DOMAIN_27604, DOMAIN_29304, and DOMAIN_30173.

TABLE 42
Exemplary protein sequences of LTRP molecules containing the top nine KRAB
domains with the ADD domain and having the #1, #4, #5, or #6 configuration

| LTRP # | Components | Domains | SEQ ID NO |
|---|---|---|---|
| LTRP1 | START codon + NLS + buffer sequence | | 1985 |
| with | START codon + DNMT3A ADD | | 1986 |
| ADD | domain | | |
| domain | DNMT3A catalytic domain | | 126 |
| | Linker (L2) | | 122 |
| | DNMT3L interaction domain | | 127 |
| | Linker (L1) | | 123 |
| | Linker (L3A) | | 2131 |
| | dCasX491 | | 4 |
| | Buffer + linker (L3B) | | 1987 |
| | Repressor domain 1 | Human ZIM3 | 1892 |
| | | Human ZNF10 | 1891 |
| | | Columba livia repressor domain | 130 |
| | | (DOMAIN_7694) | |
| | | Rattus norvegicus repressor | 131 |
| | | domain (DOMAIN_10123) | |
| | | Cebus imitator repressor | 132 |
| | | domain (DOMAIN_15507) | |
| | | Chimpanzee repressor domain | 133 |
| | | (DOMAIN_17905) | |
| | | Chlorocebus sabaeus repressor | 134 |
| | | domain (DOMAIN_20505) | |
| | | Ophiophagus hannah repressor | 135 |
| | | domain (DOMAIN_26749) | |
| | | Ailuropoda melanoleuca | 136 |
| | | repressor domain | |
| | | (DOMAIN_27604) | |
| | | Peromyscus maniculatus | 137 |
| | | bairdii repressor domain | |
| | | (DOMAIN_29304) | |
| | | Phyllostomus discolor repressor | 138 |
| | | domain (DOMAIN_30173) | |
| | Buffer + NLS | | 2132 |
| LTRP4 | START codon + NLS + buffer sequence | | 1985 |
| with | Repressor domain 1 | Human ZIM3 | 1892 |
| ADD | | Human ZNF10 | 1891 |
| domain | | Columba livia repressor domain | 130 |
| | | (DOMAIN_7694) | |
| | | Rattus norvegicus repressor | 131 |
| | | domain (DOMAIN_10123) | |
| | | Cebus imitator repressor | 132 |
| | | domain (DOMAIN_15507) | |
| | | Chimpanzee repressor domain | 133 |
| | | (DOMAIN_17905) | |
| | | Chlorocebus sabaeus repressor | 134 |
| | | domain (DOMAIN_20505) | |
| | | Ophiophagus hannah repressor | 135 |
| | | domain (DOMAIN_26749) | |
| | | Ailuropoda melanoleuca | 136 |
| | | repressor domain | |
| | | (DOMAIN_27604) | |
| | | Peromyscus maniculatus | 137 |
| | | bairdii repressor domain | |
| | | (DOMAIN_29304) | |
| | | Phyllostomus discolor repressor | 138 |
| | | domain (DOMAIN_30173) | |
| | Linker (L3A) | | 2131 |
| | START codon + DNMT3A ADD | | 1986 |
| | domain | | |
| | DNMT3A catalytic domain | | 126 |
| | Linker (L2) | | 122 |
| | DNMT3L interaction domain | | 127 |
| | Linker (L1) | | 123 |
| | dCasX491 | | 4 |
| | Buffer + linker (L3B) | | 1987 |
| | NLS | | 30 |
| LTRP5 | START codon + NLS + buffer sequence | | 1985 |
| with | START codon + DNMT3A ADD | | 1986 |
| ADD | domain | | |
| domain | DNMT3A catalytic domain | | 126 |
| | Linker (L2) | | 122 |
| | DNMT3L interaction domain | | 127 |
| | Linker (L3A) | | 124 |
| | Repressor domain 1 | Human ZIM3 | 1892 |
| | | Human ZNF10 | 1891 |
| | | Columba livia repressor domain | 130 |
| | | (DOMAIN_7694) | |
| | | Rattus norvegicus repressor | 131 |
| | | domain (DOMAIN_10123) | |
| | | Cebus imitator repressor | 132 |
| | | domain (DOMAIN_15507) | |
| | | Chimpanzee repressor domain | 133 |
| | | (DOMAIN_17905) | |
| | | Chlorocebus sabaeus repressor | 134 |
| | | domain (DOMAIN_20505) | |
| | | Ophiophagus hannah repressor | 135 |
| | | domain (DOMAIN_26749) | |
| | | Ailuropoda melanoleuca | 136 |
| | | repressor domain | |
| | | (DOMAIN_27604) | |
| | | Peromyscus maniculatus | 137 |
| | | bairdii repressor domain | |
| | | (DOMAIN_29304) | |
| | | Phyllostomus discolor repressor | 138 |
| | | domain (DOMAIN_30173) | |
| | Linker (L1) | | 123 |
| | dCasX491 | | 4 |
| | Buffer + linker (L3B) | | 1987 |
| | NLS | | 30 |
| LTRP6 | START codon + NLS + buffer sequence | | 1985 |
| with | START codon + DNMT3A ADD | | 1986 |
| ADD | domain | | |
| domain | DNMT3A catalytic domain | | 126 |
| | Linker 2 (L2) | | 122 |
| | DNMT3L interaction domain | | 127 |
| | Linker 3A (L3A) | | 124 |
| | N-terminal repressor domain | Human ZIM3 | 1892 |
| | | Human ZNF10 | 1891 |
| | | Columba livia repressor domain | 130 |
| | | (DOMAIN_7694) | |
| | | Rattus norvegicus repressor | 131 |
| | | domain (DOMAIN_10123) | |
| | | Cebus imitator repressor | 132 |
| | | domain (DOMAIN_15507) | |
| | | Chimpanzee repressor domain | 133 |
| | | (DOMAIN_17905) | |
| | | Chlorocebus sabaeus repressor | 134 |
| | | domain (DOMAIN_20505) | |
| | | Ophiophagus hannah repressor | 135 |
| | | domain (DOMAIN_26749) | |
| | | Ailuropoda melanoleuca | 136 |
| | | repressor domain | |
| | | (DOMAIN_27604) | |
| | | Peromyscus maniculatus | 137 |
| | | bairdii repressor domain | |
| | | (DOMAIN_29304) | |
| | | Phyllostomus discolor repressor | 138 |
| | | domain (DOMAIN_30173) | |
| | Linker 1 (L1) | | 123 |
| | dCasX | | 4 |
| | Buffer sequence + Linker 3B (L3B) + | | 1987 |
| | buffer sequence | | |
| | C-terminal repressor domain | Human ZIM3 | 1892 |
| | | Human ZNF10 | 1891 |
| | | Columba livia repressor domain | 130 |
| | | (DOMAIN_7694) | |
| | | Rattus norvegicus repressor | 131 |
| | | domain (DOMAIN_10123) | |
| | | Cebus imitator repressor | 132 |
| | | domain (DOMAIN_15507) | |
| | | Chimpanzee repressor domain | 133 |
| | | (DOMAIN_17905) | |
| | | Chlorocebus sabaeus repressor | 134 |
| | | domain (DOMAIN_20505) | |
| | | Ophiophagus hannah repressor | 135 |
| | | domain (DOMAIN_26749) | |
| | | Ailuropoda melanoleuca | 136 |
| | | repressor domain | |
| | | (DOMAIN_27604) | |
| | | Peromyscus maniculatus | 137 |
| | | bairdii repressor domain | |
| | | (DOMAIN_29304) | |
| | | Phyllostomus discolor repressor | 138 |
| | | domain (DOMAIN_30173) | |
| | Linker (L4) | L4v1 | 1988 |
| | | L4v2 | 2130 |
| | NLS | | 30 |

Table 43 provides exemplary amino acid sequences of components of LTRP constructs. In Table 43, the protein domains are shown without starting methionines.

TABLE 43
Exemplary protein sequences of components of LTRP constructs

| | | SEQ |
|---|---|---|
| Component | Protein sequence | ID NO |
| DNMT3A | NHDQEFDPPKVYPPVPAEKRKPIRVLSLFDGIATGLLVLKDLGIQVDRYIASEVCE | 126 |
| catalytic | DSITVGMVRHQGKIMYVGDVRSVTQKHIQEWGPFDLVIGGSPCNDLSIVNPARKGL | |
| domain | YEGTGRLFFEFYRLLHDARPKEGDDRPFFWLFENVVAMGVSDKRDISRFLESNPVM | |
| (CD) | IDAKEVSAAHRARYFWGNLPGMNRPLASTVNDKLELQECLEHGRIAKFSKVRTITT | |
| | RSNSIKQGKDQHFPVFMNEKEDILWCTEMERVFGFPVHYTDVSNMSRLARQRLLGR | |
| | SWSVPVIRHLFAPLKEYFACV | |
| DNMT3L | MGPMEIYKTVSAWKRQPVRVLSLFRNIDKVLKSLGFLESGSGSGGGTLKYVEDVTN | 127 |
| interaction | VVRRDVEKWGPFDLVYGSTQPLGSSCDRCPGWYMFQFHRILQYALPRQESQRPFFW | |
| domain | IFMDNLLLTEDDQETTTRFLQTEAVTLQDVRGRDYQNAMRVWSNIPGLKSKHAPLT | |
| | PKEEEYLQAQVRSRSKLDAPKVDLLVKNCLLPLREYFKYFSQNSLPL | |
| dCasX491 | QEIKRINKIRRRLVKDSNTKKAGKTGPMKTLLVRVMTPDLRERLENLRKKPENIPQ | 4 |
| | PISNTSRANLNKLLTDYTEMKKAILHVYWEEFQKDPVGLMSRVAQPASKKIDQNKL | |
| | KPEMDEKGNLTTAGFACSQCGQPLFVYKLEQVSEKGKAYTNYFGRCNVAEHEKLIL | |
| | LAQLKPEKDSDEAVTYSLGKFGQRALDFYSIHVTKESTHPVKPLAQIAGNRYASGP | |
| | VGKALSDACMGTIASFLSKYQDIIIEHQKVVKGNQKRLESLRELAGKENLEYPSVT | |
| | LPPQPHTKEGVDAYNEVIARVRMWVNLNLWQKLKLSRDDAKPLLRLKGFPSFPLVE | |
| | RQANEVDWWDMVCNVKKLINEKKEDGKVFWQNLAGYKRQEALRPYLSSEEDRKKGK | |
| | KFARYQLGDLLLHLEKKHGEDWGKVYDEAWERIDKKVEGLSKHIKLEEERRSEDAQ | |
| | SKAALTDWLRAKASFVIEGLKEADKDEFCRCELKLQKWYGDLRGKPFAIEAENSIL | |
| | DISGFSKQYNCAFIWQKDGVKKLNLYLIINYFKGGKLRFKKIKPEAFEANRFYTVI | |
| | NKKSGEIVPMEVNFNFDDPNLIILPLAFGKRQGREFIWNDLLSLETGSLKLANGRV | |
| | IEKTLYNRRTRQDEPALFVALTFERREVLDSSNIKPMNLIGVARGENIPAVIALTD | |
| | PEGCPLSRFKDSLGNPTHILRIGESYKEKQRTIQAKKEVEQRRAGGYSRKYASKAK | |
| | NLADDMVRNTARDLLYYAVTQDAMLIFANLSRGFGRQGKRTFMAERQYTRMEDWLT | |
| | AKLAYEGLSKTYLSKTLAQYTSKTCSNCGFTITSADYDRVLEKLKKTATGWMTTIN | |
| | GKELKVEGQITYYNRYKRQNVVKDLSVELDRLSEESVNNDISSWTKGRSGEALSLL | |
| | KKRFSHRPVQEKFVCLNCGFETHAAEQAALNIARSWLFLRSQEYKKYQTNKTTGNT | |
| | DKRAFVETWQSFYRKKLKEVWKPAV | |
| Linker 1 | GGPSSGAPPPSGGSPAGSPTSTEEGTSESATPESGPGTSTEPSEGSAPGSPAGSPT | 123 |
| (L1) | STEEGTSTEPSEGSAPGTSTEPSE | |
| Linker 2 | SSGNSNANSRGPSFSSGLVPLSLRGSH | 122 |
| (L2) | | |
| Linker 3A | GGSGGG | 124 |
| (L3A) | | |
| Linker 3B | GGSGGGS | 120 |
| (L3B) | | |
| Linker 4v1 | GSGS | 1988 |
| (L4v1) | | |
| Linker 4v2 | GSGSGSG | 2130 |
| (L4v2) | | |
| NLS | PKKKRKV | 30 |
| DNMT3A | ERLVYEVRQKCRNIEDICISCGSLNVTLEHPLFIGGMCQNCKNCFLECAYQYDDDG | 125 |
| ADD | YQSYCTICCGGREVLMCGNNNCCRCFCVECVDLLVGPGAAQAAIKEDPWNCYMCGH | |
| domain | KGTYGLLRRREDWPSRLQMFFAN | |

### Example 10: LNP Delivery of LTRP mRNA and Targeting gRNA Via LNPs to Achieve Repression of Target Locus In Vitro

Experiments are performed to assess whether delivery of lipid nanoparticles (LNPs) encapsulating LTRP mRNA and targeting gRNA induce durable repression of a target endogenous locus in a cell-based assay.

### Materials and Methods

Generation of LTRP mRNAs:

mRNA encoding LTRP molecules are be generated by IVT, as described earlier in Example 6. Sequences encoding the LTRP molecule are codon-optimized as briefly described in Example 6. Examples of DNA sequences encoding LTRP mRNA are listed in Table 35 and Table 38, with the corresponding mRNA sequences listed in Table 36 and Table 39. Additional examples of mRNA sequences encoding LTRP mRNA are presented in Table 41.

Targeting gRNAs (e.g., targeting the endogenous B2M locus) are synthesized as described above in Example 6.

LNP formulations are generated as described in Example 11, below. Delivery of LNPs encapsulating LTRP mRNA and targeting gRNAs into mouse liver Hepa1-6 cells:

Hepa1-6 cells are seeded in a 96-well plate. The next day, seeded cells are treated with varying concentrations of LNPs, which are prepared in six 2-fold serial dilutions starting at 250 ng. These LNPs are formulated to encapsulate an LTRP mRNA and a B2M-targeting gRNA. Media is changed 24 hours after LNP treatment, and cells are cultured before being harvested at multiple timepoints (e.g., 7, 14, 21, 28, and 56 days post-treatment) for gDNA extraction for editing assessment at the B2M locus by NGS and for bisulfite sequencing to assess off-target methylation at the VEGFA locus, as described in Example 1.

The results from this experiment are expected to show that LTRP mRNA and targeting gRNA can be co-encapsulated within LNPs to be delivered to target cells to induce heritable silencing of a target endogenous locus.

### Example 11: Design and Assessment of Modified gRNAs in Improving Editing when Delivered Together with CasX mRNA In Vitro and In Vivo

Experiments were performed to identify new gRNA variant sequences and demonstrate that chemical modifications of these gRNA variants enhance the editing efficiency of the CasX:gRNA system when delivered in vitro in conjunction with CasX mRNA.

### Materials and Methods

Synthesis of gRNAs:

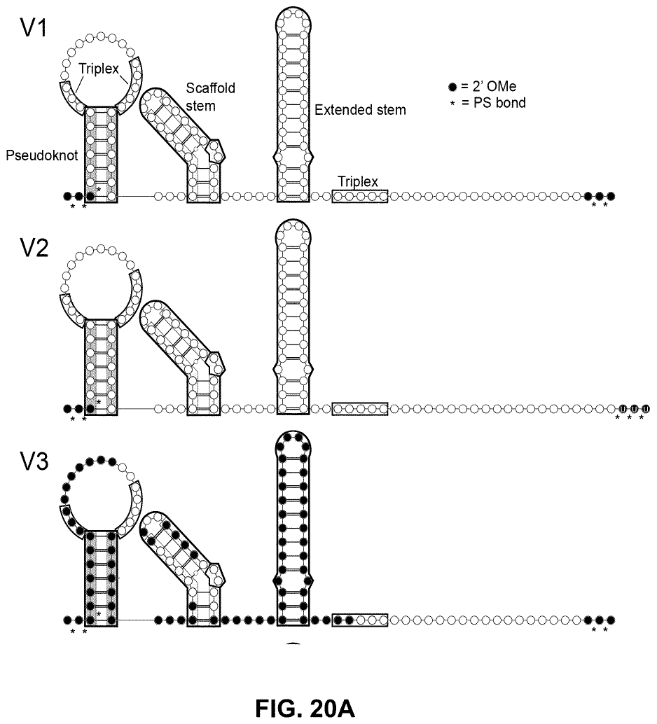

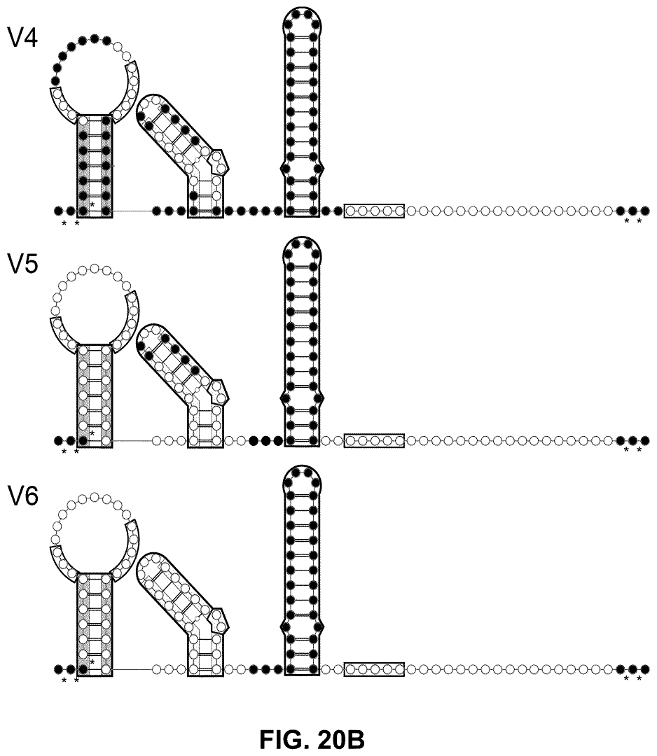

All gRNAs tested in this example were chemically-synthesized and were derived from gRNA scaffolds 174, 235, and 316. The sequences of gRNA scaffolds 174, 235, and 316 and their chemical modification profiles are listed in Table 44. The sequences of the resulting gRNAs, including spacers targeting PCSK9, B2M, or ROSA26, and their chemical modification profiles assayed in this example are listed in Table 45. A schematic of the structure of gRNA scaffold variants 174, 235, and 316 are shown in FIGS. 23A-23C, respectively, and the sites of chemical modifications of the gRNA variants are shown schematically in FIGS. 20A, 20B, 22, 28A, and 28B.

TABLE 44
Sequences of gRNA scaffolds with their different chemical
modification profiles (denoted by version number),
where “NNNNNNNNNNNNNNNNNNNN” is a spacer placeholder.
Chemical modifications: * = phosphorothioate bond;
m = 2′OMe modification

| gRNA | | SEQ |
|---|---|---|
| scaffold | | ID |
| (version) | gRNA sequence | NO: |
| 174 (v0) | ACUGGCGCUUUUAUCUGAUUACUUUGAGAGCCAUCACCAGCGACUAUGU | 2135 |
| | CGUAGUGGGUAAAGCUCCCUCUUCGGAGGGAGCAUCAAAGNNNNNNNNN | |
| | NNNNNNNNNNN | |
| 174 (v1) | mA*mC*mU*GGCGCUUUUAUCUGAUUACUUUGAGAGCCAUCACCAGCGA | 2136 |
| | CUAUGUCGUAGUGGGUAAAGCUCCCUCUUCGGAGGGAGCAUCAAAGNNN | |
| | NNNNNNNNNNNNNNmN*mN*mN | |
| 174 (v2) | mA*mC*mU*GGCGCUUUUAUCUGAUUACUUUGAGAGCCAUCACCAGCGA | 2137 |
| | CUAUGUCGUAGUGGGUAAAGCUCCCUCUUCGGAGGGAGCAUCAAAGNNN | |
| | NNNNNNNNNNNNNNNNN*mU*mU*mU | |
| 174 (v3) | mA*mC*mU*mGmGmCmGmCmUmUmUmUmAmUmCmUmGmAmUUACUUUGm | 2138 |
| | AmGmAmGmCmCmAmUmCmAmCmCAGCGAmCmUAUmGmUmCmGUAGUGmG | |
| | mGmUmAmAmAmGmCmUmCmCmCmUmCmUmUmCmGmGmAmGmGmGmAmGm | |
| | CmAmUmCmAAAGNNNNNNNNNNNNNNNNNmN*mN*mN | |
| 174 (v4) | mA*mC*mU*mGmGmCmGmCUUUUmAmUmCmUmGmAmUUACUUUGmAmGm | 2139 |
| | AmGmCmCmAmUmCmAmCmCAGCGAmCmUAUmGmUmCmGUAGUGmGmGmU | |
| | mAmAmAmGmCmUmCmCmCmUmCmUmUmCmGmGmAmGmGmGmAmGmCmAm | |
| | UCAAAGNNNNNNNNNNNNNNNNNmN*mN*mN | |
| 174 (v5) | mA*mC*mU*GGCGCUUUUAUCUGAUUACUUUGAGAGCCAUCACCAGCGA | 2140 |
| | mCmUAUmGmUmCmGUAGUGGGUAAAmGmCmUmCmCmCmUmCmUmUmCmG | |
| | mGmAmGmGmGmAmGmCAUCAAAGNNNNNNNNNNNNNNNNNmN*mN*mN | |
| 174 (v6) | mA*mC*mU*GGCGCUUUUAUCUGAUUACUUUGAGAGCCAUCACCAGCGA | 2141 |
| | CUAUGUCGUAGUGGGUAAAmGmCmUmCmCmCmUmCmUmUmCmGmGmAmG | |
| | mGmGmAmGmCAUCAAAGNNNNNNNNNNNNNNNNNmN*mN*mN | |
| 174 (v7) | mA*mC*mU*GGmCGmCmUUUUAmUmCUGAUUACUUUGmAmGAGCCmAmU | 2142 |
| | mCmAmCCAGCmGmAmCmUAUmGmUmCmGUAGUGGmGmUAmAmAmGmCmU | |
| | mCmCmCmUmCmUmUmCmGmGmAmGmGmGmAmGmCmAmUCAAAGNNNNNN | |
| | NNNNNNNNNNN*mN*mN*mN | |
| 174 (v8) | mA*mC*mU*GGCGCUUUUAUCUGAUUACUUUGAGAGCCmAmUmCmAmCC | 2143 |
| | AGCmGmAmCmUAUmGmUmCmGUAGUGGmGmUmAmAAmGmCmUmCmCmCm | |
| | UmCmUmUmCmGmGmAmGmGmGmAmGmCmAmUCAAAGNNNNNNNNNNNNN | |
| | NNNN*mN*mN*mN | |
| 174 (v9) | mA*mC*mU*GGmCmGCmUUUUAmUmCUGAUUACUUUGmAmGAGCCAUCA | 2144 |
| | CCAGCmGmAmCmUAUmGmUmCmGUAGUGGGUAAAmGmCmUmCmCmCmUm | |
| | CmUmUmCmGmGmAmGmGmGmAmGmCAUCAAAGNNNNNNNNNNNNNNNNN | |
| | *mN*mN*mN | |
| 235 (v0) | ACUGGCGCUUCUAUCUGAUUACUCUGAGCGCCAUCACCAGCGACUAUGU | 2145 |
| | CGUAGUGGGUAAAGCCGCUUACGGACUUCGGUCCGUAAGAGGCAUCAGA | |
| | GNNNNNNNNNNNNNNNNNNNN | |
| 235 (v1) | mA*mC*mU*GGCGCUUCUAUCUGAUUACUCUGAGCGCCAUCACCAGCGA | 2146 |
| | CUAUGUCGUAGUGGGUAAAGCCGCUUACGGACUUCGGUCCGUAAGAGGC | |
| | AUCAGAGNNNNNNNNNNNNNNNNNmN*mN*mN | |
| 235 (v2) | mA*mC*mU*GGCGCUUCUAUCUGAUUACUCUGAGCGCCAUCACCAGCGA | 2147 |
| | CUAUGUCGUAGUGGGUAAAGCCGCUUACGGACUUCGGUCCGUAAGAGGC | |
| | AUCAGAGNNNNNNNNNNNNNNNNNNNN*mU*mU*mU | |
| 235 (v3) | mA*mC*mU*mGmGmCmGmCmUmUmCmUmAmUmCmUmGmAmUUACUCUGm | 2148 |
| | AmGmCmGmCmCmAmUmCmAmCmCAGCGAmCmUAUmGmUmCmGUAGUGmG | |
| | mGmUmAmAmAmGmCmCmGmCmUmUmAmCmGmGmAmCmUmUmCmGmGmUm | |
| | CmCmGmUmAmAmGmAmGmGmCmAmUmCmAGAGNNNNNNNNNNNNNNNNN | |
| | mN*mN*mN | |
| 235 (v4) | mA*mC*mU*mGmGmCmGmCUUCUmAmUmCmUmGmAmUUACUCUGmAmGm | 2149 |
| | CmGmCmCmAmUmCmAmCmCAGCGAmCmUAUmGmUmCmGUAGUGmGmGmU | |
| | mAmAmAmGmCmCmGmCmUmUmAmCmGmGmAmCmUmUmCmGmGmUmCmCm | |
| | GmUmAmAmGmAmGmGmCmAmUCAGAGNNNNNNNNNNNNNNNNNmN*mN* | |
| | mN | |
| 235 (v5) | mA*mC*mU*GGCGCUUCUAUCUGAUUACUCUGAGCGCCAUCACCAGCGA | 2150 |
| | mCmUAUmGmUmCmGUAGUGGGUmAmAmAmGmCmCmGmCmUmUmAmCmGm | |
| | GmAmCmUmUmCmGmGmUmCmCmGmUmAmAmGmAmGmGmCAUCAGAGNNN | |
| | NNNNNNNNNNNNNNmN*mN*mN | |
| 235 (v6) | mA*mC*mU*GGCGCUUCUAUCUGAUUACUCUGAGCGCCAUCACCAGCGA | 2151 |
| | CUAUGUCGUAGUGGGUmAmAmAmGmCmCmGmCmUmUmAmCmGmGmAmCm | |
| | UmUmCmGmGmUmCmCmGmUmAmAmGmAmGmGmCAUCAGAGNNNNNNNNN | |
| | NNNNNNNNmN*mN*mN | |
| 235 (v7) | mA*mC*mU*GGmCGmCmUUCUAmUmCUGAUUACUCUGmAmGCGCCmAmU | 2152 |
| | mCmAmCCAGCmGmAmCmUAUmGmUmCmGUAGUGGmGmUmAmAAmGmCmC | |
| | mGmCmUmUmAmCmGmGmAmCmUmUmCmGmGmUmCmCmGmUmAmAmGmAm | |
| | GmGmCmAmUCAGAGNNNNNNNNNNNNNNNNN*mN*mN*mN | |
| 235 (v8) | mA*mC*mU*GGCGCUUCUAUCUGAUUACUCUGAGCGCCmAmUmCmAmCC | 2153 |
| | AGCmGmAmCmUAUmGmUmCmGUAGUGGmGmUmAmAAmGmCmCmGmCmUm | |
| | UmAmCmGmGmAmCmUmUmCmGmGmUmCmCmGmUmAmAmGmAmGmGmCmA | |
| | mUCAGAGNNNNNNNNNNNNNNNNN*mN*mN*mN | |
| 235 (v9) | mA*mC*mU*GGmCGmCmUUCUAmUmCUGAUUACUCUGmAmGCGCCAUCA | 2154 |
| | CCAGCmGmAmCmUAUmGmUmCmGUAGUGGGUAAAmGmCmCmGmCmUmUm | |
| | AmCmGmGmAmCmUmUmCmGmGmUmCmCmGmUmAmAmGmAmGmGmCAUCA | |
| | GAGNNNNNNNNNNNNNNNNN*mN*mN*mN | |
| 316 (v0) | ACUGGCGCUUCUAUCUGAUUACUCUGAGCGCCAUCACCAGCGACUAUGU | 2155 |
| | CGUAGUGGGUAAAGCUCCCUCUUCGGAGGGAGCAUCAGAGNNNNNNNNN | |
| | NNNNNNNNNNN | |
| 316 (v1) | mA*mC*mU*GGCGCUUCUAUCUGAUUACUCUGAGCGCCAUCACCAGCGA | 2156 |
| | CUAUGUCGUAGUGGGUAAAGCUCCCUCUUCGGAGGGAGCAUCAGAGNNN | |
| | NNNNNNNNNNNNNN*mN*mN*mN | |
| 316 (v2) | mA*mC*mU*GGCGCUUCUAUCUGAUUACUCUGAGCGCCAUCACCAGCGA | 2157 |
| | CUAUGUCGUAGUGGGUAAAGCUCCCUCUUCGGAGGGAGCAUCAGAGNNN | |
| | NNNNNNNNNNNNNNNNN*mU*mU*mU | |
| 316 (v3) | mA*mC*mU*mGmGmCmGmCmUmUmCmUmAmUmCmUmGmAmUUACUCUGm | 2158 |
| | AmGmCmGmCmCmAmUmCmAmCmCAGCGAmCmUAUmGmUmCmGUAGUGmG | |
| | mGmUmAmAmAmGmCmUmCmCmCmUmCmUmUmCmGmGmAmGmGmGmAmGm | |
| | CmAmUmCmAGAGNNNNNNNNNNNNNNNNN*mN*mN*mN | |
| 316 (v4) | mA*mC*mU*mGmGmCmGmCUUCUmAmUmCmUmGmAmUUACUCUGmAmGm | 2159 |
| | CmGmCmCmAmUmCmAmCmCAGCGAmCmUAUmGmUmCmGUAGUGmGmGmU | |
| | mAmAmAmGmCmUmCmCmCmUmCmUmUmCmGmGmAmGmGmGmAmGmCmAm | |
| | UCAGAGNNNNNNNNNNNNNNNNN*mN*mN*mN | |
| 316 (v5) | mA*mC*mU*GGCGCUUCUAUCUGAUUACUCUGAGCGCCAUCACCAGCGA | 2160 |
| | mCmUAUmGmUmCmGUAGUGGGUAAAmGmCmUmCmCmCmUmCmUmUmCmG | |
| | mGmAmGmGmGmAmGmCAUCAGAGNNNNNNNNNNNNNNNNN*mN*mN*mN | |
| 316 (v6) | mA*mC*mU*GGCGCUUCUAUCUGAUUACUCUGAGCGCCAUCACCAGCGA | 2161 |
| | CUAUGUCGUAGUGGGUAAAmGmCmUmCmCmCmUmCmUmUmCmGmGmAmG | |
| | mGmGmAmGmCAUCAGAGNNNNNNNNNNNNNNNNN*mN*mN*mN | |
| 316 (v7) | mA*mC*mU*GGmCGmCmUUCUAmUmCUGAUUACUCUGmAmGCGCCmAmU | 2162 |
| | mCmAmCCAGCmGmAmCmUAUmGmUmCmGUAGUGGmGmUmAmAAmGmCmU | |
| | mCmCmCmUmCmUmUmCmGmGmAmGmGmGmAmGmCmAmUCAGAGNNNNNN | |
| | NNNNNNNNNNN*mN*mN*mN | |
| 316 (v8) | mA*mC*mU*GGCGCUUCUAUCUGAUUACUCUGAGCGCCmAmUmCmAmCC | 2163 |
| | AGCmGmAmCmUAUmGmUmCmGUAGUGGmGmUmAmAAmGmCmUmCmCmCm | |
| | UmCmUmUmCmGmGmAmGmGmGmAmGmCmAmUCAGAGNNNNNNNNNNNNN | |
| | NNNN*mN*mN*mN | |
| 316 (v9) | mA*mC*mU*GGmCGmCmUUCUAmUmCUGAUUACUCUGmAmGCGCCAUCA | 2164 |
| | CCAGCmGmAmCmUAUmGmUmCmGUAGUGGGUAAAmGmCmUmCmCmCmUm | |
| | CmUmUmCmGmGmAmGmGmGmAmGmCAUCAGAGNNNNNNNNNNNNNNNNN | |
| | *mN*mN*mN | |

TABLE 45
Sequences of gRNAs with their different chemical
modification profiles (denoted by version number)
assayed in this example. Chemical modifications:
* = phosphorothioate bond; m = 2′OMe modification

| gRNA ID | | | |
|---|---|---|---|
| (scaffold | | | SEQ |
| variant- | | | ID |
| spacer) | Target | gRNA sequence | NO: |
| 174-6.7 | human | ACUGGCGCUUUUAUCUGAUUACUUUGAGAGCCAUCACCAGCGAC | 2165 |
| (v0) | PCSK9 | UAUGUCGUAGUGGGUAAAGCUCCCUCUUCGGAGGGAGCAUCAAA | |
| | | GUCCUGGCUUCCUGGUGAAGA | |
| 174-6.7 | human | mA*mC*mU*GGCGCUUUUAUCUGAUUACUUUGAGAGCCAUCACC | 2166 |
| (v1) | PCSK9 | AGCGACUAUGUCGUAGUGGGUAAAGCUCCCUCUUCGGAGGGAGC | |
| | | AUCAAAGUCCUGGCUUCCUGGUGAmA*mG*mA | |
| 174-6.8 | human | ACUGGCGCUUUUAUCUGAUUACUUUGAGAGCCAUCACCAGCGAC | 2167 |
| (v0) | PCSK9 | UAUGUCGUAGUGGGUAAAGCUCCCUCUUCGGAGGGAGCAUCAAA | |
| | | GUGGCUUCCUGGUGAAGAUGA | |
| 174-6.8 | human | mA*mC*mU*GGCGCUUUUAUCUGAUUACUUUGAGAGCCAUCACC | 2168 |
| (v1) | PCSK9 | AGCGACUAUGUCGUAGUGGGUAAAGCUCCCUCUUCGGAGGGAGC | |
| | | AUCAAAGUGGCUUCCUGGUGAAGAmU*mG*mA | |
| 174-7.9 | human | ACUGGCGCUUUUAUCUGAUUACUUUGAGAGCCAUCACCAGCGAC | 2169 |
| (v0) | B2M | UAUGUCGUAGUGGGUAAAGCUCCCUCUUCGGAGGGAGCAUCAAA | |
| | | GGUGUAGUACAAGAGAUAGAA | |
| 174-7.9 | human | mA*mC*mU*GGCGCUUUUAUCUGAUUACUUUGAGAGCCAUCACC | 2170 |
| (v1) | B2M | AGCGACUAUGUCGUAGUGGGUAAAGCUCCCUCUUCGGAGGGAGC | |
| | | AUCAAAGGUGUAGUACAAGAGAUAmG*mA*mA | |
| 316-6.7 | human | ACUGGCGCUUCUAUCUGAUUACUCUGAGCGCCAUCACCAGCGAC | 2171 |
| (v0) | PCSK9 | UAUGUCGUAGUGGGUAAAGCUCCCUCUUCGGAGGGAGCAUCAGA | |
| | | GUCCUGGCUUCCUGGUGAAGA | |
| 316-6.7 | human | mA*mC*mU*GGCGCUUCUAUCUGAUUACUCUGAGCGCCAUCACC | 2172 |
| (v1′) | PCSK9 | AGCGACUAUGUCGUAGUGGGUAAAGCUCCCUCUUCGGAGGGAGC | |
| | | AUCAGAGUCCUGGCUUCCUGGUGAmA*mG*mA | |
| 316-6.8 | human | ACUGGCGCUUCUAUCUGAUUACUCUGAGCGCCAUCACCAGCGAC | 2173 |
| (v0) | PCSK9 | UAUGUCGUAGUGGGUAAAGCUCCCUCUUCGGAGGGAGCAUCAGA | |
| | | GUGGCUUCCUGGUGAAGAUGA | |
| 316-6.8 | human | mA*mC*mU*GGCGCUUCUAUCUGAUUACUCUGAGCGCCAUCACC | 2174 |
| (v1′) | PCSK9 | AGCGACUAUGUCGUAGUGGGUAAAGCUCCCUCUUCGGAGGGAGC | |
| | | AUCAGAGUGGCUUCCUGGUGAAGAmU*mG*mA | |
| 316-7.9 | human | ACUGGCGCUUCUAUCUGAUUACUCUGAGCGCCAUCACCAGCGAC | 2175 |
| (v0) | B2M | UAUGUCGUAGUGGGUAAAGCUCCCUCUUCGGAGGGAGCAUCAGA | |
| | | GGUGUAGUACAAGAGAUAGAA | |
| 316-7.9 | human | mA*mC*mU*GGCGCUUCUAUCUGAUUACUCUGAGCGCCAUCACC | 2176 |
| (v1′) | B2M | AGCGACUAUGUCGUAGUGGGUAAAGCUCCCUCUUCGGAGGGAGC | |
| | | AUCAGAGGUGUAGUACAAGAGAUAmG*mA*mA | |
| 174-7.37 | human | ACUGGCGCUUUUAUCUgAUUACUUUGAGAGCCAUCACCAGCGAC | 2177 |
| (v0) | B2M | UAUGUCGUAgUGGGUAAAGCUCCCUCUUCGGAGGGAGCAUCAAA | |
| | | GGGCCGAGAUGUCUCGCUC | |
| 174-7.37 | human | mA*mC*mU*GGCGCUUUUAUCUgAUUACUUUGAGAGCCAUCACC | 2178 |
| (v1*) | B2M | AGCGACUAUGUCGUAgUGGGUAAAGCUCCCUCUUCGGAGGGAGC | |
| | | AUCAAAGGGCCGAGAUGUCUCG*mC*mU*mC | |
| 235-6.7 | human | ACUGGCGCUUCUAUCUGAUUACUCUGAGCGCCAUCACCAGCGAC | 2179 |
| (v0) | PCSK9 | UAUGUCGUAGUGGGUAAAGCCGCUUACGGACUUCGGUCCGUAAG | |
| | | AGGCAUCAGAGUCCUGGCUUCCUGGUGAAGA | |
| 235-6.7 | human | mA*mC*mU*GGCGCUUCUAUCUGAUUACUCUGAGCGCCAUCACC | 2180 |
| (v1) | PCSK9 | AGCGACUAUGUCGUAGUGGGUAAAGCCGCUUACGGACUUCGGUC | |
| | | CGUAAGAGGCAUCAGAGUCCUGGCUUCCUGGUGAmA*mG*mA | |
| 235-6.7 | human | mA*mC*mU*GGCGCUUCUAUCUGAUUACUCUGAGCGCCAUCACC | 2181 |
| (v2) | PCSK9 | AGCGACUAUGUCGUAGUGGGUAAAGCCGCUUACGGACUUCGGUC | |
| | | CGUAAGAGGCAUCAGAGUCCUGGCUUCCUGGUGAAGAU*mU*mU | |
| | | *mU | |
| 235-6.7 | human | mA*mC*mU*mGmGmCmGmCmUmUmCmUmAmUmCmUmGmAmUUAC | 2182 |
| (v3) | PCSK9 | UCUGmAmGmCmGmCmCmAmUmCmAmCmCAGCGAmCmUAUmGmUm | |
| | | CmGUAGUGmGmGmUmAmAmAmGmCmCmGmCmUmUmAmCmGmGmA | |
| | | mCmUmUmCmGmGmUmCmCmGmUmAmAmGmAmGmGmCmAmUmCmA | |
| | | GAGUCCUGGCUUCCUGGUGAmA*mG*mA | |
| 235-6.7 | human | mA*mC*mU*mGmGmCmGmCUUCUmAmUmCmUmGmAmUUACUCUG | 2183 |
| (v4) | PCSK9 | mAmGmCmGmCmCmAmUmCmAmCmCAGCGAmCmUAUmGmUmCmGU | |
| | | AGUGmGmGmUmAmAmAmGmCmCmGmCmUmUmAmCmGmGmAmCmU | |
| | | mUmCmGmGmUmCmCmGmUmAmAmGmAmGmGmCmAmUCAGAGUCC | |
| | | UGGCUUCCUGGUGAmA*mG*mA | |
| 235-6.7 | human | mA*mC*mU*GGCGCUUCUAUCUGAUUACUCUGAGCGCCAUCACC | 2184 |
| (v5) | PCSK9 | AGCGAmCmUAUmGmUmCmGUAGUGGGUmAmAmAmGmCmCmGmCm | |
| | | UmUmAmCmGmGmAmCmUmUmCmGmGmUmCmCmGmUmAmAmGmAm | |
| | | GmGmCAUCAGAGUCCUGGCUUCCUGGUGAmA*mG*mA | |
| 235-6.7 | human | mA*mC*mU*GGCGCUUCUAUCUGAUUACUCUGAGCGCCAUCACC | 2185 |
| (v6) | PCSK9 | AGCGACUAUGUCGUAGUGGGUmAmAmAmGmCmCmGmCmUmUmAm | |
| | | CmGmGmAmCmUmUmCmGmGmUmCmCmGmUmAmAmGmAmGmGmCA | |
| | | UCAGAGUCCUGGCUUCCUGGUGAmA*mG*mA | |
| 235-6.8 | human | ACUGGCGCUUCUAUCUGAUUACUCUGAGCGCCAUCACCAGCGAC | 2186 |
| (v0) | PCSK9 | UAUGUCGUAGUGGGUAAAGCCGCUUACGGACUUCGGUCCGUAAG | |
| | | AGGCAUCAGAGUGGCUUCCUGGUGAAGAUGA | |
| 235-6.8 | human | mA*mC*mU*GGCGCUUCUAUCUGAUUACUCUGAGCGCCAUCACC | 2187 |
| (v1) | PCSK9 | AGCGACUAUGUCGUAGUGGGUAAAGCCGCUUACGGACUUCGGUC | |
| | | CGUAAGAGGCAUCAGAGUGGCUUCCUGGUGAAGAmU*mG*mA | |
| 235-6.8 | human | mA*mC*mU*GGCGCUUCUAUCUGAUUACUCUGAGCGCCAUCACC | 2188 |
| (v2) | PCSK9 | AGCGACUAUGUCGUAGUGGGUAAAGCCGCUUACGGACUUCGGUC | |
| | | CGUAAGAGGCAUCAGAGUGGCUUCCUGGUGAAGAUGA*mU*mU* | |
| | | mU | |
| 235-6.8 | human | mA*mC*mU*mGmGmCmGmCmUmUmCmUmAmUmCmUmGmAmUUAC | 2189 |
| (v3) | PCSK9 | UCUGmAmGmCmGmCmCmAmUmCmAmCmCAGCGAmCmUAUmGmUm | |
| | | CmGUAGUGmGmGmUmAmAmAmGmCmCmGmCmUmUmAmCmGmGmA | |
| | | mCmUmUmCmGmGmUmCmCmGmUmAmAmGmAmGmGmCmAmUmCmA | |
| | | GAGUGGCUUCCUGGUGAAGAmU*mG*mA | |
| 235-6.8 | human | mA*mC*mU*mGmGmCmGmCUUCUmAmUmCmUmGmAmUUACUCUG | 2190 |
| (v4) | PCSK9 | mAmGmCmGmCmCmAmUmCmAmCmCAGCGAmCmUAUmGmUmCmGU | |
| | | AGUGmGmGmUmAmAmAmGmCmCmGmCmUmUmAmCmGmGmAmCmU | |
| | | mUmCmGmGmUmCmCmGmUmAmAmGmAmGmGmCmAmUCAGAGUGG | |
| | | CUUCCUGGUGAAGAmU*mG*mA | |
| 235-6.8 | human | mA*mC*mU*GGCGCUUCUAUCUGAUUACUCUGAGCGCCAUCACC | 2191 |
| (v5) | PCSK9 | AGCGAmCmUAUmGmUmCmGUAGUGGGUmAmAmAmGmCmCmGmCm | |
| | | UmUmAmCmGmGmAmCmUmUmCmGmGmUmCmCmGmUmAmAmGmAm | |
| | | GmGmCAUCAGAGUGGCUUCCUGGUGAAGAmU*mG*mA | |
| 235-6.8 | human | mA*mC*mU*GGCGCUUCUAUCUGAUUACUCUGAGCGCCAUCACC | 2192 |
| (v6) | PCSK9 | AGCGACUAUGUCGUAGUGGGUmAmAmAmGmCmCmGmCmUmUmAm | |
| | | CmGmGmAmCmUmUmCmGmGmUmCmCmGmUmAmAmGmAmGmGmCA | |
| | | UCAGAGUGGCUUCCUGGUGAAGAmU*mG*mA | |
| 316-27.107 | mouse | ACUGGCGCUUCUAUCUGAUUACUCUGAGCGCCAUCACCAGCGAC | 2193 |
| (v0) | PCSK9 | UAUGUCGUAGUGGGUAAAGCUCCCUCUUCGGAGGGAGCAUCAGA | |
| | | GCUGGCUUCUUGGUGAAGAUG | |
| 316-27.107 | mouse | mA*mC*mU*GGCGCUUCUAUCUGAUUACUCUGAGCGCCAUCACC | 2194 |
| (v1) | PCSK9 | AGCGACUAUGUCGUAGUGGGUAAAGCUCCCUCUUCGGAGGGAGC | |
| | | AUCAGAGCUGGCUUCUUGGUGAAG*mA*mU*mG | |
| 316-27.107 | mouse | mA*mC*mU*GGmCGmCmUUCUAmUmCUGAUUACUCUGmAmGCGC | 2195 |
| (v7) | PCSK9 | CmAmUmCmAmCCAGCmGmAmCmUAUmGmUmCmGUAGUGGmGmUm | |
| | | AmAAmGmCmUmCmCmCmUmCmUmUmCmGmGmAmGmGmGmAmGmC | |
| | | mAmUCAGAGCUGGCUUCUUGGUGAAG*mA*mU*mG | |
| 316-27.107 | mouse | mA*mC*mU*GGCGCUUCUAUCUGAUUACUCUGAGCGCCmAmUmC | 2196 |
| (v8) | PCSK9 | mAmCCAGCmGmAmCmUAUmGmUmCmGUAGUGGmGmUmAmAAmGm | |
| | | CmUmCmCmCmUmCmUmUmCmGmGmAmGmGmGmAmGmCmAmUCAG | |
| | | AGCUGGCUUCUUGGUGAAG*mA*mU*mG | |
| 316-27.107 | mouse | mA*mC*mU*GGmCGmCmUUCUAmUmCUGAUUACUCUGmAmGCGC | 2197 |
| (v9*) | PCSK9 | CAUCACCAGCmGmAmCmUAUmGmUmCmGUAGUGGGUAAAmGmCm | |
| | | UmCmCmCmUmCmUmUmCmGmGmAmGmGmGmAmGmCAUCAGAGCU | |
| | | GGCUUCUUGGUGAA*mG*mA*mU*mG | |
| 174-35.2 | ROSA26 | ACUGGCGCUUUUAUCUGAUUACUUUGAGAGCCAUCACCAGCGAC | 2198 |
| (v0) | | UAUGUCGUAGUGGGUAAAGCUCCCUCUUCGGAGGGAGCAUCAAA | |
| | | GAGAAGAUGGGCGGGAGUCUU | |
| 174-35.2 | ROSA26 | mA*mC*mU*GGCGCUUUUAUCUGAUUACUUUGAGAGCCAUCACC | 2199 |
| (v2) | | AGCGACUAUGUCGUAGUGGGUAAAGCUCCCUCUUCGGAGGGAGC | |
| | | AUCAAAGAGAAGAUGGGCGGGAGUCUU*mU*mU*mU | |
| 316-35.2 | ROSA26 | ACUGGCGCUUCUAUCUGAUUACUCUGAGCGCCAUCACCAGCGAC | 2200 |
| (v0) | | UAUGUCGUAGUGGGUAAAGCUCCCUCUUCGGAGGGAGCAUCAGA | |
| | | GAGAAGAUGGGCGGGAGUCUU | |
| 316-35.2 | ROSA26 | mA*mC*mU*GGCGCUUCUAUCUGAUUACUCUGAGCGCCAUCACC | 2201 |
| (v1) | | AGCGACUAUGUCGUAGUGGGUAAAGCUCCCUCUUCGGAGGGAGC | |
| | | AUCAGAGAGAAGAUGGGCGGGAGU*mC*mU*mU | |
| 316-35.2 | ROSA26 | mA*mC*mU*GGCGCUUCUAUCUGAUUACUCUGAGCGCCAUCACC | 2202 |
| (v5) | | AGCGAmCmUAUmGmUmCmGUAGUGGGUAAAmGmCmUmCmCmCmU | |
| | | mCmUmUmCmGmGmAmGmGmGmAmGmCAUCAGAGAGAAGAUGGGC | |
| | | GGGAGU*mC*mU*mU | |

Note that gRNAs annotated with a v1′ design contain one less phosphorothioate bond on the 3′ end of the gRNA. gRNAs annotated with v1* contain one extra phosphorothioate bond on the 3′end of the gRNA. gRNAs annotated with a v9* contain an extra phosphorothioate bond on the 3′ end of the gRNA.
Biochemical Characterization of gRNA Activity:

Target DNA oligonucleotides with fluorescent moieties on the 5′ ends were purchased commercially (sequences listed in Table 46). Double-stranded DNA (dsDNA) targets were formed by mixing the oligos in a 1:1 ratio in 1× cleavage buffer (20 mM Tris HCl pH 7.5, 150 mM NaCl, 1 mM TCEP, 500 glycerol, 10 mM MgCl2), following by heating to 95° C. for 10 minutes, and then allowing the solution to cool to room temperature. CasX ribonucleoproteins (RNPs) were reconstituted with CasX 491 and the indicated gRNAs at a final concentration of 1 μM with 1.2-fold excess of the indicated gRNA in 1× cleavage buffer. RNPs were allowed to form at 37° C. for 10 minutes.

The effects of various structural and chemical modifications to the gRNA scaffold on the cleavage rate of CasX 491 RNPs were determined. Cleavage reactions were prepared with final RNP concentrations of 200 nM and final target concentrations of 10 nM, and reactions were carried out at 16° C. and initiated by the addition of the labeled target DNA substrate (Table 46). Aliquots of reactions were taken at 0.25, 0.5, 1, 2, 5, and 10 minutes and quenched by adding an equal volume of 95% formamide with 20 mM EDTA. Samples were denatured at 95° C. for 10 minutes and resolved on a 10% urea-PAGE gel. Gels were imaged on a Typhoon™ laser-scanner platform and quantified using ImageQuant™ TL 8.2 image analysis software (Cytiva™). The apparent first-order rate constant of non-target strand cleavage (kcieave-) was determined for each CasX:gRNA combination.

To determine the competent fraction formed by each gRNA, cleavage reactions were prepared with final RNP concentrations of 100 nM and final target concentrations of 100 nM. Reactions were carried out at 37° C. and initiated by the addition of the labeled target substrate (Table 46). Aliquots were taken at 0.5, 1, 2, 5, 10, and 30 minutes and quenched by adding an equal volume of 95% formamide with 25 mM EDTA. Samples were denatured by heating at 95° C. for 10 minutes and resolved on a 10% urea-PAGE gel. Gels were imaged and quantified as above. CasX was assumed to act as a single-turnover enzyme under the assayed conditions, as indicated by the observation that sub-stoichiometric amounts of enzyme would fail to cleave a greater-than-stoichiometric amount of target substrate even under extended time-scales, and instead would approach a plateau that scaled with the amount of enzyme present. Thus, the fraction of target substrate cleaved over long time-scales by an equimolar amount of RNP would be indicative of the fraction of RNP that was properly formed and active for cleavage. The cleavage traces were fitted with a biphasic rate model, as the cleavage reaction clearly deviated from monophasic under this concentration regime. The plateau of each fit was determined and reported as the active fraction for each RNP in Table 49.

TABLE 46
Sequences of target DNA substrate
oligonucleotides with fluorescent moieties
on the 5′ ends used for biochemical
characterization of gRNA activity.
/700/ = IRDye700; /800/ = IRDye800

| DNA substrate | Sequence |
|---|---|
| 6.7/6.8 target | /700/catgtcttccatggccttcttc |
| top strand | ctggcttcctggtgaagatgagtggcg |
| (SEQ ID NO: | acctgctggag |
| 2203) | |
| 6.7/6.8 target | /800/ctccagcaggtcgccactcatc |
| bottom strand | ttcaccaggaagccaggaagaaggcca |
| (SEQ ID NO: | tggaagacatg |
| 2204) | |

In Vitro Transcription of CasX mRNA:

DNA templates encoding for CasX 491 (see Table 47 for encoding sequences) used for in vitro transcription were generated by PCR using forward primers containing a T7 promoter, followed by agarose gel extraction of the appropriately sized DNA. 25 ng/μL final concentration of template DNA was used in each in vitro transcription reaction that was carried out following the manufacturer's recommended protocol with slight modifications. Following in vitro transcription reaction incubation for 2-3 hours at 37° C., which were carried out with CleanCap® AG and N1-methyl-pseudouridine, DNAse digestion of template DNA and column-based purification using the Zymo RNA miniprep kit were performed. The poly(A) tail was added using E. coli PolyA Polymerase following the manufacturer's protocol, followed by column-based purification as stated above. Poly(A) tailed in vitro transcribed RNA was eluted in RNAse free water, analyzed on an Agilent TapeStation for integrity, and flash frozen prior to storage at ~80° C.

TABLE 47
Encoding sequences of the CasX mRNA
molecules assessed in this example*

| CasX 491 | | DNA sequence |
|---|---|---|
| mRNA ID | Component (ID) | SEQ ID NO: |
| CasX 491 | 5′UTR | 2017 |
| mRNA #1 | START codon + c-MYC NLS + linker | 2205 |
| | CasX 491 | 2206 |
| | Linker + c-MYC NLS | 2207 |
| | P2A mScarlet + STOP codon | 2208 |
| CasX 676 | 5′UTR | 1925 |
| mRNA #2 | START codon + c-MYC NLS | 1958 |
| | CasX 676 | 1959 |
| | c-MYC NLS + STOP codons | 1960 |
| | 3′UTR | 1936 |
| | XbaI restriction site (partial) | 1937 |
| | Poly(A) tail | 1961 |

*Components are listed in a 5′ to 3′ order within the constructs

In Vitro Delivery of gRNA and CasX mRNA Via Transfection:

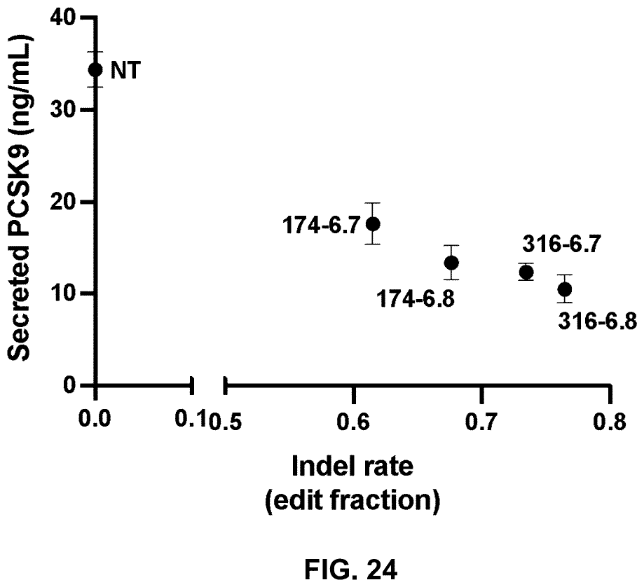

Editing at the PCSK9 locus and consequential effects on secreted PCSK9 levels were assessed for conditions using CasX 491 mRNA co-delivered with a PCSK9-targeting gRNA with scaffold variant 174 compared to conditions where a PCSK9-targeting gRNA with scaffold variant 316 was used. 100 ng of in vitro transcribed mRNA coding for CasX 491 with a P2A and mScarlet fluorescent protein was transfected into HepG2 cells with version 1 (v1) of gRNAs 174-6.7, 174-6.8, 316-6.7, and 316-6.8 (see Table 45) using lipofectamine. After a media change, the following were harvested at 28 hours post-transfection: 1) transfected cells were harvested for editing assessment at the PCSK9 locus by NGS (next generation sequencing); and 2) media supernatant was harvested to measure secreted PCSK9 protein levels by ELISA. For editing analysis by NGS, amplicons were amplified from 200 ng of extracted gDNA with a set of primers targeting the PCSK9 locus and processed as described earlier in Example 1. Secreted PCSK9 levels in the media supernatant were also analyzed using a fluorescence resonance energy transfer-based immunoassay from CISBio following the manufacturer's instructions. Here, a gRNA using scaffold 174 with spacer 7.37 (v0; see Table 45), which targeted the endogenous B2M(beta-2-microglobulin) locus, served as the non-targeting (NT) control. These results are shown in FIG. 24.

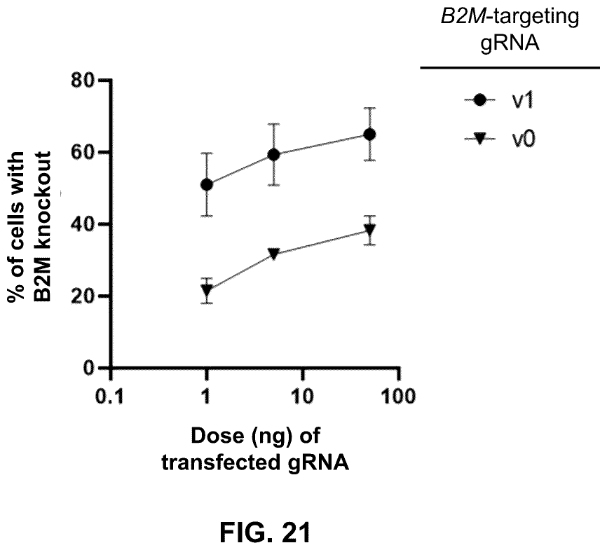

To compare the editing potency of version 0 (v0) and version 1 (v1) of B2M-targeting gRNAs, ~6E4 HepG2 hepatocytes were seeded per well of a 96-well plate. 24 hours later, seeded cells were co-transfected using lipofectamine with 100 ng of in vitro transcribed mRNA coding for CasX 491 and different doses (1, 5, or 50 ng) of either v0 or v1 of the B2M-targeting gRNA containing scaffold variant 174 and spacer 7.37 (see Table 45). Six days post-transfection, cells were harvested for B2M protein expression analysis via immunostaining of the B2M-dependent HLA protein, followed by flow cytometry using the Attune™ NxT flow cytometer. These results are shown in FIG. 21.

V1 through v6 variants of chemically-modified PCSK9-targeting gRNAs (Table 45) were assessed for their effects on editing potency and consequential effects on secreted PCSK9 levels in vitro. Briefly, 100 ng of in vitro transcribed mRNA coding for CasX variant 491 and a P2A and mScarlet fluorescent protein was transfected into HepG2 cells with 50 ng of the indicated chemically-modified gRNA using lipofectamine. After a media change, the following were harvested at 28 hours post-transfection: 1) transfected cells for editing assessment at the PCSK9 locus by NGS as described above; and 2) media supernatant to measure secreted PCSK9 protein levels by ELISA, as described above. Here, a B2M-targeting gRNA was used as a non-targeting control. These results are shown in Table 50.

Briefly, to formulate LNPs (lipid nanoparticle), equal mass ratios of XR or LTRP mRNA and gRNA are diluted in PNI Formulation Buffer, pH 4.0. GenVoy-ILM™ lipids are diluted 1:1 in anhydrous ethanol. mRNA/gRNA co-formulations are generated using a predetermined N/P ratio. The RNA and lipids are run through a PNI laminar flow cartridge at a predetermined flow rate ratio on the PNI Ignite™ Benchtop System. After formulation, the LNPs are diluted in PBS, pH 7.4, to decrease the ethanol concentration and increase the pH, which increases the stability of the particles. Buffer exchange of the mRNA/sgRNA-LNPs is achieved by overnight dialysis into PBS, pH 7.4, at 4° C. using 10k Slide-A-Lyzer™ Dialysis Cassettes (Thermo Scientific™). Following dialysis, the mRNA/gRNA-LNPs is concentrated to >0.5 mg/mL using 100 kDa Amicon®-Ultra Centrifugal Filters (Millipore) and then filter-sterilized. Formulated LNPs are analyzed on a Stunner (Unchained Labs) to determine their diameter and polydispersity index (PDI). Encapsulation efficiency and RNA concentration is determined by RiboGreen™ assay using Invitrogen's Quant-iT™ RiboGreen™ RNA assay kit.

Delivery of LNPs Encapsulating CasX mRNA and Targeting gRNAs In Vitro:

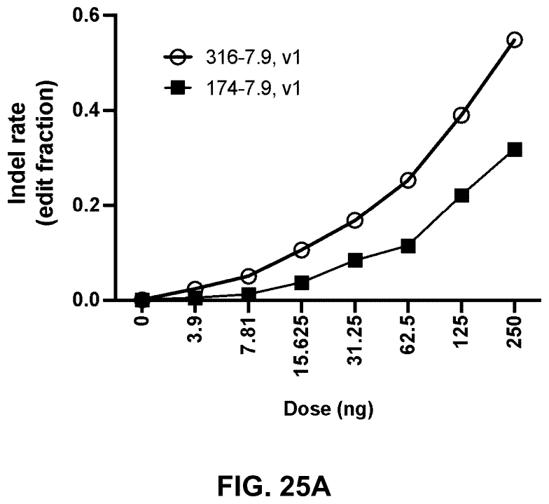

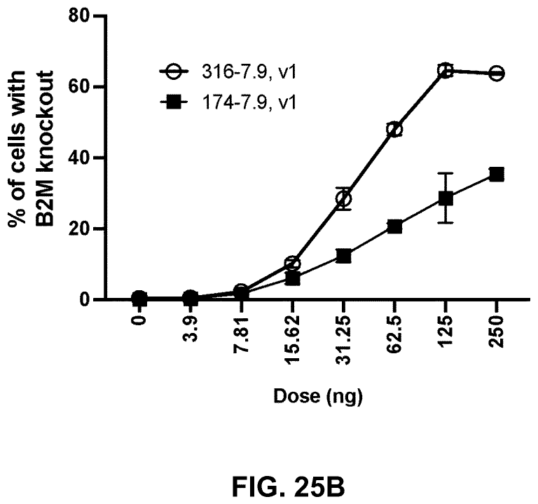

~50,000 HepG2 cells, cultured in DMEM/F-12 media containing 10% FBS and 1% PenStrep, were seeded per well in a 96-well plate. The next day, seeded cells were treated with varying concentrations of LNPs, which were prepared in six 2-fold serial dilutions starting at 250 ng. These LNPs were formulated to encapsulate CasX 491 mRNA and a B2M-targeting gRNA incorporating either scaffold variant 174 or 316 with spacer 7.9 (v1; see Table 45). Media was changed 24 hours after LNP treatment, and cells were cultured for six additional days prior to harvesting for gDNA extraction for editing assessment at the B2M locus by NGS and B2M protein expression analysis via HLA immunostaining, followed by flow cytometry using the Attune NxT flow cytometer. Briefly, for editing assessment, amplicons were amplified from 200 ng of extracted gDNA with primers targeting the human B2M locus and processed as described in Example 1. The results of these assays are shown in FIGS. 25A and 25B.

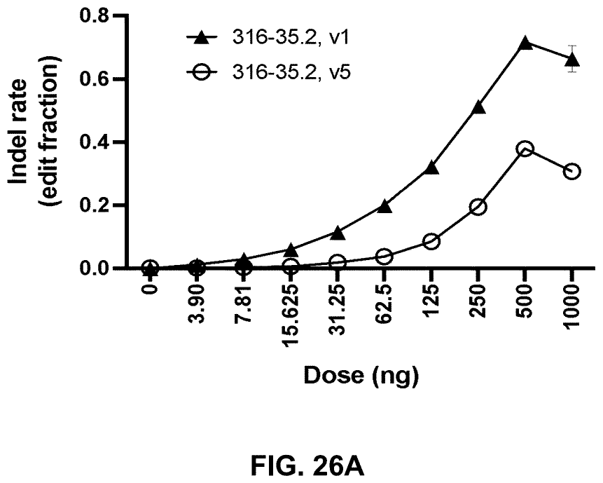

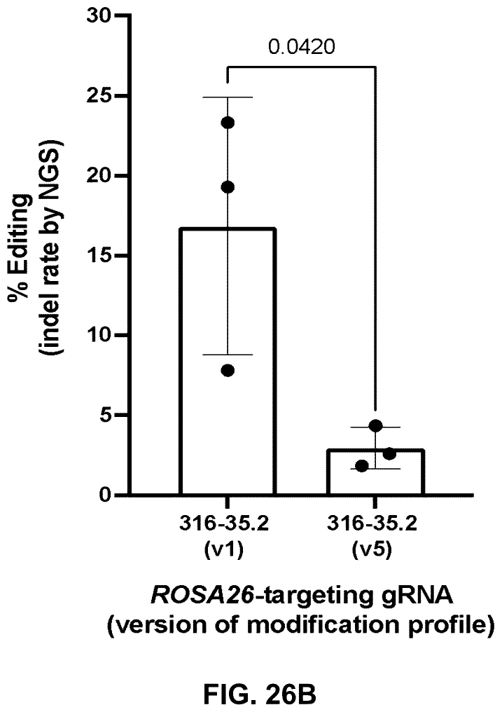

~20,000 mouse Hepa1-6 hepatocytes were seeded per well in a 96-well plate. The following day, seeded cells were treated with varying concentrations of LNPs, which were prepared in eight 2-fold serial dilutions starting at 1000 ng. These LNPs were formulated to encapsulate CasX 676 mRNA #2 (see Table 47) and a ROSA26-targeting gRNA incorporating scaffold variant 316 with spacer 35.2 (v1 or 5; see Table 45). Media was changed 24 hours post-treatment with LNPs, and cells were cultured for seven additional days prior to harvesting for gDNA extraction for editing assessment at the ROSA26 locus by NGS. Briefly, amplicons were amplified from extracted gDNA with primers targeting the mouse ROSA26 locus and processed as described in Example 4. The results of this experiment are shown in FIG. 26A. Delivery of LNPs encapsulating CasX mRNA and targeting gRNA in vivo:

LNP co-formulations were performed as described in Example 11.

To assess the effects of using v1 and v5 of scaffold 316 in vivo, CasX 676 mRNA #2 (see Table 47) and a ROSA26-targeting gRNA using scaffold 316 with spacer 35.2 (v1 or v5; see Table 45) were encapsulated within the same LNP using a 1:1 mass ratio for mRNA:gRNA. Formulated LNPs were buffer-exchanged to PBS for in vivo injection. Briefly, LNPs were administered retro-orbitally into 4-week old C57BL/6 mice. Six days post-administration, mice were euthanized, and the liver tissue was harvested for gDNA extraction using the Zymo Research Quick DNA/RNA Miniprep kit following the manufacturer's instructions. Target amplicons were then amplified from the extracted gDNA with a set of primers targeting the mouse ROSA26 locus and processed as described earlier in Example 1 for editing assessment by NGS. The results of this experiment are shown in FIG. 26B.

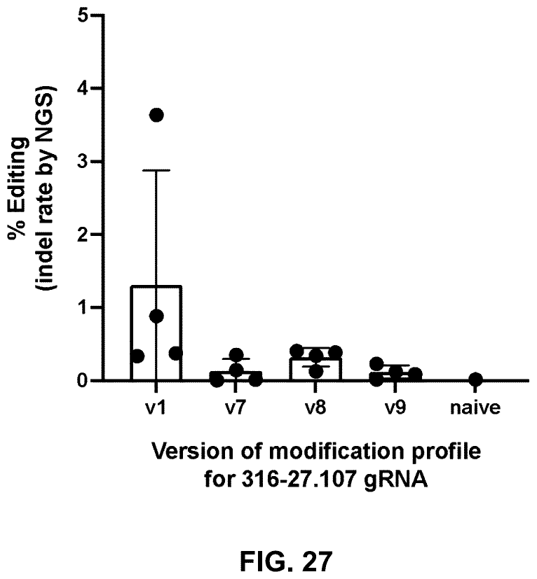

To compare the effects of using v7, v8, and v9 of scaffold 316 on editing at the PCSK9 locus in vivo, CasX 676 mRNA #1 (see Table 48 for sequences) and aPCSK9-targeting gRNA using scaffold 316 with spacer 27.107 (v1, v7, v8, or v9; see Table 45), were encapsulated within the same LNP using a 1:1 mass ratio for mRNA:gRNA for each gRNA. Briefly, LNPs were administered retro-orbitally into 6-week old C57BL/6 mice, and mice were euthanized seven days post-injection to harvest liver tissue for gDNA extraction for editing assessment by NGS at the PCSK9 locus. The results of this experiment are shown in FIG. 27.

TABLE 48
Encoding sequences of CasX 676 mRNA #1 molecule

| | | | DNA sequence |
|---|---|---|---|
| CasX ID | Component (ID) | Description | SEQ ID NO: |
| CasX 676 | 5′UTR | hHBA | 2209 |
| mRNA #1 | START codon + c-MYC NLS | | 1958 |
| | CasX 676 | | 1959 |
| | c-MYC NLS + STOP codons | | 1960 |
| | 3′UTR | hHBA | 2210 |
| | Poly(A) tail | | 1961 |

*Components are listed in a 5′ to 3′ order within the constructs

### Results:

Assessing the Effects of Various Chemical Modifications on gRNA Activity:

Several studies involving Cas9 have demonstrated that chemical modifications of the gRNA resulted in significantly improved editing activity when delivered with Cas9 mRNA. Following delivery of Cas9 mRNA and gRNA into target cells, unprotected gRNA is susceptible to degradation during the mRNA translation process. Addition of chemical modifications such as 2′O-methyl (2′OMe) groups and phosphorothioate bonds can reduce the susceptibility of the gRNA to cellular RNases, but also have the potential to disrupt folding of the gRNA and its interactions with the CRISPR-Cas protein. Given the lack of structural similarity between CasX and Cas9, as well as their respective gRNAs, appropriate chemical modification profiles must be designed and validated de novo. Using published structures of wild-type CasX from Deltaproteobacteria (PDB codes 6NY1, 6NY2, and 6NY3) as reference, residues that appeared potentially amenable to modification were selected. However, the published structures were of a wild type CasX ortholog and gRNA distinct from the species used as the basis for the engineered variants presented here, and they also lacked the resolution to confidently determine interactions between protein side-chains and the RNA backbone. These limitations introduced a significant amount of ambiguity into determining which nucleotides might be safely modified. As a result, six profiles of chemical modifications (denoted as versions) were designed for initial testing, and these six profiles are illustrated in FIGS. 20A and 20B. The v1 profile was designed as a simple end-protected structure, where the first and last three nucleotides were modified with 2′OMe and phosphorothioate bonds. In the v2 profile, 3′UUU tail was added to mimic the termination sequence used in cellular transcription systems and to move the modified nucleotides outside of the region of the spacer involved in target recognition. The v3 profile included the end protection as in v1, as well as the addition of 2′OMe modifications at all nucleotides identified to be potentially modifiable based on structural analysis. The v4 profile was modeled based on v3, but with all the modifications in the triplex region removed, as this structure was predicted to be more sensitive to any perturbation of the RNA helical structure and backbone flexibility. The v5 profile maintained chemical modifications in the scaffold stem and extended stem regions, while the v6 profile harbored modifications only in the extended stem. The extended stem is a region that would become fully exposed to solvent in the RNP and is amenable to replacement by other hairpin structures, and therefore presumably relatively insensitive to chemical modifications.

The minimally modified v1 gRNA was initially assessed against an unmodified gRNA (v0) to determine the potential benefit of such chemical modifications on editing when the gRNA was co-delivered with CasX mRNA to target cells. Modified (v1) and unmodified (v0) B2M-targeting gRNAs with spacer 7.37 were co-transfected with CasX mRNA into HepG2 cells, and editing at the B2M locus was measured by loss of surface presentation of the B2M-dependent HLA complex, as detected by flow cytometry (FIG. 21). The data demonstrate that use of the v1 gRNA resulted in substantially greater loss of B2M expression compared to the levels seen with v0 gRNA across the various doses, thereby confirming that end modifications of the gRNA increased CasX-mediated editing activity upon delivery of the CasX mRNA and gRNA.

The broader set of gRNA chemical modification profiles were assessed using PCSK9-targeting gRNAs using scaffold variant 235 and spacers 6.7 and 6.8 to determine whether the additional chemical modifications would be able to support the formation of active RNPs. In vitro cleavage assays described above were performed to determine kcieave and fraction competence for these engineered gRNAs harboring the various chemical modification profiles. The results from these in vitro cleavage assays are shown in Table 49. The data demonstrate that gRNAs with the v3 profiles exhibited no activity, an indication that the addition of some chemical modifications significantly interfered with RNP formation or activity. Adding v4 chemical modifications resulted in a reasonable cleavage rate in the excess RNP condition, but exhibited very low fraction competence. The difference between v3 and v4 modifications confirmed that modifications in the triplex region prevented the formation of any active RNP, either due to the inability of the gRNA to fold properly or a disruption in the gRNA-protein interactions. The reduced fraction competence resulting from appending v4 modifications suggest that while the gRNA was able to successfully assemble with the CasX protein to form a cleavage-competent RNP, a large majority of the gRNA was misfolded, or that the appended chemical modifications reduced the affinity of the gRNA for the CasX protein and impeded the efficiency of RNP formation. Application of the v5 or v6 profiles resulted in competent fractions that were comparable to, but slightly lower than, those obtained for reactions using the v1 and v2 modifications. While the kcieave values were relatively consistent between v5 and v6 gRNAs, both v5 and v6 gRNAs achieved nearly half of the kcieave values for v1 and v2 gRNAs. The reduced kcieave value for v6 gRNA was particularly surprising, given the lack of expected interaction between the gRNA and CasX protein in the modified extended stem. However, for both v5 and v6 gRNAs, it is possible that the reduced flexibility of the gRNA, resulting from the 2′OMe modifications, inhibited structural changes in the RNP required for efficient cleavage, or that the modified initial base-pairs of the hairpin involved in CasX protein interaction had been negatively impacted by the inclusion of the 2′OMe groups.

TABLE 49
Parameters of cleavage activity assessed for CasX
RNPs with the various PCSK9-targeting gRNAs using
scaffold 235 and harboring the indicated chemical
modification profile, denoted by version number

| gRNA | | |
|---|---|---|
| (scaffold variant-spacer, | | |
| version no.) | k_{cleave} (min⁻¹) | Fraction competence |
| 235-6.7, v1 | 0.901 | 0.398 |
| 235-6.8, v1 | 1.36 | 0.398 |
| 235-6.7, v2 | 0.454 | 0.386 |
| 235-6.8, v2 | 2.03 | 0.361 |
| 235-6.7, v3 | 0 | 0 |
| 235-6.8, v3 | 0 | 0 |
| 235-6.7, v4 | 0.434 | 0.031 |
| 235-6.8, v4 | 0.257 | 0.005 |
| 235-6.7, v5 | 0.506 | 0.313 |
| 235-6.8, v5 | 0.680 | 0.388 |
| 235-6.7, v6 | 0.462 | 0.346 |
| 235-6.8, v6 | 0.715 | 0.325 |

The chemically-modified PCSK9-targeting gRNAs based on scaffold 235 were subsequently assessed for editing in a cell-based assay. CasX mRNA and chemically modified PCSK9-targeting gRNAs were co-transfected into HepG2 cells using lipofectamine. Editing levels were measured by indel rate at the PCSK9 locus by NGS and secreted PCSK9 levels by ELISA, and the data are displayed in Table 50. The data demonstrate that use of v3 and v4 gRNAs resulted in minimal editing activity at the PCSK9 locus, consistent with findings from the biochemical in vitro cleavage assays shown in Table 49. Meanwhile, use of v5 and v6 gRNAs resulted in editing levels, measured by indel rate and PCSK9 secretion, that were slightly lower than the levels attained with use of v1 and v2 gRNAs (Table 50). Specifically, the results show that use of v1 and v2 gRNAs, which harbored end modifications, resulted in ~80-85% editing at the PCSK9 locus, indicating that adding chemical modifications to the gRNA ends was sufficient to achieve efficient editing with CasX. While the data demonstrate that use of v5 and v6 gRNAs resulted in efficient editing in vitro, near-saturating levels of editing were observed with use of the v1 gRNA in this experiment where a single dose of the gRNA was transfected. As a result, the use of a single dose rendered it challenging to assess clearly the effects of the chemical modifications on editing under guide-limiting conditions. Therefore, profiles v1 and v5 were chosen for further testing, as v1 contains the simplest modification profile, and v5 is the most heavily modified profile whose application demonstrated robust activity in vitro (Tables 49 and 50).

TABLE 50
Editing levels measured by indel rate at PCSK9 locus by NGS and
secreted PCSK9 levels by ELISA in HepG2 cells co-transfected
with CasX 491 mRNA and various chemically-modified PCSK9-targeting
gRNAs using scaffold 235 and either spacer 6.7 or 6.8

| | | |
|---|---|---|
| | Indel rate | Secreted |
| Experimental | (edit fraction) | PCSK9 (ng/mL) |

| condition | Mean | Stdev | Mean | Stdev |
|---|---|---|---|---|
| CasX mRNA only | 0.0021 | 0.003 | 52 | 14 |
| 235-6.7, v1 | 0.83 | 0.0058 | 18 | 5.7 |
| 235-6.7, v2 | 0.79 | 0.0071 | 21 | 4 |
| 235-6.7, v3 | 0.024 | 0.02 | 48 | 19 |
| 235-6.7, v4 | 0.12 | 0.006 | 34 | 5.5 |
| 235-6.7, v5 | 0.73 | 0.023 | 21 | 9 |
| 235-6.7, v6 | 0.75 | 0.0069 | 22 | 8.8 |
| 235-6.8, v1 | 0.85 | 0.017 | 16 | 4.4 |
| 235-6.8, v2 | 0.83 | 0.0028 | 20 | 1.5 |
| 235-6.8, v3 | 0.023 | 0.0027 | 39 | 2.7 |
| 235-6.8, v4 | 0.088 | 0.0086 | 42 | 10 |
| 235-6.8, v5 | 0.77 | 0.017 | 19 | 1.6 |
| 235-6.8, v6 | 0.78 | 0.014 | 24 | 6.9 |
| Non-targeting ctrl | 0.0019 | 0.0026 | 42 | 12 |

The v1 and v5 profiles were further tested in another cell-based assay to assess their effects on editing efficiency. LNPs were formulated to co-encapsulate CasX 676 mRNA #2 and v1 and v5 chemically-modified ROSA26-targeting gRNAs using the newly-designed gRNA scaffold 316 (described further in the following sub-section). The “v5” profile was modified slightly for application to the 316 scaffold. Three 2′ OMe modifications in the non-base-paired region immediately 5′ of the extended stem were removed to restrict modifications to the two stem-loop regions. Hepa1-6 hepatocytes were treated with the resulting LNPs at various doses and harvested eight days post-treatment to assess editing at the ROSA26 locus, measured as indel rate detected by NGS (FIG. 26A). The data demonstrate that treatment with LNPs delivering the v5 ROSA 26-targeting gRNA resulted in markedly lower editing levels across the range of doses compared to the levels achieved with the v1 counterpart (FIG. 26A). There are several possible explanations for the differences in relative activity observed with use of v5 gRNA in FIG. 26A relative to that observed in Table 50. The first and most likely possible explanation is that the single dose used to achieve editing shown in Table 50 was too high to measure differences in activity accurately between use of v5 gRNA and v1 gRNA. It is also possible that the removal of the modifications outside the stemloop motifs in the 316 version of v5 negatively impacted guide activity. While it is possible that these modifications provide stability benefits that outweigh an activity cost imparted by the stem-loop modifications, this seems unlikely given that increasing levels of modification have so far resulted in decreased activity. A final possible explanation is that the modifications in the v5 profile might negatively impact LNP formulation or behavior through differential interactions between the modified nucleotide backbone and the ionizable lipid of the LNP, potentially resulting in less efficient gRNA encapsulation or in less efficient gRNA release following internalization.

LNPs co-encapsulating the CasX mRNA #2 and v1 and v5 chemically-modified ROSA26-targeting gRNAs based on scaffold 316 were further tested in vivo. FIG. 26B shows the results of the editing assay as percent editing measured as indel rate at the ROSA26 locus. The data demonstrate that use of the v5 gRNA resulted in ~5-fold lower editing compared to that achieved with use of the v1 gRNA, under more relevant testing conditions of in vivo LNP delivery. These findings support the reduced cleavage rate observed biochemically for the v5 gRNA in Table 49, an indication that the v5 modifications have interfered with some aspect of CasX activity. Given the consistent decrease in activity detected in v5 and v6 profiles (Table 49), the reduced editing may be attributed to modifications in the extended stem region. Although the extended stem of the gRNA has minimal interactions with the CasX protein, it is possible that addition of 2′OMe groups at the first base-pair disrupted either the CasX protein-gRNA interactions or the complex RNA fold where the extended stem meets the pseudoknot and triplex regions. More specifically, inclusion of the 2′OMe groups might have adversely affected the basal base-pairs of the gRNA extended stem and residues R49, K50, and K51 of the CasX protein. Finally, structural studies of CasX have suggested that flexibility of the gRNA is required for efficient DNA cleavage (Liu J, et al, CasX enzymes comprise a distinct family of RNA-guided genome editors. Nature 566:218-223 (2019); Tsuchida C A, et al, Chimeric CRISPR-CasX enzymes and guide RNAs for improved genome editing activity. Mol Cell 82(6): 1199-1209 (2022)). Thus, the addition of the 2′OMe groups throughout the extended stem might have enforced a more rigid A-form helical structure and prevented the needed flexibility for the gRNA for efficient cleavage. Furthermore, it is possible that the additional modifications in the scaffold stem in the v5 and v6 profiles might be detrimental to activity, though this is currently unclear given the limited comparisons between the v5 and v6 profiles.

Additional modification profiles were designed with the goal of enhancing gRNA stability while mitigating the adverse effects on RNP cleavage activity. Using recently published structures of wild-type CasX from Planctomycetes (PDB codes 7WAY, 7WAZ, 7WB0, 7WB1), which has a higher homology to the engineered CasX variants being assessed, additional chemical modification profiles for gRNAs were designed and are illustrated in FIG. 22. These profiles illustrate the addition of 2′OMe groups and phosphorothioate bonds to a newly-designed gRNA scaffold variant, which is described in the ensuing sub-section. These new gRNA chemical modification profiles were designed based on the initial data demonstrating sufficient editing activity observed in Table 50 with use of the v5 gRNA that suggested that modifications to the extended stem and scaffold stem regions would not negatively impact activity. The v7 profile was designed to include 2′OMe at residues likely to be modifiable throughout the gRNA structure, which excluded the triplex region, given the dramatic negative effects of adding such modifications observed earlier with the v3 profile. More conservative profiles, v8 and v9, were also designed, as illustrated in FIG. 22. For the v8 construct, modifications were removed in the pseudoknot and triplex loop region, but were retained in the scaffold stem, extended stem, and their flanking single-stranded regions, in addition to the 5′ and 3′ termini. For the v9 profile, modifications were removed in the single-stranded regions flanking the stem-loops, but were retained in the stem-loops themselves, in addition to the pseudoknot, triplex loop, and 5′ and 3′ termini. The additional chemical modification profiles v7, v8, and v9 of the newly designed gRNA scaffold variant 316 (discussed further below) were assessed in vivo at the PCSK9 locus. The results of the editing assay in vivo quantified as percent editing at the PCSK9 locus measured as indel rate as detected NGS are illustrated in FIG. 27. Despite the fact that low editing efficiency was detected overall, the data demonstrate that use of v7, v8, and v9 gRNAs resulted in lower editing levels at the PCSK9 locus compared to the indel rate achieved with use of the v1 gRNA (FIG. 27). Given the findings in FIGS. 26A-26B showing inferior editing activity attained with the v5 gRNA, it is unsurprising that v7, v8, and v9 profiles similarly demonstrated comparatively lower editing activity. As illustrated in FIG. 22, the v7, v8, and v9 profiles include modifications throughout the extended stem region, which might have interfered with RNP activity. Comparison of gRNA scaffold variant 174 and 316 using an in vitro cleavage assay:

Previous work had established gRNA scaffold variant 235 as the top-performing scaffold variant across multiple delivery conditions. However, the longer length of scaffold 235 (119 bp, when using a 20 bp spacer) relative to gRNAs including scaffold 174 (109 bp, when using a 20 bp spacer) increased the difficulty of solid-phase RNA synthesis, which would result in increased manufacturing costs, decreased purity and yield, and higher rates of synthesis failures. To address these issues but retain the improved activity of using scaffold variant 235, a chimeric gRNA scaffold was designed primarily on the basis of the scaffold 235 sequence, but the extended stemloop of scaffold 235 was replaced with the shorter extended stemloop of scaffold variant 174 (FIGS. 23A-C). The resulting chimeric scaffold, named scaffold 316, was synthesized in parallel with scaffold 174 and PCSK9-targeting spacers 6.7 and 6.8, and B2M-targeting spacer 7.9 harboring the v1 chemical modification profile, with 2′OMe and phosphorothioate bonds on the first and last three nucleotides of all gRNAs (see Table 45). Scaffold variant 174 was chosen as the comparator rather than variant 235 because variant 174 was the best previously characterized scaffold with the same length as variant 316.

In vitro cleavage activity was assessed for gRNAs with scaffold 174 and 316 and spacers 6.7 and 6.8. Cleavage assays were carried out with 20-fold excess RNP over a matching dsDNA target. Cleavage rates were quantified for all four guides, and the results are shown in Table 51. The data demonstrate that in the context of spacer 6.7, use of either scaffold 174 or 316 resulted in similar cleavage rates, with scaffold 316 resulting in marginally faster cleavage than that achieved with scaffold 174. In the context of spacer 6.8, the difference in cleavage activity was more pronounced: CasX RNPs using scaffold 316 were able to cleave DNA nearly twice as quickly as CasX RNPs using scaffold 174 (Table 51).

Assays were also performed with equimolar amounts of RNP and DNA target over a longer time course to assess the fraction of expected RNP active for cleavage. As the CasX RNP is essentially single-turnover over the tested timescale, and the concentrations used are expected to be substantially higher than the K_{D} of the DNA-binding reaction, the amount of cleaved DNA should approximate the amount of active RNP. For either spacer 6.7 or 6.8, the active fraction of CasX RNPs incorporating scaffold 316 was 25-30% higher than for CasX RNPs using scaffold 174 (Table 51). These data suggest that a higher fraction of gRNA using scaffold 316 was properly folded for association with the CasX protein, or that the gRNA using scaffold 316 was able to associate more strongly with the CasX protein. Compared to scaffold 174, scaffold 316 harbors mutations expected to stabilize the pseudoknot and triplex structures required for proper gRNA folding. The increased stability of these motifs in particular, which were more likely to misfold than the simple hairpins found elsewhere in the gRNA structure, might result in a slightly higher fraction of the gRNAs folding into an active conformation.

TABLE 51
Parameters of cleavage activity assessed for CasX RNPs
with gRNAs containing scaffold variant 174 or 316 with
the version 1 (v1) chemical modification profile

| gRNA | | |
|---|---|---|
| (scaffold variant-spacer) | k_{cleave} (min⁻¹) | Fraction competence |
| 174-6.7, v1 | 0.236 | 0.194 |
| 174-6.8, v1 | 0.142 | 0.165 |
| 316-6.7, v1 | 0.264 | 0.244 |
| 316-6.8, v1 | 0.272 | 0.213 |

Comparison of gRNA Scaffold Variant 174 and 316 in a Cell-Based Assay:

An editing assessment using gRNA scaffold variant 174 compared to variant 316 was performed in a cell-based assay. CasX 491 mRNA and the version 1 (v1) of PCSK9-targeting gRNAs using spacers 6.7 and 6.8 were lipofected into HepG2 cells. Treated cells were harvested 28 hours post-transfection for analysis of editing levels at the PCSK9 locus by NGS and secreted PCSK9 levels by ELISA, and the data are presented in FIG. 24. The data demonstrate that use of any of the PCSK9-targeting gRNA tested resulted in efficient editing at the PCSK9 locus and substantial reduction in PCSK9 secretion compared to the non-targeting control using the B2M-targeting gRNA. The results also show that use of scaffold 316 resulted in more effective editing at the PCSK9 locus than that observed with use of scaffold 174 (~10 percentage point increase in editing rate achieved with scaffold 316 over scaffold 174). This finding is further supported by the ELISA results, such that use of scaffold 316 resulted in more effective reduction of PCSK9 secretion compared to that achieved with use of scaffold 174.

Scaffold variants 174 and 316 were also assessed in an editing assay where LNPs were formulated to co-encapsulate CasX 491 mRNA and B2M-targeting gRNA harboring either scaffold variant. HepG2 cells were treated with the resulting LNPs at various doses and harvested seven days post-treatment to assess editing at the B2M locus, measured as indel rate detected by NGS (FIG. 25A) and loss of surface presentation of the B2M-dependent HLA complex, as detected by flow cytometry (FIG. 25B). The results from both assays demonstrate that treatment with LNPs to deliver the B2M-targeting gRNA using scaffold 316 resulted in higher editing potency at the B2M locus compared to LNPs delivering the gRNA using scaffold 174 at each dose (FIGS. 25A and 25B). Specifically, at the highest dose of 250 ng, use of scaffold 316 resulted in an editing level that was nearly two-fold higher than the level attained with using scaffold 174. This substantial increase in editing efficacy when using scaffold 316 versus scaffold 174, compared to the comparatively modest difference in activity observed from the in vitro cleavage assays, might be attributed to the destabilization of gRNA structure and folding during LNP formulation. The low pH conditions and association of cationic lipids during LNP formulation could adversely affect parts of the gRNA structure and result in unfolding. Consequently, it would be necessary for the gRNA to refold quickly in the cytoplasm upon delivery, both to bind the CasX protein to form the RNP and to evade RNase degradation. The stability-increasing mutations in scaffold 316 compared to scaffold 174 might provide a substantial benefit in supporting proper gRNA refolding in the cytoplasm after LNP delivery, while the deliberate folding protocol carried out for the gRNA prior to biochemical experiments likely reduced the impact of these mutations.

### Example 12: Evaluation of ADD Domain Function in a Mouse Model

Experiments were performed to assess durability of methylation upon treatment with a CasX:gRNA system when delivered in vivo in a mouse model. LTRP1 orientation molecules were evaluated for durability of methylation, mPCSK9 transcript knockdown, and mPCSK9 protein production in the presence and absence of the ADD domain.

### Materials and Methods

### Lnp Preparation:

LTRP mRNA and gRNA were encapsulated into LNPs using ALC-0315 based lipid mix using a custom-made T-mixer micro mixing device, at a flow rate of 20 mL/min and 3:1 mixing ratio of aqueous to organic phase. The composition is the following: ionizable lipid:DSPC:cholesterol:DMG-PEG2000 at 50:10:38.5:1.5 mol %. Briefly, to formulate LNPs, equal mass ratios of LTRP mRNA and gRNA are diluted in 25 mM sodium acetate, pH 4.0. The lipid mix is made at a 10 mM concentration in anhydrous ethanol. mRNA/gRNA co-formulations are generated using a predetermined N/P ratio. The RNA and lipids are run through a custom-made T-mixer device at a predetermined flow rate ratio using syringe pump infusers. After formulation, the LNPs are dialyzed into PBS, pH 7.4, to decrease the ethanol concentration and increase the pH, which increases the stability of the particles. Buffer exchange of the mRNA/gRNA-LNPs is achieved by overnight dialysis into PBS, pH 7.4, at 4° C. using 10k Slide-A-Lyzer™ Dialysis or Cassettes (Thermo Scientific™) or 12-14 kDa dialysis tubing (Repligen). Following dialysis, the mRNA/gRNA-LNPs is concentrated to >0.2 mg/mL using 30-100 kDa Amicon®-Ultra Centrifugal Filters (Millipore) and then sterile-filtered using Acrodisc PES membrane filters. Formulated LNPs are analyzed on a Malvern Zetasizer to determine their diameter and polydispersity index (PDI). Encapsulation efficiency and RNA concentration is determined by RiboGreen™ assay using Invitrogen's Quant-iT™ RiboGreen™ RNA assay kit. The LNPs described above are used in various experiments to deliver LTRP mRNA and gRNA for delivery to target tissues in vivo.

Delivery of LNPs Encapsulating CasX mRNA and Targeting gRNA In Vivo:

To assess the effects of using LTRP1A and LTRP1B in vivo, an LTRP mRNA (see Table 52) and a mPCSK9-targeting gRNA using scaffold 316 v1 with spacer 27.94 (SEQ ID: 21877, Table 53) were encapsulated within the same LNP using a 1:1 mass ratio for mRNA:gRNA. Formulated LNPs were buffer-exchanged to PBS for in vivo injection. Briefly, LNPs were administered at 3.0 mg per kg intravenously through the tail-vein into 4-week-old C57BL/6 mice. Mice were observed for five minutes after injection to ensure recovery from anesthesia before being placed into their home cage. Vehicle (PBS) injected animals served as negative experimental controls and mice injected with mRNA:gRNA encoding for LTRP1 and the mPCSK9 targeting guide above will serve as positive controls. Baseline was taken via fasted blood draw. Further blood draws were taken every three weeks. Seven, fourteen, and forty-two days post-administration, 3 mice from each condition were euthanized, the blood and liver tissue were harvested. Blood serum was collected for mPCSK9 ELISA, liver tissue was homogenized for mRNA extraction and for gDNA extraction using the Zymo Research Quick DNA/RNA Miniprep kit following the manufacturer's instructions.

### Enzymatic Methylation Sequencing (EM-Seq):

To determine on-target methylation levels at the PCSK9 locus, gDNA from harvested tissues was extracted using the Zymo Quick-DNA Miniprep Plus kit following the manufacturer's instructions. The extracted gDNA was then subjected to enzymatic methylation conversion using the Enzymatic Methyl-seq Conversion Module (NEB) following the manufacturer's protocol, converting any non-methylated cytosine into uracil. The resulting treated DNA was subsequently sequenced using next-generation sequencing (NGS) to determine the levels of on-target methylation.

### NGS Processing and Analysis:

Target amplicons were amplified from 50 ng EM-treated DNA via PCR with a set of primers specific to the EM-converted target locations of interest (mouse PCSK9 locus). These gene-specific primers contained an additional sequence at the 5′ end to introduce an Illumina™ adapter. Amplified DNA products were purified with the Cytiva Sera-Mag Select DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were processed using Bismark Bisulfite Read Mapper and Methylation caller. PCR amplification of the EM-treated DNA would convert all uracil nucleotides into thymine, and sequencing of the PCR product would determine the rate of cytosine-to-thymine conversion as a readout of the level of on-target methylation at the PCSK9 locus mediated by each LTRP molecule.

qPCR Assay for mPCSK9 mRNA Knockdown:

Liver pieces were flash frozen after necropsy. These were then immersed in excess volume of Zymo DNA/RNA Shield Reagent and homogenized using bead beating tubes (Bullet Blender instrument, with ceramic or steel beads in tubes). After homogenization, lysates were treated with Proteinase K. Proteinase K-treated liver lysates had mRNA extracted using Zymo Quick RNA Miniprep Kit (cat #R1055) according to manufacturer's instructions and assayed using the TaqMan gene expression assay with mPCSK9 and Eukaryotic 18S probes following manufacturer's instructions.

### ELISA Method:

Serum from animal samples were assayed using LEGEND MAX mouse PCSK9 ELISA kits (BioLegend cat #443207) following manufacturer's instructions.

### Results:

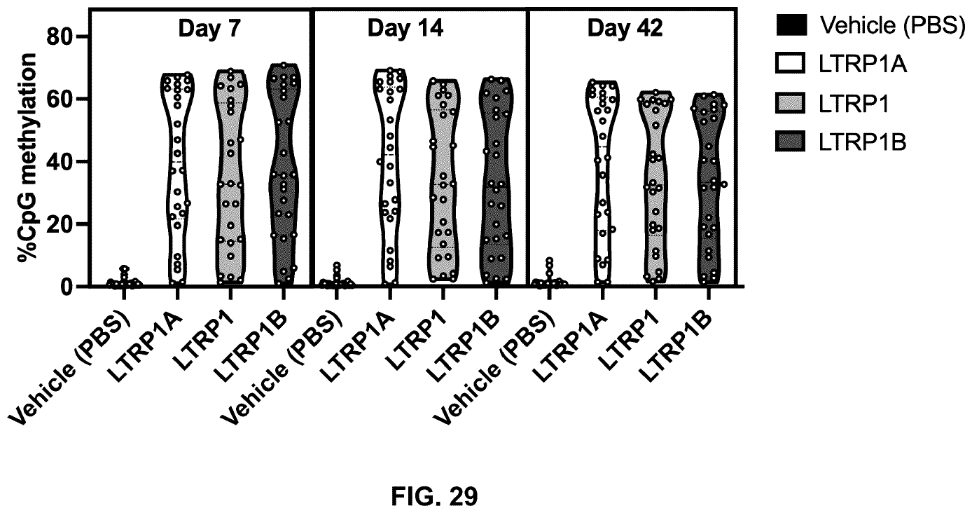

To assess durability of methylation upon treatment with a CasX:gRNA system when delivered in vivo in a mouse model, mRNAs encoding LTRP1 (SEQ ID NO. 21876), LTRP1A (SEQ ID NO. 2409), or LTRP1B (SEQ ID NO. 2419) and gRNA 316v1.27.94 (SEQ ID NO. 21877) were encapsulated in LNPs and injected into C57BL/6 mice. The quantification of the resulting PCSK9 mRNA knockdown is shown in Table 54. The data demonstrate comparable levels of knockdown across all three LTRP1 orientation molecules at 7 and 14 days, with LTRP1 and LTRP1A showing a higher level of suppression at day 42. Supporting this result, methylation data from EM-seq (FIG. 29) shows high levels of methylation across all three LTRP1 orientation molecules tested, with slight decrease total percent methylation of mice treated with LTRP1 and LTRP1B at days 14 and 42.

PCSK9 secretion quantification shows similar levels of PCSK9 suppression at day 7 (week 1, Table 55) across all LTRP molecules tested. LTRP1A shows increased durability of repression through week 6 of testing, while LTRP1B shows increased durability of repression through week 8 as compared to LTRP1.

These experiments demonstrate that LTRP1 orientation molecules with and without the ADD domain delivered via LNPs as mRNA and are able to induce effective silencing in vivo.

TABLE 52
mRNA sequences of LTRP constructs tested in this example

| | SEQ ID | |
|---|---|---|
| LTRP | NO. | Components |
| LTRP1 | 21876 | LTRP1 ZIM3, 80 poly(A), SV40, C-terminal |
| | | 2xFLAG |
| LTRP1A | 2409 | LTRP1 ZIM3, split poly(A), SV40 |
| LTRP1B | 2419 | LTRP1 ZIM3, ADD, split poly(A), SV40 |

TABLE 53
gRNA and targeting sequence used in this example

| gRNA ID | SEQ ID NO. |
|---|---|
| 27.94 | 2014 |
| 316v1.27.94 | 21877 |

TABLE 54
Liver lysate mRNA qPCR

| | Days post-dose | | | | | |
|---|---|---|---|---|---|---|
| | 7 Days | | 14 Days | | 42 Days | |
| LTRP | Mean | std | Mean | std | Mean | std |
| Vehicle (N = 3) | 7.14 | 49.81 | 5.24 | 42.63 | 1.00 | 17.10 |
| LTRP1 (N = 3) | −96.55 | 1.77 | −96.96 | 1.21 | −93.65 | 2.60 |
| LTRPIA (N = 3) | −99.04 | 0.66 | −99.76 | 0.09 | −93.19 | 4.73 |
| LTRP1B (N = 3) | −96.13 | 1.47 | −92.39 | 5.65 | −80.55 | 5.79 |

TABLE 55
Change in Secreted PCSK9 by ELISA
Percent secreted PCSK9 by serum ELISA, normalized to baseline

| Study | Vehicle | | LTRP1 | | LTRP1A | | LTRP1B | |
|---|---|---|---|---|---|---|---|---|
| Week | Mean | SEM | Mean | SEM | Mean | SEM | Mean | SEM |
| 1 | 23.80 | 45.23 | −94.45 | 0.45 | −93.50 | 0.91 | −94.30 | 0.60 |
| 2 | −15.06 | 14.48 | −92.20 | 1.96 | −85.10 | 7.07 | −93.69 | 0.44 |
| 5 | 4.98 | 10.38 | −90.18 | 2.78 | −95.80 | 0.78 | −93.37 | 0.42 |
| 6 | −11.92 | 27.13 | −92.65 | 1.72 | −95.81 | 0.79 | −90.36 | 2.14 |
| 8 | 17.84 | 9.53 | −80.96 | 6.72 | NA | NA | −90.58 | 1.20 |

Values assayed below LOQ were set to LOQ (6.25 ng/mL), LTRPIA week 8 data was not collected.

### Example 13: Efficacy and Durability of LTRP with RD1 Domains

Experiments were performed to assess durability of methylation upon treatment with LTRP 1 orientation molecules containing RD1I domains in vivo in a mouse model.

### Materials and Methods

LNPs were prepared as described in example 12.

An in vivo experiment examined the efficacy of LTRP1 orientation molecules containing a King Cobra RD1 domain (SEQ ID NO. 18642). LTRPIA-Cobra, and LTRPIB-Cobra (SEQ ID NO. 2411, SEQ ID NO. 2421) were built by substituting the ZIM3 domain in LTRP1A and LTRP 1B for the RD1 domain. Formulated LNPs containing an LTRP mRNA (Table 56) and mPCSK9 targeting gRNA (SEQ ID: 21877, Table 56) were buffer-exchanged to PBS for in vivo injection. Briefly, LNPs were administered intravenously through the tail-vein into 4-week old C57BL/6 mice. Mice were observed for five minutes after injection to ensure recovery from anesthesia before being placed into their home cage. Vehicle (PBS) injected animals served as negative experimental controls and mice injected with mRNA:gRNA encoding for LTRP 1A (SEQ ID: 2409) and mPCSK9-targeting gRNA using scaffold 316 with spacer 27.94 (SEQ ID: 21877) served as positive controls. Baseline was taken via fasted blood draw. Further blood draws were taken every three weeks. Seven and forty-two days post-administration, mice were euthanized, the blood and liver tissue were harvested. Blood serum was collected for mPCSK9 ELISA, liver tissue was homogenized for mRNA extraction and for gDNA extraction using the Zymo Research Quick DNA/RNA Miniprep kit following the manufacturer's instructions.

### Enzymatic Methylation Sequencing (EM-Seq):

To determine on-target methylation levels at the PCSK9 locus, gDNA from harvested tissues was extracted using the Zymo Quick-DNA Miniprep Plus kit following the manufacturer's instructions. The extracted gDNA was then subjected to enzymatic methylation conversion using the Enzymatic Methyl-seq Conversion Module (NEB) following the manufacturer's protocol, converting any non-methylated cytosine into uracil. The resulting treated DNA was subsequently sequenced using next-generation sequencing (NGS) to determine the levels of on-target methylation.

### NGS Processing and Analysis:

Target amplicons were amplified from 50 ng EM-treated DNA via PCR with a set of primers specific to the EM-converted target locations of interest (mouse PCSK9 locus). These gene-specific primers contained an additional sequence at the 5′ end to introduce an Illumina™ adapter. Amplified DNA products were purified with the Cytiva Sera-Mag Select DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were processed using Bismark Bisulfite Read Mapper and Methylation caller. PCR amplification of the EM-treated DNA converts all uracil nucleotides into thymine, and sequencing of the PCR product determines the rate of cytosine-to-thymine conversion as a readout of the level of on-target methylation at the PCSK9 locus mediated by each LTRP molecule. qPCR assay for mPCSK9 mRNA knockdown:

Liver pieces were flash frozen after necropsy. These were then immersed in excess volume of Zymo DNA/RNA Shield Reagent and homogenized using bead beating tubes (Bullet Blender instrument, with ceramic or steel beads in tubes). After homogenization, lysates were treated with Proteinase K. Proteinase K-treated liver lysates had mRNA extracted using Zymo Quick RNA Miniprep Kit (cat #R1055) according to manufacturer's instructions and assayed using the TaqMan gene expression assay with mPCSK9 and Eukaryotic 18S probes following manufacturer's instructions.

### ELISA Method:

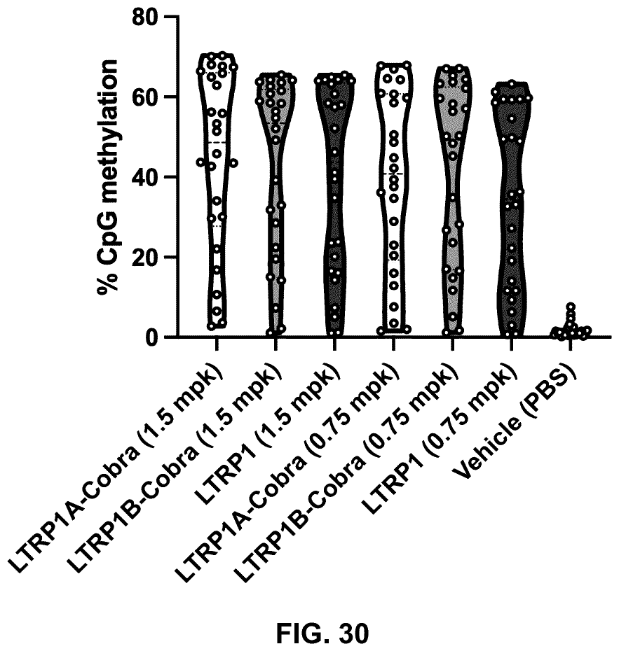

Transcriptional start site (TSS)-proximal DNA methylation levels measured from homogenized liver-extracted gDNA by amplicon enzymatic methylation sequencing (EM-seq) from N=3 mice sacrificed at day 7 post-treatment demonstrate similar levels of methylation across all constructs, with higher median methylation trending with the higher dose (FIG. 30).

Table 57 quantifies the serum PCSK9 ELISA results, demonstrating that all LTRP1 constructs show similar initial effect, with knockdown of serum PCSK9 at ~90% for groups dosed with either 0.75 or 1.5mpk through 42 days post dosing.

Table 58 quantifies the mPCSK9 mRNA qPCR assay results, demonstrating robust transcript reduction of greater than 80% knockdown is maintained by all 3 LTRP1 constructs at both doses. LTRP1A, LTRP1A-Cobra, and LTRP1B-Cobra appear potent and durable at the mRNA transcript level up to 42 days post-dose. Both LTRP1A and LTRP1A-Cobra show a slight uptick in mPcsk9 expression at 42 days at the lower dose (0.75 mg/kg), but it is not clear if this represents a trend.

These experiments demonstrate that LTRP1 orientation molecules can produce durable methylation leading to reduction in PCSK9 expression with RD1 domains in a mouse model.

TABLE 56
Sequences of mRNA and gRNA assessed

| | | | Dose mg |
|---|---|---|---|
| mRNA | SEQ ID NO. | gRNA | per kg |
| LTRP1A | 2409 | 316v1 27.94 (SEQ ID | 1.5 |
| | | NO. 21877) | 0.75 |
| LTRP1A-Cobra | 2411 | | 1.5 |
| | | | 0.75 |
| LTRP1B-Cobra | 2421 | | 1.5 |
| | | | 0.75 |
| PBS (neg. | NA | NA | NA |
| control) | | | |

TABLE 57
Serum PCSK9 ELISA at Day 7, 14, 35, and 42*
Serum PCSK9 % change from baseline

| Day | Mean | SEM | n | Mean | SEM | n |
|---|---|---|---|---|---|---|
| | Vehicle (PBS) | | | Vehicle (PBS) | | |
| 0 | 0 | 0 | 9 | N/A | N/A | N/A |
| 7 | 50.87 | 79.48 | 3 | N/A | N/A | N/A |
| 14 | 89.78 | 86.04 | 6 | N/A | N/A | N/A |
| 35 | −19.45 | 80.54 | 6 | N/A | N/A | N/A |
| 42 | 111.26 | 6.19 | 2 | N/A | N/A | N/A |
| | LTRP1A-Cobra (0.75 mpk) | | | LTRP1A-Cobra (1.5 mpk) | | |
| 0 | 0 | 0 | 9 | 0 | 0 | 9 |
| 7 | −90.42 | 3.47 | 3 | −87.40 | 2.37 | 3 |
| 14 | −87.51 | 9.17 | 6 | −87.03 | 5.74 | 6 |
| 35 | −83.73 | 12.37 | 6 | −87.03 | 5.74 | 6 |
| 42 | −91.31 | 2.87 | 2 | −93.43 | 1.15 | 2 |
| | LTRP1B-Cobra (0.75 mpk) | | | LTRP1B-Cobra (1.5 mpk) | | |
| 0 | 0 | 0 | 9 | 0 | 0 | 9 |
| 7 | −90.22 | 4.27 | 3 | −87.99 | 4.45 | 3 |
| 14 | −88.43 | 6.86 | 6 | −91.63 | 2.61 | 6 |
| 35 | −89.11 | 3.79 | 6 | −91.63 | 2.61 | 6 |
| 42 | −88.33 | 1.86 | 2 | −90.18 | 2.72 | 2 |
| | LTRP1A (0.75 mpk) | | | LTRP1A (1.5 mpk) | | |
| 0 | 0 | 0 | 9 | 0 | 0 | 9 |
| 7 | −91.57 | 1.12 | 3 | −87.94 | 2.11 | 3 |
| 14 | −88.21 | 4.21 | 6 | −90.87 | 2.15 | 6 |
| 35 | −86.95 | 4.41 | 6 | −90.87 | 2.15 | 6 |
| 42 | −70.89 | 21.14 | 2 | −89.01 | 1.36 | 2 |

*Day 7 data comes from only animals sampled at that timepoint. Day 7 and 35 data comes from different individuals that continued in-life to Day 42. Day 42 data comes from only animals sampled at that timepoint.

TABLE 58
Percent repression of mPCSK9 mRNA at Days 7 and 42 via qPCR
mPCSK9 transcript/18S percent repression

| Day | Mean | SEM | n | Mean | SEM | n |
|---|---|---|---|---|---|---|
| | Vehicle | | | Vehicle | | |
| 0 | 0 | 0 | 3 | N/A | N/A | N/A |
| 7 | 19.65 | 49.23 | 3 | N/A | N/A | N/A |
| 42 | 9.38 | 44.25 | 2 | N/A | N/A | N/A |
| | LTRP1A-Cobra (0.75 mpk) | | | LTRP1A-Cobra (1.5 mpk) | | |
| 0 | 0 | 0 | 3 | 0 | 0 | 3 |
| 7 | −92.90 | 1.48 | 3 | −96.68 | 2.01 | 3 |
| 42 | −79.37 | 0.57 | 2 | −87.63 | 6.35 | 2 |
| | LTRP1B-Cobra (0.75 mpk) | | | LTRP1B-Cobra (1.5 mpk) | | |
| 0 | 0 | 0 | 3 | 0 | 0 | 3 |
| 7 | −91.54 | 4.95 | 3 | −98.15 | 0.55 | 3 |
| 42 | −90.86 | 0.57 | 2 | −96.02 | 1.31 | 2 |
| | LTRP1A (0.75 mpk) | | | LTRP1A (1.5 mpk) | | |
| 0 | 0 | 0 | 3 | 0 | 0 | 3 |
| 7 | −92.79 | 1.21 | 3 | −95.36 | 1.81 | 3 |
| 42 | −84.55 | 5.15 | 2 | −91.95 | 4.47 | 2 |

### Example 14: Functional Assessments of CasX:gRNA Systems Targeting the Human PCSK9 Locus

Experiments were performed to demonstrate that delivery of LNPs encapsulating LTRP6 mRNA and a PCSK9-targeting gRNA induces repression at the endogenous human PCSK9 locus in primary human hepatocytes (PHH1) and thus, induce a reduction of secreted PCSK9 and PCSK9 mRNA. Four LTRP molecules (Table 59) and guide scaffold 316v1 (SEQ ID NO: 2156) were selected for assessment in this Example.

### Materials and Methods

PCSK9 knockdown in primary human hepatocytes, provided an experimental basis for lipofection of gRNA comprising one of the four top editing targeting sequences (Table 59) into PHH cells for assessment of PCSK9 secretion and mRNA reduction. Dose response curves were generated from two lots of PHH cells with three rounds of triplicate wells for each condition. The highest tested dose, 2400 ng/well total encapsulated RNA, was used to measure mRNA expression changes between treated and naïve cells via RNA-seq.

Synthesis of gRNAs:

gRNAs targeting the human PCSK9 locus were chemically synthesized by methods known in the art that, in each case, were converted into the chemically-modified v1 versions for delivery. The PCSK9-targeting sequences are listed in Table 59.

LTRP mRNA was generated by IVT. Briefly, constructs encoding for a 5′UTR region, a codon-optimized LTRP, and a 3′UTR region were cloned into a plasmid containing a T7 promoter and poly(A) tail. The resulting plasmid was linearized prior to use for IVT reactions, which were carried out with CleanCap® AG and N1-methyl-pseudouridine. Formulation of lipid nanoparticles (LNPs):

LTRP mRNA and targeting gRNAs were encapsulated into LNPs made from Ionizable lipid mix containing ALC0315 Ionizable lipid:18:0 PC (DSPC): Cholesterol: DMG-PEG2000 using an in-house custom-made T-mixer at N/P 6. Briefly, to formulate LNPs, as a split formulation (containing either mRNA or only sgRNA) were diluted at a fixed ratio for coformulation or separately for split formulation in a 25 mM sodium acetate buffer, pH 4.0. ALC315 Ionizable lipid mix at a molar ratio of 50:10:38.5:1.5% of above-mentioned lipids using anhydrous ethanol. The RNA and lipid phases were run through a custom-made T-mixer at a flow rate ratio of 3:1 and a flow rate of 20 mL/min. After formulation, the LNPs were transferred and dialyzed using a 10 KDa membrane dialysis cassette (Thermo Scientific™) and buffer exchanged into 1×PBS to decrease the ethanol concentration and increase the pH to 7.4, leading to formation of a mature and stabilized particles. Following dialysis, the RNA-LNPs were buffer exchanged into 300 mM sucrose in PBS at pH 7.4 storage buffer and concentrated to appropriate concentration using 100 kDa Amicon®-Ultra Centrifugal Filters (Millipore) and sterile-filtered. Formulated LNPs were then subjected to one free-thaw cycle at −80° C. and analyzed on a Stunner (Unchained Labs) to determine their average particle size (d. nm.) and polydispersity index (PDI). Encapsulation efficiency and RNA concentration was determined by RiboGreen™ assay using Invitrogen's Quant-iT™ Ribogreen™ RNA assay kit. LNPs were used in various experiments as described herein to deliver mRNA and gRNA to target cells and tissue by mixing a 1:1 mass ratio of mRNA containing LNP and gRNA containing LNP. Delivery of LNPs encapsulating CasX mRNA and targeting gRNA into primary human hepatocytes:

Two lots (lot #271 and lot #31) of primary human hepatocytes (Lonza Biologics) were plated at density of 65K cells/well. Twenty-four hours later, plates were stamped with the indicated total encapsulated RNA dose of single formulations ALC-0315 LNP (1:1 ratio, pre-incubated with human serum overnight) at the indicated dose in triplicate wells for each condition. Doses tested for each mRNA:gRNA pair (Table 60) were 2400, 800, 270, 90, 30, 10, 0.1, and 0.01 ng/well total encapsulated RNA.

Cells were maintained in Geltrex-sandwich culture, and media supernatants collected on day 5 post-treatment were assayed for secreted PCSK9 by HTRF ELISA with CISBio Human PCSK9 HTRF ELISA Kit (Revvity). The cell culture experiment was repeated for 3 rounds.

For rounds 1 and 2, cells were immediately lysed in 100 uL DNA RNA shield™ and stored at ~80C after the day 5 media collection.

Naïve, CasX 515 with targeting sequence 6.1, and LTRP5 with targeting sequence 27.94 control treatments were included on each plate to assess robustness of measurements across the experiment.

qPCR Assay for PCSK9 mRNA Knockdown:

Extracted mRNA was processed and analyzed by RT-qPCR. Measurement ofPCSK9 mRNA expression is performed using a single-step RT-qPCR approach. 50 uL lysate was used to extract total RNA using Zymo 96 RNA extraction kits. Total RNA was then reverse transcribed into cDNA and PCSK9 transcript quantified in reference to a GAPDH endogenous control by quantitative PCR using TaqMan probes. Percent change in mRNA was calculated by comparing to samples treated with a non-targeting sequence.

### ELISA:

Secreted PCSK9 levels in the media supernatant were analyzed using the BioLegend® ELISA MAX™ kit following the manufacturer's instructions.

### RNA Seq:

mRNA levels were analyzed using the NEBNext Ultra II Directional RNA Library Prep Kit for Illumina (NEB #E7760) following the manufacturer's instructions. RNA from the highest dose samples treated with the indicated mRNA-gRNA pairs (N=2-6 wells/condition) and naïve wells across plates (N=4) wells was analyzed using paired end short-read RNA sequencing. Gene expression changes versus naïve cells were computed from read counts using DEseq2. Cutoffs used were |log2(Fold Change)|>1 and Benjamini and Hochberg-adjusted p value (padj)<0.05. Results:

The ability of pairs of mRNA:gRNA comprising scaffold 316 and an PCSK9-targeting sequence to reduce secretion of PCSK9 and PCSK9 mRNA reduction was assessed. Tables 61-64, below, provide the half-maximal inhibitory concentration (ICSo), 90% maximum inhibition (IC₉₀), and Maximum response (Emax) in primary human hepatocytes with each individual mRNA:sgRNA pair, rounded to the nearest hundredth. Tables 61 and 62 provide results of secreted PCSK9 reduction from the serum ELISA dose response testing in units of ng of encapsulated RNA/well and μM encapsulated RNA respectively. Tables 63 and 64 provide results of PCSK9 mRNA reduction from qPCR dose response testing in units of ng of encapsulated RNA/well and μM encapsulated RNA respectively.

LTRP5 and LTRP6-Rat-Cobra were among the most potent molecules in reducing secreted protein, with secreted protein reduction similar to positive control editors CasX 515 and SpyCas9, 6.154 the most potent LTRP spacer (Tables 61 and 62).

LTRP5 and LTRP6-Rat-Cobra were among the most potent molecules in reducing PCSK9 mRNA levels, with reduction similar to positive control editors CasX 515 and SpyCas9, 6.154 the most potent LTRP spacer (Tables 63 and 64).

LTRP constructs LTRP6-Rat-Cobra, LTRP6-Rat-Cobra-linker set 3, and LTRP6-2xCobra, paired with gRNA 316.6.154 (V1) showed the highest specificity, as measured by mRNA expression change (Table 65). LTRP6-Rat-Cobra-linker set 3 and LTRP6-2xCobra had no significant off-target effects as measured with cutoff of padj <0.05.

The results from these experiments demonstrate that delivery of LNPs encapsulating a LTRP mRNA and PCSK9-targeting gRNA induced editing at the endogenous human PCSK9 locus in primary human hepatocytes that resulted in substantial reduction in secreted PCSK9 and mRNA levels.

TABLE 59
Sequences of mRNA and gRNA

| | SEQ ID | | | SEQ ID |
|---|---|---|---|---|
| mRNA | NO. | gRNA | Target | NO. |
| LTRP5 | 21886 | 316.6.1 (V1) | PCSK9 | 21898 |
| LTRP6-2xCobra | 21889 | 316.6.154 (V1) | PCSK9 | 21899 |
| LTRP6-Rat-Cobra | 21888 | 316.6.157 (V1) | PCSK9 | 21900 |
| LTRP6-Rat-Cobra-linker | 21890 | 316.6.141 (V1) | PCSK9 | 21902 |
| set 3 | | | | |
| CasX 515 | 21885 | 316.27.94 (V1) | NT | 21897 |
| SpyCas9 | 21887 | Spy.50.1 (vInt) | PCSK9 | 21901 |

TABLE 60
mRNA: gRNA pairs tested

| Construct | gRNA |
|---|---|
| CasX 515 | 316.6.1 (V1) |
| LTRP5 | 316.6.141 (V1) |
| LTRP5 | 316.6.154 (V1) |
| LTRP5 | 316.6.157 (V1) |
| LTRP5 | 316.6.154 (V1), 316.6.157 (V1) |
| LTRP5 | 316.27.94 (V1) |
| LTRP6-2xCobra | 316.6.141 (V1) |
| LTRP6-2xCobra | 316.6.154 (V1) |
| LTRP6-2xCobra | 316.6.157 (V1) |
| LTRP6-Rat-Cobra | 316.6.141 (V1) |
| LTRP6-Rat-Cobra | 316.6.154 (V1) |
| LTRP6-Rat-Cobra | 316.6.157 (V1) |
| LTRP6-Rat-Cobra-linker set 3 | 316.6.141 (V1) |
| LTRP6-Rat-Cobra-linker set 3 | 316.6.154 (V1) |
| LTRP6-Rat-Cobra-linker set 3 | 316.6.157 (V1) |
| SpyCas9 | Spy.50.1 (vInt) |

TABLE 61
Levels of PCSK9 secreted protein repression mediated by LTRP constructs with
various repressor domains by total cargo mass

| | | IC50 (ng/well) | | IC90* (ng/well) | | Emax* (Δ %) | |
|---|---|---|---|---|---|---|---|
| mRNA | gRNA | Mean | SEM | Mean | SEM | Mean | SEM |
| | | PHH Lot 271 | | | | | |
| CasX 515 | 316.6.1 (V1) | 27.7 | 5 | 190.1 | 84.2 | −94.6 | 4 |
| LTRP5 | 316.6.154 (V1) | 40 | 4 | 119.9 | 27.1 | −98.8 | 3.1 |
| SpyCas9 | Spy.50.1 (vInt) | 39.5 | 3.8 | 226.6 | 50.9 | −99.1 | 2.8 |
| LTRP5 | 316.6.154 (V1), | 42.6 | 4.6 | 114.5 | 26.5 | −97.8 | 3.6 |
| | 316.6.157 (V1) | | | | | | |
| LTRP6-Rat-Cobra | 316.6.154 (V1) | 57.4 | 5.4 | 217.5 | 43.6 | −98.9 | 2.7 |
| LTRP5 | 316.6.141 (V1) | 79.9 | 6.3 | 289.7 | 52.8 | −97.2 | 2.5 |
| LTRP6-2xCobra | 316.6.154 (V1) | 81.8 | 9.2 | 286.6 | 74.6 | −96.3 | 3.8 |
| LTRP5 | 316.6.157 (V1) | 96 | 10 | 491.6 | 122.3 | −100.8 | 4 |
| LTRP6-2xCobra | 316.6.141 (V1) | 115.8 | 21.4 | 494.7 | 215.8 | −99.5 | 7 |
| LTRP6-Rat-Cobra | 316.6.141 (V1) | 131.5 | 14.8 | 552.5 | 146.2 | −100.8 | 4.4 |
| LTRP6-Rat-Cobra- | 316.6.154 (V1) | 133.3 | 22.8 | 624 | 252.8 | −98.3 | 5.9 |
| linker set 3 | | | | | | | |
| LTRP6-Rat-Cobra | 316.6.157 (V1) | 177.6 | 38 | 1216.6 | 635 | −99 | 8.6 |
| LTRP6-2xCobra | 316.6.157 (V1) | 173.3 | 37.7 | 1054.8 | 558.8 | −91.5 | 8.5 |
| LTRP6-Rat-Cobra- | 316.6.141 (V1) | 247 | 56.6 | 933.6 | 530.2 | −92.7 | 9.4 |
| linker set 3 | | | | | | | |
| LTRP6-Rat-Cobra- | 316.6.157 (V1) | 253.4 | 66.9 | 1195.8 | 780.8 | −69.2 | 8.4 |
| linker set 3 | | | | | | | |
| | | PHH Lot 31 | | | | | |
| CasX 515 | 316.6.1 (V1) | 23.2 | 5.4 | 180 | 107.8 | −93.2 | 5.2 |
| LTRP5 | 316.6.154 (V1) | 13.7 | 1.9 | 73.4 | 25.5 | −97.4 | 3.4 |
| SpyCas9 | Spy.50.1 (vInt) | 24.8 | 5.1 | 244.2 | 134.2 | −100.1 | 5.7 |
| LTRP5 | 316.6.154 (V1), | 29.1 | 2.3 | 80.1 | 15.6 | −97.2 | 2.7 |
| | 316.6.157 (V1) | | | | | | |
| LTRP6-Rat-Cobra | 316.6.154 (V1) | 24.4 | 4.1 | 153.9 | 63.4 | −100.7 | 4.6 |
| LTRP5 | 316.6.141 (V1) | 49.6 | 7.2 | 364.6 | 126.9 | −100.6 | 4.1 |
| LTRP6-2xCobra | 316.6.154 (V1) | 72 | 16.6 | 327.5 | 171.8 | −100.5 | 8 |
| LTRP5 | 316.6.157 (V1) | 58 | 8.1 | 222.9 | 66.9 | −96.2 | 4.9 |
| LTRP6-2xCobra | 316.6.141 (V1) | 98.6 | 12.7 | 310 | 98 | −98.8 | 5.8 |
| LTRP6-Rat-Cobra | 316.6.141 (V1) | 89.4 | 9.3 | 284.2 | 71.6 | −98.5 | 4.2 |
| LTRP6-Rat-Cobra- | 316.6.154 (V1) | 88.8 | 18.6 | 406.6 | 199.3 | −96.2 | 7.1 |
| linker set 3 | | | | | | | |
| LTRP6-Rat-Cobra | 316.6.157 (V1) | 101.3 | 18.1 | 634 | 271.4 | −98.5 | 7 |
| LTRP6-2xCobra | 316.6.157 (V1) | 181.6 | 36.2 | 718.8 | 335.1 | −92.5 | 9.4 |
| LTRP6-Rat-Cobra- | 316.6.141 (V1) | 158.9 | 29.9 | 527 | 218 | −89.1 | 7.8 |
| linker set 3 | | | | | | | |
| LTRP6-Rat-Cobra- | 316.6.157 (V1) | 261.2 | 77.3 | 997.8 | 738.1 | −72.3 | 12.7 |
| linker set 3 | | | | | | | |

TABLE 62
Levels of PCSK9 secreted protein repression mediated by LTRP constructs with various
repressor domains by mRNA molarity

| | | IC50 (pM) | | IC90* (pM) | | Emax (Δ %) | |
|---|---|---|---|---|---|---|---|
| mRNA | gRNA | Mean | SEM | Mean | SEM | Mean | SEM |
| | | PHH Lot 271 | | | | | |
| LTRP5 | 316.6.154 (V1) | 110.4 | 11.2 | 330.7 | 74.9 | −98.8 | 3.1 |
| LTRP5 | 316.6.154 (V1), | 117.6 | 12.6 | 315.8 | 73 | −97.8 | 3.6 |
| | 316.6.157 (V1) | | | | | | |
| SpyCas9 | Spy.50.1 (vInt) | 128.1 | 12.5 | 734.7 | 165.1 | −99.1 | 2.8 |
| LTRP6-Rat- | 316.6.154 (V1) | 153.8 | 14.4 | 582.4 | 116.8 | −98.9 | 2.7 |
| Cobra | | | | | | | |
| CasX 515 | 316.6.1 (V1) | 138.1 | 25.1 | 947 | 419.5 | −94.6 | 4 |
| LTRP5 | 316.6.141 (V1) | 220.4 | 17.5 | 798.8 | 145.5 | −97.2 | 2.5 |
| LTRP6-2xCobra | 316.6.154 (V1) | 219 | 24.6 | 767.7 | 199.8 | −96.3 | 3.8 |
| LTRP5 | 316.6.157 (V1) | 264.6 | 27.6 | 1355.5 | 337.3 | −100.8 | 4 |
| LTRP6-2xCobra | 316.6.141 (V1) | 310 | 57.3 | 1325.1 | 578 | −99.5 | 7 |
| LTRP6-Rat- | 316.6.141 (V1) | 352.1 | 39.7 | 1479.3 | 391.5 | −100.8 | 4.4 |
| Cobra | | | | | | | |
| LTRP6-Rat- | 316.6.154 (V1) | 365.9 | 62.5 | 1713.1 | 694 | −98.3 | 5.9 |
| Cobra-linker set 3 | | | | | | | |
| LTRP6-Rat- | 316.6.157 (V1) | 475.6 | 101.7 | 3257.6 | 1700.3 | −99 | 8.6 |
| Cobra | | | | | | | |
| LTRP6-2xCobra | 316.6.157 (V1) | 464.2 | 101 | 2825 | 1496.7 | −91.5 | 8.5 |
| LTRP6-Rat- | 316.6.141 (V1) | 678.1 | 155.5 | 2563 | 1455.4 | −92.7 | 9.4 |
| Cobra-linker set 3 | | | | | | | |
| LTRP6-Rat- | 316.6.157 (V1) | 695.8 | 183.7 | 3282.8 | 2143.6 | −69.2 | 8.4 |
| Cobra-linker set 3 | | | | | | | |
| | | PHH Lot 31 | | | | | |
| LTRP5 | 316.6.154 (V1) | 37.9 | 5.2 | 202.4 | 70.3 | −97.4 | 3.4 |
| LTRP5 | 316.6.154 (V1), | 80.2 | 6.4 | 221 | 42.9 | −97.2 | 2.7 |
| | 316.6.157 (V1) | | | | | | |
| SpyCas9 | Spy.50.1 (vInt) | 80.5 | 16.6 | 791.8 | 435.2 | −100.1 | 5.7 |
| LTRP6-Rat- | 316.6.154 (V1) | 65.2 | 11.1 | 412.2 | 169.7 | −100.7 | 4.6 |
| Cobra | | | | | | | |
| CasX 515 | 316.6.1 (V1) | 115.5 | 27.1 | 896.6 | 537 | −93.2 | 5.2 |
| LTRP5 | 316.6.141 (V1) | 136.8 | 19.9 | 1005.3 | 350 | −100.6 | 4.1 |
| LTRP6-2xCobra | 316.6.154 (V1) | 192.7 | 44.4 | 877.1 | 460.3 | −100.5 | 8 |
| LTRP5 | 316.6.157 (V1) | 160.1 | 22.3 | 614.7 | 184.5 | −96.2 | 4.9 |
| LTRP6-2xCobra | 316.6.141 (V1) | 264 | 34 | 830.3 | 262.5 | −98.8 | 5.8 |
| LTRP6-Rat- | 316.6.141 (V1) | 239.3 | 24.9 | 761 | 191.6 | −98.5 | 4.2 |
| Cobra | | | | | | | |
| LTRP6-Rat- | 316.6.154 (V1) | 243.9 | 51.1 | 1116.2 | 547.1 | −96.2 | 7.1 |
| Cobra-linker set 3 | | | | | | | |
| LTRP6-Rat- | 316.6.157 (V1) | 271.3 | 48.5 | 1697.7 | 726.8 | −98.5 | 7 |
| Cobra | | | | | | | |
| LTRP6-2xCobra | 316.6.157 (V1) | 486.2 | 97 | 1925.3 | 897.6 | −92.5 | 9.4 |
| LTRP6-Rat- | 316.6.141 (V1) | 436.2 | 82.1 | 1446.8 | 598.5 | −89.1 | 7.8 |
| Cobra-linker set 3 | | | | | | | |
| LTRP6-Rat- | 316.6.157 (V1) | 717.1 | 212.2 | 2739.2 | 2026.2 | −72.3 | 12.7 |
| Cobra-linker set 3 | | | | | | | |

TABLE 63
Levels of PCSK9 mRNA repression mediated by LTRP constructs with various
repressor domains by total cargo mass

| | | IC50 (ng/well) | | IC90* (ng/well) | | Emax* (Δ %) | |
|---|---|---|---|---|---|---|---|
| mRNA | gRNA | Mean | SEM | Mean | SEM | Mean | SEM |
| | | PHH Lot 271 | | | | | |
| CasX 515 | 316.6.1 (V1) | 29.6 | 5.7 | 103.3 | 45.2 | −60.1 | 2.7 |
| SpyCas9 | Spy.50.1 (vInt) | 50.3 | 8.2 | 161.2 | 53.9 | −59.8 | 2.6 |
| LTRP5 | 316.6.154 (V1) | 38.2 | 7.8 | 211.2 | 101 | −96.7 | 5.5 |
| LTRP5 | 316.6.154 (V1), | 45.7 | 4.5 | 143.1 | 30.4 | −95.1 | 2.9 |
| | 316.6.157 (V1) | | | | | | |
| LTRP6-Rat- | 316.6.154 (V1) | 92.9 | 13.7 | 357.9 | 124.5 | −94.9 | 4.3 |
| Cobra | | | | | | | |
| LTRP5 | 316.6.157 (V1) | 101.3 | 14.5 | 504.4 | 173.5 | −94.7 | 5 |
| LTRP6-2xCobra | 316.6.154 (V1) | 123.7 | 19.8 | 528.3 | 199.1 | −91.9 | 5 |
| LTRP5 | 316.6.141 (V1) | 112.8 | 13.8 | 354.6 | 104.1 | −88.9 | 3.7 |
| LTRP6-Rat- | 316.6.141 (V1) | 153.1 | 22.4 | 755 | 264.3 | −92.9 | 4.5 |
| Cobra | | | | | | | |
| LTRP6-Rat- | 316.6.154 (V1) | 168.6 | 36 | 793.8 | 405.5 | −83.3 | 6.4 |
| Cobra-linker set | | | | | | | |
| 3 | | | | | | | |
| LTRP6-2xCobra | 316.6.141 (V1) | 159.7 | 47.4 | 695.8 | 486.5 | −92.7 | 8.7 |
| LTRP6-Rat- | 316.6.157 (V1) | 182.7 | 35.7 | 957 | 453.1 | −80.3 | 5.8 |
| Cobra | | | | | | | |
| LTRP6-2xCobra | 316.6.157 (V1) | 189.8 | 42.8 | 817.3 | 439.1 | −70.7 | 6.2 |
| LTRP6-Rat- | 316.6.157 (V1) | 135.1 | 37.9 | 470.1 | 300.4 | −41.1 | 4.3 |
| Cobra-linker set | | | | | | | |
| 3 | | | | | | | |
| LTRP6-Rat- | 316.6.141 (V1) | 233.2 | 67.6 | 1054.7 | 750.9 | −73.1 | 8.1 |
| Cobra-linker set | | | | | | | |
| 3 | | | | | | | |
| | | PHH Lot 31 | | | | | |
| CasX 515 | 316.6.1 (V1) | 42.7 | 10.6 | 149.3 | 80.3 | −54.4 | 3.3 |
| SpyCas9 | Spy.50.1 (vInt) | 37.5 | 28.2 | 293.3 | 539.3 | −44.4 | 9.4 |
| LTRP5 | 316.6.154 (V1) | 53.8 | 10.9 | 202.5 | 87.7 | −97 | 4.6 |
| LTRP5 | 316.6.154 (V1), | 59.7 | 9.2 | 194.2 | 61.6 | −96.5 | 3.9 |
| | 316.6.157 (V1) | | | | | | |
| LTRP6-Rat- | 316.6.154 (V1) | 39.9 | 5.7 | 387 | 139.8 | −96.1 | 3.6 |
| Cobra | | | | | | | |
| LTRP5 | 316.6.157 (V1) | 115.9 | 10.6 | 438.4 | 93.9 | −93.4 | 2.5 |
| LTRP6-2xCobra | 316.6.154 (V1) | 106.8 | 16.8 | 327.2 | 125.7 | −89.6 | 5.2 |
| LTRP5 | 316.6.141 (V1) | 122 | 30.2 | 701.4 | 417.1 | −92.1 | 7.3 |
| LTRP6-Rat- | 316.6.141 (V1) | 115 | 13.2 | 407.9 | 110.7 | −88.6 | 3.6 |
| Cobra | | | | | | | |
| LTRP6-Rat- | 316.6.154 (V1) | 129.6 | 22.2 | 461.3 | 182.3 | −83.1 | 5.2 |
| Cobra-linker set | | | | | | | |
| 3 | | | | | | | |
| LTRP6-2xCobra | 316.6.141 (V1) | 148.6 | 18.7 | 377.4 | 93.9 | −89.7 | 4.1 |
| LTRP6-Rat- | 316.6.157 (V1) | 172.1 | 42.4 | 1053.3 | 631.5 | −87.3 | 8 |
| Cobra | | | | | | | |
| LTRP6-2xCobra | 316.6.157 (V1) | 196.7 | 35.5 | 659.9 | 271.6 | −77.4 | 6 |
| LTRP6-Rat- | 316.6.157 (V1) | 254.2 | 69.8 | 728.3 | 523.5 | −55.4 | 7.6 |
| Cobra-linker set | | | | | | | |
| 3 | | | | | | | |
| LTRP6-Rat- | 316.6.141 (V1) | 228 | 37 | 536.7 | 210 | −73.7 | 5.4 |
| Cobra-linker set | | | | | | | |
| 3 | | | | | | | |

TABLE 64
Levels of PCSK9 mRNA repression mediated by LTRP constructs with various repressor
domains by mRNA molarity

| | | IC50 (pM) | | IC90* (pM) | | Emax* (Δ %) | |
|---|---|---|---|---|---|---|---|
| mRNA | gRNA | Mean | SEM | Mean | SEM | Mean | SEM |
| | | PHH Lot 271 | | | | | |
| LTRP5 | 316.6.154 (V1) | 105.3 | 21.4 | 582.3 | 278.4 | −96.7 | 5.5 |
| SpyCas9 | Spy.50.1 (vInt) | 163.2 | 26.6 | 522.5 | 174.8 | −59.8 | 2.6 |
| LTRP5 | 316.6.154 (V1), | 126 | 12.5 | 394.7 | 83.8 | −95.1 | 2.9 |
| | 316.6.157 (V1) | | | | | | |
| LTRP6-Rat- | 316.6.154 (V1) | 248.7 | 36.7 | 958.4 | 333.5 | −94.9 | 4.3 |
| Cobra | | | | | | | |
| CasX 515 | 316.6.1 (V1) | 147.5 | 28.6 | 514.9 | 225 | −60.1 | 2.7 |
| LTRP5 | 316.6.154 (V1) | 279.3 | 40 | 1390.8 | 478.5 | −94.7 | 5 |
| LTRP6-2xCobra | 316.6.154 (V1) | 331.4 | 53 | 1415.1 | 533.1 | −91.9 | 5 |
| LTRP5 | 316.6.154 (V1) | 311 | 38.1 | 977.8 | 286.9 | −88.9 | 3.7 |
| LTRP6-Rat- | 316.6.154 (V1) | 409.9 | 60 | 2021.7 | 707.6 | −92.9 | 4.5 |
| Cobra | | | | | | | |
| LTRP6-Rat- | 316.6.154 (V1) | 462.8 | 99 | 2179.2 | 1113.3 | −83.3 | 6.4 |
| Cobra-linker set | | | | | | | |
| 3 | | | | | | | |
| LTRP6-2xCobra | 316.6.154 (V1) | 427.7 | 126.9 | 1863.5 | 1303 | −92.7 | 8.7 |
| LTRP6-Rat- | 316.6.154 (V1) | 489.3 | 95.5 | 2562.4 | 1213.2 | −80.3 | 5.8 |
| Cobra | | | | | | | |
| LTRP6-2xCobra | 316.6.154 (V1) | 508.4 | 114.6 | 2188.9 | 1175.9 | −70.7 | 6.2 |
| LTRP6-Rat- | 316.6.154 (V1) | 370.8 | 103.9 | 1290.5 | 824.5 | −41.1 | 4.3 |
| Cobra-linker set | | | | | | | |
| 3 | | | | | | | |
| LTRP6-Rat- | 316.6.154 (V1) | 640.1 | 185.5 | 2895.5 | 2061.3 | −73.1 | 8.1 |
| Cobra-linker set | | | | | | | |
| 3 | | | | | | | |
| | | PHH Lot 31 | | | | | |
| LTRP5 | 316.6.154 (V1) | 148.3 | 30.1 | 558.3 | 241.9 | −97 | 4.6 |
| SpyCas9 | Spy.50.1 (vInt) | 121.5 | 91.3 | 950.9 | 1748.5 | −44.4 | 9.4 |
| LTRP5 | 316.6.154 (V1), | 164.6 | 25.3 | 535.5 | 169.9 | −96.5 | 3.9 |
| | 316.6.157 (V1) | | | | | | |
| LTRP6-Rat- | 316.6.154 (V1) | 106.8 | 15.2 | 1036.2 | 374.3 | −96.1 | 3.6 |
| Cobra | | | | | | | |
| CasX 515 | 316.6.1 (V1) | 212.8 | 52.8 | 743.7 | 399.9 | −54.4 | 3.3 |
| LTRP5 | 316.6.154 (V1) | 319.5 | 29.1 | 1208.7 | 259 | −93.4 | 2.5 |
| LTRP6-2xCobra | 316.6.154 (V1) | 286.1 | 44.9 | 876.3 | 336.5 | −89.6 | 5.2 |
| LTRP5 | 316.6.154 (V1) | 336.3 | 83.3 | 1934.1 | 1150.2 | −92.1 | 7.3 |
| LTRP6-Rat- | 316.6.154 (V1) | 307.8 | 35.5 | 1092.1 | 296.5 | −88.6 | 3.6 |
| Cobra | | | | | | | |
| LTRP6-Rat- | 316.6.154 (V1) | 355.7 | 61 | 1266.3 | 500.4 | −83.1 | 5.2 |
| Cobra-linker set | | | | | | | |
| 3 | | | | | | | |
| LTRP6-2xCobra | 316.6.154 (V1) | 398 | 50 | 1010.9 | 251.4 | −89.7 | 4.1 |
| LTRP6-Rat- | 316.6.154 (V1) | 460.9 | 113.4 | 2820.4 | 1690.8 | −87.3 | 8 |
| Cobra | | | | | | | |
| LTRP6-2xCobra | 316.6.154 (V1) | 526.9 | 95.2 | 1767.5 | 727.6 | −77.4 | 6 |
| LTRP6-Rat- | 316.6.154 (V1) | 697.9 | 191.7 | 1999.3 | 1437.2 | −55.4 | 7.6 |
| Cobra-linker set | | | | | | | |
| 3 | | | | | | | |
| LTRP6-Rat- | 316.6.154 (V1) | 625.9 | 101.5 | 1473.4 | 576.4 | −73.7 | 5.4 |
| Cobra-linker set | | | | | | | |
| 3 | | | | | | | |

TABLE 65
Gene expression change by RNA-seq

| | | Dose | | log2(Fold | |
|---|---|---|---|---|---|
| mRNA | gRNA | (ng*) | Gene | Change) | p-value |
| CasX 515 | 316.6.1 (V1) | 2400 | TUBB3 | −1.05 | 5.90E−08 |
| | | | MARCO | −2.67 | 8.61E−07 |
| | | | ESAM | −1.04 | 1.07E−06 |
| | | | SUSD2 | −1.14 | 3.07E−06 |
| | | | C1QB | −1.67 | 2.87E−05 |
| | | | KIF23 | −1.19 | 5.82E−05 |
| SpyCas9 | Spy.50.1 (vInt) | 2400 | PLD4 | −1.36 | 4.14E−07 |
| | | | SLC7A7 | −1.03 | 8.09E−07 |
| | | | TSPAN15 | −1.28 | 1.54E−06 |
| | | | ANXA8 | −1.28 | 3.39E−06 |
| | | | RAC2 | −1.08 | 4.94E−06 |
| | | | CYP7A1 | 1.45 | 1.09E−05 |
| LTRP5 | 316.6.154 | 2400 | PLD4 | −1.36 | 4.14E−07 |
| | (V1), | | SLC7A7 | −1.03 | 8.09E−07 |
| | 316.6.157 (V1) | | TSPAN15 | −1.28 | 1.54E−06 |
| | | | ANXA8 | −1.28 | 3.39E−06 |
| | | | RAC2 | −1.08 | 4.94E−06 |
| | | | CYP7A1 | 1.45 | 1.09E−05 |
| LTRP5 | 316.6.154 (V1) | 2400 | PCSK9 | −3.87 | 1.93E−64 |
| | | | C1QB | −2.92 | 1.78E−08 |
| | | | GPNMB | −1.96 | 3.06E−08 |
| | | | CD163 | −1.49 | 3.89E−07 |
| | | | ITGAX | −2.02 | 5.74E−07 |
| | | | LAPTM5 | −1.60 | 1.33E−06 |
| | | | RNASE1 | −2.12 | 2.05E−06 |
| | | | MSR1 | −2.30 | 2.10E−06 |
| | | | RGCC | −2.20 | 2.88E−06 |
| | | | PLEK | −2.46 | 1.88E−05 |
| | | | MIR6501 | 1.04 | 0.00011389 |
| | | | HCK | −2.84 | 0.00013665 |
| LTRP5 | 316.6.157 (V1) | 2400 | PCSK9 | −2.97 | 9.32E−41 |
| | | | TM4SF19 | −2.59 | 1.26E−06 |
| | | | UCP2 | −2.07 | 3.00E−05 |
| | | | MIR6501 | 1.09 | 8.01E−05 |
| LTRP5 | 316.27.94 (V1) | 2400 | NA | NA | NA |
| LTRP6-Rat-Cobra | 316.6.154 (V1) | 2400 | PCSK9 | −3.34 | 5.86E−47 |
| | | | DNMT3A | 1.83 | 1.55E−08 |
| | | | G6PD | 1.01 | 4.75E−05 |
| | | | SDS | 1.47 | 0.00012577 |
| LTRP6-Rat-Cobra- | 316.6.154 (V1) | 2400 | PCSK9 | −1.45 | 3.00E−12 |
| linker set 3 | | | | | |
| LTRP6-2xCobra | 316.6.154 (V1) | 2400 | PCSK9 | −2.34 | 2.85E−27 |

*Dose is reported as ng of encapsulated RNA/well

1-36. (canceled)

37. An mRNA encoding a long-term-repressor fusion protein (LTRP),
wherein the LTRP comprises, from N- to C-terminus, a DNA methyltransferase (DNMT) 3A catalytic domain (DNMT3A), a DNMT3 like interaction domain (DNMT3L), a DNA binding protein comprising a catalytically-dead CasX (dCasX), and a first repressor domain (RD1), and
wherein the mRNA comprises a sequence selected from the group consisting of SEQ ID NOS: 2410-2428, 2466-2484, 2521, 3214, 3382, 4118, 4811, 4979, 5715, 6408, 6576, 7312, 8005, and 8173, or a sequence having at least about 90% sequence identity thereto.

38. The mRNA of claim 37, wherein the mRNA comprises a sequence selected from the group consisting of SEQ ID NOS: 2411, 2421, 2467, and 2477, or a sequence having at least about 90% sequence identity thereto.

39. The mRNA of claim 37, wherein the mRNA comprises:
(a) a sequence selected from the group consisting of SEQ ID NOS: 2521, 4118, 5715, and 7312, or a sequence having at least about 90% sequence identity thereto;
(b) a sequence selected from the group consisting of SEQ ID NOS: 3214, 4811, 6408, and 8005, or a sequence having at least about 90% sequence identity thereto; or
(c) a sequence selected from the group consisting of SEQ ID NOS: 3382, 4979, 6576, and 8173, or a sequence having at least about 90% sequence identity thereto.

40. The mRNA of claim 37, wherein the mRNA comprises a sequence selected from the group consisting of SEQ ID NOS: 2410, 2420, 2466, and 2476, or a sequence having at least about 90% sequence identity thereto.

41. The mRNA of claim 37, wherein the mRNA comprises a sequence selected from the group consisting of SEQ ID NOS: 2412, 2422, 2468, and 2478, or a sequence having at least about 90% sequence identity thereto.

42. The mRNA of claim 37, wherein the LTRP further comprises a DNMT3A ATRX-DNMT3-DNMT3L domain (ADD) linked N-terminal to the DNMT3A.

43. The mRNA of claim 42, wherein the sequence encoding the ADD comprises the sequence of SEQ ID NO: 1954, or a sequence having at least about 90% sequence identity thereto.

44. The mRNA of claim 37, wherein the sequence encoding the dCasX is selected from the group consisting of SEQ ID NOS: 1948, 2405, and 2406, or a sequence having at least about 90% sequence identity thereto.

45. The mRNA of claim 37, wherein the sequence encoding the DNMT3A is selected from the group consisting of SEQ ID NO: 1955 and SEQ ID NO: 21878, or a sequence having at least about 90% sequence identity thereto.

46. The mRNA of claim 37, wherein the sequence encoding the DNMT3L is selected from the group consisting of SEQ ID NO: 1945 or SEQ ID NO: 21879, or a sequence having at least about 90% sequence identity thereto.

47. The mRNA of claim 37, wherein the sequence encoding the RD1 is selected from the group consisting of SEQ ID NOS: 18637-18646 and 20234-20243, or a sequence having at least about 90% sequence identity thereto.

48. The mRNA of claim 37, wherein the LTRP comprises one or more nuclear localization sequences (NLS).

49. The mRNA of claim 48, wherein the sequence encoding the one or more NLS comprises the sequence of SEQ ID NO: 21875.

50. The mRNA of claim 37, wherein the LTRP comprises one or more linker peptides.

51. The mRNA of claim 37, wherein:
(a) the dCasX comprises the amino acid sequence of SEQ ID NO: 4, or a sequence having at least about 90% sequence identity thereto;
(b) the RD1 comprises an amino acid sequence selected from the group consisting of SEQ ID NOS: 130, 131, 135, 143, or a sequence having at least about 90% sequence identity thereto;
(c) the DNMT3A comprises the amino acid sequence of SEQ ID NO: 126, or a sequence having at least about 90% sequence identity thereto; and/or
(d) the DNMT3L comprises the amino acid sequence of SEQ ID NO: 127, or a sequence having at least about 90% sequence identity thereto.

52. The mRNA of claim 37, wherein the mRNA sequence encoding the LTRP is codon-optimized.

53. The mRNA of claim 37, wherein the mRNA comprises a 5′ cap.

54. A system for transcriptional repression of a gene comprising the mRNA of claim 37, and a guide ribonucleic acid (gRNA) comprising a targeting sequence complementary to a target nucleic acid sequence of a gene in a cell.

55. The system of claim 54, wherein the targeting sequence of the gRNA is complementary to a target nucleic acid sequence within 1 kb of a transcription start site (TSS) in the gene.

56. The system of claim 54, wherein the gRNA comprises a scaffold comprising a sequence of SEQ ID NOS: 1746, or a sequence having at least about 90% sequence identity thereto.

57. The system of claim 56, wherein the gRNA is chemically modified.

58. The system of claim 57, wherein the chemical modification comprises an addition of a 2′O-methyl group to one or more nucleotides of the gRNA.

59. The system of claim 57, wherein one or more nucleotides located 1, 2, 3, or 4 nucleotides from the 5′ terminal end, the 3′ terminal, or both terminal ends of the gRNA are modified by an addition of a 2′O-methyl group.

60. The system of claim 57, wherein the chemical modification comprises a substitution of phosphorothioate bonds between two or more nucleotides located 1, 2, 3 or 4 nucleotides the from the 5′ terminal end, the 3′ terminal, or both terminal ends of the gRNA.

61. The system of claim 57, wherein the gRNA comprises a sequence selected from SEQ ID NOS: 2156-2164, and wherein the targeting sequence complementary to a target nucleic acid is substituted for the 20 nucleotides on the 3′ end of SEQ ID NOS: 2156-2164.

62. A lipid nanoparticle (LNP) comprising the system of claim 54, wherein the LNP comprises an ionizable lipid, a helper phospholipid, a polyethylene glycol (PEG)-modified lipid, and cholesterol or a derivative thereof.

63. A pharmaceutical composition comprising the LNP of claim 62 and a pharmaceutically acceptable carrier, diluent or excipient.

64. A method of repressing transcription of a gene in a population of cells, the method comprising contacting cells of the population with the LNP of claim 62, wherein the contacting results in the repression of transcription of the gene in the population of cells.

65. The method of claim 64, wherein the repression of the gene of the population of cells occurs in vitro, ex vivo, or in vivo.
